SEQUENCE LISTING (1009 sequences)

SEQ ID NO: 1 - protein, 21 aa, Artificial Sequence
  source: /note=“Description of Artificial Sequence: Synthetic peptide”
MALPVTALLLPLALLLHAARP

SEQ ID NO: 2 - protein, 45 aa, Artificial Sequence
  source: /note=“Description of Artificial Sequence: Synthetic polypeptide”
TTTPAPRPPTPAPTIASQPLSLRPEACRPAAGGAVHTRGLDFACD

SEQ ID NO: 3 - protein, 119 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVT

SEQ ID NO: 4 - protein, 111 aa, Artificial Sequence
  source: /note=“Description of Artificial Sequence: Synthetic polypeptide”
ELVLTQSPKFMSTSVGDRVSVTCKASQNVGTNVAWYQQKPGQSPKPLIYSATYRNSGVPDRFTGSGSGTDFTLTITNVQSKDLADYFYFCQYNRYPYTSGGGTKLEIKRRS

SEQ ID NO: 5 - protein, 10 aa, Artificial Sequence
  source: /note=“Description of Artificial Sequence: Synthetic peptide”
GGGGSGGGGS

SEQ ID NO: 6 - protein, 24 aa, Artificial Sequence
  source: /note=“Description of Artificial Sequence: Synthetic peptide”
IYIWAPLAGTCGVLLLSLVITLYC

SEQ ID NO: 7 - protein, 42 aa, Artificial Sequence
  source: /note=“Description of Artificial Sequence: Synthetic polypeptide”
KRGRKKLLYIFKQPFMRPVQTTQEEDGCSCRFPEEEEGGCEL

SEQ ID NO: 8 - DNA, 30 bp, Artificial Sequence
  source: /note=“Description of Artificial Sequence: Synthetic oligonucleotide”
ggtggcggaggttctggaggtggaggttcc

SEQ ID NO: 9 - protein, 112 aa, Artificial Sequence
  source: /note=“Description of Artificial Sequence: Synthetic polypeptide”
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1184 bp, Artificial Sequence
  source: /note=“Description of Artificial Sequence: Synthetic polynucleotide”
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 12 - DNA, 63 bp, Artificial Sequence
  source: /note=“Description of Artificial Sequence: Synthetic oligonucleotide”
atggccctgcctgtgacagccctgctgctgcctctggctctgctgctgcatgccgctagaccc

SEQ ID NO: 13 - DNA, 135 bp, Artificial Sequence
  source: /note=“Description of Artificial Sequence: Synthetic polynucleotide”
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 123 bp, Artificial Sequence
  source: /note=“Description of Artificial Sequence: Synthetic polynucleotide”
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 15 - protein, 40 aa, Artificial Sequence
  source: /note=“Description of Artificial Sequence: Synthetic polypeptide”
  SITE (1)..(40): /note=“This sequence may encompass 1-10 ′Gly Gly Gly Ser′ repeating units”
GGGSGGGSGGGSGGGSGGGSGGGSGGGSGGGSGGGSGGGS

SEQ ID NO: 16 - protein, 48 aa, Artificial Sequence
  source: /note=“Description of Artificial Sequence: Synthetic polypeptide”
QRRKYRSNKGESPVEPAEPCRYSCPREEEGSTIPIQEDYRKPEPACSP

SEQ ID NO: 17 - DNA, 72 bp, Artificial Sequence
  source: /note=“Description of Artificial Sequence: Synthetic oligonucleotide”
atctacatctgggcgcccttggccgggacttgtggggtccttctcctgtcactggttatcaccctttactgc

SEQ ID NO: 18 - DNA, 126 bp, Artificial Sequence
  source: /note=“Description of Artificial Sequence: Synthetic polynucleotide”
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - skipped

SEQ ID NO: 20 - DNA, 336 bp, Artificial Sequence
  source: /note=“Description of Artificial Sequence: Synthetic polynucleotide”
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 336 bp, Homo sapiens
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - DNA, 813 bp, Artificial Sequence
  source: /note=“Description of Artificial Sequence: Synthetic polynucleotide”
atggccctgcccgtcaccgctctgctgctgccccttgctctgcttcttcatgcagcaaggccggacatccagatgacccaaaccacctcatccctctctgcctctcttggagacagggtgaccatttcttgtcgcgccagccaggacatcagcaagtatctgaactggtatcagcagaagccggacggaaccgtgaagctcctgatctaccatacctctcgcctgcatagcggcgtgccctcacgcttctctggaagcggatcaggaaccgattattctctcactatttcaaatcttgagcaggaagatattgccacctatttctgccagcagggtaataccctgccctacaccttcggaggagggaccaagctcgaaatcaccggtggaggaggcagcggcggtggagggtctggtggaggtggttctgaggtgaagctgcaagaatcaggccctggacttgtggccccttcacagtccctgagcgtgacttgcaccgtgtccggagtctccctgcccgactacggagtgtcatggatcagacaacctccacggaaaggactggaatggctcggtgtcatctggggtagcgaaactacttactacaattcagccctcaaaagcaggctgactattatcaaggacaacagcaagtcccaagtctttcttaagatgaactcactccagactgacgacaccgcaatctactattgtgctaagcactactactacggaggatcctacgctatggattactggggacaaggtacttccgtcactgtctcttcacaccatcatcaccatcaccatcac

SEQ ID NO: 23 - DNA, 4027 bp, Homo sapiens
caggcagcgtggtcctgctgcgcacgtgggaagccctggccccggccacccccgcgatgccgcgcgctccccgctgccgagccgtgcgctccctgctgcgcagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgttgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcggggctgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatggccacccgcccacagccaggccgagagcagacaccagcagccctgtcacgccgggctctacgtcccagggagggaggggcggcccacacccaggcccgcaccgctgggagtctgaggcctgagtgagtgtttggccgaggcctgcatgtccggctgaaggctgagtgtccggctgaggcctgagcgagtgtccagccaagggctgagtgtccagcacacctgccgtcttcacttccccacaggctggcgctcggctccaccccagggccagcttttcctcaccaggagcccggcttccactccccacataggaatagtccatccccagattcgccattgttcacccctcgccctgccctcctttgccttccacccccaccatccaggtggagaccctgagaaggaccctgggagctctgggaatttggagtgaccaaaggtgtgccctgtacacaggcgaggaccctgcacctggatgggggtccctgtgggtcaaattggggggaggtgctgtgggagtaaaatactgaatatatgagtttttcagttttgaaaaaaaaaaaaaaaaaa

SEQ ID NO: 24 - skipped

SEQ ID NO: 25 - protein, 5 aa, Artificial Sequence
  source: /note=“Description of Artificial Sequence: Synthetic peptide”
GGGGS

SEQ ID NO: 26 - protein, 30 aa, Artificial Sequence
  source: /note=“Description of Artificial Sequence: Synthetic polypeptide”
  SITE (1)..(30): /note=“This sequence may encompass 1-6 ′Gly Gly Gly Gly Ser′ repeating units”
GGGGSGGGGSGGGGSGGGGSGGGGSGGGGS

SEQ ID NO: 27 - protein, 20 aa, Artificial Sequence
  source: /note=“Description of Artificial Sequence: Synthetic peptide”
GGGGSGGGGSGGGGSGGGGS

SEQ ID NO: 28 - protein, 15 aa, Artificial Sequence
  source: /note=“Description of Artificial Sequence: Synthetic peptide”
GGGGSGGGGSGGGGS

SEQ ID NO: 29 - protein, 4 aa, Artificial Sequence
  source: /note=“Description of Artificial Sequence: Synthetic peptide”
GGGS

SEQ ID NO: 30 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
  source: /note=“See specification as filed for detailed description of substitutions and preferred embodiments”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 100 bp, Artificial Sequence
  source: /note=“Description of Artificial Sequence: Synthetic polynucleotide”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 32 - DNA, 5000 bp, Artificial Sequence
  source: /note=“Description of Artificial Sequence: Synthetic polynucleotide”
  misc_feature (1)..(5000): /note=“This sequence may encompass 50-5000 nucleotides”
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 33 - DNA, 5000 bp, Artificial Sequence; identical to SEQ ID NO: 30
  source: /note=“Description of Artificial Sequence: Synthetic polynucleotide”
  misc_feature (1)..(5000): /note=“This sequence may encompass 100-5000 nucleotides”

SEQ ID NO: 34 - DNA, 400 bp, Artificial Sequence
  source: /note=“Description of Artificial Sequence: Synthetic polynucleotide”
  misc_feature (1)..(400): /note=“This sequence may encompass 100-400 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - DNA, 2000 bp, Artificial Sequence
  source: /note=“Description of Artificial Sequence: Synthetic polynucleotide”
  misc_feature (1)..(2000): /note=“This sequence may encompass 50-2000 nucleotides”
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - protein, 230 aa, Artificial Sequence
  source: /note=“Description of Artificial Sequence: Synthetic polypeptide”
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 37 - DNA, 690 bp, Artificial Sequence
  source: /note=“Description of Artificial Sequence: Synthetic polynucleotide”
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 38 - protein, 282 aa, Artificial Sequence
  source: /note=“Description of Artificial Sequence: Synthetic polypeptide”
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 39 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 40 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 41 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 42 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 43 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 44 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 45 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 46 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 47 - protein, 247 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 48 - protein, 247 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 49 - protein, 242 aa, Artificial Sequence
  source: /note=“Description of Artificial Sequence: Synthetic polypeptide”
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 50 - protein, 242 aa, Artificial Sequence
  source: /note=“Description of Artificial Sequence: Synthetic polypeptide”
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 51 - protein, 242 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 52 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 53 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 54 - DNA, 813 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaacatcaccaccatcatcaccatcac

SEQ ID NO: 55 - DNA, 813 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaacatcaccaccatcatcaccatcac

SEQ ID NO: 56 - DNA, 828 bp, Artificial Sequence
  source: /note=“Description of Artificial Sequence: Synthetic polynucleotide”
atggccctcccagtgaccgctctgctgctgcctctcgcacttcttctccatgccgctcggcctgagatcgtcatgacccaaagccccgctaccctgtccctgtcacccggcgagagggcaaccctttcatgcagggccagccaggacatttctaagtacctcaactggtatcagcagaagccagggcaggctcctcgcctgctgatctaccacaccagccgcctccacagcggtatccccgccagattttccgggagcgggtctggaaccgactacaccctcaccatctcttctctgcagcccgaggatttcgccgtctatttctgccagcaggggaatactctgccgtacaccttcggtcaaggtaccaagctggaaatcaagggaggcggaggatcaggcggtggcggaagcggaggaggtggctccggaggaggaggttcccaagtgcagcttcaagaatcaggacccggacttgtgaagccatcagaaaccctctccctgacttgtaccgtgtccggtgtgagcctccccgactacggagtctcttggattcgccagcctccggggaagggtcttgaatggattggggtgatttggggatcagagactacttactactcttcatcacttaagtcacgggtcaccatcagcaaagataatagcaagaaccaagtgtcacttaagctgtcatctgtgaccgccgctgacaccgccgtgtactattgtgccaaacattactattacggagggtcttatgctatggactactggggacaggggaccctggtgactgtctctagccatcaccatcaccaccatcatcac

SEQ ID NO: 57 - DNA, 828 bp, Artificial Sequence
  source: /note=“Description of Artificial Sequence: Synthetic polynucleotide”
atggccctcccagtgaccgctctgctgctgcctctcgcacttcttctccatgccgctcggcctgagatcgtcatgacccaaagccccgctaccctgtccctgtcacccggcgagagggcaaccctttcatgcagggccagccaggacatttctaagtacctcaactggtatcagcagaagccagggcaggctcctcgcctgctgatctaccacaccagccgcctccacagcggtatccccgccagattttccgggagcgggtctggaaccgactacaccctcaccatctcttctctgcagcccgaggatttcgccgtctatttctgccagcaggggaatactctgccgtacaccttcggtcaaggtaccaagctggaaatcaagggaggcggaggatcaggcggtggcggaagcggaggaggtggctccggaggaggaggttcccaagtgcagcttcaagaatcaggacccggacttgtgaagccatcagaaaccctctccctgacttgtaccgtgtccggtgtgagcctccccgactacggagtctcttggattcgccagcctccggggaagggtcttgaatggattggggtgatttggggatcagagactacttactaccagtcatcacttaagtcacgggtcaccatcagcaaagataatagcaagaaccaagtgtcacttaagctgtcatctgtgaccgccgctgacaccgccgtgtactattgtgccaaacattactattacggagggtcttatgctatggactactggggacaggggaccctggtgactgtctctagccatcaccatcaccaccatcatcac

SEQ ID NO: 58 - DNA, 828 bp, Artificial Sequence
  source: /note=“Description of Artificial Sequence: Synthetic polynucleotide”
atggcactgcctgtcactgccctcctgctgcctctggccctccttctgcatgccgccaggccccaagtccagctgcaagagtcaggacccggactggtgaagccgtctgagactctctcactgacttgtaccgtcagcggcgtgtccctccccgactacggagtgtcatggatccgccaacctcccgggaaagggcttgaatggattggtgtcatctggggttctgaaaccacctactactcatcttccctgaagtccagggtgaccatcagcaaggataattccaagaaccaggtcagccttaagctgtcatctgtgaccgctgctgacaccgccgtgtattactgcgccaagcactactattacggaggaagctacgctatggactattggggacagggcactctcgtgactgtgagcagcggcggtggagggtctggaggtggaggatccggtggtggtgggtcaggcggaggagggagcgagattgtgatgactcagtcaccagccaccctttctctttcacccggcgagagagcaaccctgagctgtagagccagccaggacatttctaagtacctcaactggtatcagcaaaaaccggggcaggcccctcgcctcctgatctaccatacctcacgccttcactctggtatccccgctcggtttagcggatcaggatctggtaccgactacactctgaccatttccagcctgcagccagaagatttcgcagtgtatttctgccagcagggcaatacccttccttacaccttcggtcagggaaccaagctcgaaatcaagcaccatcaccatcatcaccaccat

SEQ ID NO: 59 - DNA, 828 bp, Artificial Sequence
  source: /note=“Description of Artificial Sequence: Synthetic polynucleotide”
atggcactgcctgtcactgccctcctgctgcctctggccctccttctgcatgccgccaggccccaagtccagctgcaagagtcaggacccggactggtgaagccgtctgagactctctcactgacttgtaccgtcagcggcgtgtccctccccgactacggagtgtcatggatccgccaacctcccgggaaagggcttgaatggattggtgtcatctggggttctgaaaccacctactaccagtcttccctgaagtccagggtgaccatcagcaaggataattccaagaaccaggtcagccttaagctgtcatctgtgaccgctgctgacaccgccgtgtattactgcgccaagcactactattacggaggaagctacgctatggactattggggacagggcactctcgtgactgtgagcagcggcggtggagggtctggaggtggaggatccggtggtggtgggtcaggcggaggagggagcgagattgtgatgactcagtcaccagccaccctttctctttcacccggcgagagagcaaccctgagctgtagagccagccaggacatttctaagtacctcaactggtatcagcaaaaaccggggcaggcccctcgcctcctgatctaccatacctcacgccttcactctggtatccccgctcggtttagcggatcaggatctggtaccgactacactctgaccatttccagcctgcagccagaagatttcgcagtgtatttctgccagcagggcaatacccttccttacaccttcggtcagggaaccaagctcgaaatcaagcaccatcaccatcatcatcaccac

SEQ ID NO: 60 - DNA, 828 bp, Artificial Sequence
  source: /note=“Description of Artificial Sequence: Synthetic polynucleotide”
atggccctcccagtgaccgctctgctgctgcctctcgcacttcttctccatgccgctcggcctgagatcgtcatgacccaaagccccgctaccctgtccctgtcacccggcgagagggcaaccctttcatgcagggccagccaggacatttctaagtacctcaactggtatcagcagaagccagggcaggctcctcgcctgctgatctaccacaccagccgcctccacagcggtatccccgccagattttccgggagcgggtctggaaccgactacaccctcaccatctcttctctgcagcccgaggatttcgccgtctatttctgccagcaggggaatactctgccgtacaccttcggtcaaggtaccaagctggaaatcaagggaggcggaggatcaggcggtggcggaagcggaggaggtggctccggaggaggaggttcccaagtgcagcttcaagaatcaggacccggacttgtgaagccatcagaaaccctctccctgacttgtaccgtgtccggtgtgagcctccccgactacggagtctcttggattcgccagcctccggggaagggtcttgaatggattggggtgatttggggatcagagactacttactacaattcatcacttaagtcacgggtcaccatcagcaaagataatagcaagaaccaagtgtcacttaagctgtcatctgtgaccgccgctgacaccgccgtgtactattgtgccaaacattactattacggagggtcttatgctatggactactggggacaggggaccctggtgactgtctctagccatcaccatcaccaccatcatcac

SEQ ID NO: 61 - DNA, 828 bp, Artificial Sequence
  source: /note=“Description of Artificial Sequence: Synthetic polynucleotide”
atggcactgcctgtcactgccctcctgctgcctctggccctccttctgcatgccgccaggccccaagtccagctgcaagagtcaggacccggactggtgaagccgtctgagactctctcactgacttgtaccgtcagcggcgtgtccctccccgactacggagtgtcatggatccgccaacctcccgggaaagggcttgaatggattggtgtcatctggggttctgaaaccacctactacaactcttccctgaagtccagggtgaccatcagcaaggataattccaagaaccaggtcagccttaagctgtcatctgtgaccgctgctgacaccgccgtgtattactgcgccaagcactactattacggaggaagctacgctatggactattggggacagggcactctcgtgactgtgagcagcggcggtggagggtctggaggtggaggatccggtggtggtgggtcaggcggaggagggagcgagattgtgatgactcagtcaccagccaccctttctctttcacccggcgagagagcaaccctgagctgtagagccagccaggacatttctaagtacctcaactggtatcagcaaaaaccggggcaggcccctcgcctcctgatctaccatacctcacgccttcactctggtatccccgctcggtttagcggatcaggatctggtaccgactacactctgaccatttccagcctgcagccagaagatttcgcagtgtatttctgccagcagggcaatacccttccttacaccttcggtcagggaaccaagctcgaaatcaagcaccatcaccatcatcaccaccat

SEQ ID NO: 62 - DNA, 813 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaattcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagccaccaccatcatcaccatcaccat

SEQ ID NO: 63 - DNA, 813 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaacatcaccaccatcatcaccatcac

SEQ ID NO: 64 - protein, 271 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSHHHHHHHH

SEQ ID NO: 65 - protein, 271 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSHHHHHHHH

SEQ ID NO: 66 - protein, 271 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 67 - protein, 271 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 68 - protein, 276 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSHHHHHHHH

SEQ ID NO: 69 - protein, 276 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSHHHHHHHH

SEQ ID NO: 70 - protein, 276 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 71 - protein, 276 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 72 - protein, 276 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSHHHHHHHH

SEQ ID NO: 73 - protein, 276 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 74 - protein, 271 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSHHHHHHHH

SEQ ID NO: 75 - protein, 271 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKHHHHHHHH

SEQ ID NO: 76 - protein, 271 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSHHHHHHHH

SEQ ID NO: 77 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 78 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 79 - protein, 486 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 80 - protein, 486 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 81 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 82 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 84 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 85 - protein, 491 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 86 - protein, 491 aa, Artificial Sequence; identical to SEQ ID NO: 85
  source: /note=“Description of Artificial Sequence: Synthetic polypeptide”

SEQ ID NO: 87 - protein, 491 aa, Artificial Sequence
  source: /note=“Description of Artificial Sequence: Synthetic polypeptide”
MALPVTALLLPLALLLHAARPQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 88 - protein, 486 aa, Artificial Sequence
  source: /note=“Description of Artificial Sequence: Synthetic polypeptide”
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 89 - protein, 486 aa, Artificial Sequence
  source: /note=“Description of Artificial Sequence: Synthetic polypeptide”
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 90 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 91 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 92 - DNA, 1458 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 93 - DNA, 1458 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 94 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggcggaggcgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactactcttcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 95 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggagggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 96 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactattcatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 97 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactatcaatcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggcggtgggtcagaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 98 - DNA, 1473 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagcggaggcggtgggagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 99 - DNA, 1473 bp, Artificial Sequence; identical to SEQ ID NO: 98
  source: /note=“Description of Artificial Sequence: Synthetic polynucleotide”

SEQ ID NO: 100 - DNA, 1473 bp, Artificial Sequence
  source: /note=“Description of Artificial Sequence: Synthetic polynucleotide”
atggctctgcccgtgaccgcactcctcctgccactggctctgctgcttcacgccgctcgcccacaagtccagcttcaagaatcagggcctggtctggtgaagccatctgagactctgtccctcacttgcaccgtgagcggagtgtccctcccagactacggagtgagctggattagacagcctcccggaaagggactggagtggatcggagtgatttggggtagcgaaaccacttactataactcttccctgaagtcacgggtcaccatttcaaaggataactcaaagaatcaagtgagcctcaagctctcatcagtcaccgccgctgacaccgccgtgtattactgtgccaagcattactactatggagggtcctacgccatggactactggggccagggaactctggtcactgtgtcatctggtggaggaggtagcggaggaggcgggagcggtggaggtggctccggaggtggcggaagcgaaatcgtgatgacccagagccctgcaaccctgtccctttctcccggggaacgggctaccctttcttgtcgggcatcacaagatatctcaaaatacctcaattggtatcaacagaagccgggacaggcccctaggcttcttatctaccacacctctcgcctgcatagcgggattcccgcacgctttagcgggtctggaagcgggaccgactacactctgaccatctcatctctccagcccgaggacttcgccgtctacttctgccagcagggtaacaccctgccgtacaccttcggccagggcaccaagcttgagatcaaaaccactactcccgctccaaggccacccacccctgccccgaccatcgcctctcagccgctttccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 101 - DNA, 1458 bp, Artificial Sequence
  source: /note=“Description of Artificial Sequence: Synthetic polynucleotide”
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactacaactcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 102 - DNA, 1458 bp, Artificial Sequence
  source: /note=“Description of Artificial Sequence: Synthetic polynucleotide”
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 103 - DNA, 847 bp, Artificial Sequence
  source: /note=“Description of Artificial Sequence: Synthetic polynucleotide”
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 104 - protein, 18 aa, Artificial Sequence
  source: /note=“Description of Artificial Sequence: Synthetic peptide”
GSTSGSGKPGSGEGSTKG

SEQ ID NO: 105 - protein, 394 aa, Artificial Sequence
  source: /note=“Description of Artificial Sequence: Synthetic polypeptide”
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 106 - protein, 373 aa, Artificial Sequence
  source: /note=“Description of Artificial Sequence: Synthetic polypeptide”
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 107 - DNA, 1182 bp, Artificial Sequence
  source: /note=“Description of Artificial Sequence: Synthetic polynucleotide”
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 108 - protein, 1132 aa, Homo sapiens [sequence omitted]

SEQ ID NO: 109 - DNA, 521 bp, Unknown
  source: /note=“Description of Unknown: PGK promoter”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttggggcaccataagct

SEQ ID NO: 110 - DNA, 118 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtg

SEQ ID NO: 111 - DNA, 221 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 112 - DNA, 324 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 113 - DNA, 422 bp, Artificial Sequence
  source: /note=“Description of Artificial Sequence: Synthetic polynucleotide”
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 114 - protein, 132 aa, Artificial Sequence
  source: /note=“Description of Artificial Sequence: Synthetic polypeptide”
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 115 - protein, 108 aa, Artificial Sequence
  source: /note=“Description of Artificial Sequence: Synthetic polypeptide”
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 116 - protein, 93 aa, Artificial Sequence [sequence omitted]

SEQ ID NO: 117 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 118 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 119 - protein, 95 aa, Artificial Sequence
  source: /note=“Description of Artificial Sequence: Synthetic polypeptide”
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 120 - protein, 95 aa, Artificial Sequence
  source: /note=“Description of Artificial Sequence: Synthetic polypeptide”
  MOD_RES (12)..(12): Any amino acid
  MOD_RES (78)..(78): Any amino acid
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 121 - protein, 95 aa, Artificial Sequence
  source: /note=“Description of Artificial Sequence: Synthetic polypeptide”
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 122 - protein, 95 aa, Artificial Sequence
  source: /note=“Description of Artificial Sequence: Synthetic polypeptide”
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 123 - DNA, 100 bp, Artificial Sequence
  source: /note=“Description of Artificial Sequence: Synthetic polynucleotide”
tttttttttttttttttttttttttttttttttttttttttttttttttttttttttttttttttttttttttttttttttttttttttttttttttttt

SEQ ID NO: 124 - protein, 244 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRS

SEQ ID NO: 125 - protein, 246 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSE

SEQ ID NO: 126 - protein, 245 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 127 - DNA, 63 bp, Artificial Sequence
  source: /note=“Description of Artificial Sequence: Synthetic oligonucleotide”
atggccctccctgtcaccgccctgctgcttccgctggctcttctgctccacgccgctcggccc

SEQ ID NO: 128 - DNA, 63 bp, Artificial Sequence
  source: /note=“Description of Artificial Sequence: Synthetic oligonucleotide”
atggccttaccagtgaccgccttgctcctgccgctggccttgctgctccacgccgccaggccg

SEQ ID NO: 129 - DNA, 135 bp, Artificial Sequence
  source: /note=“Description of Artificial Sequence: Synthetic polynucleotide”
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 130 - DNA, 72 bp, Artificial Sequence
  source: /note=“Description of Artificial Sequence: Synthetic oligonucleotide”
atctacatttgggcccctctggctggtacttgcggggtcctgctgctttcactcgtgatcactctttactgt

SEQ ID NO: 131 - DNA, 126 bp, Artificial Sequence
  source: /note=“Description of Artificial Sequence: Synthetic polynucleotide”
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 132 - DNA, 336 bp, Artificial Sequence
  source: /note=“Description of Artificial Sequence: Synthetic polynucleotide”
cgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 133 - protein, 5 aa, Artificial Sequence
  source: /note=“Description of Artificial Sequence: Synthetic peptide”
DYGVS

SEQ ID NO: 134 - protein, 16 aa, Artificial Sequence
  source: /note=“Description of Artificial Sequence: Synthetic peptide”
VIWGSETTYYNSALKS

SEQ ID NO: 135 - protein, 16 aa, Artificial Sequence
  source: /note=“Description of Artificial Sequence: Synthetic peptide”
VIWGSETTYYSSSLKS

SEQ ID NO: 136 - protein, 16 aa, Artificial Sequence
  source: /note=“Description of Artificial Sequence: Synthetic peptide”
VIWGSETTYYQSSLKS

SEQ ID NO: 137 - protein, 16 aa, Artificial Sequence
  source: /note=“Description of Artificial Sequence: Synthetic peptide”
VIWGSETTYYNSSLKS

SEQ ID NO: 138 - protein, 12 aa, Artificial Sequence
  source: /note=“Description of Artificial Sequence: Synthetic peptide”
HYYYGGSYAMDY

SEQ ID NO: 139 - protein, 11 aa, Artificial Sequence
  source: /note=“Description of Artificial Sequence: Synthetic peptide”
RASQDISKYLN

SEQ ID NO: 140 - protein, 7 aa, Artificial Sequence
  source: /note=“Description of Artificial Sequence: Synthetic peptide”
HTSRLHS

SEQ ID NO: 141 - protein, 9 aa, Artificial Sequence
  source: /note=“Description of Artificial Sequence: Synthetic peptide”
QQGNTLPYT

SEQ ID NO: 142-999 - skipped (858 entries)

SEQ ID NO: 1000 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 1001 - DNA, 4978 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 1002 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 1003 - DNA, 1197 bp, Homo sapiens
gtctcaatattagagtctcaacccccaataaatataggactggagatgtctgaggctcattctgccctcgagcccaccgggaacgaaagagaagctctatctcccctccaggagcccagctatgaactccttctccacaagcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggagacatgtaacaagagtaacatgtgtgaaagcagcaaagaggcactggcagaaaacaacctgaaccttccaaagatggctgaaaaagatggatgcttccaatctggattcaatgaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaaaaaaaaaa

SEQ ID NO: 1004 - protein, 918 aa, Homo sapiens [sequence omitted]

SEQ ID NO: 1005 - DNA, 2985 bp, Homo sapiens
cggcctgagtgaaacccaatggaaaaagcatgacatttagaagtagaagacttagcttcaaatccctactccttcacttactaattttgtgatttggaaatatccgcgcaagatgttgacgttgcagacttggctagtgcaagccttgtttattttcctcaccactgaatctacaggtgaacttctagatccatgtggttatatcagtcctgaatctccagttgtacaacttcattctaatttcactgcagtttgtgtgctaaaggaaaaatgtatggattattttcatgtaaatgctaattacattgtctggaaaacaaaccattttactattcctaaggagcaatatactatcataaacagaacagcatccagtgtcacctttacagatatagcttcattaaatattcagctcacttgcaacattcttacattcggacagcttgaacagaatgtttatggaatcacaataatttcaggcttgcctccagaaaaacctaaaaatttgagttgcattgtgaacgaggggaagaaaatgaggtgtgagtgggatcgtggaagggaaacacacttggagacaaacttcactttaaaatctgaatgggcaacacacaagtttgctgattgcaaagcaaaacgtgacacccccacctcatgcactgttgattattctactgtgtattttgtcaacattgaagtctgggtagaagcagagaatgcccttgggaaggttacatcagatcatatcaattttgatcctgtatataaagtgaagcccaatccgccacataatttatcagtgatcaactcagaggaactgtctagtatcttaaaattgacatggaccaacccaagtattaagagtgttataatactaaaatataacattcaatataggaccaaagatgcctcaacttggagccagattcctcctgaagacacagcatccacccgatcttcattcactgtccaagaccttaaaccttttacagaatatgtgtttaggattcgctgtatgaaggaagatggtaagggatactggagtgactggagtgaagaagcaagtgggatcacctatgaagatagaccatctaaagcaccaagtttctggtataaaatagatccatcccatactcaaggctacagaactgtacaactcgtgtggaagacattgcctccttttgaagccaatggaaaaatcttggattatgaagtgactctcacaagatggaaatcacatttacaaaattacacagttaatgccacaaaactgacagtaaatctcacaaatgatcgctatgtagcaaccctaacagtaagaaatcttgttggcaaatcagatgcagctgttttaactatccctgcctgtgactttcaagctactcaccctgtaatggatcttaaagcattccccaaagataacatgctttgggtggaatggactactccaagggaatctgtaaagaaatatatacttgagtggtgtgtgttatcagataaagcaccctgtatcacagactggcaacaagaagatggtaccgtgcatcgcacctatttaagagggaacttagcagagagcaaatgctatttgataacagttactccagtatatgctgatggaccaggaagccctgaatccataaaggcataccttaaacaagctccaccttccaaaggacctactgttcggacaaaaaaagtagggaaaaacgaagctgtcttagagtgggaccaacttcctgttgatgttcagaatggatttatcagaaattatactatattttatagaaccatcattggaaatgaaactgctgtgaatgtggattcttcccacacagaatatacattgtcctctttgactagtgacacattgtacatggtacgaatggcagcatacacagatgaaggtgggaaggatggtccagaattcacttttactaccccaaagtttgctcaaggagaaattgaagccatagtcgtgcctgtttgcttagcattcctattgacaactcttctgggagtgctgttctgctttaataagcgagacctaattaaaaaacacatctggcctaatgttccagatccttcaaagagtcatattgcccagtggtcacctcacactcctccaaggcacaattttaattcaaaagatcaaatgtattcagatggcaatttcactgatgtaagtgttgtggaaatagaagcaaatgacaaaaagccttttccagaagatctgaaatcattggacctgttcaaaaaggaaaaaattaatactgaaggacacagcagtggtattggggggtcttcatgcatgtcatcttctaggccaagcatttctagcagtgatgaaaatgaatcttcacaaaacacttcgagcactgtccagtattctaccgtggtacacagtggctacagacaccaagttccgtcagtccaagtcttctcaagatccgagtctacccagcccttgttagattcagaggagcggccagaagatctacaattagtagatcatgtagatggcggtgatggtattttgcccaggcaacagtacttcaaacagaactgcagtcagcatgaatccagtccagatatttcacattttgaaaggtcaaagcaagtttcatcagtcaatgaggaagattttgttagacttaaacagcagatttcagatcatatttcacaatcctgtggatctgggcaaatgaaaatgtttcaggaagtttctgcagcagatgcttttggtccaggtactgagggacaagtagaaagatttgaaacagttggcatggaggctgcgactgatgaaggcatgcctaaaagttacttaccacagactgtacggcaaggcggctacatgcctcagtgaaggactagtagttcctgctacaacttcagcagtacctataaagtaaagctaaaatgattttatctgtgaattcagattttaaaaagtcttcactctctgaagatgatcatttgccc

SEQ ID NO: 1006 - protein, 464 aa, Artificial Sequence
  source: /note=“Description of Artificial Sequence: Synthetic polypeptide”
QVQLLESGAELVRPGSSVKISCKASGYAFSSYWMNWVKQRPGQGLEWIGQIYPGDGDTNYNGKFKGQATLTADKSSSTAYMQLSGLTSEDSAVYSCARKTISSVVDFYFDYWGQGTTVTGGGSGGGSGGGSGGGSELVLTQSPKFMSTSVGDRVSVTCKASQNVGTNVAWYQQKPGQSPKPLIYSATYRNSGVPDRFTGSGSGTDFTLTITNVQSKDLADYFCQYNRYPYTSFFFTKLEIKRRSKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 1007 - protein, 439 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRL

SEQ ID NO: 1008 - protein, 819 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMLLLVTSLLLCELPHPAFLLIPRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCNLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFM

SEQ ID NO: 1009 - protein, 467 aa, Artificial Sequence
  source: /note=“Description of Artificial Sequence: Synthetic polypeptide”
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

(19) United States
(12) Patent Application Publication
(10) Pub. No.: US 2020/0370012 A1
(43) Pub. Date: Nov. 26, 2020

(54) METHODS OF MAKING CHIMERIC ANTIGEN RECEPTOR-EXPRESSING CELLS

(71) Applicants: Novartis AG, Basel (CH); The Trustees of the University of Pennsylvania, Philadelphia, PA (US)

(72) Inventors: Joseph A. Fraietta, Williamstown, NJ (US); Jan J. Melenhorst, Cherry Hill, NJ (US); Elena Orlando, Somerville, MA (US); Roderick O'Connor, Philadelphia, PA (US); Carl H. June, Merion Station, PA (US)

(21) Appl. No.: 16/759,251

(22) PCT Filed: Oct. 25, 2018
(86) PCT No.: PCT/US2018/057545

§ 371 Date: Apr. 24, 2020

Related U.S. Application Data
(60) Provisional application No. 62/637,854, filed on Mar. 2, 2018.
(60) Provisional application No. 62/577,099, filed on Oct. 25, 2017.

(51) Int. Cl. C12N 5/0783; C12Q 1/54; C07K 14/705; G01N 33/50

(52) U.S. Cl. CPC C12N 5/0636; C12N 2501/2328; C07K 14/70503; G01N 33/5091; G01N 2800/52; C12N 2501/2306; C12N 2501/231; C12N 2501/2311; C12N 2501/2317; C12N 2501/2319; C12N 2501/2322; C12N 2501/2324; C12N 2501/2323; C12N 2501/2326; C12Q 1/54

(57) ABSTRACT

The disclosure provides methods of making CAR-expressing immune effector cells (e.g., T cells, or NK cells), and compositions and reaction mixtures comprising the same. The disclosure further provides methods of using said CAR-expressing immune effector cells.

### FIELD OF THE INVENTION

The present invention relates generally to methods of making Chimeric Antigen Receptor (CAR) expressing immune effector cells (e.g., T cells, or NK cells), and compositions and reaction mixtures comprising the same.

### BACKGROUND OF THE INVENTION

Adoptive cell transfer (ACT) therapy with autologous T cells, especially with T cells transduced with Chimeric Antigen Receptors (CARs), has shown promise in several hematologic cancer trials.

The manufacture of autologous gene-modified T cells is currently a complex process that starts with the patient's material (e.g., obtained from leukapheresis) from which the engineered therapeutic T cells that express a CAR are derived. Patient leukapheresis material can have a high level of cell component variability. This starting material can vary greatly in cellular composition from patient to patient and within one disease state. Cell impurities can include granulocytes, monocytes, red blood cells, circulating blast cells, and platelets. Autologous cell therapy product manufacturing processes must also contend with patients' different treatment histories, state of disease, etc., which will further impact the cellular content of the starting material (Burger et al. 2014, Kaiser et al. 2015, Rα mos et al. 2009). Furthermore, such impurities from the starting material can negatively impact the manufacturing process, ultimate product quality, and therapeutic efficacy of the product.

Thus, there exists a need for methods and processes to provide a more consistent production of the CAR-expressing cell therapy product, thereby streamlining the manufacturing process, improving product quality, and maximizing the therapeutic efficacy of the product.

### SUMMARY OF THE INVENTION

The present disclosure pertains to methods of making CAR-expressing immune effector cells (e.g., T cells, NK cells), and compositions and reaction mixtures comprising the same. In some embodiments, the method of making comprises contacting a population of immune effector cells with (i) a Stat3 activator, e.g., as described herein, (ii) an inhibitor of glycolysis, e.g., a small molecule inhibitor of glycolysis, e.g., a small molecule hexokinase inhibitor, e.g., a glucose analog, e.g., 2-deoxy-D-glucose (2-DG), or both (i) and (ii). The disclosure also provides, in some aspects, methods of evaluating, predicting, selecting, or monitoring, a subject who will receive, is about to receive, has received or is receiving a therapeutic treatment with a CAR-expressing cell. Described herein are also methods of evaluating or predicting the responsiveness of a subject having a cancer (e.g., a cancer described herein), to a therapeutic treatment with a CAR-expressing cell.

In some aspects, disclosed herein is a method of making a population of Chimeric Antigen Receptor (CAR)-expressing immune effector cells, comprising

a) providing a population of immune effector cells, e.g., T cells;

b) contacting the population of immune effector cells with a nucleic acid encoding a CAR polypeptide;

c) contacting the population of immune effector cells with a Stat3 activator; and

d) maintaining the cells under conditions that allow expression of the CAR polypeptide,

thereby making a population of CAR-expressing immune effector cells.

In some embodiments, the Stat3 activator is chosen from, one, two, three, four, five, six, seven, eight, or all of, or any combination of:

i) a gp130 activator, e.g., an antibody molecule that binds to gp130, e.g., an anti-gp130 antibody as described herein, or an IL-6 molecule, an IL-11 molecule, an IL-27 molecule, a CNTF molecule, a CT-1 molecule, a CLC molecule, a LIF molecule, a NP molecule, an OSM molecule;

ii) a soluble IL-6 receptor (sIL-6R), e.g., as described herein;

iii) an IL-6/IL-6R complex, e.g., a dimer, e.g., as described herein;

iv) an IL-6 family cytokine (e.g., an IL-6 molecule, an IL-11 molecule, an IL-27 molecule, an IL-31 molecule, a CNTF molecule, a CT-1 molecule, a CLC molecule, a LIF molecule, a NP molecule or an OSM molecule);

v) a CCL20 molecule;

vi) an IL-10R2 receptor (IL-10R2) activator, e.g., an IL-10 molecule, an IL-22 molecule, an IL-26 molecule, an IL-28A molecule, an IL-28B molecule, an IL-29 molecule, or an antibody molecule that binds to IL-10R2, e.g., as described herein;

vii) an IL-10 family cytokine (e.g., an IL-10 molecule, an IL-19 molecule, an IL-20 molecule, an IL-22 molecule, an IL-24 molecule, an IL-26 molecule, an IL-28A molecule, an IL-28B molecule or an IL-29 molecule);

viii) an IL-17 family cytokine (e.g., an IL17A molecule, an IL17B molecule, an IL17C molecule, an IL17D molecule, an IL17E molecule or an IL17F molecule); or

ix) an IL-23 molecule.

In some embodiments, the Stat3 activator is a gp130 activator, e.g., an antibody molecule that binds to gp130, e.g., an anti-gp130 antibody as described herein.

In some embodiments, a method herein comprises adding, or a reaction mixture herein comprises, an IL-21 molecule, e.g., IL-21. In some embodiments, a reaction mixture herein does not comprise, or a method herein does not comprise adding, an IL-21 molecule, e.g., IL-21. In some embodiments, a method herein comprises adding, or a reaction mixture herein comprises, an IL-30 molecule. In some embodiments, a method herein comprises adding, or a reaction mixture herein comprises, an IL-6Rα activator, e.g., an antibody molecule that binds to IL-6Rα.

In some embodiments, the IL-6 family cytokine does not comprise an IL-6 molecule.

In some embodiments, the method further comprises introducing into at least one cell of the population of immune effector cells:

a gp130 molecule, e.g., by introducing into the at least one cell of the population of immune effector cells a nucleic acid encoding the gp130 molecule under conditions that allow for translation of the gp130 molecule; or

a Stat3 molecule (e.g., a constitutively active Stat3 molecule (STAT3C)), e.g., by introducing into the at least one cell of the population of immune effector cells a nucleic acid encoding the Stat3 molecule under conditions that allow for translation of the Stat3 molecule.

In some aspects, disclosed herein is a method of making a population of Chimeric Antigen Receptor (CAR)-expressing immune effector cells, comprising

a) providing a population of immune effector cells, e.g., T cells;

b) contacting the population of immune effector cells with a nucleic acid encoding a CAR polypeptide;

c) introducing into at least one cell of the population of immune effector cells:

a gp130 molecule, e.g., by introducing into the at least one cell of the population of immune effector cells a nucleic acid encoding the gp130 molecule under conditions that allow for translation of the gp130 molecule; or

a Stat3 molecule (e.g., a constitutively active Stat3 molecule (STAT3C)), e.g., by introducing into the at least one cell of the population of immune effector cells a nucleic acid encoding the Stat3 molecule under conditions that allow for translation of the Stat3 molecule; and

d) maintaining the cells under conditions that allow expression of the CAR polypeptide, gp130 molecule or Stat3 molecule,

thereby making a population of CAR-expressing immune effector cells.

In some embodiments, the method further comprises contacting the population of immune effector cells with a Stat3 activator chosen from, one, two, three, four, five, six, seven, eight, or all of, or any combination of:

i) a gp130 activator, e.g., an antibody molecule that binds to gp130, e.g., an anti-gp130 antibody as described herein, or an IL-6 molecule, an IL-11 molecule, an IL-27 molecule, a CNTF molecule, a CT-1 molecule, a CLC molecule, a LIF molecule, a NP molecule, an OSM molecule;

ii) a soluble IL-6 receptor (sIL-6R), e.g., as described herein;

iii) an IL-6/IL-6R complex, e.g., a dimer, e.g., as described herein;

iv) an IL-6 family cytokine (e.g., an IL-6 molecule, an IL-11 molecule, an IL-27 molecule, an IL-31 molecule, a CNTF molecule, a CT-1 molecule, a CLC molecule, a LIF molecule, a NP molecule or an OSM molecule);

v) a CCL20 molecule;

vi) an IL-10R2 receptor (IL-10R2) activator, e.g., an IL-10 molecule, an IL-22 molecule, an IL-26 molecule, an IL-28A molecule, an IL-28B molecule, an IL-29 molecule, or an antibody molecule that binds to IL-10R2, e.g., as described herein;

vii) an IL-10 family cytokine (e.g., an IL-10 molecule, an IL-19 molecule, an IL-20 molecule, an IL-22 molecule, an IL-24 molecule, an IL-26 molecule, an IL-28A molecule, an IL-28B molecule or an IL-29 molecule);

viii) an IL-17 family cytokine (e.g., an IL17A molecule, an IL17B molecule, an IL17C molecule, an IL17D molecule, an IL17E molecule or an IL17F molecule); or

ix) an IL-23 molecule.

In some embodiments, the Stat3 activator is a gp130 activator, e.g., an antibody molecule that binds to gp130, e.g., an anti-gp130 antibody as described herein.

In one embodiment, the IL-6 family cytokine does not comprise an IL-6 molecule.

In some embodiments of a method of manufacturing disclosed herein, the expression of the gp130 molecule or the Stat3 molecule is transient (e.g., inducible or non-inducible) or constitutive.

In some embodiments of a method of manufacturing disclosed herein, the gp130 molecule or the Stat3 molecule is introduced into the population of immune effector cells, prior to, concurrently, or after contacting the population of immune effector cells with:

a nucleic acid encoding a CAR polypeptide; or

a Stat3 activator, e.g., as described herein.

In some embodiments of a method of manufacturing disclosed herein, the nucleic acid comprising a nucleotide encoding a Stat3 molecule (e.g., a constitutively active Stat3 (STAT3C)), further comprises a nucleotide sequence encoding a CAR, e.g., a CD19 CAR.

In some embodiments of a method of manufacturing disclosed herein, the Stat3 activator is an antibody molecule that binds to gp130, e.g., an anti-gp130 antibody as described herein. In some embodiments, the method results in a population of T cells, e.g., CD4+ or CD8+ T cells, that is enriched for (e.g., has increased levels of), e.g., early memory T cells or non-exhausted early memory T cells. In some embodiments, the method results in enrichment of CD4+ or CD8+ early memory T cells, e.g., as described herein. In some embodiments, early memory T cells have one or both of the following characteristics: CD27+ and/or CD45RO^{dim/neg}, e.g., CD27+ CD45RO^{dim/neg}. In some embodiments, the method results in enrichment of CD4+ or CD8+ non-exhausted early memory T cells, e.g., as described herein. In some embodiments, non-exhausted early memory T cells have one or more, e.g., all, of the following characteristics: (i) PD-1 negative; (ii) CD27^{hi}; (iii) CCR7^{hi}; or (iv) CD45RO^{dim/neg}. In some embodiments, non-exhausted early memory T cells are PD-1 negative CD27^{hi} CCR7^{hi} CD45RO^{dim/neg}. In some embodiments, the enriched population of T cells, e.g., early memory T cells or non-exhausted early memory T cells, e.g., has an increased level of, e.g., at least 5-90% more (e.g., at least 5-10, 10-20, 20-30, 30-50, 50-70, or 70-90% more, or 5-90, 10-85, 15-80, 20-75, 25-70, 30-70, 35-65, 40-60, or 45-55% more) early memory T cells or non-exhausted early memory T cells. In some embodiments, the enriched population of T cells, e.g., early memory T cells or non-exhausted early memory T cells, has an increased level of, e.g., at least 5, 6, 7, 8, 9, 10, 11, 12, 13, 14, 15, 16, 17, 18, 19, 20, 21, 22, 23, 24, 25, 26, 27, 28, 29 or 30% more), early memory T cells or non-exhausted early memory T cells. In some embodiments, the increased level of early memory T cells or non-exhausted early memory T cells is compared to an otherwise similar population of T cells that was not contacted with the Stat3 activator, e.g., as described in Example 2. In some embodiments, the otherwise similar population of T cells that was not contacted with the Stat3 activator is the same population of T cells, e.g., on which the enrichment was performed, e.g., a pre-enrichment population, e.g., a starting population, e.g., as described in Example 2. In some embodiments, the otherwise similar population of T cells that was not contacted with the Stat3 activator is a different population of T cells, e.g., a population on which the enrichment was not performed.

In some embodiments, the enriched population of CD4+ T cells, e.g., early memory T cells or non-exhausted early memory T cells, has an increased level of, e.g., at least 5, 6, 7, 8, 9, 10, 11, 12, 13, 14, 15, 16, 17, 18, 19, or 20% more (e.g., at least 8%), early memory T cells or non-exhausted early memory T cells compared to an otherwise similar population of T cells that was not contacted with the Stat3 activator.

In some embodiments, the enriched population of CD8+ T cells, e.g., early memory T cells or non-exhausted early memory T cells, has an increased level of, e.g., at least 15, 16, 17, 18, 19, 20, 21, 22, 23, 24, 25, 26, 27, 28, 29 or 30% more (e.g., at least 20%), early memory T cells or non-exhausted early memory T cells compared to an otherwise similar population of T cells that was not contacted with the Stat3 activator.

In some embodiments of a method of manufacturing disclosed herein, the Stat3 activator comprises one, two, three, or all of: an IL-6 molecule, an IL-17 molecule, an IL-22 molecule or a CCL20 molecule. In one embodiment, the Stat3 activator is a naturally occurring molecule, a recombinant molecule or a purified molecule. In one embodiment, the Stat3 activator is not present in serum, e.g., not present in an amount sufficient to activate Stat3, e.g., phosphorylate Stat3, e.g., on tyrosine 705 (Y705), e.g., as measured by an assay of Example 2

In some embodiments of a method of manufacturing disclosed herein, the Stat3 activator is soluble (e.g., not bound to a substrate), and in some embodiments, the Stat3 activator is situated on, e.g., immobilized on, a substrate (e.g., a bead or cell).

In some embodiments of a method of manufacturing disclosed herein the Stat3 activator is situated, e.g., immobilized, on a substrate, e.g., bead or cell. In one embodiment, the Stat3 activator is situated on a Stat3 activator cell, e.g., an artificial antigen-presenting cell (APC), e.g., as described herein.

In some embodiments, an artificial APC comprises, one two or all of:

(i) an MHC molecule (e.g., expresses an MHC molecule on its surface);

(ii) a co-stimulatory protein (e.g., expresses a co-stimulatory protein on its surface, or to which a co-stimulatory protein is conjugated); and/or

(iii) an antigen, e.g., as described herein, e.g., an antigen that is recognized by a CAR-expressing cell, e.g., a CAR-expressing cell described herein.

In some embodiments of a method of manufacturing disclosed herein, the Stat3 activator is expressed by the Stat3 activator cell or is conjugated to the surface of the Stat3 activator cell.

In some embodiments of a method of manufacturing disclosed herein, the Stat3 activator, e.g., as described herein, is provided in an amount sufficient to activate Stat3, e.g., phosphorylate Stat3, e.g., on tyrosine 705 (Y705), e.g., as measured by an assay of Example 2.

In some embodiments of a method of manufacturing disclosed herein, the Stat3 activator, e.g., as described herein, is provided in an amount sufficient to expand the population of immune effector cells, by at least 1.1, 1.2, 1.3, 1.4, 1.5, 2, 3, 4, 5, 6, 7, 8, 9 fold or more after a 12 day culture period, e.g., as measured by an assay of Example 2, compared to an otherwise similar population of cells cultured under similar conditions but not contacted with the Stat3 activator.

In some embodiments of a method of manufacturing disclosed herein, the Stat3 activator, e.g., as described herein, is provided in an amount sufficient to increase the percentage of cells in the immune effector cell population that are CD27+ PD-1−, e.g., by at least 1.1, 1.2, 1.3, 1.4, 1.5, 2, 3, 4, 5, fold or greater, compared to an otherwise similar population of cells cultured under similar conditions but not contacted with the Stat3 activator.

In some embodiments of a method of manufacturing disclosed herein, the Stat3 activator, e.g., as described herein, is provided in an amount sufficient to increase the expression level of gp130 by at least 1.5, 2, 3, 4, 5, 10 fold or more, in the immune effector cell population, e.g., as measured by an assay of Example 2, compared to an otherwise similar population of cells cultured under similar conditions but not contacted with the Stat3 activator.

In some embodiments of a method of manufacturing disclosed herein, the Stat3 activator, e.g., as described herein, is chosen from one, two, three, four, or all (e.g., five) of: an IL-6 molecule, an IL-17 molecule, an IL-22 molecule, an IL31 molecule, and a CCL20 molecule.

In some embodiments of a method of manufacturing disclosed herein, the Stat3 activator, e.g., as described herein, comprises an IL-6 molecule, e.g., recombinant IL-6. In one embodiment, the IL-6 molecule, e.g., recombinant IL-6 is provided at an amount of at least 1, 5, 10, 15, 20, or 30 ng/ml, or in a range of 1-20, 1-15, or 5-15 ng/ml, e.g., at least 10 ng/ml.

In some embodiments of a method of manufacturing disclosed herein, the anti-gp130 antibody molecule is chosen from B-S12 or B-P8 or an antibody molecule having 1, 2, 3, 4, 5, or 6 CDRs from B-S12 or B-P8. In one embodiment, the method comprises contacting the population of immune effector cells with both of B-S12 and B-P8. In one embodiment, the total amount of anti-gp130 antibody molecule is 0.1-1000, 0.5-500. or 1-100 ug/ml. In one embodiment, the anti-gp130 antibody molecule is provided at an amount of at least 0.5, 0.6, 0.7, 0.8, 0.9, 1.0, 1.1, 1.2, 1.3, 1.4, 1.5, 1.6, 1.7, 1.8, 1.9, or 2 ug/ml, e.g., about 1 ug/ml.

In some embodiments, the anti-gp130 antibody:

induces gp130 mediated signaling, as measured by phosphorylation of STATS; or

induces dimerization, e.g., homodimerization of gp130, or heterodimerization of gp130, e.g., with LIF, OSM or CNTF.

In some embodiments of a method of manufacturing disclosed herein, the population of cells cultured in the presence of the Stat3 activator, e.g., as described herein, exhibits:

activation of Stat3, e.g., phosphorylation of Stat3, e.g., on tyrosine 705 (Y705), e.g., as measured by an assay of Example 2;

expansion of the population of immune effector cells, by at least 1.1, 1.2, 1.3, 1.4, 1.5, 2, 3, 4, 5, 6. 7, 8, 9 fold or more after a 12 day culture period, e.g., as measured by an assay of Example 2;

increase in the percentage of cells in the immune effector cell population that are CD27+ PD-1−, e.g., by at least 1.1, 1.2, 1.3, 1.4, 1.5, 2, 3, 4, 5, fold or greater;

increase in the expression level of gp130 by at least 1.5, 2, 3, 4, 5, or 10 fold or more, in the immune effector cell population, e.g., as measured by an assay of Example 2,

compared to an otherwise similar population of cells cultured under similar conditions but not contacted with the Stat3 activator.

In some embodiments, a CCL20 molecule comprises a full length naturally-occurring CCL20 (e.g., a mammalian CCL20, e.g., human CCL20), an active fragment of CCL20, or an active variant having at least 80%, 85%, 90%, 95%, 96%, 97%, 98%, or 99% sequence identity to a naturally-occurring wild type polypeptide of CCL20 or fragment thereof.

In some embodiments, a soluble IL-6 receptor, comprises a full length naturally-occurring IL-6 receptor (e.g., a mammalian IL-6 receptor, e.g., human IL-6 receptor), an active fragment of IL-6 receptor, or an active variant having at least 80%, 85%, 90%, 95%, 96%, 97%, 98%, or 99% sequence identity to a naturally-occurring wild type polypeptide of IL-6 receptor or fragment thereof.

In some embodiments, an IL-10R2 receptor activator comprises a molecule that activates IL-10R2 signaling pathway. In some embodiments, the IL-10R2 receptor activator comprises, e.g., a polypeptide or a small molecule.

In some embodiments, an IL-6/IL-6R complex comprises a complex between an IL-6 molecule and an IL-6 receptor (IL-6R) molecule. In some embodiments, an IL-6R molecule comprises a full length naturally-occurring IL-6 receptor (e.g., a mammalian IL-6 receptor, e.g., human IL-6 receptor), an active fragment of IL-6 receptor, or an active variant having at least 80%, 85%, 90%, 95%. 96%, 97%, 98%, or 99% sequence identity to a naturally-occurring wild type polypeptide of IL-6 receptor or fragment thereof.

In some embodiments a method of manufacturing disclosed herein, comprises expanding the population, e.g., for at least 1, 2, 3, 4, 5, 6, 7, 8, 9, 10, 11, 12, 13, 14, 15, 16, 17, 18, 19, 20, or 21 days or for 1-7, 7-14, or 14-21 days.

In some embodiments a method of manufacturing disclosed herein, further comprises assaying Stat3 pathway activation in the population of immune effector cells by measuring the level or activity of Stat3 transcriptional targets, e.g., c-Myc, c-Fos, Sox2, Bcl-2, or RORC to determine a value for Stat3 pathway activation. In one embodiment, the method comprises comparing the Stat3 pathway activation value with a reference value, wherein the reference value is obtained from an otherwise similar population of immune effector cells cultured under similar conditions but not contacted with the Stat3 activator, e.g., as described herein.

In some embodiments, responsive to the comparison of the Stat3 pathway activation value with reference value, performing one or more of:

classifying the population as suitable or not suitable for use as a therapeutic;

formulating or packaging the population, or an aliquot thereof, for therapeutic use; or

altering a culture parameter, e.g., i) altering the length of time in culture or ii) increasing or decreasing the concentration of the Stat3 activator, e.g., as described herein.

In some embodiments of a method of manufacturing disclosed herein, (b) is performed before (c), (c) is performed before (b), or (b) and (c) are performed simultaneously.

In some embodiments, the nucleic acid is DNA or RNA.

In some embodiments, (b) comprises performing lentiviral transduction to deliver the nucleic acid to the immune effector cells.

In some embodiments, the method further comprises contacting the population of immune effector cells with a population of cells that expresses an antigen (e.g., CD19) that binds the CAR.

In some embodiments, the method further comprises contacting the population of immune effector cells with an agent that stimulates a CD3/TCR complex associated signal and a ligand that stimulates a costimulatory molecule on the surface of the cells, e.g., wherein the agent is a bead conjugated with anti-CD3 antibody, or a fragment thereof, and/or anti-CD28 antibody, or a fragment thereof.

In some embodiments, the CAR polypeptide is a CD19 CAR, a CD22 CAR, a CD123 CAR or a CD33 CAR. In one embodiment, the CAR is a CD19 CAR, e.g., a CAR comprising an scFv amino acid sequence of SEQ ID NO: 39-51 or a CAR comprising the amino acid sequence of SEQ ID NO: 77-89.

In some embodiments, the CAR comprises an antibody molecule which includes an anti-CD19 binding domain, a transmembrane domain, and an intracellular signaling domain comprising a stimulatory domain, and wherein said anti-CD19 binding domain comprises one or more of light chain complementary determining region 1 (LC CDR1), light chain complementary determining region 2 (LC CDR2), and light chain complementary determining region 3 (LC CDR3) of any anti-CD19 light chain binding domain amino acid sequence listed in Table 3B, and one or more of heavy chain complementary determining region 1 (HC CDR1), heavy chain complementary determining region 2 (HC CDR2), and heavy chain complementary determining region 3 (HC CDR3) of any anti-CD19 heavy chain binding domain amino acid sequence listed in Table 3A.

In some embodiments, the anti-CD19 binding domain comprises a sequence of SEQ ID NO: 40, or SEQ ID NO:51.

In some embodiments, the CAR comprises a polypeptide having a sequence of SEQ ID NO:78, or SEQ ID NO: 89.

In one aspect, disclosed herein is a method of making a population of Chimeric Antigen Receptor (CAR)-expressing immune effector cells, comprising:

a) providing a population of immune effector cells, e.g., T cells;

b) contacting the population of immune effector cells with a nucleic acid encoding a CAR polypeptide;

c) contacting the population of immune effector cells with an inhibitor of glycolysis, e.g., a small molecule inhibitor of glycolysis, e.g., a small molecule hexokinase inhibitor, e.g., a glucose analog, e.g., 2-deoxy-D-glucose (2-DG), and

d) maintaining the cells under conditions that allow expression of the CAR polypeptide,

thereby making a population of CAR-expressing immune effector cells.

In one embodiment, (b) is performed before (c), (c) is performed before (b), or (b) and (c) are performed simultaneously.

In one embodiment, the nucleic acid is DNA or RNA.

In one embodiment, (b) comprises performing lentiviral transduction to deliver the nucleic acid to the immune effector cells.

In one embodiment, the inhibitor of glycolysis, e.g., a small molecule inhibitor of glycolysis, e.g., a small molecule hexokinase inhibitor, e.g., a glucose analog, e.g., 2-DG, is added in an amount sufficient to:

increase the population of immune effector cells at least 10%, 20%, 30%, 40%, 50%, 60%, 70%, 80%, 90%, 95%, 100% or greater; or

increase the percentage of cells in the immune effector cell population that have a central memory phenotype, e.g., are CD45RO+CCR7+, e.g., by about at least 10%, 20%, 30%, 40%, 50%, 60%, 70%, 80%, 90%, 95%, 100% or greater;

compared to an otherwise similar population of cells cultured under similar conditions but not treated with the inhibitor of glycolysis.

In one embodiment, the inhibitor of glycolysis, e.g., 2-DG, is added at a concentration of at least 0.5, 1, 1.5, 2, or 2.5 mM, 0.5-2.5 mM, or 1-2 mM.

In one embodiment, the population of cells cultured in the presence of the glycolysis inhibitor exhibits:

an increase the population of immune effector cells at least 10%, 20%, 30%, 40%, 50%, 60%, 70%, 80%, 90%, 95%, 100% or greater; or

an increase the percentage of cells in the immune effector cell population that have a central memory phenotype, e.g., are CD45RO+CCR7+, e.g., by about at least 10%, 20%, 30%, 40%, 50%, 60%, 70%, 80%, 90%, 95%, 100% or greater;

compared to an otherwise similar population of cells cultured under similar conditions but not treated with the inhibitor of glycolysis.

In one embodiment, the method comprises:

expanding the population, e.g., for at least 1, 2, 3, 4, 5, 6, 7, 8, 9, 10, 11, 12, 13, 14, 15, 16, 17, 18, 19, 20, or 21 days or for 1-7, 7-14, or 14-21 days; or expanding the population, e.g., by at least a 1.5, 2, 2.5, 3, 4, 5, 5, 7, 8, 9, 10, 20, 30, 40, 50-fold change in cell number or more, e.g, up to about 40 or 50-fold, e.g., under growth conditions of Example 1.

In one embodiment, the method further comprises, assaying glucose metabolism in the population of immune effector cells to determine a glucose metabolism value, e.g., using 2-NBDG uptake assay, e.g., an assay of Example 1.

In one embodiment, the method further comprises comparing the glucose metabolism value with a reference value.

In one embodiment, the method further comprises, responsive to the comparison of the glucose metabolism value with reference value, performing one or more of:

classifying the population as suitable or not suitable for use as a therapeutic;

formulating or packaging the population, or an aliquot thereof, for therapeutic use; or

altering a culture parameter, e.g., i) altering the length of time in culture or ii) increasing or decreasing the concentration of the inhibitor of glycolysis, e.g., the small molecule inhibitor of glycolysis, e.g., the small molecule hexokinase inhibitor, e.g., the glucose analog, e.g., 2-deoxy-D-glucose (2-DG).

In one embodiment, the method further comprises contacting the population of immune effector cells with a population of cells that expresses an antigen (e.g., CD19) that binds the CAR.

In one embodiment, the method further comprises contacting the population of immune effector cells with an agent that stimulates a CD3/TCR complex associated signal and a ligand that stimulates a costimulatory molecule on the surface of the cells, e.g., wherein the agent is a bead conjugated with anti-CD3 antibody, or a fragment thereof, and/or anti-CD28 antibody, or a fragment thereof.

In one embodiment, the CAR is a CD19 CAR, a CD22 CAR, a CD123 CAR or a CD33 CAR. In one embodiment, the CAR is a CD19 CAR, e.g., a CAR comprising an scFv amino acid sequence of SEQ ID NO: 39-51 or a CAR comprising the amino acid sequence of SEQ ID NO: 77-89.

In one embodiment, the CAR comprises an antibody or antibody fragment which includes a anti-CD19 binding domain, a transmembrane domain, and an intracellular signaling domain comprising a stimulatory domain, and wherein said anti-CD19 binding domain comprises one or more of light chain complementary determining region 1 (LC CDR1), light chain complementary determining region 2 (LC CDR2), and light chain complementary determining region 3 (LC CDR3) of any anti-CD19 light chain binding domain amino acid sequence listed in Table 3, and one or more of heavy chain complementary determining region 1 (HC CDR1), heavy chain complementary determining region 2 (HC CDR2), and heavy chain complementary determining region 3 (HC CDR3) of any anti-CD19 heavy chain binding domain amino acid sequence listed in Table 3.

In one embodiment, the CAR comprises an antibody or antibody fragment which includes a anti-CD19 binding domain, a transmembrane domain, and an intracellular signaling domain comprising a stimulatory domain, and wherein said anti-CD19 binding domain comprises one or more of light chain complementary determining region 1 (LC CDR1), light chain complementary determining region 2 (LC CDR2), and light chain complementary determining region 3 (LC CDR3) of any anti-CD19 light chain binding domain amino acid sequence listed in Table 3B, and one or more of heavy chain complementary determining region 1 (HC CDR1), heavy chain complementary determining region 2 (HC CDR2), and heavy chain complementary determining region 3 (HC CDR3) of any anti-CD19 heavy chain binding domain amino acid sequence listed in Table 3A.

In one embodiment, the anti-CD19 binding domain comprises a sequence of SEQ ID NO: 40, or SEQ ID NO:51.

In one embodiment, the CAR comprises a polypeptide having a sequence of SEQ ID NO:78, or SEQ ID NO: 89.

In some aspects, disclosed herein is a method of making a population of Chimeric Antigen Receptor (CAR)-expressing immune effector cells, comprising:

a) providing a population of immune effector cells, e.g., T cells;

b) contacting the population of immune effector cells with a nucleic acid encoding a CAR polypeptide;

c) contacting the population of immune effector cells with:

c-i) a Stat3 activator, e.g., as described herein; and

c-iii) an inhibitor of glycolysis, e.g., a small molecule inhibitor of glycolysis, e.g., a small molecule hexokinase inhibitor, e.g., a glucose analog, e.g., 2-deoxy-D-glucose (2-DG), and

d) maintaining the cells under conditions that allow expression of the CAR polypeptide,

thereby making a population of CAR-expressing immune effector cells.

In some embodiments, the Stat3 activator is chosen from, one, two, three, four, five, six, seven, eight, or all of, or any combination of:

i) a gp130 activator, e.g., an antibody molecule that binds to gp130, e.g., an anti-gp130 antibody as described herein, or an IL-6 molecule, an IL-11 molecule, an IL-27 molecule, a CNTF molecule, a CT-1 molecule, a CLC molecule, a LIF molecule, a NP molecule, an OSM molecule;

ii) a soluble IL-6 receptor (sIL-6R), e.g., as described herein;

iii) an IL-6/IL-6R complex, e.g., a dimer, e.g., as described herein;

iv) an IL-6 family cytokine (e.g., an IL-6 molecule, an IL-11 molecule, an IL-27 molecule, an IL-31 molecule, a CNTF molecule, a CT-1 molecule, a CLC molecule, a LW molecule, a NP molecule or an OSM molecule);

v) a CCL20 molecule;

vi) an IL-10R2 receptor (IL-10R2) activator, e.g., an IL-10 molecule, an IL-22 molecule, an IL-26 molecule, an IL-28A molecule, an IL-28B molecule, an IL-29 molecule, or an antibody molecule that binds to IL-10R2, e.g., as described herein;

vii) an IL-10 family cytokine (e.g., an IL-10 molecule, an IL-19 molecule, an IL-20 molecule, an IL-22 molecule, an IL-24 molecule, an IL-26 molecule, an IL-28A molecule, an IL-28B molecule or an IL-29 molecule);

viii) an IL-17 family cytokine (e.g., an IL17A molecule, an IL17B molecule, an IL17C molecule, an IL17D molecule, an IL17E molecule or an IL17F molecule); or six) an IL-23 molecule.

In some aspects, disclosed herein is a method of making a population of Chimeric Antigen Receptor (CAR)-expressing immune effector cells, comprising

a) providing a population of immune effector cells, e.g., T cells;

b) contacting the population of immune effector cells with a nucleic acid encoding a CAR polypeptide;

c) contacting the population of immune effector cells with:

c-i) an inhibitor of glycolysis, e.g., a small molecule inhibitor of glycolysis, e.g., a small molecule hexokinase inhibitor, e.g., a glucose analog, e.g., 2-deoxy-D-glucose (2-DG), and

c-ii) a gp130 molecule, e.g., by introducing into the at least one cell of the population of immune effector cells a nucleic acid encoding the gp130 molecule under conditions that allow for translation of the gp130 molecule; or a Stat3 molecule (e.g., a constitutively active Stat3 molecule (STAT3C)), e.g., by introducing into the at least one cell of the population of immune effector cells a nucleic acid encoding the Stat3 molecule under conditions that allow for translation of the Stat3 molecule; and

d) maintaining the cells under conditions that allow expression of the CAR polypeptide, gp130 molecule or Stat3 molecule,

thereby making a population of CAR-expressing immune effector cells.

In some embodiments, a method of manufacturing comprising a Stat3 activator or a population of cells comprising a gp130 molecule or a Stat3 molecule, further comprises contacting the population of immune effector cells with an inhibitor of glycolysis, e.g., a small molecule inhibitor of glycolysis, e.g., a small molecule hexokinase inhibitor, e.g., a glucose analog, e.g., 2-deoxy-D-glucose (2-DG).

In some embodiments, a method of manufacturing comprising an inhibitor of glycolysis, e.g., as described herein, further comprises contacting the population of immune effector cells with a Stat3 activator or a population of cells comprising a gp130 molecule or a Stat3 molecule.

In some aspects, disclosed herein is a reaction mixture comprising:

a) (i) a population of CAR-expressing immune effector cells (e.g., a CAR-expressing cell described herein, e.g., a CD19 CAR-expressing cell) or (ii) an immune effector cell and a nucleic acid encoding a CAR (e.g., a CAR described herein, e.g., a CD19 CAR); and

b) an agent selected from:
(i) a Stat3 activator;
(ii) a cell or population of cells expressing a gp130 molecule or a Stat3 molecule; or
(iii) a gp130 molecule, or a Stat3 molecule, or nucleic acid encoding same.

In one embodiment of a reaction mixture disclosed herein, the Stat3 activator is chosen from:

b-i-i) a gp130 activator, e.g., an antibody molecule that binds to gp130, e.g., an anti-gp130 antibody as described herein, or an IL-6 molecule, an IL-11 molecule, an IL-27 molecule, a CNTF molecule, a CT-1 molecule, a CLC molecule, a LIF molecule, a NP molecule, an OSM molecule;

b-i-ii) a soluble IL-6 receptor (sIL-6R), e.g., as described herein;

b-i-iii) an IL-6/IL-6R complex, e.g., a dimer, e.g., as described herein;

b-i-iv) an IL-6 family cytokine (e.g., an IL-6 molecule, an IL-11 molecule, an IL-27 molecule, an IL-31 molecule, a CNTF molecule, a CT-1 molecule, a CLC molecule, a LIF molecule, a NP molecule or an OSM molecule);

b-i-v) a CCL20 molecule;

b-i-vi) an IL-10R2 receptor (IL-10R2) activator, e.g., an IL-10 molecule, an IL-22 molecule, an IL-26 molecule, an IL-28A molecule, an IL-28B molecule, an IL-29 molecule, or an antibody molecule that binds to IL-10R2, e.g., as described herein;

b-i-vii) an IL-10 family cytokine (e.g., an IL-10 molecule, an IL-19 molecule, an IL-20 molecule, an IL-22 molecule, an IL-24 molecule, an IL-26 molecule, an IL-28A molecule, an IL-28B molecule or an IL-29 molecule); or

b-i-viii) an IL-17 family cytokine (e.g., an IL17A molecule, an IL17B molecule, an IL17C molecule, an IL17D molecule, an IL17E molecule or an IL17F molecule).

In some embodiments, a reaction mixture disclosed herein comprises (a)(i) a population of CAR-expressing immune effector cells.

In some embodiments, a reaction mixture disclosed herein comprises (a)(ii) a nucleic acid encoding a CAR (e.g., a CAR described herein, e.g., a CD19 CAR).

In some embodiments, a reaction mixture disclosed herein comprises (b)(i) a Stat3 activator, e.g., as described herein.

In some embodiments, a reaction mixture disclosed herein comprises (b)(ii) the cell or population of cells comprising a gp130 molecule or Stat3 molecule.

In some embodiments, a reaction mixture disclosed herein comprises (b)(iii) a gp130 molecule, or a Stat3 molecule, or nucleic acid encoding same.

In some embodiments, a reaction mixture disclosed herein comprises: (a)(i) a population of CAR-expressing immune effector cells; and (b)(i) a Stat3 activator , e.g., as described herein.

In some embodiments, a reaction mixture disclosed herein comprises: (a)(i) a population of CAR-expressing immune effector cells; and (b)(ii) the cell or population of cells comprising a gp130 molecule or Stat3 molecule.

In some embodiments, a reaction mixture disclosed herein comprises: (a)(i) a population of CAR-expressing immune effector cells; and(b)(iii) a gp130 molecule, or a Stat3 molecule, or nucleic acid encoding same.

In some embodiments, a reaction mixture disclosed herein comprises: (a)(ii) a nucleic acid encoding a CAR; and (b)(i) a Stat3 activator, e.g., as described herein.

In some embodiments, a reaction mixture disclosed herein comprises: (a)(ii) a nucleic acid encoding a CAR; and (b)(ii) the cell or population of cells comprising a gp130 molecule or Stat3 molecule.

In some embodiments, a reaction mixture disclosed herein comprises: (a)(ii) a nucleic acid encoding a CAR; and (b)(iii) a gp130 molecule, or a Stat3 molecule, or nucleic acid encoding same.

In some embodiments, a reaction mixture disclosed herein comprises one or more of: (a)(i) and b-i-i); (a)(i) and b-i-ii); (a)(i) and b-i-iii); (a)(i) and b-i-iv); (a)(i) and b-i-v); (a)(i) and b-i-vi); (a)(i) and b-i-vii); (a)(i) and b-i-viii); (a)(ii) and b-i-i); (a)(ii) and b-i-ii); (a)(ii) and b-i-iii); (a)(ii) and b-i-iv); (a)(ii) and b-i-v); (a)(ii) and b-i-vi); (a)(ii) and b-i-vii); and (a)(ii) and b-i-viii).

In one aspect, disclosed herein is a reaction mixture comprising:
a) a population of CAR-expressing immune effector cells, e.g., a CAR-expressing cell described herein, e.g., a CD19 CAR-expressing cell, and
b) an inhibitor of glycolysis, e.g., a small molecule inhibitor of glycolysis, e.g., a small molecule hexokinase inhibitor, e.g., a glucose analog, e.g., 2-deoxy-D-glucose (2-DG).

In another aspect, disclosed herein is a reaction mixture comprising:
a) a population of immune effector cells,
b) a nucleic acid encoding a CAR, e.g., a CAR described herein, e.g., a CD19 CAR, and
c) an inhibitor of glycolysis, e.g., a small molecule inhibitor of glycolysis, e.g., a small molecule hexokinase inhibitor, e.g., a glucose analog, e.g., 2-deoxy-D-glucose (2-DG).

In certain aspects, a reaction mixture disclosed herein comprises:
(i) a Stat3 activator;
(ii) a cell or population of cells expressing a gp130 molecule or a Stat3 molecule;
(iii) a gp130 molecule, or a Stat3 molecule, or nucleic acid encoding same; and
(iv) an inhibitor of glycolysis, e.g., a small molecule inhibitor of glycolysis, e.g., a small molecule hexokinase inhibitor, e.g., a glucose analog, e.g., 2-deoxy-D-glucose (2-DG).

In one embodiment, a reaction mixture disclosed herein further comprises a lentivirus, e.g., wherein the nucleic acid encoding a CAR is packaged in a lentivirus.

In one embodiment of a reaction mixture disclosed herein, the nucleic acid is DNA or RNA.

In one embodiment, a reaction mixture disclosed herein further comprises a population of cells that expresses an antigen (e.g., CD19) that binds the CAR.

In one embodiment of a reaction mixture disclosed herein, the inhibitor of glycolysis, e.g., 2-DG, is present at a concentration of at least 0.5, 1, 1.5, 2, 2.5 mM, 0.5-2.5 mM, or 1-2 mM.

In some aspects, the disclosed provides a method of evaluating or predicting the responsiveness of a subject having a cancer (e.g., a cancer described herein), to a therapeutic treatment with a CAR-expressing cell, e.g., prior to administration of the CAR-expressing cell, comprising evaluating in an immune effector cell from the subject:
i) a level of glucose metabolism, wherein:

a level of glucose metabolism that is lower than a glucose metabolism reference value is indicative that the subject is likely to respond to treatment with the CAR-expressing cell, e.g., to exhibit a complete response, or a partial response and

a level of glucose metabolism that is higher than a glucose metabolism reference value is indicative that the subject is less likely to respond to treatment with the CAR-expressing cell, e.g., does not exhibit a complete response or partial response; or
ii) a level of Stat3 activation as measured by, e.g., phosphorylation of Stat3 (e.g., on tyrosine 705 (Y705)) or level or activity of Stat3 transcriptional targets (e.g., c-Myc, c-Fos, Sox2, Bcl-2, or RORC), wherein:

a level of Stat3 activation that is higher than a Stat3 activation reference value is indicative that the subject is likely to respond to treatment with the CAR-expressing cell, e.g., to exhibit a complete response, or a partial response and

a level of Stat3 activation that is lower than a Stat3 activation reference value is indicative that the subject is less likely to respond to treatment with the CAR-expressing cell, e.g., does not exhibit a complete response or partial response,

thereby evaluating the subject, or predicting the responsiveness of the subject to the CAR-expressing cell.

In some embodiments, the immune effector cell has not been contacted with a nucleic acid encoding a CAR.

In some embodiments, the immune effector cell has been contacted with a nucleic acid encoding a CAR, e.g., expresses a CAR polypeptide.

In some embodiments, the immune effector cell has been contacted with:
i) a Stat3 activator, e.g., as described herein;
ii) a cell or population of cells comprising a gp130 molecule or a Stat3 molecule;
iii) a gp130 molecule, or a Stat3 molecule, or nucleic acid encoding the same; or
iv) an inhibitor of glycolysis, e.g., a small molecule inhibitor of glycolysis, e.g., a small molecule hexokinase inhibitor, e.g., a glucose analog, e.g., 2-deoxy-D-glucose (2-DG) at a concentration of at least 0.5, 1, 1.5, 2, or 2.5 mM.

In some embodiments, the method further comprises determining a fold change in cell number, e.g., number of CAR-expressing cells.

In some embodiments, the subject that is less likely to respond to treatment with the CAR-expressing cell is predicted to exhibit No Response (NR) or a Partial Response (PR).

In some embodiments, responsive to determination that:
i) the level of glucose metabolism is lower than the glucose metabolism reference value; or
ii) the level of Stat3 activation is higher than the Stat3 activation reference value,
   the subject is selected for administration of, or is administered, a CAR-expressing therapy.

In some embodiments, responsive to determination that:
i) the level of glucose metabolism is higher than the glucose metabolism reference value; or
ii) the level of Stat3 activation is lower than the Stat3 activation reference value,
   the subject is selected for administration of, or is administered, a therapy other than a CAR-expressing therapy.

In some embodiments, the glucose metabolism reference value is the glucose metabolism value of a cell of a complete responder subject, e.g., as described in Example 1, e.g., wherein the cell (e.g., a sample containing the cell) is contacted with mock stimulation, e.g., stimulation with an antigen other than the CAR antigen, e.g., as described in Example 1.

In some embodiments, the Stat3 activation reference value is the Stat3 activation value of a cell of a non-responder subject, e.g., as described in Example 2.

In one aspect, the disclosed provides a method of evaluating or predicting the responsiveness of a subject having a cancer (e.g., a cancer described herein), to a therapeutic treatment with a CAR-expressing cell, e.g., prior to administration of the CAR-expressing cell, comprising

evaluating a level of glucose metabolism in an immune effector cell from the subject, wherein:

a level of glucose metabolism that is lower than a reference value is indicative that the subject is likely to respond to treatment with the CAR-expressing cell, e.g., to exhibit a complete response, or a partial response and

a level of glucose metabolism that is higher than a reference value is indicative that the subject is less likely to respond to treatment with the CAR-expressing cell, e.g., does not exhibit a complete response or partial response,

thereby evaluating the subject, or predicting the responsiveness of the subject to the CAR-expressing cell.

In one embodiment, the immune effector cell has not been contacted with a nucleic acid encoding a CAR.

In one embodiment, the immune effector cell has been contacted with a nucleic acid encoding a CAR, e.g., expresses a CAR polypeptide.

In one embodiment, the immune effector cell has been contacted with an inhibitor of glycolysis, e.g., a small molecule inhibitor of glycolysis, e.g., a small molecule hexokinase inhibitor, e.g., a glucose analog, e.g., 2-deoxy-D-glucose (2-DG) at a concentration of at least 0.5, 1, 1.5, 2, or 2.5 mM.

In one embodiment, the method further comprises determining a fold change in cell number.

In one embodiment, the subject that is less likely to respond to treatment with the CAR-expressing cell is predicted to exhibit No Response (NR) or a Partial Response (PR).

In one embodiment, responsive to determination that the level of glucose metabolism is lower than the reference value, the subject is selected for administration of, or is administered a CAR-expressing therapy.

In one embodiment, responsive to determination that the level of glucose metabolism is higher than the reference value, the subject is selected for administration of, or is administered a therapy other than a CAR-expressing therapy.

In one embodiment, the reference value is the glucose metabolism value of a cell of a complete responder subject as described in Example 1, e.g., wherein the cell (e.g., a sample containing the cell) is contacted with mock stimulation, e.g., stimulation with an antigen other than the CAR antigen, e.g., as described in Example 1.

In some aspects, disclosed herein is a method of evaluating or predicting the responsiveness of a subject having a cancer (e.g., a cancer described herein), wherein the subject has been treated with a CAR-expressing cell, comprising evaluating in a CAR-expressing cell from the subject:
i) a level of glucose metabolism, wherein:

a level of glucose metabolism that is lower than a glucose metabolism reference value is indicative that the subject is likely to respond to treatment with the CAR-expressing cell, e.g., to exhibit a complete response, or a partial response and

a level of glucose metabolism that is higher than a glucose metabolism reference value is indicative that the subject is less likely to respond to treatment with the CAR-expressing cell, e.g., does not exhibit a complete response or partial response; or
ii) a level of Stat3 activation as measured by, e.g., phosphorylation of Stat3 (e.g., on tyrosine 705 (Y705)) or level or activity of Stat3 transcriptional targets (e.g., c-Myc, c-Fos, Sox2, Bcl-2, or RORC), wherein:

a level of Stat3 activation that is higher than a Stat3 activation reference value is indicative that the subject is likely to respond to treatment with the CAR-expressing cell, e.g., to exhibit a complete response, or a partial response and

a level of Stat3 activation that is lower than a Stat3 activation reference value is indicative that the subject is less likely to respond to treatment with the CAR-expressing cell, e.g., does not exhibit a complete response or partial response,

thereby evaluating the subject, or predicting the responsiveness of the subject to the CAR-expressing cell.

In some embodiments, the method further comprises obtaining the CAR-expressing cell from the subject prior to evaluating the level of glucose metabolism, or the level of Stat3 activation in the CAR-expressing cell.

In some embodiments, the subject that is less likely to respond to treatment with the CAR-expressing cell is predicted to exhibit NR or PR.

In some embodiments, responsive to determination that:
i) the level of glucose metabolism is lower than the glucose metabolism reference value; or
ii) the level of Stat3 activation is higher than the Stat3 activation reference value,
   the subject is selected for administration of, or is administered, one or more additional doses of the CAR-expressing therapy.

In some embodiments, responsive to determination that:
i) the level of glucose metabolism is higher than the glucose metabolism reference value; or
ii) the level of Stat3 activation is lower than the Stat3 activation reference value,
   the subject is selected for administration of, or is administered, a therapy other than a CAR-expressing therapy.

In some embodiments, the glucose metabolism reference value is the glucose metabolism value of a cell of a complete responder subject as described in Example 1, e.g., wherein the cell (e.g., a sample containing the cell) is contacted with mock stimulation, e.g., stimulation with an antigen other than the CAR antigen, e.g., as described in Example 1.

In some embodiments, the Stat3 activation reference value is the Stat3 activation value of a cell of a non-responder subject, e.g., as described in Example 2.

In one aspect, disclosed herein is a method of evaluating or predicting the responsiveness of a subject having a cancer (e.g., a cancer described herein), wherein the subject has been treated with a CAR-expressing cell, comprising:

evaluating a level of glucose metabolism in a CAR-expressing cell from the subject, wherein:

a level of glucose metabolism that is lower than a reference value is indicative that the subject is likely to respond to treatment with the CAR-expressing cell, e.g., to exhibit a complete response, or a partial response (e.g., PRTD), and

a level of glucose metabolism that is higher than a reference value is indicative that the subject is less likely to respond to treatment with the CAR-expressing cell, e.g., does not exhibit a complete response or a partial response e.g., PRTD,

thereby evaluating the subject, or predicting the responsiveness of the subject to the CAR-expressing cell.

In one embodiment, the method further comprises obtaining the CAR-expressing cell from the subject prior to evaluating the level of glucose metabolism in the CAR-expressing cell.

In one embodiment, the subject that is less likely to respond to treatment with the CAR-expressing cell is predicted to exhibit NR or PR.

In one embodiment, responsive to determination that the level of glucose metabolism is lower than the reference value, the subject is selected for administration of, or is administered one or more additional doses of the CAR-expressing therapy.

In one embodiment, responsive to determination that the level of glucose metabolism is higher than the reference value, the subject is selected for administration of, or is administered a therapy other than a CAR-expressing therapy.

In one embodiment, the reference value is the glucose metabolism value of a cell of a complete responder subject as described in Example 1, e.g., wherein the cell (e.g., a sample containing the cell) is contacted with mock stimulation, e.g., stimulation with an antigen other than the CAR antigen, e.g., as described in Example 1.

In some aspects, the disclosure provides a method of evaluating a CAR-expressing cell, e.g., CAR19− expressing cell, (e.g., CTL019), said method comprising evaluating in the CAR-expressing cell in a sample from a subject:

i) a level of glucose metabolism, wherein:

a level of glucose metabolism that is lower than a glucose metabolism reference value is indicative that the sample is suitable for treatment, and

a level of glucose metabolism that is higher than a glucose metabolism reference value is indicative that the sample is less suitable for treatment; or

ii) a level of Stat3 activation, wherein:

a level of Stat3 activation that is higher than a Stat3 activation reference value is indicative that the sample is suitable for treatment, and

a level of Stat3 activation that is lower than a Stat3 activation reference value is indicative that the sample is less suitable for treatment,

thereby evaluating the CAR-expressing cell.

In some embodiments, the method further comprises selecting a cell, or enriching for a plurality of cells, which cell or plurality is suitable for treatment. In some embodiments, the method further comprises removing a cell, or de-enriching for a plurality of cells, which cell or plurality is less suitable for treatment.

In some embodiments, the method further comprises selecting a cell, or enriching for a plurality of cells, in which:

the level of glucose metabolism is lower than a glucose metabolism reference value; or

a level of Stat3 activation that is higher than a Stat3 activation reference value.

In some embodiments, the method further comprises administering the cell of the plurality of cells to a subject.

In some embodiments, the method further comprises obtaining the CAR-expressing cell from the subject prior to evaluating the level of glucose metabolism or Stat3 activation in the CAR-expressing cell.

In some embodiments, responsive to determination that:
i) the level of glucose metabolism is lower than the glucose metabolism reference value; or
ii) the level of Stat3 activation is higher than the Stat3 activation reference value,
   the sample is selected for administration of, or is administered, to the subject.

In some embodiments, responsive to determination that:
i) the level of glucose metabolism is higher than the glucose metabolism reference value,
ii) the level of Stat3 activation is lower than the Stat3 activation reference value,
   the sample is not selected for administration of, or is not administered, to the subject.

In some embodiments, the glucose metabolism reference value is the glucose metabolism value of a cell of a complete responder subject as described in Example 1, e.g., wherein the cell (e.g., a sample containing the cell) is contacted with mock stimulation, e.g., stimulation with an antigen other than the CAR antigen, e.g., as described in Example 1.

In some embodiments, the Stat3 activation reference value is the Stat3 activation value of a cell of a non-responder subject, e.g., as described in Example 2.

In one aspect, the disclosure provides a method of evaluating a CAR-expressing cell, e.g., CAR19− expressing cell, (e.g., CTL019), said method comprising:

evaluating a level of glucose metabolism in the CAR-expressing cell in a sample from a subject, wherein:

a level of glucose metabolism that is lower than a reference value is indicative that the sample is suitable for treatment, and

a level of glucose metabolism that is higher than a reference value is indicative that the sample is less suitable for treatment,

thereby evaluating the CAR-expressing cell.

In one embodiment, the method further comprises obtaining the CAR-expressing cell from the subject prior to evaluating the level of glucose metabolism in the CAR-expressing cell.

In one embodiment, responsive to determination that the level of glucose metabolism is lower than the reference value, the sample is selected for administration of, or is administered, to the subject.

In one embodiment, responsive to determination that the level of glucose metabolism is higher than the reference value, the sample is not selected for administration of, or is not administered, to the subject.

In one embodiment, the reference value is the glucose metabolism value of a cell of a complete responder subject as described in Example 1, e.g., wherein the cell (e.g., a sample containing the cell) is contacted with mock stimulation, e.g., stimulation with an antigen other than the CAR antigen, e.g., as described in Example 1.

Any of the aspects herein, e.g., the immune effector cell compositions and methods above, can be combined with one or more of the embodiments herein, e.g., an embodiment below.

In an embodiment, a method herein comprises making or enriching a population of immune effector cells (e.g., T cells) that can be engineered to express a chimeric antigen receptor (CAR), wherein the method includes performing elutriation. The method can comprise providing a frozen input sample comprising immune effector cells, thawing the frozen input sample, to produce a thawed sample, and performing elutriation on the thawed sample and collecting immune effector cells, thereby producing an output sample comprising immune effector cells that are suitable for expression of a CAR.

In one embodiment, the frozen input sample is a plasma apheresis sample.

In one embodiment, the method further comprises one, two, three or all of:
i) depleting CD19+ cells under flow conditions;
ii) performing density centrifugation using a medium comprising iodixanol, e.g., 60% iodixanol in water (e.g., Optiprep medium), and/or having a density greater than Ficoll (e.g., greater than 1.077 g/ml, e.g., about 1.32 g/ml));
iii) performing a wash step (e.g., on the thawed sample) with a buffer comprising dextrose and/or sodium chloride, e.g., D5 ½ NS medium (5% dextrose and 0.45% sodium chloride), e.g., wherein the wash step is performed using a cell processing device, e.g., a cell washing device or the device used for density gradient centrifugation, e.g., a CS5 (CellSaver5+) instrument; and
iv) performing a positive selection of CD3/CD28+ cells under flow conditions.

In one embodiment, the method further comprises a step of adjusting the viscosity of the thawed sample, e.g., by adding an isotonic solution, e.g., PBS, to the thawed sample.

In one embodiment, the elutriation is performed using a flow rate of from about 30-82 mL/min or 50-80 mL/min and/or the collection volume is about 250-1250 mL or 300-1000 mL for each fraction. In one embodiment, the elutriation is performed using a flow rate of about 30, 40, 50, 60, 70, 72, or 82 mL/min, e.g., about 70 or 72 mL/min. In one embodiment, the elutriation is performed using a flow rate of about 30-40, 40-50, 50-60, 60-70, 70-72, 70-82, 72-82 mL/min. In one embodiment, the elutriation is performed using a collection volume of about 250, 400, 500, 900, or 975 mL, e.g., about 400 or 975 mL. In one embodiment, the elutriation is performed using a collection volume of about 250-400, 400-500, 500-900, 900-1000, or 1000-1259 mL. In one embodiment, the elutriation is performed at about 2400 rpm. In one embodiment, the elutriation is performed at about 2000-2800, 2200-2600, or 2300-2500 rpm.

In one embodiment, the input sample comprises at least 10%, 15%, 20%, 21%, 22%, 23%, 24%, 25%, 26%, 27%, 28%, 29%, 30%, 31%, 35%, or 40% monocytes. In one embodiment, the input sample comprises less than 60%, 55%, 50%, 45%, 40%, 35%, 30%, 25%, or 20% T cells. In one embodiment, the input sample comprises at least 1%, 2%, 5%, 10%, 15%, 20%, 30%, 35%, 40%, 45%, 50%, 55%, 60%, 65%, 70%, 75%, 80%, 85%, 90%, or 95% B cells.

In one embodiment, output sample comprises less than 20%, 15%, 10%, 9%, 8%, 7%, 6%, 5%, 4%, 2%, 2%, 1%, 0.5%,0 .2%, or 0.1% monocytes. In one embodiment, the output sample comprises at least 50%, 55%, 60%, 65%, 70%, 75%, 80%, 85%, 90%, or 95% T cells. In one embodiment, the output sample comprises less than 20%, 15%, 10%, 9%, 8%, 7%, 6%, 5%, 4%, 2%, 2%, 1%, 0.5%,0 .2%, or 0.1% B cells. In one embodiment, the output sample comprises at least 50%, 55%, 60%, 65%, 70%, 75%, 80%, 85%, 90%, 95%, 96%, 97%, 98%, 99%, 99.5%, 99.7%, or 99.9% CD4+CD25+ cells. In one embodiment, the output sample comprises at least 50%, 55%, 60%, 65%, 70%, 75%, 80%, 85%, 90%, 95%, 96%, 97%, 98%, 99%, 99.5%, 99.7%, or 99.9% CD8+CD25+ cells.

In one embodiment, the method results in a T cell yield recovery of at least 50%, 55%, 60%, 65%, 70%, 75%, 80%, 85%, 90%, or 95% T cells.

In one embodiment, the output sample is contacted with a nucleic acid encoding a CAR. In one embodiment, after contacting the output sample with a nucleic acid encoding a CAR, the output sample comprises at least 10%, 15%, 20%, 25%, 30%, 35%, 40%, 45%, or 50% CAR+ cells. In such embodiments, the output sample comprises at least 40%, 45%, 50%, 55%, 60%, 65%, 70%, 75%, 80%. 85%, 90%, or 95% CAR+CD4+ central memory cells. In such embodiments, the output sample comprises at least 40%, 45%, 50%, 55%, 60%, 65%, 70%, 75%, 80%, 85%, 90%, or 95% CAR+CD8+ central memory cells.

In one embodiment, after contacting the output sample with a nucleic acid encoding a CAR, the output sample produces less than 1, 0.9, 0.8, 0.7, 0.6, 0.5, 0.4, 0.3, 0.2, or 0.1 pg of IFN-gamma (IFN-γ) per CAR-expressing cell, e.g., transduced cell. IFN-gamma (IFN-γ) release assays are described herein, e.g., in the Examples. In one embodiment, after contacting the output sample with a nucleic acid encoding a CAR, the output sample comprises a cytotoxicity level (e.g., an EC50rec) of at least 5, 6, 7, 8, 9, 10, 11, 12, 13, 14, 15, 16, 17, 18. 19, 20, 25, or 30. Cyotoxicity assays are described herein, e.g., in the Examples.

In an embodiment, a method herein comprises making or enriching a population of immune effector cells (e.g., T cells) that can be engineered to express a CAR, wherein the method includes performing density gradient centrifugation (also referred to herein as density centrifugation). The method can include providing an input sample comprising immune effector cells, and performing a density centrifugation step using a medium comprising iodixanol, e.g., 60% iodixanol in water, e.g., Optiprep medium and/or having a density greater than Ficoll (e.g., greater than 1.077 g/ml, e.g., about 1.32 g/ml), thereby producing an output sample comprising immune effector cells that are suitable for expression of a CAR.

In one embodiment, the density gradient centrifugation method described herein further comprises performing one, two, three, or all of:
i) depleting CD19+ cells under flow conditions;
ii) elutriation on the input sample, wherein the input sample is optionally a thawed input sample;
iii) performing a wash step (e.g., before density centrifugation) with a buffer comprising dextrose and/or sodium chloride, e.g., D5 ½ NS medium (5% dextrose and 0.45% sodium chloride), e.g., wherein the wash step is performed using a CS5 (CellSaver5+) instrument; and positive selection of CD3/CD28+ cells under flow conditions.

In one embodiment, the density gradient centrifugation method described herein does not comprise one or more of: using a solution comprising glycol, e.g., a Ficoll solution; or performing a wash step in a buffer comprising dextrose and/or sodium chloride, e.g., D5 ½ NS medium, e.g., wherein the wash step is performed using a CS5 instrument; or performing a positive selection step.

In one embodiment, the density centrifugation is performed using a cell separation device, e.g., a Sepax2 device.

In one embodiment, the input sample comprises less than 60%, 55%, 50%, 45%, 40%, 35%, 30%, 25%, 20%, 19%, 18%, 17%, 16%, or 15% T cells. In one embodiment, the input sample comprises at least 10%, 15%, 20%, 21%, 22%, 23%, 24%, 25%, 26%, 27%, 28%, 29%, 30%, 31%, 35%, 40%, 45%, 50%, 55%, 60%, 65%, or 70% monocytes. In one embodiment, the input sample comprises at least 1%, 2%, 5%, 10%, 15%, 20%, 30%, 35%, 40%, 45%, 50%, 55%, 60%, 65%, 70%, 75%, or 80% B cells.

In one embodiment, the output sample comprises at least 25%, 30%, 35%, 40%, 45%, 50%, 55%, 60%, 65%, 70%, 75%, 80%, 85%, 90%, or 95% T cells. In one embodiment, the output sample comprises less than 50%, 45%, 40%, 35%, 30%, 25%, 20%, 15%, 10%, 9%, 8%, 7%, 6%, 5%, 4%, 2%, 2%, 1%, 0.5%, 0.2%, or 0.1% monocytes. In one embodiment, the output sample comprises less than 20%, 15%, 10%, 9%, 8%, 7%, 6%, 5%, 4%, 2%, 2%, 1%, 0.5%, 0.2%, 0.1%, 0.05%, or 0.01% B cells.

In one embodiment, the density gradient centrifugation method described herein results in a T cell yield recovery of at least 5%, 10%, 15%, 20%, 25%, 30%, 35%, 40%, 45%, 50%, 55%, 60%, 65%, 70%, 75%, 80%, 85%, 90%, or 95% T cells.

In an embodiment, a method herein comprises making a population of immune effector cells (e.g., T cells) that can be engineered to express a CAR, wherein the method includes a negative selection step to remove cancer-associated antigen-expressing cells, e.g., CD19− expressing (CD19+) cells. The method can include providing an input sample comprising immune effector cells, and removing CD19+ cells from the input sample under flow conditions, e.g., using a flow-through device, e.g., a cell processing system described herein, thereby producing an output sample comprising immune effector cells that are suitable for expression of a CAR. In one embodiment, the CD19+ cells comprise B cells. In one embodiment, the CD19+ cells comprise lymphoblasts.

In one embodiment, the negative selection method described herein further comprises performing one, two, three or all of:
i) elutriation on the input sample, wherein the input sample is optionally a thawed input sample;
ii) a density centrifugation step using a medium comprising iodixanol, e.g., 60% iodixanol in water (e.g., Optiprep medium), and/or having a density greater than Ficoll (e.g., greater than 1.077 g/ml, e.g., about 1.32 g/ml);
iii) performing a wash step (e.g., before removing CD19+ cells and/or after the input sample is thawed) with a buffer comprising dextrose and/or sodium chloride, e.g., D5 ½ NS medium (5% dextrose and 0.45% sodium chloride), e.g., wherein the wash step is performed using a CS5 (CellSaver5+) instrument; and
iv) positive selection of CD3/CD28+ cells under flow conditions.

In one embodiment, the negative selection method described herein does not comprise performing elutriation or density centrifugation.

In one embodiment, the CD19+ cells are removed from the input sample by magnetic separation. In one embodiment, the magnetic separation comprising contacting the cells with a separation reagent. In one embodiment, the separation reagent comprises a magnetic or paramagnetic member and a CD19-binding member. In one embodiment, the magnetic separation comprises flow cytometry or FACS. In one embodiment, the CD19+ cells are removed by FACS. In one embodiment, the magnetic separation comprises use of a magnetic cell separation device, e.g., CliniMACs device. In one embodiment, the CD19+ cells are removed by a CliniMACs device. In one embodiment, the CD19+ cells are removed by a flow-through device as described herein, e.g., a cell processing system as described herein.

In one embodiment, the input sample comprises at least 1%, 2%, 5%, 10%, 15%, 20%, 30%, 35%, 40%, 45%, 50%, 55%, 60%, 65%, 70%, 75%, 80%, 85%, 90%, 95%, or 99% CD19+ cells. In one embodiment, the output sample comprises less than 20%, 15%, 10%, 9%, 8%, 7%, 6%, 5%, 4%, 2%, 2%, 1%, 0.5%,0 .2%, 0.1%, 0.05%, or 0.01% CD19+ cells. In one embodiment, the output sample comprises less than 50%, 45%, 40%, 40%, 35%, 30%, 25%, 20%, 15%, 10%, 5%, 4%, 2%, 2%, or 1% the percentage of CD19+ cells compared to the input sample.

In one embodiment, the input sample comprises at least 10%, 15%, 20%, 21%, 22%, 23%, 24%, 25%, 26%, 27%, 28%, 29%, 30%, 31%, 35%, or 40% monocytes. In one embodiment, the input sample comprises less than 60%, 55%, 50%, 45%, 40%, 35%, 30%, 25%, or 20% T cells. In one embodiment, the input sample (e.g., the input sample post-wash) comprises at least 1%, 2%, 5%, 10%, 15%, 20%, 30%, 35%, 40%, 45%, 50%, 55%, 60%, 65%, 70%, 75%, or 80% B cells.

In one embodiment, the output sample comprises less than 20%, 15%, 10%, 9%, 8%, 7%, 6%, 5%, 4%, 2%, 2%, 1%, 0.5%,0 .2%, or 0.1% monocytes. In one embodiment, the output sample comprises at least 50%, 55%, 60%, 65%, 70%, 75%, 80%, 85%, 90%, or 95% T cells. In one embodiment, the output sample comprises less than 20%, 15%, 10%, 9%, 8%, 7%, 6%, 5%, 4%, 2%, 2%, 1%, 0.5%,0 .2%, 0.1%, 0.05%, or 0.01% B cells.

In one embodiment, the negative selection method described herein results in a T cell yield recovery of at least 50%, 55%, 60%, 65%, 70%, 75%, 80%, 85%, 90%, or 95% T cells.

In an embodiment, a method herein comprises making a population of immune effector cells (e.g., T cells) that can be engineered to express a CAR, wherein the method comprises positive selection. The method can include providing an input sample comprising immune effector cells, and positively selecting for CD3+/CD28+ cells from the input sample under flow conditions, thereby producing an output sample comprising immune effector cells that are suitable for expression of a CAR, e.g., wherein the positive selection is performed under flow conditions.

In one embodiment, the positive selection method described herein further comprises performing one, two, three, or all of:
i) depleting CD19+ cells, e.g., under flow conditions; elutriation on the input sample, wherein the input sample is optionally a thawed input sample;
ii) performing density centrifugation using a medium comprising iodixanol, e.g., 60% iodixanol in water (e.g., Optiprep medium), and/or having a density greater than Ficoll (e.g., greater than 1.077 g/ml, e.g., about 1.32 g/ml); and
iii) performing a wash step (e.g., before removing CD19+ cells and/or after the input sample is thawed) with a buffer comprising dextrose and/or sodium chloride, e.g., D5 ½ NS medium (5% dextrose and 0.45% sodium chloride), e.g., wherein the wash step is performed using a CS5 (CellSaver5+) instrument.

In one embodiment, the positive selection method described herein further comprises performing elutriation on the input sample (e.g., wherein the input sample is a thawed input sample). Optionally, the elutriation is performed together with one or more of (e.g., 1, 2, or all of) depleting CD19+ cells (e.g., as described in (i) above), performing density centrifugation (e.g., as described in (ii) above), and performing a wash step (e.g., as described in (iii) above).

In one embodiment, the positive selection method described herein further comprises performing elutriation, a wash step (optionally), and density centrifugation (e.g., using Ficoll or OptiPrep medium) prior to performing positive selection. In one embodiment, the positive selection method described herein further comprises performing a wash step (optionally) and density centrifugation (e.g., using Ficoll or OptiPrep medium) prior to performing positive selection. In one embodiment, the positive selection method described herein does not comprise performing elutriation. In one embodiment, the positive selection method described herein further comprises performing a wash with a buffer comprising dextrose and/or sodium chloride, e.g., D5 ½ NS buffer, e.g., using a CS5+ instrument.

In one embodiment, the positive selection comprises contacting the input sample with a separation reagent, which separation reagent comprises a magnetic or paramagnetic member and a CD3 and/or CD28-binding member. In one embodiment, the positive selection for CD3+/CD28+ cells comprises incubating the input sample with a separation reagent for about 10 to 90 minutes, about 10 to 60 minutes, about 10 to 45 minutes, about 12 to 90 minutes, about 12 to 60 minutes, about 12 to 45 minutes, about 15 to 90 minutes, about 15 to 60 minutes, about 15 to 45 minutes, e.g., about 30 minutes or about 20 minutes. In one embodiment, the separation reagent comprises a bead that is coupled (e.g., covalently or non-covalently coupled) to an anti-CD3 and/or anti-CD28 antibody. In one embodiment, the positive selection uses an about 3:1 ratio of magnetic separation members (e.g., beads) to T cells.

In one embodiment, the positive selection comprises flowing a fluid that comprises the immune effector cells and magnetic separation members within an enclosed system, e.g., a chamber or a bag, where magnetic separation occurs. In one embodiment, the flowing is performed at a speed such that magnetic separation of the members (optionally bound to immune effector cells) occurs. In one embodiment, the positive selection for CD3+/CD28+ cells comprises a separation or dwell time of less than about 6, 5, 6, 3, 2, or 1 minute, or less than about 50, 40, 30, 20, 10, 5, 4, 3, 2, or 1 second.

In one embodiment, the positive selection is performed with a magnetic device, e.g., Dynamag CTS, a flow-through device comprising magnetic elements as described herein, or other arrangement of magnetic elements.

In one embodiment, the positive selection is performed using a device that includes at least one cell suspension module; at least one flow-through magnetic separation/debeading module; at least one non-magnetic output module; at least one magnetic output module; optionally, at least one magnetic component, external to the magnetic separation/debeading module, that creates magnetic forces and/or gradients; and optionally, at least one buffer module. In one embodiment, the device further comprises at least one magnetic component, external to the magnetic separation/debeading module, which creates magnetic forces and/or gradients. In one embodiment, the device further comprises at least one buffer module. In one embodiment, the magnetic separation/debeading module comprises a chamber defined by walls and having an x-direction, a y-direction, and a z-direction; an inlet and an outlet arranged on opposite ends of the chamber, e.g., in the x-direction, in the y-direction, or in the z-direction; at least two magnets adjacent or proximate to a wall of the chamber and arranged to establish a zero gradient line within the chamber between the inlet and the outlet. In one embodiment, the immune effector cells flow through the chamber, wherein each point in the chamber is within 2 cm of the magnets.

In one embodiment, the positive selection method comprises (e.g., between steps a) and b)), contacting the immune effector cells with a solution comprising dextrose and/or sodium chloride, e.g., D5 ½ NS medium (5% dextrose and 0.45% sodium chloride), optionally, wherein the solution is at ambient temperature, e.g., at about 20-25° C. In one embodiment, the immune effector cells are present in a flexible container, e.g., a bag, e.g., during steps a) and b). In one embodiment, the method comprises (e.g., between steps a) and b), e.g., after contacting the immune effector cells with the saline solution), placing the bag on a thermal insulating material, e.g., a plurality of layers comprising paper, e.g., paper towels or wipes. In an embodiment, the method comprises (e.g., after step b)), incubating the cells at about 37° C. for about 10 minutes. In an embodiment, the method comprises (e.g., after step b)), incubating the cells at about 36-38, 35-39, or 34-40° C., e.g., for about 10 minutes. In an embodiment, the incubation step lasts about 8-12, 5-15, or 5-20 minutes. In an embodiment, the incubation is performed in a Plasmatherm device.

In an embodiment of the positive selection method, the input sample comprising immune effector cells comprises at least 20% monocytes. In an embodiment, the input sample comprising immune effector cells comprises at least 10%, 15%, 20%, 25%, 30%, 35%, 40%, 50%, 60% monocytes.

In one embodiment, the positive selection method comprises one or more of (e.g., 2, 3, 4, or all of), e.g., in the order listed:
a) thawing a frozen input sample (e.g., a leukapheresis sample) comprising immune effector cells from a patient having a hematologic malignancy, optionally wherein the sample comprises >20% lymphoblasts;
b) washing the immune effector cells, e.g., at ambient temperature, e.g., 20-25° C., in a wash solution, e.g., X-VIV015 medium (Lonza), called ‘Modified Medium’ (MM).
c) contacting the input sample with a separation reagent, which separation reagent comprises a magnetic or paramagnetic member and a CD3 and/or CD28-binding member;
d) rotating the input sample and separation reagent on a rotator, e.g., at 2-6 rpm, e.g., at 4 rpm, wherein the rotation lasts for, e.g., 10-30 minutes, e.g., 20 minutes; and
e) performing positive selection to enrich for cells that bind the separation reagent, e.g., for 30 sec to 2 minutes, e.g., for 1 minute.

In one embodiment, the positive selection method comprises one or more of (e.g., 2, 3, 4, 5, 6, or all of), e.g., in the order listed:
a) thawing a frozen input sample (e.g., a leukapheresis sample) comprising immune effector cells from a patient having a hematologic malignancy, optionally wherein the sample comprises >20% monocytes;
b) washing the immune effector cells, e.g., at ambient temperature, e.g., 20-25° C., in a wash solution, e.g., comprising about 5% dextrose and 0.45% sodium chloride, e.g., D5 ½ NS;
c) placing a flexible container comprising the cells on a thermal insulating material, e.g., a plurality of layers comprising paper, e.g., paper towels or wipes;
d) contacting the input sample with a separation reagent, which separation reagent comprises a magnetic or paramagnetic member and a CD3 and/or CD28-binding member;
e) incubating the input sample and separation reagent e.g., at 37° C., e.g., for 5-15 minutes, e.g., 10 minutes;
f) rotating the input sample and separation reagent on a rotator, e.g., at 2-6 rpm, e.g., at 4 rpm, wherein the rotation lasts for, e.g., 10-30 minutes, e.g., 20 minutes; and
g) performing positive selection to enrich for cells that bind the separation reagent, e.g., for 30 sec to 2 minutes, e.g., for 1 minute.

In an embodiment, the sample, e.g., the input sample, is from a patient having a hematologic malignancy, e.g., a hematologic malignancy described herein, e.g., ALL or DLBCL.

In one embodiment, the input sample comprises about 1×10⁵ nucleated cells/ml, 2×10⁵ nucleated cells/ml, 5×10⁵ nucleated cells/ml, 7×10⁵ nucleated cells/ml, 1×10⁶ nucleated cells/ml, 2×10⁶ nucleated cells/ml, 5×10⁶ nucleated cells/ml, 7×10⁶ nucleated cells/ml, 1×10⁷ nucleated cells/ml , 2×10⁷ nucleated cells/ml, 5×10⁷ nucleated cells/ml, 7×10⁷ nucleated cells/ml, 1×10⁷ nucleated cells/ml, 2×10⁵ nucleated cells/ml, 5×10⁵ nucleated cells/ml, and 7×10⁸ nucleated cells/ml. In one embodiment, the input sample comprises about 1-1.5×10⁷ T cells.

In one embodiment, the input sample comprises at least 5%, 10%, 15%, 20%, 25%, 30%, 35%, 40%, 45%, 50%, 55%, 60%, 65%, 70%, 75%, 80%, 85%, 90%, or 95% monocytes.

In one embodiment, the input sample comprises at least 5%, 10%, 15%, 20%, 25%, 30%, 35%, 40%, 45%, 50%, 55%, 60%, 65%, 70%, 75%, 80%, 85%, 90%, or 95% tumor cells, e.g., lymphoblasts. In one embodiment, the input sample comprises less than 60%, 55%, 50%, 45%, 40%, 35%, 30%, 25%, or 20% immune effector cells, e.g., T cells. In one embodiment, the input sample comprises at least about 5%, 10%, 15%, 18%, 20%, 25%, 30%, 35%, 40%, 45%, 50%, 55%, 60%, 65%, 70%, 75%, 80%, 85%, 90%, or 95% B cells, e.g., CD45+CD19+ B cells. In one embodiment, the input sample comprises at least about 5%, 10%, 15%, 18%, 20%, 25%, 30%, 35%, 40%, 45%, 50%, 55%, 60%, 65%, 70%, 75%, 80%, 85%, 90%, or 95% B cells, e.g., CD45−CD19+ B cells.

In one embodiment, the output sample comprises less than 20%, 15%, 10%, 9%, 8%, 7%, 6%, 5%, 4%, 2%, 2%, 1%, 0.5%,0 .2%, or 0.1% monocytes. In one embodiment, the output sample comprises less than 20%, 15%, 10%, 9%, 8%, 7%, 6%, 5%, 4%, 2%, 2%, 1%, 0.5%,0 .2%, or 0.1% tumor cells. In one embodiment, the output sample comprises at least 50%, 55%, 60%, 65%, 70%, 75%, 80%, 85%, 90%, 95%, 96%, 97%, 98%, 99%, 99.5%, 99.8%, or 99.9% immune effector cells, e.g., T cells. In one embodiment, the output sample comprises at least 25%, 30%, 35%, 40%, 45%, 50%, 55%, 60%, 65%, 70%, 75%, 80%, 85%, 90%, 95%, 96%, 97%, 98%, or 99% T cells, e.g., CD3+CD45+ T cells. In one embodiment, the output sample comprises less than about 10%, 9%, 8%, 7%, 6%, 5%, 4%, 3%, 2%, or 1% B cells, e.g., CD45+CD19+ B cells. In one embodiment, the output sample comprises less than about 10%, 9%, 8%, 7%, 6%, 5%, 4%, 3%, 2%, or 1% B cells, e.g., CD45−CD19+ B cells. In an embodiment, the output sample comprises at least 5%, 6%, 7%, 8%, 9%, 10%, 11%, 12%, 13%, 14%, 15%, or 20% T cells.

In one embodiment, a method herein comprises making a population of immune effector cells (e.g., T cells) that can be engineered to express a chimeric antigen receptor (CAR), wherein the method includes:
i) providing an input sample, e.g., a frozen input sample or a fresh input sample comprising immune effector cells; optionally, wherein the input sample is a frozen input sample, thawing the frozen input sample, to produce a thawed sample;
ii) performing an enrichment step, wherein the enrichment step comprises: performing elutriation on the input sample, wherein the input sample is optionally a thawed input sample; or performing density centrifugation step using a medium comprising iodixanol, e.g., 60% iodixanol in water, e.g., Optiprep medium, and/or having a density greater than Ficoll (e.g., greater than 1.077 g/ml, e.g., about 1.32 g/ml); and
iii) performing a selection step, wherein the selection is a positive selection, e.g., for CD3/CD28+ cells, or a negative selection, e.g., for CD19+, CD25+, or CD14+ cells;
   thereby producing an output sample comprising immune effector cells that are suitable for expression of a CAR.

In one embodiment, a method herein comprises making a population of immune effector cells (e.g., T cells) that can be engineered to express a chimeric antigen receptor (CAR), the method comprising:
i) providing an input sample, e.g., a frozen input sample or a fresh input sample comprising immune effector cells;
ii) optionally, wherein the input sample is a frozen input sample, thawing the frozen input sample, to produce a thawed sample;
iii) performing an enrichment step, wherein the enrichment step comprises:
   1) performing elutriation on the input sample, wherein the input sample is optionally a thawed input sample; or
   2) performing density centrifugation step using a medium comprising iodixanol, e.g., 60% iodixanol in water, e.g., Optiprep medium, and/or having a density greater than Ficoll (e.g., greater than 1.077 g/ml, e.g., about 1.32 g/ml); and
iv) performing a selection step, wherein the selection is a positive selection, e.g., for CD3/CD28+ cells, or a negative selection, e.g., for CD19+, CD25+, or CD14+ cells;
   thereby producing an output sample comprising immune effector cells that are suitable for expression of a CAR.

In one embodiment, a method herein comprises making a population of immune effector cells (e.g., T cells) that can be engineered to express a chimeric antigen receptor (CAR), wherein the method includes:
i) providing an input sample, e.g., a frozen input sample or a fresh input sample, comprising immune effector cells;
ii) performing an enrichment step, wherein the enrichment step comprises: performing elutriation or density centrifugation (e.g., using Ficoll or a Optiprep medium);
iii) performing a selection step, wherein the selection is a positive selection, e.g., for CD3/CD28+ cells, or a negative selection, e.g., for CD19+, CD25+, or CD14+ cells;
   thereby producing an output sample comprising immune effector cells that are suitable for expression of a CAR. In one embodiment, the selection step is performed under flow conditions, e.g., by using a flow-through device.

Additional features or embodiments of any of the methods or compositions described herein include one or more of the following:

An input sample in any of the embodiments of any of the methods described herein is a biological sample from a subject that comprises immune effector cells, e.g., T cells and/or NK cells. In an embodiment, the input sample is a blood sample, e.g., a whole blood sample. In an embodiment, the input sample is an apheresis sample, e.g., a leukapheresis sample. In one embodiment, the input sample is a fresh sample, in which the sample has been obtained from the subject and is processed using any of the methods described herein within 1 day, 2 days, 5 days, or 7 days of obtaining from the subject. In one embodiment, the input sample is a frozen or cryopreserved sample, e.g., frozen at −20° C. or in liquid nitrogen or frozen to −80° C. at a rate of 1° per minute and stored in the vapor phase of a liquid nitrogen storage tank.

In embodiments of any of the methods described herein, the input sample comprises at least 5%, 10%, 15%, 20%, 25%, 30%, 35%, 40%, 45%, 50%, 55%, 60%, 65%, 70%, 75%, 80%, 85%, 90%, or 95% monocytes (and optionally up to 40%, 70%, or 95% monocytes). In embodiments of any of the methods described herein, the input sample comprises at least 5%, 10%, 15%, 20%, 25%, 30%, 35%, 40%, 45%, 50%, 55%, 60%, 65%, 70%, 75%, 80%, 85%, 90%, or 95% tumor cells, e.g., lymphoblasts (and optionally up to 50% or 95% monocytes). In embodiments of any of the methods described herein, the input sample comprises less than 60%, 55%, 50%, 45%, 40%, 35%, 30%, 25%, or 20% immune effector cells, e.g., T cells (and optionally greater than 20% T cells).

In embodiments of any of the methods described herein, the output sample comprises less than 20%, 15%, 10%, 9%, 8%, 7%, 6%, 5%, 4%, 2%, 2%, 1%, 0.5%,0 .2%, or 0.1% monocytes (and optionally greater than 1% or 0.1% monocytes). In embodiments of any of the methods described herein, the output sample comprises less than 20%, 15%, 10%, 9%, 8%, 7%, 6%, 5%, 4%, 2%, 2%, 1%, 0.5%,0 .2%, or 0.1% tumor cells (and optionally greater than 1% or 0.1% tumor cells). In embodiments of any of the methods described herein, the output sample comprises at least 50%, 55%, 60%, 65%, 70%, 75%, 80%, 85%, 90%, 95%, 96%, 97%, 98%, 99%, 99.5%, 99.8%, or 99.9% immune effector cells, e.g., T cells (and optionally up to 60% or 95% T cells).

In embodiments of any of the methods described herein, the output sample comprises less than 50%, 45%, 40%, 40%, 35%, 30%, 25%, 20%, 15%, 10%, 5%, 4%, 2%, 2%, or 1% the percentage of monocytes compared to the input sample. In embodiments of any of the methods described herein, the output sample comprises less than 50%, 45%, 40%, 40%, 35%, 30%, 25%, 20%, 15%, 10%, 5%, 4%, 2%, 2%, or 1% the percentage of tumor cells compared to the input sample. In embodiments of any of the methods described herein, the output sample comprises at least 50%, 45%, 40%, 40%, 35%, 30%, 25%, 20%, 15%, 10%, 5%, 4%, 2%, 2%, or 1% the percentage of immune effector cells, e.g., T cells, compared to the input sample.

In embodiments of any of the methods described herein, the method further comprises introducing, e.g., by transduction, a nucleic acid encoding a CAR into one or more of the immune effector cells in the output sample. Other methods for introducing a nucleic acid encoding a CAR are described herein.

In embodiments of any of the methods described herein, the CAR comprises an antigen binding domain, a transmembrane domain, and an intracellular signaling domain, e.g., comprising a primary signaling domain and/or a costimulatory signaling domain.

In embodiments of any of the methods described herein, the methods further comprise a step of assaying the transduction efficiency. In embodiments of any of the methods described herein, the transduction results in a transduction efficiency of at least about 10%, 15%, 20%, 25%, 30%, 35%, 40%. 45%, or 50%.

In embodiments of any of the methods described herein, the methods further comprise performing a wash step on the input sample with a buffer comprising dextrose and/or sodium chloride, e.g., D5 medium, e.g., using a CS5+ instrument.

In embodiments of any of the methods described herein, the immune effector cells are human immune effector cells.

In embodiments of any of the methods described herein, the output sample comprises CD8+ T cells. In embodiments of any of the methods described herein, the output sample comprises CD4+ T cells.

In embodiments of any of the methods described herein, the input sample is from a patient that has a disease associated with a tumor antigen, e.g., a tumor antigen described herein, e.g., CD19, is selected from a proliferative disease such as a cancer or malignancy or a precancerous condition such as a myelodysplasia, a myelodysplastic syndrome or a preleukemia, or is a non-cancer related indication associated with expression of a tumor antigen described herein. In one embodiment, the disease is a cancer described herein, e.g., a cancer described herein as being associated with a target described herein. In one embodiment, the hematologic cancer is leukemia. In one embodiment, the cancer is selected from the group consisting of one or more acute leukemias including but not limited to B-cell acute lymphoid leukemia (“BALL”), T-cell acute lymphoid leukemia (“TALL”), acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and to disease associated with expression of a tumor antigen described herein include, but not limited to, atypical and/or non-classical cancers, malignancies, precancerous conditions or proliferative diseases expressing a tumor antigen as described herein; and any combination thereof. In another embodiment, the disease associated with a tumor antigen described herein is a solid tumor, e.g., a solid tumor described herein, e.g., prostatic, colorectal, pancreatic, cervical, gastric, ovarian, head, or lung cancer.

In embodiments of any of the methods described herein, the input sample is from a patient that has a cancer selected from the group consisting of one or more acute leukemias including but not limited to B-cell acute lymphoid leukemia (BALL), T-cell acute lymphoid leukemia (TALL), acute lymphoid leukemia (ALL); one or more chronic leukemias including but not limited to chronic myelogenous leukemia (CML), chronic lymphocytic leukemia (CLL); additional hematologic cancers or hematologic conditions including, but not limited to B cell prolymphocytic leukemia, blastic plasmacytoid dendritic cell neoplasm, Burkitt's lymphoma, diffuse large B cell lymphoma, follicular lymphoma, hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm. Waldenstrom macroglobulinemia, preleukemia, atypical and/or non-classical cancers, malignancies, precancerous conditions or proliferative diseases, and any combination thereof.

In embodiments of any of the methods described herein, the input sample is from a patient that has ALL.

In embodiments of any of the methods described herein, the method further comprises a step of assaying one or more cell surface markers on cells in the output sample, e.g., CD45, CD19, CD3, CD28, CD25, or CD14.

In embodiments of any of the methods described herein, the method further comprises stimulating the output sample with an agent that stimulates proliferation of the immune effector cells, e.g., stimulates a CD3/TCR complex associated signal and/or a ligand that stimulates a costimulatory molecule on the surface of the T cells, e.g., an anti-CD3 antibody and an anti-CD28 antibody.

In embodiments of any of the methods described herein, the method further comprises introducing a nucleic acid encoding a CAR, e.g., by transduction, transfection, or electroporation.

In another aspect, the present disclosure features a reaction mixture produced by a method disclosed herein, e.g., a method disclosed above.

In another embodiment, a reaction mixture herein comprises at least 80%, 85%, 90%, or 95% T cells and less than 10%, 9%, 8%, 7%, 6%, 5%, 4%, 3%, 2%, or 1% monocytes, wherein the total number of cells in the reaction mixture adds up to 100%. In one embodiment, the reaction mixture comprises at least 1×10⁶, 2×10⁶, 5×10⁶, 1×10⁷, 2×10⁷, 5×10⁷, 1×10⁸, 2×10⁸, or 5×10⁸ cells total. In one embodiment, the reaction mixture comprises less than 10%, 9%, 8%, 7%, 6%, 5%, 4%, 3%, 2%, or 1% B cells. In one embodiment, the reaction mixture comprises less than 10%, 9%, 8%, 7%, 6%, 5%, 4%, 3%, 2%, or 1% cancer cells, e.g. lymphoblasts.

In any of the reaction mixtures described herein, one or more of the T cells expresses a CAR, e.g., any CAR described herein.

In any of the reaction mixtures described herein, the reaction mixture further comprises a nucleic acid encoding a CAR, e.g., wherein the nucleic acid is disposed inside a T cell or outside a T cell.

Although methods and materials similar or equivalent to those described herein can be used in the practice or testing of the present invention, suitable methods and materials are described below. All publications, patent applications, patents, and other references (e.g., sequence database reference numbers) mentioned herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of Oct. 25, 2017. When one gene or protein references a plurality of sequence accession numbers, all of the sequence variants are encompassed.

In addition, the materials, methods, and examples are illustrative only and not intended to be limiting. Headings, sub-headings or numbered or lettered elements, e.g., (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE FIGURES

FIGS. 1A-1D how transcriptional profiles of CAR T cellular products which reveal T cell-intrinsic quality attributes associated with clinical response. Each point represents the relative enrichment of these signatures in individual patient cellular product samples and bars reflect minimum to maximum values. The normalized enrichment score for each gene set is plotted on the y-axis (*P<0.05; **P<0.01; ***P<0.001 by two-tailed Welch's t-test). FIG. 1A shows relative enrichment for the Early Memory-Late Memory gene set. FIG. 1B shows relative enrichment for the Memory-Effector gene set. FIG. 1C shows relative enrichment for the High Glycolysis-Low Glycolysis gene set. FIG. 1D shows relative enrichment for the High Exhaustion-Low Exhaustion gene set.

FIG. 2 shows uptake of the fluorescent glucose analog 2-NBDG in mock- or CAR-stimulated retrospective patient CTL019 cells as assessed by flow cytometry (**P<0.01, paired two-tailed t-test).

FIG. 3 shows a comparison of 2-NBDG uptake between responder (CR, n=4; PRTD, n=2; PR, n=2) and non-responder (NR, n=6) CTL019 samples (**P<0.01, un-paired two-tailed t-test; NS, non-significant).

FIG. 4 shows representative flow cytometry depicting the differentiation phenotype of CD8+ CAR T cells following 9 days of culture in the absence (control) or presence of 2-deoxy-D-glucose (2-DG), which inhibits glycolysis.

FIG. 5 shows the frequency (%) of T-cell subsets within CD8+ and CD4+ CAR T cells following culture in the presence or absence of 2-DG (**P<0.01, *P<0.05, paired two-tailed t-test). The following T-cell subsets are shown: naïve-like (CCR7+CD45RO−); central memory (CCR7+CD45RO+); effector memory (CCR7−CD45RO+); and effector (CCR7−CD45RO−).

FIG. 6 shows the proliferative capacity of CTL019 cells manufactured in the presence or absence of 2-DG. CAR T cells were serially re-stimulated with K562 cells engineered to express CD19 or mesothelin (irrelevant target antigen) on days 0, 7 and 12 of the culture, as indicated by the arrows. Data from two representative subjects is shown.

FIGS. 7A-7D show IL-6/STAT3 pathway enrichment in CAR T cells. FIG. 7A shows levels of soluble cytokines produced from CAR-stimulated CTL019 cells over baseline levels elaborated by matched, unstimulated controls in evaluable patient samples from each response category (CR, n=6; PRTD, n=3; PR, n=5; NR, n=21; *P<0.05; **P<0.01 by a two-tailed Mann-Whitney test). Graphs show mean with s.e.m. FIG. 7B shows single-sample enrichment analysis of the IL-6/STAT3 pathway in CAR-stimulated CTL019 cells from patients in each response group (*P<0.05 using a two-tailed un-paired t-test). Bars represent the mean and s.e.m. FIG. 7C shows representative flow cytometry plots showing levels of pSTAT3 in pre-infusion CTL019 cells from a CR and NR patient after overnight stimulation with isotype control antibody-coated beads (mock stimulated) or beads coated with an anti-idiotypic antibody against CAR19 (CAR19 stimulated) (left panel). Pooled data from patients with highly functional (CR, n=3; PR_{TD}, n=2) versus poorly functional (PR, n=3; NR, n=11) CAR T cells is shown in the box plots (right panel). Whiskers represent min. to max.; boxes represent 25-75 percentiles; the middle line indicates the median. The change in fluorescence intensity (MFI) was calculated by subtracting the MFI of pSTAT3 in stimulated cells from that in unstimulated cells. FIG. 7D shows Spearman's rho correlation between the maximum in vivo proliferative capacity of adoptively-transferred CAR T cells and peak levels of serum IL-6 (within 28 days post-CTL019 infusion; left panel) or IL-6/STAT3 gene enrichment in patient-matched CAR-stimulated (as above) CTL019 cells (right panel).

FIGS. 8A-8C show inhibition of the STAT3 pathway in CAR T cells. FIG. 8A shows representative flow cytometry plots depicting levels of pSTAT3 in CTL019 cells that were stimulated overnight with isotype control beads (mock) or beads coated with an anti-idiotypic antibody against CAR19. CAR-specific stimulations were performed in the presence or absence of 5 μM Stattic, a small molecule inhibitor of STAT3 activation. FIG. 8B shows expansion capacity of CTL019 cells manufactured in the presence or absence of 5 μM Stattic or an equivalent amount of DMSO (control). CAR T cells from this representative subject were then serially re-stimulated with K562 cells engineered to express CD19 on days 0, 7 and 12 of the culture, as indicated by the arrows. The fold change in CAR T cell number from baseline is displayed with solid lines (left y-axis) in parallel with cell viability displayed with dashed lines (right y-axis). FIG. 8C shows the summary of cell proliferation and viability data on CTL019 cells from n=8 different subjects that were expanded with or without 5 μM Stattic prior to re-stimulation.

FIG. 9 shows a graph depicting expansion capacity of CTL019 cells manufactured in the presence or absence of recombinant IL-6 or an equivalent amount of DMSO (control). CTL019 cells from this representative subject were then serially re-stimulated with K562 cells engineered to express CD19 or mesothelin (negative control) on days 0, 7, and 12 of the culture, as indicted by the arrows.

FIG. 10 shows receiver operating characteristic (ROC) curves based on total doses of

CAR T cells (cells/kg) possessing different phenotypes that were infused into responding (n=14) versus non-responding (n=21) patients.

FIGS. 11A-11B show levels of pSTAT3 in CD27+ PD-1− CD8 T cells in response to IL-6. FIG. 11A shows representative histograms (left panel) showing levels of pSTAT3 in CD8+ T cell populations that were purified by fluorescence-activated cell sorting and stimulated with recombinant IL-6 (10 ng/ml). Summary of IL-6-induced pSTAT3 levels in CD8+ T cell subsets defined by CD27 and PD-1 expression from n=4 different subjects is shown in the right panel. The change in mean fluorescence intensity (AMFI) was determined by subtracting the MFI of pSTAT3 in IL-6-treated cells from the same parameter in matched, unstimulated cells. FIG. 11B shows representative flow cytometry (left panel) and pooled data from n=7 different subjects (right panel) showing levels of the interleukin-6 receptor subunit beta (CD130) in CD8+ T cell populations defined by CD27 and PD-1 expression.

FIG. 12 shows graphs depicting gp130 expression on CD8+ T cell subsets (left panel) or frequencies of CD8+ T cell subsets expressing gp130 (right panel). CD8+ T cell subsets are grouped into: CD27+ CD45RO−, CD27+ CD45RO+, CD27− CD45RO+ and CD27− CD45RO− cells.

FIG. 13 shows a vector comprising a constitutively active STATS (STAT3C) construct.

FIGS. 14A-14B show gp130 expression on T cells and selection of T cells with gp130. FIG. 14A shows expression of gp130 in CD4+ T cells (top row) or CD8+ T cells (bottom row). The dot plots show flow cytometry data with gp130 on the x-axis and CCR7, CD27, PD1 or CD45RO on the y-axis. FIG. 14B shows gp130-based positive selection of CD4+ T cells (top row) or CD8+ T cells (bottom row). The panels on the left show expression of CD27 and CD45RO in the T cells before selection and the panels on the right show expression of the same markers after selection with gp130.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “glucose metabolism” refers to a process, e.g., one or more biochemical processes, involving the formation, breakdown or interconversion of glucose in a living organism. As used herein, a “glucose metabolism value” refers to a measure of glucose metabolism, e.g., as assayed by a glucose uptake cell-based kit with 2-NBDG (a fluorescently-labeled deoxyglucose analog), or a glucose colorimetric assay kit.

The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. In some embodiments, the set of polypeptides are in the same polypeptide chain (e.g., comprise a chimeric fusion protein). In some embodiments, the set of polypeptides are not contiguous with each other, e.g., are in different polypeptide chains. In some embodiments, the set of polypeptides include a dimerization switch that, upon the presence of a dimerization molecule. can couple the polypeptides to one another, e.g., can couple an antigen binding domain to an intracellular signaling domain. In one embodiment, the stimulatory molecule of the CAR is the zeta chain associated with the T cell receptor complex. In one aspect, the cytoplasmic signaling domain comprises a primary signaling domain (e.g., a primary signaling domain of CD3-zeta). In one embodiment, the cytoplasmic signaling domain further comprises one or more functional signaling domains of at least one costimulatory molecule as defined below. In one embodiment, the costimulatory molecule is a costimulatory molecule described herein, e.g., 4-1BB (i.e., CD137), CD27, ICOS, and/or CD28. In one embodiment, the CAR comprises a chimeric fusion protein comprising an extracellular antigen binding domain, a transmembrane domain and an intracellular signaling domain comprising a functional signaling domain of a stimulatory molecule. In one embodiment, the CAR comprises a chimeric fusion protein comprising an extracellular antigen binding domain, a transmembrane domain and an intracellular signaling domain comprising a functional signaling domain of a co-stimulatory molecule and a functional signaling domain of a stimulatory molecule. In one embodiment, the CAR comprises a chimeric fusion protein comprising an extracellular antigen binding domain, a transmembrane domain and an intracellular signaling domain comprising two functional signaling domains of one or more co-stimulatory molecule(s) and a functional signaling domain of a stimulatory molecule. In one embodiment, the CAR comprises a chimeric fusion protein comprising an extracellular antigen binding domain, a transmembrane domain and an intracellular signaling domain comprising at least two functional signaling domains of one or more co-stimulatory molecule(s) and a functional signaling domain of a stimulatory molecule. In one embodiment, the CAR comprises an optional leader sequence at the amino-terminus (N-terminus) of the CAR fusion protein. In one embodiment, the CAR further comprises a leader sequence at the N-terminus of the extracellular antigen binding domain, wherein the leader sequence is optionally cleaved from the antigen binding domain (e.g., a scFv) during cellular processing and localization of the CAR to the cellular membrane.

A CAR that comprises an antigen binding domain (e.g., a scFv, or TCR) that targets a specific tumor antigen X, such as those described herein, is also referred to as XCAR. For example, a CAR that comprises an antigen binding domain that targets CD19 is referred to as CD19CAR.

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an antibody, that retains the ability to specifically interact with (e.g., by binding, steric hindrance, stabilizing/destabilizing, spatial distribution) an epitope of an antigen. Examples of antibody fragments include, but are not limited to, Fab, Fab′, F(ab′)₂, Fv fragments, scFv antibody fragments, disulfide-linked Fvs (sdFv), a Fd fragment consisting of the VH and CH1 domains, linear antibodies, single domain antibodies such as sdAb (either VL or VH), camelid VHH domains, multi-specific antibodies formed from antibody fragments such as a bivalent fragment comprising two Fab fragments linked by a disulfide bridge at the hinge region, and an isolated CDR or other epitope binding fragments of an antibody. An antigen binding fragment can also be incorporated into single domain antibodies, maxibodies, minibodies, nanobodies, intrabodies, diabodies, triabodies, tetrabodies, v-NAR and bis-scFv (see, e.g., Hollinger and Hudson, Nature Biotechnology 23:1126-1136, 2005). Antigen binding fragments can also be grafted into scaffolds based on polypeptides such as a fibronectin type TIT (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL.

The portion of a CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv) and a humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988, Proc. Natl. Acad. Sci. USA 85:5879-5883; Bird et al., 1988, Science 242:423-426). In one embodiment, the antigen binding domain of a CAR comprises an antibody fragment. In a further embodiment, the CAR comprises an antibody fragment that comprises a scFv.

As used herein, the term “binding domain” or “antibody molecule” refers to a protein, e.g., an immunoglobulin chain or fragment thereof, comprising at least one immunoglobulin variable domain sequence. The term “binding domain” or “antibody molecule” encompasses antibodies and antibody fragments. In an embodiment, an antibody molecule is a multispecific antibody molecule, e.g., it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The portion of the CAR of the invention comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, N.Y.; Houston et al., 1988. Proc. Natl. Acad. Sci. USA 85:5879-5883; Bird et al., 1988, Science 242:423-426). In one aspect, the antigen binding domain of a CAR composition of the invention comprises an antibody fragment. In a further aspect, the CAR comprises an antibody fragment that comprises a scFv.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (κ) and lambda (λ) light chains refer to the two major antibody light chain isotypes.

The term “complementarity determining region” or “CDR,” as used herein, refers to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, Md. (“Kabat” numbering scheme), Al-Lazikani et al., (1997) JMB 273,927-948 (“Chothia” numbering scheme), or a combination thereof. Under the Kabat numbering scheme, in some embodiments, the CDR amino acid residues in the heavy chain variable domain (VH) are numbered 31-35 (HCDR1), 50-65 (HCDR2), and 95-102 (HCDR3); and the CDR amino acid residues in the light chain variable domain (VL) are numbered 24-34 (LCDR1), 50-56 (LCDR2), and 89-97 (LCDR3). Under the Chothia numbering scheme, in some embodiments, the CDR amino acids in the VH are numbered 26-32 (HCDR1), 52-56 (HCDR2), and 95-102 (HCDR3); and the CDR amino acid residues in the VL are numbered 26-32 (LCDR1), 50-52 (LCDR2), and 91-96 (LCDR3). In a combined Kabat and Chothia numbering scheme, in some embodiments, the CDRs correspond to the amino acid residues that are part of a Kabat CDR, a Chothia CDR, or both. For instance, in some embodiments, the CDRs correspond to amino acid residues 26-35 (HCDR1), 50-65 (HCDR2), and 95-102 (HCDR3) in a VH, e.g., a mammalian VH, e.g., a human VH; and amino acid residues 24-34 (LCDR1), 50-56 (LCDR2), and 89-97 (LCDR3) in a VL, e.g., a mammalian VL, e.g., a human VL.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically

The term “xenogeneic” refers to any material derived from an animal of a different species.

The term “cancer” refers to a disease characterized by the uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. The terms “tumor” and “cancer” are used interchangeably herein, e.g., both terms encompass solid and liquid, e.g., diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connote or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connote or include a limitation to a particular process of producing the intracellular signaling domain, e.g., it does not mean that, to provide the intracellular signaling domain, one must start with a CD3zeta sequence and delete unwanted sequence, or impose mutations, to arrive at the intracellular signaling domain.

The phrase “disease associated with expression of a tumor antigen as described herein” includes, but is not limited to, a disease associated with expression of a tumor antigen as described herein or condition associated with cells which express a tumor antigen as described herein including, e.g., proliferative diseases such as a cancer or malignancy or a precancerous condition such as a myelodysplasia, a myelodysplastic syndrome or a preleukemia; or a noncancer related indication associated with cells which express a tumor antigen as described herein. In one embodiment, a cancer associated with expression of a tumor antigen as described herein is a hematological cancer. In one embodiment, a cancer associated with expression of a tumor antigen as described herein is a solid cancer. Further diseases associated with expression of a tumor antigen as described herein include, but not limited to, e.g., atypical and/or non-classical cancers, malignancies, precancerous conditions or proliferative diseases associated with expression of a tumor antigen as described herein. Non-cancer related indications associated with expression of a tumor antigen as described herein include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels or reduced levels. In an embodiment, the tumor antigen-expressing cells produced detectable levels of a tumor antigen protein at one point, and subsequently produced substantially no detectable tumor antigen protein.

The phrase “disease associated with expression of CD19” includes, but is not limited to, a disease associated with expression of CD19 or condition associated with cells which express CD19 including, e.g., proliferative diseases such as a cancer or malignancy or a precancerous condition such as a myelodysplasia, a myelodysplastic syndrome or a preleukemia; or a noncancer related indication associated with cells which express CD19. In one aspect, a cancer associated with expression of CD19 is a hematological cancer. In one aspect, the hematological cancer is a leukemia or a lymphoma. In one aspect, a cancer associated with expression of CD19 includes cancers and malignancies including, but not limited to, e.g., one or more acute leukemias including but not limited to, e.g., acute myeloid leukemia (AML), B-cell acute Lymphoid Leukemia (BALL), T-cell acute Lymphoid Leukemia (TALL), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of CD19 comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma (MCL), Marginal zone lymphoma, multiple myeloma, myelodysplasia and myelodysplastic syndrome, non-Hodgkin lymphoma, Hodgkin lymphoma, plasmablastic lymphoma, plasmacytoid dendritic cell neoplasm. Waldenstrom macroglobulinemia, myeloproliferative neoplasm; a histiocytic disorder (e.g., a mast cell disorder or a blastic plasmacytoid dendritic cell neoplasm); a mast cell disorder, e.g., systemic mastocytosis or mast cell leukemia; B-cell prolymphocytic leukemia, plasma cell myeloma, and “preleukemia” which are a diverse collection of hematological conditions united by ineffective production (or dysplasia) of myeloid blood cells, and the like. Further diseases associated with expression of CD19expression include, but not limited to, e.g., atypical and/or non-classical cancers, malignancies, precancerous conditions or proliferative diseases associated with expression of CD19. Non-cancer related indications associated with expression of CD19 include, but are not limited to, e.g., autoimmune disease, (e.g., lupus), inflammatory disorders (allergy and asthma) and transplantation. In some embodiments, the tumor antigen-expressing cells express, or at any time expressed, mRNA encoding the tumor antigen. In an embodiment, the tumor antigen-expressing cells produce the tumor antigen protein (e.g., wild-type or mutant), and the tumor antigen protein may be present at normal levels or reduced levels. In an embodiment, the tumor antigen-expressing cells produced detectable levels of a tumor antigen protein at one point, and subsequently produced substantially no detectable tumor antigen protein. In other embodiments, the disease is a CD19-negative cancer, e.g., a CD19-negative relapsed cancer. In some embodiments, the tumor antigen (e.g., CD19)-expressing cell expresses, or at any time expressed, mRNA encoding the tumor antigen. In an embodiment, the tumor antigen (e.g., CD19)-expressing cell produces the tumor antigen protein (e.g., wild-type or mutant), and the tumor antigen protein may be present at normal levels or reduced levels. In an embodiment, the tumor antigen (e.g., CD19)-expressing cell produced detectable levels of a tumor antigen protein at one point, and subsequently produced substantially no detectable tumor antigen protein.

The phrase “disease associated with expression of a B-cell antigen” includes, but is not limited to, a disease associated with expression of one or more of CD19, CD20, CD22 or ROR1, or a condition associated with cells which express, or at any time expressed, one or more of CD19, CD20, CD22 or ROR1, including, e.g., proliferative diseases such as a cancer or malignancy or a precancerous condition such as a myelodysplasia, a myelodysplastic syndrome or a preleukemia; or a noncancer related indication associated with cells which express one or more of CD19, CD20, CD22 or ROR1. For the avoidance of doubt, a disease associated with expression of the B-cell antigen may include a condition associated with cells which do not presently express the B-cell antigen, e.g., because the antigen expression has been downregulated, e.g., due to treatment with a molecule targeting the B-cell antigen, e.g., a B-cell targeting CAR, but which at one time expressed the antigen. The phrase “disease associated with expression of a B-cell antigen” includes a disease associated with expression of CD19, as described herein. In embodiments, the CAR-expressing cells are used to treat a disease associated with a B-cell antigen. In embodiments, a CAR produced by a method herein comprises an antigen binding domain that targets a B-cell antigen.

The term “relapse” as used herein refers to reappearance of a disease (e.g., cancer) after an initial period of responsiveness, e.g., after prior treatment with a therapy, e.g., cancer therapy (e.g., complete response or partial response). The initial period of responsiveness may involve the level of cancer cells falling below a certain threshold, e.g., below 20%, 15%, 10%, 5%, 4%, 3%, 2%, or 1%. The reappearance may involve the level of cancer cells rising above a certain threshold, e.g., above 20%, 15%, 10%, 5%, 4%, 3%, 2%, or 1%. For example, e.g., in the context of B-ALL, the reappearance may involve, e.g., a reappearance of blasts in the blood, bone marrow (>5%), or any extramedullary site, after a complete response. A complete response, in this context, may involve <5% BM blast. More generally, in an embodiment, a response (e.g., complete response or partial response) can involve the absence of detectable MRD (minimal residual disease). In an embodiment, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

“Refractory” as used herein refers to a disease, e.g., cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a CAR described herein can be replaced with other amino acid residues from the same side chain family and the altered CAR can be tested using the functional assays described herein.

The term “stimulation,” refers to a primary response induced by binding of a stimulatory molecule (e.g., a TCR/CD3 complex or CAR) with its cognate ligand (or tumor antigen in the case of a CAR) thereby mediating a signal transduction event, such as, but not limited to, signal transduction via the TCR/CD3 complex or signal transduction via the appropriate NK receptor or signaling domains of the CAR. Stimulation can mediate altered expression of certain molecules.

The term “stimulatory molecule,” refers to a molecule expressed by an immune cell (e.g., T cell, NK cell, B cell) that provides the cytoplasmic signaling sequence(s) that regulate activation of the immune cell in a stimulatory way for at least some aspect of the immune cell signaling pathway. In one aspect, the signal is a primary signal that is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing cytoplasmic signaling sequence that is of particular use in the invention includes, but is not limited to, those derived from CD3 zeta, common FcR gamma (FCER1G), Fc gamma RIIa, FcR beta (Fc Epsilon R1b), CD3 gamma, CD3 delta , CD3 epsilon, CD79a, CD79b, DAP10, and DAP12. In a specific CAR of the invention, the intracellular signaling domain in any one or more CARS of the invention comprises an intracellular signaling sequence, e.g., a primary signaling sequence of CD3-zeta. In a specific CAR of the invention, the primary signaling sequence of CD3-zeta is the sequence provided as SEQ ID NO:9 (mutant CD3 zeta), or the equivalent residues from a non-human species, e.g., mouse, rodent, monkey, ape and the like. In a specific CAR of the invention, the primary signaling sequence of CD3-zeta is the sequence as provided in SEQ ID NO:10 (wild-type human CD3 zeta), or the equivalent residues from a non-human species, e.g., mouse, rodent, monkey, ape and the like.

The term “antigen presenting cell” or “APC” refers to an immune system cell such as an accessory cell (e.g., a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. The intracellular signaling domain can generate a signal that promotes an immune effector function of the CAR containing cell, e.g., a CART cell. Examples of immune effector function, e.g., in a CART cell, include cytolytic activity and helper activity, including the secretion of cytokines. In embodiments, the intracellular signaling domain is the portion of a protein which transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In an embodiment, the intracellular signaling domain can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (“ICOS”), FcεRI, and CD66d, CD32, DAP10, and DAP12.

The term “zeta” or alternatively “zeta chain”, “CD3-zeta” or “TCR-zeta” is defined as the protein provided as GenBank Acc. No. BAG36664.1, or the equivalent residues from a non-human species, e.g., mouse, rodent, monkey, ape and the like, and a “zeta stimulatory domain” or alternatively a “CD3-zeta stimulatory domain” or a “TCR-zeta stimulatory domain” is defined as the amino acid residues from the cytoplasmic domain of the zeta chain that are sufficient to functionally transmit an initial signal necessary for T cell activation. In one aspect the cytoplasmic domain of zeta comprises residues 52 through 164 of GenBank Acc. No. BAG36664.1 or the equivalent residues from a non-human species, e.g., mouse, rodent, monkey, ape and the like, that are functional orthologs thereof. In one aspect, the “zeta stimulatory domain” or a “CD3-zeta stimulatory domain” is the sequence provided as SEQ ID NO:9. In one aspect, the “zeta stimulatory domain” or a “CD3-zeta stimulatory domain” is the sequence provided as SEQ ID NO:10.

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

A costimulatory intracellular signaling domain refers to an intracellular portion of a costimulatory molecule. The intracellular signaling domain can comprise the entire intracellular portion, or the entire native intracellular signaling domain, of the molecule from which it is derived, or a functional fragment thereof.

The intracellular signaling domain can comprise the entire intracellular portion, or the entire native intracellular signaling domain, of the molecule from which it is derived, or a functional fragment thereof.

The term “4-1BB” refers to a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank Acc. No. AAA62478.2, or the equivalent residues from a non-human species, e.g., mouse, rodent, monkey, ape and the like. In one aspect, the “4-1BB costimulatory domain” is the sequence provided as SEQ ID NO:7 or the equivalent residues from a non-human species, e.g., mouse, rodent, monkey, ape and the like.

“Immune effector cell,” as that term is used herein, refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

“Immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “depletion” or “depleting”, as used interchangeably herein, refers to the decrease or reduction of the level or amount of a cell, a protein, or macromolecule in a sample after a process, e.g., a selection step, e.g., a negative selection, is performed. The depletion can be a complete or partial depletion of the cell, protein, or macromolecule. In an embodiment, the depletion is at least a 1%, 2%, 5%, 10%, 15%, 20%, 25%, 30%, 35%, 40%, 45%, 50%, 55%, 60%, 65%, 70%, 75%, 80%, 85%, 90%, 95%, or 99% decrease or reduction of the level or amount of a cell, a protein, or macromolecule, as compared to the level or amount of the cell, protein or macromolecule in the sample before the process was performed.

The term “enriched” or “enrichment”, as used interchangeably herein, refers to the increase of the level or amount of a cell, a protein, or macromolecule in a sample after a process, e.g., a selection step, e.g., a positive selection, is performed. The enrichment can be a complete or partial enrichment of the cell, protein, or macromolecule. In an embodiment, the enrichment is at least 1%, e.g., at least 1-200%, e.g., at least 1-10, 10-20, 20-30, 30-50, 50-70, 70-90, 90-110, 110-130, 130-150, 150-170, or 170-200% increase of the level or amount of a cell, a protein, or macromolecule, as compared to the level or amount of the cell, protein or macromolecule in a reference sample. In some embodiments, the enrichment is at least 5%, e.g., at least 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, or 99% increase of the level or amount of a cell, a protein, or macromolecule, as compared to the level or amount of the cell, protein or macromolecule in a reference sample. In some embodiments, the enrichment is at least 1.1 fold, e.g., 1.1-200 fold, e.g., 1.1-10, 10-20, 20-30, 30-50, 50-70, 70-90, or 90-100 fold increase of the level or amount of a cell, a protein, or macromolecule, as compared to the level or amount of the cell, protein or macromolecule in a reference sample. In some embodiments, the reference sample can be a same sample, e.g., the sample before the process was performed. In some embodiments, the same sample refers to the sample on which the enrichment is subsequently performed, e.g., a pre-enrichment population, e.g., a starting population. In some embodiments, the reference sample can be a different sample, e.g., a sample on which the process is not performed.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence driven by a promoter.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (e.g., naked or contained in liposomes) and viruses (e.g., lentiviruses, retroviruses, adenoviruses, and adeno-associated viruses) that incorporate the recombinant polynucleotide.

The term “lentivirus” refers to a genus of the Retroviridae family Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, e.g., the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, e.g., between two nucleic acid molecules, such as, two DNA molecules or two RNA molecules, or between two polypeptide molecules. When a subunit position in both of the two molecules is occupied by the same monomeric subunit; e.g., if a position in each of two DNA molecules is occupied by adenine, then they are homologous or identical at that position. The homology between two sequences is a direct function of the number of matching or homologous positions; e.g., if half (e.g., five positions in a polymer ten subunits in length) of the positions in two sequences are homologous, the two sequences are 50% homologous; if 90% of the positions (e.g., 9 of 10), are matched or homologous, the two sequences are 90% homologous.

“Humanized” forms of non-human (e.g., murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, e.g., where necessary to join two protein coding regions, are in the same reading frame.

The term “parenteral” administration of an immunogenic composition includes, e.g., subcutaneous (s.c.), intravenous (i.v.), intramuscular (i.m.), or intrasternal injection, intratumoral, or infusion techniques.

The term “nucleic acid” or “polynucleotide” refers to deoxyribonucleic acid (DNA) or ribonucleic acid (RNA), or a combination of a DNA or RNA thereof, and polymers thereof in either single- or double-stranded form. The term “nucleic acid” includes a gene, cDNA or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen” or “tumor antigen” interchangeably refers to a molecule (typically protein, carbohydrate or lipid) that is preferentially expressed on the surface of a cancer cell, either entirely or as a fragment (e.g., MHC/peptide), in comparison to a normal cell, and which is useful for the preferential targeting of a pharmacological agent to the cancer cell. In some embodiments, a tumor antigen is a marker expressed by both normal cells and cancer cells, e.g., a lineage marker, e.g., CD19 on B cells. In certain aspects, the tumor antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like. In some embodiments, a cancer-associated antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a cancer-associated antigen is a cell surface molecule that is inappropriately synthesized in the cancer cell, for instance, a molecule that contains deletions, additions or mutations in comparison to the molecule expressed on a normal cell. In some embodiments, a cancer-associated antigen will be expressed exclusively on the cell surface of a cancer cell, entirely or as a fragment (e.g., MHC/peptide), and not synthesized or expressed on the surface of a normal cell. In some embodiments, the CARs of the present invention includes CARs comprising an antigen binding domain (e.g., antibody or antibody fragment) that binds to a MHC presented peptide. Normally, peptides derived from endogenous proteins fill the pockets of Major histocompatibility complex (MHC) class I molecules, and are recognized by T cell receptors (TCRs) on CD8+ T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, e.g., Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “flexible polypeptide linker” or “linker” as used in the context of a scFv refers to a peptide linker that consists of amino acids such as glycine and/or serine residues used alone or in combination, to link variable heavy and variable light chain regions together. In one embodiment, the flexible polypeptide linker is a Gly/Ser linker and comprises the amino acid sequence (Gly-Gly-Gly-Ser)n (SEQ ID NO: 15), where n is a positive integer equal to or greater than 1. For example, n=1, n=2, n=3, n=4, n=5, n=6, n=7, n=8, n=9 and n=10. In one embodiment, the flexible polypeptide linkers include, but are not limited to, (Gly₄ Ser)₄ (SEQ ID NO:27) or (Gly₄ Ser)₃ (SEQ ID NO:28). In another embodiment, the linkers include multiple repeats of (Gly₂Ser), (GlySer) or (Gly₃Ser) (SEQ ID NO:29). Also included within the scope of the invention are linkers described in WO2012/138475, incorporated herein by reference).

As used herein, a 5′ cap (also termed an RNA cap, an RNA 7-methylguanosine cap or an RNA m⁷G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA, e.g., mRNA, that has been synthesized in vitro. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In some embodiments of a construct for transient expression, the polyA is between 50 and 5000 (SEQ ID NO: 30), e.g., greater than 64, e.g., greater than 100, e.g., greater than 300 or 400 poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

Apheresis is the process in which whole blood is removed from an individual, separated into select components, and the remainder returned to circulation. Generally, there are two methods for the separation of blood components, centrifugal and non-centrifugal. Leukapheresis results in the active selection and removal of the patient's white blood cells.

As used herein, the terms “treat”, “treatment” and “treating” refer to the reduction or amelioration of the progression, severity and/or duration of a proliferative disorder, or the amelioration of one or more symptoms (e.g., one or more discernible symptoms) of a proliferative disorder resulting from the administration of one or more therapies (e.g., one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (e.g., mammals, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some aspects, the cells are cultured in vitro. In other aspects, the cells are not cultured in vitro.

In the context of the present invention, “tumor antigen” or “hyperproliferative disorder antigen” or “antigen associated with a hyperproliferative disorder” refers to antigens that are common to specific hyperproliferative disorders. In certain embodiments, the tumor antigen is derived from a cancer including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer, ovarian cancer, pancreatic cancer, and the like.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner protein present in a sample, but which antibody or ligand does not substantially recognize or bind other molecules in the sample.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98% or 99% identity, and includes subranges such as 96-99%, 96-98%, 96-97%, 97-99%, 97-98% and 98-99% identity. This applies regardless of the breadth of the range.

As used herein, the term “Stat3 activator” refers to a molecule that activates the Stat3 pathway, e.g., causing increased phosphorylation of Stat3 (e.g., on tyrosine 705 (Y705)), or increasing transcription of a Stat3-activated gene, or by decreasing transcription of a Stat3-inhibited gene. The Stat3 activator may comprise, e.g., a polypeptide or a small molecule. In some embodiments, the Stat3 activator acts upstream of Stat3, e.g., by binding gp130. In some embodiments, the Stat3 activator binds Stat3. A Stat3 activator, includes but is not limited to: an IL-6 family cytokine; an IL-10 family cytokine; an IL-17 family cytokine; a CCL20 molecule; a gp130 activator; an IL-10R2 receptor activator; a soluble IL-6 receptor; and an IL-6/IL-6R complex. In some embodiments, a Stat3 activator can result in CAR T cell expansion, e.g., in vitro or in vivo.

The term “Stat3 activator cell” as used herein, refers to a cell which comprises (e.g., expresses) a Stat3 activator (e.g., as a soluble protein or on the surface of the cell), or a cell to which a Stat3 activator is conjugated, e.g., situated on, the surface of the cell.

The term “IL-6 family cytokine”, as used herein, refers to a molecule in the IL-6 cytokine family, and refers to a full length naturally-occurring IL-6 cytokine family member, an active fragment thereof, or an active variant thereof. In embodiments, the IL-6 family cytokine is chosen from an IL-6 molecule, an IL-11 molecule, an IL-27 molecule, an IL-31 molecule, a CNTF molecule, a CT-1 molecule, a CLC molecule, a LIF molecule, a NP molecule or an OSM molecule. In some embodiments, an IL-6 family cytokine binds to a receptor, e.g., an a receptor (e.g., IL-6Rα, IL-11Rα or CNTFRα). In some embodiments, an IL-6 family cytokine bound to an a receptor results in the formation of a complex, e.g., a complex comprising an a receptor and a signal-transducing β receptor, e.g., gp130. In embodiments, an IL-6 family cytokine signals via a signal-transducing β receptor, e.g., gp130. In some embodiments, an IL-6 family cytokine activates the Stat3 pathway, e.g., phosphorylates tyrosine 705; or increases transcription of a Stat3-activated gene, or decreases transcription of a Stat3-inhibited gene. In some embodiments, an IL-6 family cytokine results in CAR T cell expansion, e.g., in vitro, or in vivo.

The term “IL-6 molecule”, as used herein, refers to a full length naturally-occurring IL-6 (e.g., a mammalian IL-6, e.g., human IL-6, e.g., GenBank Accession Number CAA68278.1), an active fragment of IL-6, or an active variant having at least 80%, 85%, 90%, 95%, 96%. 97%, 98%, or 99% sequence identity to a naturally-occurring wild type polypeptide of IL-6 or fragment thereof. In some embodiments, the variant, e.g., active variant, is a derivative, e.g., a mutant, of a wild type polypeptide or nucleic acid encoding the same. In some embodiments, the IL-6 variant, e.g., active variant of IL-6, has at least 50%, 60%, 70%, 80%, 85%, 90%, 95%, 96%, 97%, 98%. 99% or 100% activity of wild type IL-6 polypeptide, e.g., as measured by an assay of Example 2. In some embodiments, an IL-6 molecule signals via a gp130 receptor. In some embodiments, an IL-6 molecule activates the Stat3 pathway, e.g., phosphorylates tyrosine 705; or increases transcription of a Stat3-activated gene, or decreases transcription of a Stat3-inhibited gene. In some embodiments, the IL-6 molecule comprises one or more post-translational modifications.

As used herein, an “active variant” of a cytokine molecule refers to a cytokine variant having at least 50%, 60%, 70%, 80%, 85%, 90%, 95%, 96%, 97%, 98%, 99% or 100% activity of wild type cytokine, e.g., as measured by an art-recognized assay.

The term “IL-10 family cytokine”, as used herein, refers to a molecule in the IL-10 cytokine family, and refers to a full length naturally-occurring IL-10 cytokine family member, an active fragment thereof, or an active variant thereof. In embodiments, the IL-10 family cytokine is chosen from an IL-10 molecule, an IL-19 molecule, an IL-20 molecule, an IL-22 molecule, an IL-24 molecule, an IL-26 molecule, an IL-28A molecule, an IL-28B molecule or an IL-29 molecule. In some embodiments, an IL-10 family cytokine signals via a IL-10R2 receptor. In some embodiments, an IL-10 family cytokine results in CAR T cell expansion, e.g., in vitro, or in vivo.

The term “IL-17 family cytokine”, as used herein, refers to a molecule in the IL-17 cytokine family, and refers to a full length naturally-occurring IL-17 cytokine family member, an active fragment thereof, or an active variant thereof. In embodiments, the IL-17 family cytokine is chosen form an IL17A molecule, an IL17B molecule, an IL17C molecule, an IL17D molecule, an IL17E molecule or an IL17F molecule. In some embodiments, an IL-17 family cytokine results in CAR T cell expansion, e.g., in vitro, or in vivo.

The term “gp130 activator”, as used herein, refers to a molecule that activates gp130, e.g., causing dimerization, e.g., homodimerization of gp130, or heterodimerization of gp130, e.g., with LIF, OSM or CNTF. In some embodiments, the gp130 activator is chosen from an IL-6 molecule, an IL-11 molecule, an IL-27 molecule, a CNTF molecule, a CT-1 molecule, a CLC molecule, a LIF molecule, a NP molecule, an OSM molecule, or an antibody molecule that binds to gp130, e.g., an anti-gp130 antibody molecule. In some embodiments, a gp130 activator results in signaling via gp130. In some embodiments, a gp130 activator, activates the Stat3 pathway, e.g., phosphorylates tyrosine 705; or increases transcription of a Stat3-activated gene, or decreases transcription of a Stat3-inhibited gene. In some embodiments, a gp130 activator results in CAR T cell expansion, e.g., in vitro, or in vivo.

The term “gp130 molecule” refers to a full length naturally-occurring gp130 (e.g., a mammalian gp130, e.g., human gp130, e.g., GenBank Accession Number AAI17403), an active fragment of gp130, or an active variant having at least 80%, 85%, 90%, 95%, 96%, 97%, 98%, or 99% sequence identity to a naturally-occurring wild type polypeptide of gp130 or fragment thereof. In some embodiments, the variant is a derivative, e.g., a mutant, of a wild type polypeptide or nucleic acid encoding the same. In some embodiments, the gp130 variant, e.g., active variant of gp130, has at least 50%, 60%, 70%, 80%, 85%, 90%, 95%, 96%, 97%, 98%, 99% or 100% activity of the wild type gp130 polypeptide. In some embodiments, a gp130 molecule activates the Stat3 pathway, e.g., phosphorylates tyrosine 705; or increases transcription of a Stat3-activated gene, or decreases transcription of a Stat3-inhibited gene. In some embodiments, a gp130 activator results in CAR T cell expansion, e.g., in vitro, or in vivo. gp130 is also referred to as CD130 or IL-6 receptor subunit beta (IL-6RB).

The term “Stat3 molecule”, as used herein, refers to a molecule that activates the Stat3 pathway, e.g., causing increased phosphorylation of Stat3 (e.g., on tyrosine 705 (Y705)), or by increasing transcription of a Stat3-activated gene, or by decreasing transcription of a Stat3-inhibited gene. The term Stat3 molecule includes a full length naturally-occurring Stat3 (e.g., mammalian Stat3, e.g., human Stat3, e.g., GenBank AAS66986.1), an active fragment of Stat3, or an active variant having at least 80%, 85%, 90%, 95%, 96%, 97%, 98%, or 99% sequence identity to a naturally-occurring wild type polypeptide of Stat3, or a nucleic acid encoding the same. In some embodiments, the variant is a derivative, e.g., a mutant, of a wild type polypeptide or nucleic acid encoding the same. In some embodiments, the Stat3 variant, e.g., active variant of Stat3, has at least 50%, 60%, 70%, 80%, 85%, 90%, 95%, 96%, 97%, 98%, 99% or 100% activity of wild type Stat3 polypeptide, e.g., as measured by an assay of Example 2. In some embodiments, a Stat3 molecule results in CAR T cell expansion, e.g., in vitro or in vivo.

### Description

The present disclosure provides, inter alia, improved methods of making, e.g., method of manufacturing, CAR-expressing cells (e.g., CAR19 expressing cells). The disclosure also provides compositions and reaction mixtures comprising the same. In some embodiments, the method of making comprises contacting a population of immune effector cells with (i) a Stat3 activator, e.g., as described herein, (ii) an inhibitor of glycolysis, e.g., a small molecule inhibitor of glycolysis, e.g., a small molecule hexokinase inhibitor, e.g., a glucose analog, e.g., 2-deoxy-D-glucose (2-DG), or both (i) and (ii). The disclosure also provides, in some aspects, methods of evaluating, predicting, selecting, or monitoring, a subject who will receive, is about to receive, has received or is receiving a therapeutic treatment with a CAR-expressing cell. Described herein are also methods of evaluating or predicting the responsiveness of a subject having a cancer (e.g., a cancer described herein), to a therapeutic treatment with a CAR-expressing cell.

In one aspect, the present disclosure provides improved methods of manufacturing CAR-expressing cells. As described herein in Example 1, lowering glucose metabolism can lead to improved efficacy of CAR-expressing cells. Thus, immune effector cells can be selected for CAR therapy on the basis of having lower glucose metabolism, or an inhibitor of glucose metabolism can be added to the manufacturing process, to improve efficacy of the CAR-expressing cells. Furthermore, Example 2 herein describes Stat3 pathway activation as a way of improving efficacy of CAR-expressing cells.

The disclosure also describes methods of manufacturing immune effector cells (e.g., T cells, NK cells) that can be engineered with a CAR, e.g., a CAR described herein, and reaction mixtures and compositions comprising such cells. The methods provided herein improve the yield and quality, e.g., purity, of cells suitable for expression of a CAR. Without wishing to be bound by theory, the improved yield and quality of the cells that can be engineered to express a CAR is believed to improve the efficiency of introducing a nucleic acid encoding a CAR and improve the expansion of the resulting CAR-expressing cell. Accordingly, the methods and compositions described herein provide improved CAR-expressing cell products for use in treating a disease in a subject.

The disclosure also describes methods that remove unwanted materials, non-target cells, or cells that can negatively impact the expression of a CAR or therapeutic efficacy of the CAR-expressing cell. For example, the methods featured herein can be used to remove or deplete one or more of any of the following: monocytes, granulocytes, red blood cells, platelets, B cells, cancer cells, e.g., lyphoblasts, cryoprotectant (from frozen samples), hemoglobin, or cellular debris. For example, the methods featured herein can be used to enrich or increase the number of one or more of any of the following: T cells (CD4+ and/or CD8+ T cells), NK cells, dendritic cells. Implementation of each method described herein alone or in any combination with each of or all of the methods described herein results in improved starting material suitable for engineering to express a CAR.

Fresh apheresis materials are commonly used in manufacturing cells suitable for expressing a CAR. Use of frozen, e.g., cryopreserved, apheresis materials provides the advantage of being easily transported, thereby removing any restriction on the proximity of location of the patient to a CAR-expressing cell product manufacturing facility, and allowing industrialization of the CAR-expressing cell manufacturing process and greater accessibility of the therapeutic product to patients in need thereof. Methods currently used for manufacturing CAR-expressing cells are optimized for processing of fresh apheresis materials, and cannot be used to obtain similar quality or yield of cells suitable for CAR expression from frozen apheresis samples. In contrast, the methods described herein can be used to process and manufacture cells suitable for CAR expression from a frozen, e.g., cryopreserved, apheresis sample. In embodiments in which the starting material is frozen, e.g., cryopreserved, the methods described herein optionally include a thawing step in which the frozen cells are allowed to thaw, e.g., without interference by an operator or a device to accelerate the thawing process, or the frozen cells are subjected to a device or process that accelerates the thawing process, e.g., by use of a thawing device, e.g., PlasmaTherm. In such embodiments, the thawed material has the same temperature as the surrounding environment, e.g., the same temperature as the ambient temperature of the room or the same temperature of the buffer into which the thawed material is added to, washed with, or incubated with. The methods described herein are particularly useful for generating or enriching a population of immune effector cells that can be engineered to express a CAR from a frozen or thawed input sample, e.g., a frozen or thawed apheresis sample.

Process B, as referred to herein, is a standard protocol for enriching immune effector cells that can be engineered to express a CAR that is currently used. Process B comprises performing density gradient purification with Ficoll, and a positive selection using CD3/CD28 Dynabeads, wherein the input sample is fresh apheresis material. The methods described herein provide greater enrichment, improved quality and yield of the desired immune effector cells suitable for expressing a CAR.

In another aspect, the disclosure features an immune effector cell (e.g., T cell, NK cell), e.g., made by any of the manufacturing methods described herein, engineered to express a CAR, wherein the engineered immune effector cell exhibits an antitumor property. In one embodiment, the CAR comprises an antigen binding domain, a transmembrane domain, and an intracellular signaling domain. An exemplary antigen is a cancer associated antigen (i.e., tumor antigen) described herein. In one aspect, a cell is transformed with the CAR and the CAR is expressed on the cell surface. In some embodiments, the cell (e.g., T cell, NK cell) is transduced with a viral vector encoding a CAR. In some embodiments, the viral vector is a retroviral vector. In some embodiments, the viral vector is a lentiviral vector. In some such embodiments, the cell may stably express the CAR. In another embodiment, the cell (e.g., T cell, NK cell) is transfected with a nucleic acid, e.g., mRNA, cDNA, DNA, encoding a CAR. In some such embodiments, the cell may transiently express the CAR.

Furthermore, the present disclosure provides CAR-expressing cell compositions and their use in medicaments or methods for treating, among other diseases, cancer or any malignancy or autoimmune diseases involving cells or tissues which express a tumor antigen as described herein.

### Elutriation

In one aspect, the methods described herein feature an elutriation method that removes unwanted cells, e.g., monocytes and blasts, thereby resulting in an improved enrichment of desired immune effector cells suitable for CAR expression. In one embodiment, the elutriation method described herein is optimized for the enrichment of desired immune effector cells suitable for CAR expression from a previously frozen sample, e.g., a thawed sample. In one embodiment, the elutriation method described herein provides a preparation of cells with improved purity as compared to a preparation of cells collected from the elutriation protocols known in the art.

In order to facilitate manufacturing logistics (remote sample collection, shipping, storage, and production unit scheduling), the cellular raw material is typically cryopreserved whole blood or apheresis materials which need to be thawed prior to the start of manufacturing. However, the density and size of cells from thawed previously frozen materials are quite different from those of fresh materials. As such, the standard elutriation protocol commonly used for isolating cells for engineering CAR expression largely fails to remove monocytes, granulocytes or any larger-sized cells from cryopreserved and thawed whole blood or apheresis materials. This situation negatively affects the outcome of subsequent CART manufacturing steps, leading to poor yields, product quality concerns, and out-of-specification process deviations. While elutriation can remove monocytes, it is not efficient in removing blast cells, since the blast cells have similar densities and sizes as T lymphocytes.

In an embodiment, the elutriation method described herein includes using an optimized viscosity of the starting sample, e.g., cell sample, e.g., thawed cell sample, by dilution with certain isotonic solutions (e.g., PBS), and using an optimized combination of flow rates and collection volume for each fraction collected by an elutriation device. An example of the modified elutriation program is described in Example 1.

Exemplary ranges of elutriation settings for separation of lymphocytes, e.g., T cells, from monocytes are provided in Table 4. The settings for flow rate, centrifugation, and volume for an exemplary elutriation program is also provided in Table 4 in the columns designated “Ex.”.

TABLE 4
Range of elutriation settings

| | Flow Rate | | Centrifugation | | | |
|---|---|---|---|---|---|---|
| | (mL/min) | | (rpm) | | Volume (mL) | |
| Fraction | Range | Ex. | Range | Ex. | Range | Ex. |
| F1 | 30.0-50.0 | 30.0 | 1800-2400 | 2400 | 100-1000 | 900 |
| F2 | 0.0-50.0 | 30.0 | 0-2400 | 2400 | 0-500 | 500 |
| F3 | 50.0-80.0 | 70.0 | 1800-2400 | 2400 | 500-1000 | 975 |
| F4 | 50.0-80.0 | 72.0 | 1800-2400 | 2400 | 500-1000 | 400 |
| F5 | 80.0-150.0 | 82.0 | 0 | 0 | 0-500 | 250 |

In one embodiment, one, two, three, four, five, six, seven, eight, nine, or ten, or more, fractions are collected from the elutriation step. In one embodiment, five fractions are collected from the elutriation step. In an embodiment where five fractions arc collected, the third fraction (F3) or the fourth fraction (F4), or a combination of the third fraction and the fourth fraction, contain the desired lymphocyte population with the minimal amount of monocytes, granulocytes and other non-lymphocyte cells. In one embodiment, each fraction is collected using a different flow rate. In one embodiment, for each fraction, the flow rate is increased from the flow rate used to collect the previous fraction. In one embodiment, one or more of the fractions is collected using a different collection volume.

In one embodiment, the elutriation is performed using a flow rate of from about 20-90 mL/min, from about 30-90 mL/min, from about 40-90 mL/min, from about 50-90 mL/min, from about 60-90 mL/min, from about 70-90 mL/min, from about 40-85 mL/min, from about 50-82 mL/min, from about 60-82 mL/min, from about 70-82 mL/min, from about 50-80 mL/min, from about 60-80 mL/min, from about 70-80 mL/min. In one embodiment, the elutriation is performed using a flow rate of from about 30-82 mL/min, or from about 50-80 mL/min. In one embodiment, the elutriation is performed using a flow rate of about 30, 40, 50, 60, 70, 72, 80, or 82 mL/min. In one embodiment, the elutriation is performed using a flow rate of about 70 mL/min or 72 mL/min.

In one embodiment, the flow rate for the one or more fractions that contain the desired lymphocyte population with the minimal amount of monocytes, granulocytes, other non-lymphocyte cells, and other undesired components, is from about 20-90 mL/min, from about 30-90 mL/min, from about 40-90 mL/min, from about 50-90 mL/min, from about 60-90 mL/min, from about 70-90 mL/min, from about 40-85 mL/min, from about 50-82 mL/min, from about 60-82 mL/min, from about 70-82 mL/min, from about 50-80 mL/min, from about 60-80 mL/min, from about 70-80 mL/min. In one embodiment, the flow rate for the one or more fractions that contain the desired lymphocyte population is from about 50-82 mL/min, from about 50-80 mL/min, from about 60-82 mL/min, from about 60-80 mL/min, from about 70-82 mL/min, from about 70-80 mL/min, from about 70-75 mL/min, from about 70-72 mL/min. In one embodiment, the flow rate for the one or more fractions that contain the desired lymphocyte population is about 70 mL/min or 72 mL/min.

In one embodiment, the elutriation is performed using a collection volume of about 250-1250 mL, about 250-1000 mL, about 300-1000 mL, about 400-1000 mL, about 500-1000 mL, about 600-1000 mL, about 700-1000 mL, about 800-1000 mL, about 900-1000 mL, about 250-975 mL, about 300-975 mL, about 400-975 mL, about 500-975 mL, about 600-975 mL, about 700-975 mL, about 800-975 ml, about 300-900 mL, about 300-800 mL, about 300-700 mL, about 300-600 mL, about 300-500 mL, or about 300-400 mL. In one embodiment, the elutriation is performed using a collection volume of about 250, 400, 500, 900, or 975 mL. In one embodiment, the elutriation is performed using a collection volume of about 400 mL or about 975 mL.

In one embodiment, the collection volume for the one or more fractions that contain the desired lymphocyte population with the minimal amount of monocytes, granulocytes, other non-lymphocyte cells, and other undesired components, is from about 250-1250 mL, about 250-1000 mL, about 300-1000 mL, about 400-1000 mL, about 500-1000 mL, about 600-1000 mL, about 700-1000 mL, about 800-1000 mL, about 900-1000 mL, about 250-975 mL, about 300-975 mL, about 400-975 mL, about 500-975 mL, about 600-975 mL, about 700-975 mL, about 800-975 ml, about 300-900 mL, about 300-800 mL, about 300-700 mL, about 300-600 mL, about 300-500 mL, or about 300-400 mL. In one embodiment, the collection volume for the one or more fractions that contain the desired lymphocyte population is about 250, 400, 500, 900, or 975 mL. In one embodiment, the collection volume for the one or more fractions that contain the desired lymphocyte population is about 400 mL or about 975 mL.

In one embodiment, the elutriation method described herein is performed by an elutriation device. For example, the elutriation device is the Caridian BCT Elutra™ Cell Separation System (Terumo BCT Model 71800). The Caridian BCT Elutra™ Cell Separation System (Terumo BCT Model 71800) is a closed system that utilizes continuous counter-flow elutriation technology to perform cell separation based primarily by size and secondarily by specific gravity. The opposing forces, generated by the flow of media into the separation chamber and the sedimentation velocity created by the centrifugal force, cause the cells to arrange themselves by size and density within the separation chamber, where they are automatically siphoned into the collection bags. The customized Elutra settings are designed to allow for the distribution of lymphocytes and monocytes combined with granulocytes in different fractions. The Elutra can be operated according to the manufacturer's directions.

### Density Gradient Centrifugation

Manufacturing of adoptive cell therapeutic product requires processing the desired cells, e.g., immune effector cells, away from a complex mixture of blood cells and blood elements present in peripheral blood apheresis starting materials. Peripheral blood-derived lymphocyte samples have been successfully isolated using density gradient centrifugation through Ficoll solution. However, Ficoll is not a preferred reagent for isolating cells for therapeutic use, as Ficoll is not qualified for clinical use. In addition, Ficoll contains glycol, which has toxic potential to the cells. Furthermore, Ficoll density gradient centrifugation of thawed apheresis products after cryopreservation yields a suboptimal T cell product, e.g., as described in the Examples herein. For example, a loss of T cells in the final product, with a relative gain of non-T cells, especially undesirable B cells, blast cells and monocytes was observed in cell preparations isolated by density gradient centrifugation through Ficoll solution.

Without wishing to be bound by theory, it is believed that immune effector cells, e.g., T cells, dehydrate during cryopreservation to become denser than fresh cells. Without wishing to be bound by theory, it is also believed that immune effector cells, e.g., T cells, remain denser longer than the other blood cells, and thus are more readily lost during Ficoll density gradient separation as compared to other cells. Accordingly, without wishing to be bound by theory, a medium with a density greater than Ficoll is believed to provide improved isolation of desired immune effector cells in comparison to Ficoll or other mediums with the same density as Ficoll, e.g., 1.077 g/mL.

In one embodiment, the density gradient centrifugation method described herein includes the use of a density gradient medium comprising iodixanol. In one embodiment, the density gradient medium comprises about 60% iodixanol in water.

In one embodiment, the density gradient centrifugation method described herein includes the use of a density gradient medium having a density greater than Ficoll. In one embodiment, the density gradient centrifugation method described herein includes the use of a density gradient medium having a density greater than 1.077 g/mL, e.g., greater than 1.077 g/mL, greater than 1.1 g/mL, greater than 1.15 g/mL, greater than 1.2 g/mL, greater than 1.25 g/mL, greater than 1.3 g/mL, greater than 1.31 g/mL. In one embodiment, the density gradient medium has a density of about 1.32 g/mL.

In one embodiment, the density gradient centrifugation method described herein includes the use of a density gradient medium comprising iodixanol, e.g., about 60% iodixanol in water, and has a density greater than Ficoll, e.g., greater than 1.077 g/mL, e.g., about 1.32 g/mL. In one embodiment, the density gradient centrifugation method described herein includes the use of a density gradient medium OptiPrep™ (Sigma). OptiPrep™ is a ready-made, sterile and endotoxin-tested solution of 60% (w/v) iodixanol, with a density of 1.320±0.001 g/ml. In contrast, Ficoll density gradient solution has a density of only 1.077 g/ml. Another advantage of OptiPrep™ over Ficoll is that OptiPrep™ is available in GMP grade, and therefore, qualified for therapeutic use.

Without wishing to be bound by theory, the utilization of the OptiPrep density gradient centrifugation step, e.g., with thawed apheresis material, is believed to be less likely to retain undesirable B cells and monocytes, thus is believed to further improve the collection of desired target immune effector cells, e.g., T cells, for subsequent activation and transduction steps. Accordingly, without wishing to be bound by theory, it is believed that the greater density of OptiPrep as compared to Ficoll allows both an enhanced purification and recovery of desired immune effector cells, e.g., T cells, and the concomitant removal of undesirable non-T cell types which can otherwise interfere with consistently successful outcomes of CAR-expressing immune effector cell, e.g., T cell, product manufacturing.

In one embodiment, the density gradient centrifugation is performed using a cell separation device. Examples of cell separation devices include the Sepax2 (Biosafe). In embodiments where a wash step, e.g., an improved wash step as described herein, is performed, e.g., prior to or after the density gradient centrifugation step, the wash step can be performed using the same device as used in the density gradient centrifugation step.

### Enrichment by Selection

Provided herein are methods for selection of specific cells to improve the enrichment of the desired immune effector cells suitable for CAR expression. In one embodiment, the selection comprises a positive selection, e.g., selection for the desired immune effector cells. In another embodiment, the selection comprises a negative selection, e.g., selection for unwanted cells, e.g., removal of unwanted cells. In embodiments, the positive or negative selection methods described herein are performed under flow conditions, e.g., by using a flow-through device, e.g., a flow-through device described herein.

Current selection methods, e.g., positive selection, e.g., using Dynabeads® CD3/CD28 CTS™, can be further optimized for enrichment. First, the amount of Dynabeads used during the selection is typically not based on the percentage of CD45+/3+ cells, e.g., CD45+/3+ cells, present in the post-density gradient centrifugation, e.g., Sepax Ficoll, sample, but rather is based on the percentage of cells, e.g., CD45+/3+ cells, present in the original patient material. Given the significant change in composition caused by the density gradient centrifugation step, e.g., Sepax Ficoll separation procedure, this calculation typically results in a decrease in T cell percentage and increase in monocyte content. Second, the two hour incubation time provides ample opportunity for both non-specific binding of Dynabeads® onto non-target cells (i.e., non CD3 and/or non CD28 cells) and for bead uptake by non-target cells via endocytosis (e.g., monocytes), issues which can compromise T cell yield and purity in the positive selection product. Finally, the magnetic apparatus and operation used the selection can be sub-optimal. Currently, magnetic separation occurs within a large volume of fluid (200 ml), which in turn results in a large distance between magnetically-labeled cells and the magnetic surfaces. This limits the magnetic force available for separation, hence reducing separation sensitivity and requiring longer separation times. In addition, magnetic separation is currently performed statistically, with the sample placed on top the magnetic surface for 5 minutes prior to removal of the negative fraction. Such an extended separation time is detrimental to cells, whose viability is often negatively affected during the procedure due to “pile-up” effects, and provides further opportunity for non-target cells to either bind or internalize the beads.

In contrast to the current selection methods, the selection methods described herein includes a separation that occurs “dynamically”, e.g., under flow conditions, as opposed to the current “static” separation procedure. In an embodiment, separation under flow conditions comprises a magnetic separation reagent, e.g., magnetic beads that selectively bind a target antigen, an input sample, and a magnet, wherein the magnetic separation reagent and the input sample pass, e.g., flow, over a magnet. In certain embodiments, the magnetic separation reagent and the input sample pass, e.g., flow, over the magnet in continuously. Without being bound by theory, this dynamic technique enables the reduction of incubation time (i.e., contacting the sample with the separation reagent) and separation time, thus minimizing negative impacts on target cells and significantly reducing the likelihood of non-specific binding and\or head uptake by non-target populations. In addition, the selection methods described herein do not require any modification to selection reagents (Dynabeads® CD3/CD28 CTS™) or to the amount of reagents used for the selection (3-to-1 bead-to-Tcell ratio).

In an embodiment, the separation or selection under flow conditions, as described herein, comprises the Flow-through Antibody-based Selection Technique (FAST) protocol. Table 12 exhibits an overview of the parameters that differ between the current selection technique and the FAST protocol.

TABLE 12
Comparison between current positive selection
and FAST positive selection

| Unchanged parameters | Modified parameters |
|---|---|
| Reagents (CD3/28 Dynabeads | Incubation time (from 2 hrs to |
| CTS) | 20 min) |
| Bead-to-T cell ratio (3:1) | Flow-through enrichment kit rather |
| | than static configuration |
| Magnetic plate (DynaMag CTS, | Plastic lid for magnetic plate |
| flatbed magnet) | |

In one embodiment, the selection method described herein comprises a shorter incubation period than current standard protocols of the separation reagent and the input sample, followed by magnetic separation. In one embodiment, the incubation period is less than 2 hours, e.g., less than 110 minutes, less than 100 minutes, less than 90 minutes, less than 80 minutes, less than 70 minutes, less than 60 minutes, less than 50 minutes, less than 40 minutes, less than 30 minutes, less than 25 minutes, less than 20 minutes, less than 15 minutes, less than 10 minutes, or less than 5 minutes. In one embodiment, the incubation is performed under gentle rotation.

Exemplary kits for selection are described, e.g., in International Application WO2017/117112, which is incorporated herein by reference in its entirety. For instance, in one embodiment, the kit comprises of an assembly of three bags which can be connected to additional sample/buffer bags via spikes, or through sterile welding. In addition, a modified DynaMag lid, is employed in the separation, which limits the maximum volume during separation to a low volume, e.g., less than 100 mL, less than 90 mL less than 80 mL, less than 70 mL, less than 60 mL, less than 50 mL, less than 40 ml, e.g., about 50 ml. Without wishing to be bound by theory, the low volume used during separation is believed to optimize the magnetic forces acting during the separation procedure and minimize separation times. The modified DynaMag lid also limits the maximum distance that bead:cell conjugates are displaced from the magnet, and standardizes the magnetic for experienced during position selection.

In one embodiment, one or more of the bags of the kit described herein is a triangular bag. In one embodiment, the selection bag is a triangular bag, and enables magnetic separation in “flow-through” mode, as the bag provides ports at opposite ends of the selection bag. In such embodiments, cells can be continuously flown over a magnetic element (e.g., a magnetic plate such as the DynaMag), thus enabling real-time separation of magnetically-labeled particles, while non-labeled cells will not be attracted by the magnetic field and will flow outwards. This flow-through configuration makes the system particularly amenable to automation. The modified separation bag comprises a modified lid to accommodate the additional ports.

In one embodiment, the selection bag is not a triangular bag. In the embodiment where the selection bag is not a triangular bag, the incubation time is less than 2 hours, e.g., less than 110 minutes, less than 100 minutes, less than 90 minutes, less than 80 minutes, less than 70 minutes, less than 60 minutes, less than 50 minutes, less than 40 minutes, less than 30 minutes, less than 25 minutes, less than 20 minutes, less than 15 minutes, less than 10 minutes, or less than 5 minutes.

### Positive Selection

In embodiments, the positive selection methods described herein comprise selecting for, e.g., enriching, the desired immune effector cells. In one embodiment, the positive selection methods described herein comprise selecting for CD3+/CD28+ cells. In other embodiments, the positive selection methods described herein comprise selecting one or more of the following: CD3+ cells, CD28+ cells, CD4+ cells, CD8+ cells, or CD45+ cells.

Separation reagents used in the selection methods described herein comprises a magnetic or paramagnetic member, and an antigen binding member. In one embodiment, the separation reagent comprises a bead, e.g., having magnetic or paramagnetic properties that is coupled to (e.g., covalently, or non-covalently) to an antigen binding member. In one embodiment, the antigen binding member is an antibody or antibody fragment thereof. In one embodiment, the separation reagent used in positive selection for CD3+/CD28+ cells comprises a bead that is coupled to (e.g., covalently, or non-covalently) to a CD3 and/or CD28-binding member, e.g., an anti-CD3 and/or anti-CD28 antibody or antibody fragment.

### Negative Selection

Also provided herein are negative selection methods for negatively selecting for, or depleting, the input sample of unwanted cells, e.g., monocytes, granulocytes, red blood cells. platelets, and B cells, thereby enriching the resulting output sample with the desired immune effector cells, e.g., T cells. In an embodiment, the negative selection methods described herein are performed under flow conditions, e.g., using a flow through device, e.g., a flow through device described herein.

In one embodiment, the negative selection methods described herein comprise negatively selecting for one or more of monocytes, granulocytes, red blood cells, platelets, B cells, or cancer cells, e.g., lymphoblasts.

In embodiments where depletion or removal of one or more of monocytes, granulocytes, red blood cells, platelets, or B cells is desired, the negative selection method selecting for a cell expressing one or more of the following: CD19, CD25, CD14, or other surface marker or protein expressed by a monocyte, granulocyte, red blood cell, platelet, or B cell.

In embodiments where the subject has a hematological cancer, cancer cells may be present in the apheresis samples, and removal of the cancer cells may be desired. In one embodiment, the negative selection method described herein comprises negatively selecting for a CD19+ cell, e.g., a lymphoblast. In another embodiment, the negative selection method described herein comprises negatively selecting for a cancer cell expressing one or more of the following: CD19, CD33, CD123, CLL-1, BCMA, ROR1, or FLT3.

Separation reagents used in the selection methods described herein comprises a magnetic or paramagnetic member, and an antigen binding member. In one embodiment, the separation reagent comprises a bead, e.g., having magnetic or paramagnetic properties that is coupled to (e.g., covalently, or non-covalently) to an antigen binding member. In one embodiment, the antigen binding member is an antibody or antibody fragment thereof. In one embodiment, the separation reagent used in negative selection for CD19+ cells comprises a head that is coupled to (e.g., covalently, or non-covalently) to a CD19-binding member, e.g., an anti-CD19 antibody or antibody fragment. In one embodiment, the separation reagent used in negative selection for CD14+ cells comprises a bead that is coupled to (e.g., covalently, or non-covalently) to a CD14-binding member, e.g., an anti-CD14 antibody or antibody fragment. In one embodiment, the separation reagent used in negative selection for CD25+ cells comprises a bead that is coupled to (e.g., covalently, or non-covalently) to a CD25-binding member, e.g., an anti-CD25 antibody or antibody fragment.

In some embodiments, selection methods can be performed under flow conditions, e.g., by using a flow-through device. Exemplary flow-through devices are described on pages 57-86 of International Application WO 2017/117112 filed on Dec. 27, 2016, which is hereby expressly incorporated by reference.

### Improved Wash Step

The cellular composition of apheresis, e.g., leukapheresis, products vary greatly from patient to patient. Leukapheresis products with high percentages of granulocytes (e.g. neutrophils) have been correlated with instances of elevated cell clumping during CAR T cell manufacturing using Process B. Without wishing to be bound by theory, such irreversible clumping is believed to reduce available cell numbers and negatively impacts cell yields by interfering with the enrichment process (e.g. positive selection) which results in an overall reduction in cell numbers and purity. In addition, without wishing to be bound by theory, reduction in cell purity and yield directly impacts subsequent process performance (e.g., transduction efficiency and expansion), and final product cell numbers and quality. The net outcome of this reduces the ability to manufacture product able to meet dose specifications at the end of the processing cycle. Thus, without wishing to be bound by theory, it is believed that prevention of clumping can reduce the cell loss and improve the T cell purity which can generate better quality and quantity of starting material for the subsequent processing steps and result in an overall improved therapeutic product.

In the current manufacturing processes in the art, e.g., Process B, patient cellular leukapheresis material is thawed on the Plasmatherm (Genesis), washed using the CellSaver 5+ instrument (Haemonetics), and is then resuspended in either a cell expansion medium based on X-VIVO15 medium (Lonza), called ‘Modified Medium’, or into a buffered isotonic saline solution such as phosphate-buffered saline (PBS) for the subsequent Ficoll selection of lymphocytes. Modified Medium is prepared according to the protocol provided in Example 2. However, as described in Example 2, transfer of thawed cells into either Modified Medium or into PBS solution can cause the cells to clump.

Accordingly, also provided herein are improved methods for washing cells to prevent clumping, is compatible with subsequent manufacturing steps, e.g., positive selection by stimulation, e.g., with anti-CD3/CD28 CTS Dynabeads (Thermo Fisher). In addition, the improved wash step described herein is performed, e.g., on thawed cells, to remove subcellular debris, free hemoglobin and cryoprotectants, to achieve volume reduction, and to enable subsequent density gradient separation. In an embodiment, the wash step is performed with an alternative cell resuspension buffer to Modified Medium or PBS solution. In an embodiment, the wash step is performed with a buffer comprising dextrose and/or sodium chloride. In an embodiment, the buffer comprises about 5% and about 0.45% sodium chloride, e.g., D5 ½ NS medium. In an embodiment, the buffer stabilizes the cell suspension and prevents clumping, e.g., for at least 30 minutes, 45 minutes, 1 hour, 1.5 hours, 2 hours, 2.5 hours, 3 hours, 3.5 hours, 4 hours, 4.5 hours, 5 hours, 5.5 hours, or 6 hours.

In one embodiment, the improved wash step described herein is performed using a device, e.g., a cell separation device, e.g., the same device used for density gradient centrifugation. For example, the improved wash step is performed using the Sepax 2 RM device (Biosafe).

In an embodiment, the wash step disclosed herein can be used for a fresh apheresis sample or a previously frozen, e.g., thawed, apheresis sample. In embodiments, the wash step disclosed herein can be used before or after any of the elutriation, density gradient centrifugation, or selection methods described herein. In another embodiment, the wash step disclosed herein is performed after a density gradient centrifugation step, e.g., a density gradient centrifugation using OptiPrep medium.

### Improved Manufacturing Process

Provided herein are methods for improving the quality and yield of immune effector cells suitable for expressing CAR to a greater degree than methods currently used in the art. In one embodiment, the elutriation, density gradient centrifugation, positive and negative selection under flow conditions, and improved wash step described in the preceding sections can be used in any combination with each other or with additional methods currently used in the art or described herein to isolate or enrich for the desired immune effector cells that are suitable for expressing a CAR.

Generally, a method for generating or enriching for a population of immune effector cells that can be engineered to express a CAR includes: providing an input sample, performing an enrichment step, and performing a selection step, thereby producing an output sample comprising the immune effector cells that are suitable for expression of a CAR. Methods for producing a population of immune effector cells that express a CAR comprise the methods for generating or enriching the population of immune effector cells that can be engineered to express a CAR, and further comprise a stimulation step, e.g., wherein the cells are stimulated to proliferate or persist, and further comprises the introduction of a nucleic acid encoding a CAR. Additional disclosure regarding the stimulation and introduction/expression of a CAR are further described in the following sections.

In one embodiment, the input sample is a fresh sample, e.g., a fresh apheresis, leukapheresis, or whole blood sample, obtained from a subject. In another embodiment, the input sample is a frozen sample. In embodiments where the input sample is a frozen sample, e.g., a frozen or cryopreserved apheresis, leukapheresis, or whole blood sample, the method comprises thawing the frozen sample or providing a thawed sample. Frozen, e.g., cryopreserved, samples can be thawed by passive or active means. Thawing by passive means includes allowing the sample to thaw, e.g., reach the temperature of the surrounding environment, e.g., reach room temperature or reach the temperature of the buffer or solution in which the sample is transferred to or mixed with. Thawing by active means includes using a device that thaws the sample, e.g., brings the sample to the temperature of the surrounding environment faster than if thawing by passive means.

In one embodiment, the enrichment step comprises performing elutriation or density gradient centrifugation. The elutriation can be performed using elutriation conditions known in the art, or the improved settings described herein for elutriation of a frozen or previously frozen sample. The density gradient centrifugation can be performed using Ficoll or a media comprising iodixanol, e.g., about 60% iodixanol in water, e.g., OptiPrep™.

In any of the methods described herein, the selection step comprises performing a positive selection step and/or a negative selection step. The positive selection step can comprise selecting for CD3+/CD28+ cells, e.g., using a separation agent, e.g., a bead coupled to an anti-CD3 and/or anti-CD28 antibody, either under static or flow conditions, e.g., using a. The negative selection step can comprise negatively selecting for CD19+ B cells or CD19+ lymphoblasts, e.g., using a separation agent, e.g., a bead coupled to an anti-CD19 antibody.

In any of the methods described herein, a wash step can be performed after sample collection, after thawing of the sample, before the enrichment step, after the enrichment step, before the selection step, or after the selection step, or any combination thereof.

Exemplary methods for generating or enriching for a population of immune effector cells that can be engineered to express a CAR that include one or more of the elutriation, density gradient centrifugation, positive or negative selection, e.g., under flow conditions, or improved wash step are further described herein.

In one embodiment, a method for generating or enriching for a population of immune effector cells that can be engineered to express a CAR includes providing a frozen input sample comprising immune effector cells; thawing the frozen input sample, to produce a thawed sample; performing an enrichment step, wherein the enrichment step comprises performing elutriation on the input sample, wherein the input sample is optionally a thawed input sample; and performing a selection step, wherein the selection is a positive selection, e.g., for CD3/CD28+ cells, or a negative selection, e.g., for CD19+, CD25+, or CD14+ cells.

In another embodiment, a method for generating or enriching for a population of immune effector cells that can be engineered to express a CAR includes providing a fresh or frozen input sample comprising immune effector cells; and optionally, wherein the input sample is a frozen input sample, thawing the frozen input sample, to produce a thawed sample; performing an enrichment step, wherein the enrichment step comprises performing density centrifugation step using a medium comprising iodixanol, e.g., 60% iodixanol in water, e.g., OptiPrep medium, and/or having a density greater than Ficoll (e.g., greater than 1.077 g/ml, e.g., about 1.32 g/ml); and performing a selection step, wherein the selection is a positive selection, e.g., for CD3/CD28+ cells, or a negative selection, e.g., for CD19+, CD25+, or CD14+ cells.

In another embodiment, a method for generating or enriching for a population of immune effector cells that can be engineered to express a CAR includes providing a fresh or frozen input sample comprising immune effector cells; performing an enrichment step, wherein the enrichment step comprises performing elutriation or density centrifugation (e.g., using Ficoll or a Optiprep medium); and performing a positive selection step under flow conditions, e.g., for CD3/CD28+ cells.

In another embodiment, a method for generating or enriching for a population of immune effector cells that can be engineered to express a CAR includes providing a fresh or frozen input sample comprising immune effector cells; performing an enrichment step, wherein the enrichment step comprises performing elutriation or density centrifugation (e.g., using Ficoll or a Optiprep medium); and performing a negative selection step under flow conditions, e.g., for CD19+, CD25+, or CD14+ cells;

In any of the methods described herein, a wash step can be performed after sample collection, after thawing of the sample, before the enrichment step, after the enrichment step, before the selection step, or after the selection step, or any combination thereof.

Control limits can be defined that identifies the range or threshold of a property of the input sample or after one or more steps in the methods described herein, and dictates or determines the next step, in order to optimize enrichment of the desired immune effector cells, and ensure manufacturing success and product quality. In embodiments, the control limits may be different depending on the type of cancer of the subject from which the input sample is obtained from. By way of example, the control limits for the presence of monocytes in the input sample obtained from a subject having ALL or DLBCL, are as follows: if the monocytes are >20% of the input sample, e.g., leukapheresis whole blood cell, the optimal method comprises elutriation and/or CD3/CD28 positive selection under flow conditions; or if the monocytes are <20% of the input sample, the input sample is washed and the optimal method is determined based on blast content. In another example, the control limits for the presence of blast cells in the input sample obtained from a subject having ALL or DLBCL are as follows: if blast cells are ≥20% of incoming leukapheresis WBC, elutriation (to remove monocytes, granulocytes and cell debris) and/or modified CD19 negative selection (to remove blasts), or other technologies to deplete blasts should be performed; or if blast cells are <20% of incoming leukapheresis, then leukapheresis material will be washed and process will be determined based on the monocyte content.

After enrichment of the immune effector cells suitable for expressing a CAR, in one embodiment, the immune effector cells are stimulated, e.g., to proliferate, using any of the methods known in the art or described herein, e.g., as described in the section titled “Activation and Expansion of Immune Effector Cells”.

After enrichment of the immune effector cells suitable for expressing a CAR, and optionally, after stimulation and/or expansion as described herein, a nucleic acid encoding a CAR, e.g., a CAR described herein, can be introduced to the immune effector cells. Methods for introducing a nucleic acid, e.g., encoding a CAR, are well known in the art and described herein, e.g., as described in the sections titled “Nucleic Acid Constructs Encoding a CAR”, “RNA Transfection”, and “Non-viral Delivery Methods”.

### Sources of Immune Effector Cells

This section provides additional methods or steps for obtaining an input sample comprising desired immune effector cells, isolating and processing desired immune effector cells, e.g., T cells, and removing unwanted materials, e.g., unwanted cells. The additional methods or steps described in this section can be used in combination with any of the elutriation, density gradient centrifugation, selection under flow conditions, or improved wash step described in the preceding sections.

A source of cells, e.g., T cells or natural killer (NK) cells, can be obtained from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In certain aspects of the present disclosure, immune effector cells, e.g., T cells, can be obtained from a unit of blood collected from a subject using any number of techniques known to the skilled artisan, and any of the methods disclosed herein, in any combination of steps thereof. In one aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In one embodiment, the cells are washed with phosphate buffered saline (PBS). In an alternative embodiment, the wash solution lacks calcium and may lack magnesium or may lack many if not all divalent cations. In another embodiment, the cells are washed using the improved wash step described herein.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate, or the Haemonetics Cell Saver 5) according to the manufacturer's instructions. After washing, the cells may be resuspended in a variety of biocompatible buffers, such as, for example, Ca-free, Mg-free PBS, PlasmaLyte A, or other saline solution with or without buffer. Alternatively, the undesirable components of the apheresis sample may be removed and the cells directly resuspended in culture media.

In one aspect, desired immune effector cells, e.g., T cells, are isolated from peripheral blood lymphocytes by lysing the red blood cells and depleting the monocytes, for example, by centrifugation through a PERCOLL™ gradient or by counterflow centrifugal elutriation.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. In some embodiments, the population of T regulatory-depleted cells contains less than 30%, 25%, 20%, 15%, 10%, 5%, 4%, 3%, 2%, 1% of CD25+ cells.

In one embodiment, T regulatory cells, e.g., CD25+ T cells, are removed from the population using an anti-CD25 antibody, or fragment thereof, or a CD25-binding ligand, e.g. IL-2. In one embodiment, the anti-CD25 antibody, or fragment thereof, or CD25-binding ligand is conjugated to a substrate, e.g., a bead, or is otherwise coated on a substrate, e.g., a bead. In one embodiment, the anti-CD25 antibody, or fragment thereof, is conjugated to a substrate as described herein.

In one embodiment, the T regulatory cells, e.g., CD25+ T cells, are removed from the population using CD25 depleting reagent from Miltenyi™. In one embodiment, the ratio of cells to CD25 depletion reagent is 1e7 cells to 20 uL, or 1e7 cells to15 uL, or 1e7 cells to 10 uL, or 1e7 cells to 5 uL, or 1e7 cells to 2.5 uL, or 1e7 cells to 1.25 uL. In one embodiment, e.g., for T regulatory cells, e.g., CD25+ depletion, greater than 500 million cells/ml is used. In a further aspect, a concentration of cells of 600, 700, 800, or 900 million cells/ml is used.

In one embodiment, the population of immune effector cells to be depleted includes about 6×10⁹ CD25+ T cells. In other aspects, the population of immune effector cells to be depleted include about 1×10⁹ to 1×10¹⁰ CD25+ T cell, and any integer value in between. In one embodiment, the resulting population T regulatory-depleted cells has 2×10⁹ T regulatory cells, e.g., CD25+ cells, or less (e.g., 1×10⁹, 5×10⁸, 1×10⁸, 5×10⁷, 1×10⁷, or less CD25+ cells).

In one embodiment, the T regulatory cells, e.g., CD25+ cells, are removed from the population using the CliniMAC system with a depletion tubing set, such as, e.g., tubing 162-01. In one embodiment, the CliniMAC system is run on a depletion setting such as, e.g., DEPLETION2.1.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product significantly reduces the risk of subject relapse. For example, methods of depleting T_{REG} cells are known in the art. Methods of decreasing T_{REG} cells include, but are not limited to, cyclophosphamide, anti-GITR antibody (an anti-GITR antibody described herein), CD25-depletion, mTOR inhibitor, and combinations thereof.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

Without wishing to be bound by a particular theory, decreasing the level of negative regulators of immune cells (e.g., decreasing the number of unwanted immune cells, e.g., T_{REG} cells), in a subject prior to apheresis or during manufacturing of a CAR-expressing cell product can reduce the risk of a subject's relapse. In an embodiment, a subject is pre-treated with one or more therapies that reduce T_{REG} cells prior to collection of cells for CAR-expressing cell product manufacturing, thereby reducing the risk of subject relapse to CAR-expressing cell treatment. In an embodiment, methods of decreasing T_{REG} cells include, but are not limited to, administration to the subject of one or more of cyclophosphamide, anti-GITR antibody. CD25-depletion, or a combination thereof. In an embodiment, methods of decreasing T_{REG} cells include, but are not limited to, administration to the subject of one or more of cyclophosphamide, anti-GITR antibody, CD25-depletion, mTOR inhibitor, or a combination thereof. Administration of one or more of cyclophosphamide, anti-GITR antibody, CD25-depletion, or a combination thereof, can occur before, during or after an infusion of the CAR-expressing cell product. Administration of one or more of cyclophosphamide, anti-GITR antibody, CD25-depletion, mTOR inhibitor, or a combination thereof, can occur before, during or after an infusion of the CAR-expressing cell product.

In some embodiments, the manufacturing methods comprise reducing the number of (e.g., depleting) T_{REG} cells prior to manufacturing of the CAR-expressing cell. For example, manufacturing methods comprise contacting the sample, e.g., the apheresis sample, with an anti-GITR antibody and/or an anti-CD25 antibody (or fragment thereof, or a CD25-binding ligand), e.g., to deplete T_{REG} cells prior to manufacturing of the CAR-expressing cell (e.g., T cell, NK cell) product.

In an embodiment, a subject is pre-treated with cyclophosphamide prior to collection of cells for CAR-expressing cell product manufacturing, thereby reducing the risk of subject relapse to CAR-expressing cell treatment (e.g., CTL019 treatment). In an embodiment, a subject is pre-treated with an anti-GITR antibody prior to collection of cells for CAR-expressing cell (e.g., T cell or NK cell) product manufacturing, thereby reducing the risk of subject relapse to CAR-expressing cell treatment.

In an embodiment, the CAR-expressing cell (e.g., T cell, NK cell) manufacturing process is modified to deplete T_{REG} cells prior to manufacturing of the CAR-expressing cell (e.g., T cell, NK cell) product (e.g., a CTL019 product). In an embodiment, CD25-depletion is used to deplete T_{REG} cells prior to manufacturing of the CAR-expressing cell (e.g., T cell, NK cell) product (e.g., a CTL019 product).

In one embodiment, the population of cells to be removed are neither the regulatory T cells or tumor cells, but cells that otherwise negatively affect the expansion and/or function of CART cells, e.g. cells expressing CD14, CD11b, CD33, CD15, or other markers expressed by potentially immune suppressive cells. In one embodiment, such cells are envisioned to be removed concurrently with regulatory T cells and/or tumor cells, or following said depletion, or in another order.

The methods described herein can include more than one selection step, e.g., more than one depletion step. Enrichment of a T cell population by negative selection can be accomplished, e.g., with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, e.g., a tumor antigen that does not comprise CD25, e.g., CD19, CD30, CD38, CD123, CD20, CD14 or CD11b, to thereby provide a population of T regulatory-depleted, e.g., CD25+ depleted, and tumor antigen depleted cells that are suitable for expression of a CAR, e.g., a CAR described herein. In one embodiment, tumor antigen expressing cells are removed simultaneously with the T regulatory, e.g., CD25+ cells. For example, an anti-CD25 antibody, or fragment thereof, and an anti-tumor antigen antibody, or fragment thereof, can be attached to the same substrate, e.g., bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, e.g., CD25+ cells, and the removal of the tumor antigen expressing cells is sequential, and can occur, e.g., in either order.

Also provided are methods that include removing cells from the population which express a check point inhibitor, e.g., a check point inhibitor described herein, e.g., one or more of PD1+ cells, LAG3+ cells, and TIM3+ cells, to thereby provide a population of T regulatory-depleted, e.g., CD25+ depleted cells, and check point inhibitor depleted cells, e.g., PD1+, LAG3+ and/or TIM3+ depleted cells. Exemplary check point inhibitors include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), 137-H4 (VTCN1), HVEM (TNFRSF14 or CD270), KIR, A2aR, MHC class I, MHC class II, GALS, adenosine, and TGF (e.g., TGF beta), e.g., as described herein. In one embodiment, check point inhibitor expressing cells are removed simultaneously with the T regulatory, e.g., CD25+ cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells. In other embodiments, the removal of T regulatory cells, e.g., CD25+ cells, and the removal of the check point inhibitor expressing cells is sequential, and can occur, e.g., in either order.

Methods described herein can include a positive selection step. For example, T cells can isolated by incubation with anti-CD3/anti-CD28 (e.g., 3×28)-conjugated beads, such as DYNABEADS® M-450 CD3/CD28 T, for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another embodiment, the time period is 10 to 24 hours, e.g., 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of heads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In one embodiment, a T cell population can be selected that expresses one or more of IFN-γ, TNFα, IL-17A, IL-2, IL-3, IL-4, GM-CSF, IL-10, IL-13, granzyme B, and perforin, or other appropriate molecules, e.g., other cytokines. Methods for screening for cell expression can be determined, e.g., by the methods described in PCT Publication No.: WO 2013/126712.

For isolation of a desired population of cells by positive or negative selection, the concentration of cells and surface (e.g., particles such as beads) can be varied. In certain aspects, it may be desirable to significantly decrease the volume in which beads and cells are mixed together (e.g., increase the concentration of cells), to ensure maximum contact of cells and beads. For example, in one aspect, a concentration of 10 billion cells/ml, 9 billion/ml, 8 billion/ml, 7 billion/ml, 6 billion/ml, or 5 billion/ml is used. In one aspect, a concentration of 1 billion cells/ml is used. In yet one aspect, a concentration of cells from 75, 80, 85, 90. 95, or 100 million cells/ml is used. In further aspects, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (e.g., leukemic blood, tumor tissue, etc.). Such populations of cells may have therapeutic value and would be desirable to obtain. For example, using high concentration of cells allows more efficient selection of CD8+ T cells that normally have weaker CD28 expression.

In a related aspect, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one aspect, the concentration of cells used is 5×10⁶/ml. In other aspects, the concentration used can be from about 1×10⁵/ml to 1×10⁶/ml, and any integer value in between.

In other aspects, the cells may be incubated on a rotator for varying lengths of time at varying speeds at either 2-10° C. or at room temperature.

In one embodiment, a plurality of the immune effector cells of the population do not express diaglycerol kinase (DGK), e.g., is DGK-deficient. In one embodiment, a plurality of the immune effector cells of the population do not express Ikaros, e.g., is Ikaros-deficient. In one embodiment, a plurality of the immune effector cells of the population do not express DGK and Ikaros, e.g., is both DGK and Ikaros-deficient.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In certain aspects, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In one aspect a blood sample or an apheresis is taken from a generally healthy subject. In certain aspects, a blood sample or an apheresis is taken from a generally healthy subject who is at risk of developing a disease, but who has not yet developed a disease, and the cells of interest are isolated and frozen for later use. In certain aspects, the T cells may be expanded, frozen, and used at a later time. In certain aspects, samples are collected from a patient shortly after diagnosis of a particular disease as described herein but prior to any treatments. In a further aspect, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In a further aspect of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in certain aspects, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In one embodiment, the immune effector cells expressing a CAR molecule, e.g., a CAR molecule described herein, are obtained from a subject that has received a low, immune enhancing dose of an mTOR inhibitor. In an embodiment, the population of immune effector cells, e.g., T cells, to be engineered to express a CAR, are harvested after a sufficient time, or after sufficient dosing of the low, immune enhancing, dose of an mTOR inhibitor, such that the level of PD1 negative immune effector cells, e.g., T cells, or the ratio of PD1 negative immune effector cells, e.g., T cells/PD1 positive immune effector cells, e.g., T cells, in the subject or harvested from the subject has been, at least transiently, increased.

In other embodiments, population of immune effector cells, e.g., T cells, which have, or will be engineered to express a CAR, can be treated ex vivo by contact with an amount of an mTOR inhibitor that increases the number of PD1 negative immune effector cells, e.g., T cells or increases the ratio of PD1 negative immune effector cells, e.g., T cells/PD1 positive immune effector cells, e.g., T cells.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS Immune Cell Serum Replacement” Clinical & Translational Immunology (2015) 4, e31; doi:10.1038/cti.2014.31.

In one embodiment, the methods of the application can utilize culture media conditions comprising serum-free medium. In one embodiment, the serum free medium is OpTmizer CTS (LifeTech), Immunocult XF (Stemcell technologies), CellGro (CellGenix), TexMacs (Miltenyi), Stemline (Sigma), Xvivo15 (Lonza), PrimeXV (Irvine Scientific), or StemXVivo (RandD systems). The serum-free medium can be supplemented with a serum substitute such as ICSR (immune cell serum replacement) from LifeTech. The level of serum substitute (e.g., ICSR) can be, e.g., up to 5%, e.g., about 1%, 2%, 3%, 4%, or 5%.

In one embodiment, a T cell population is diaglycerol kinase (DGK)-deficient. DGK-deficient cells include cells that do not express DGK RNA or protein, or have reduced or inhibited DGK activity. DGK-deficient cells can be generated by genetic approaches, e.g., administering RNA-interfering agents, e.g., siRNA, shRNA, miRNA, to reduce or prevent DGK expression. Alternatively, DGK-deficient cells can be generated by treatment with DGK inhibitors described herein.

In one embodiment, a T cell population is Ikaros-deficient. Ikaros-deficient cells include cells that do not express Ikaros RNA or protein, or have reduced or inhibited Ikaros activity, Ikaros-deficient cells can be generated by genetic approaches, e.g., administering RNA-interfering agents, e.g., siRNA, shRNA, miRNA, to reduce or prevent Ikaros expression. Alternatively, Ikaros-deficient cells can be generated by treatment with Ikaros inhibitors, e.g., lenalidomide.

In embodiments, a T cell population is DGK-deficient and Ikaros-deficient, e.g., does not express DGK and Ikaros, or has reduced or inhibited DGK and Ikaros activity. Such DGK and Ikaros-deficient cells can be generated by any of the methods described herein.

In an embodiment, the NK cells are obtained from the subject. In another embodiment, the NK cells are an NK cell line, e.g., NK-92 cell line (Conkwest).

Allogeneic CAR-Expressing Cells

In embodiments described herein, the immune effector cell can be an allogeneic immune effector cell, e.g., T cell or NK cell. For example, the cell can be an allogeneic T cell, e.g., an allogeneic T cell lacking expression of a functional T cell receptor (TCR) and/or human leukocyte antigen (HLA), e.g., HLA class I and/or HLA class II.

A T cell lacking a functional TCR can be, e.g., engineered such that it does not express any functional TCR on its surface, engineered such that it does not express one or more subunits that comprise a functional TCR (e.g., engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some embodiments, downregulation of HLA may be accomplished by reducing or eliminating expression of beta-2 microglobulin (B2M).

In some embodiments, the T cell can lack a functional TCR and a functional HLA, e.g., HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, e.g. by any method described herein. For example, the cell can be a cell that does not express or expresses at low levels an inhibitory molecule, e.g., that can decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta). Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA , and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

Expression systems for siRNA and shRNAs, and exemplary shRNAs, are described, e.g., in paragraphs 649 and 650 of International Application WO2015/142675. filed Mar. 13, 2015, which is incorporated by reference in its entirety.

CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

The CRISPR/Cas system, and uses thereof, are described, e.g., in paragraphs 651-658 of International Application WO2015/142675, filed Mar. 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

TALENs , and uses thereof, are described, e.g., in paragraphs 659-665 of International Application WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, e.g., T cell.

ZFNs, and uses thereof, are described, e.g., in paragraphs 666-671 of International Application WO2015/142675, filed Mar. 13, 2015, which is incorporated by reference in its entirety.

### Telomerase Expression

Telomeres play a crucial role in somatic cell persistence, and their length is maintained by telomerase (TERT). Telomere length in CLL cells may be very short (Roth et al., “Significantly shorter telomeres in T-cells of patients with ZAP-70+/CD38 chronic lymphocytic leukaemia” British Journal of Haematology, 143, 383-386., Aug. 28, 2008), and may be even shorter in manufactured CAR-expressing cells, e.g., CART19 cells, limiting their potential to expand after adoptive transfer to a patient. Telomerase expression can rescue CAR-expressing cells from replicative exhaustion.

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in an embodiment, an immune effector cell, e.g., a T cell, ectopically expresses a telomerase subunit, e.g., the catalytic subunit of telomerase, e.g., TERT, e.g., hTERT. In some aspects, this disclosure provides a method of producing a CAR-expressing cell, comprising contacting a cell with a nucleic acid encoding a telomerase subunit, e.g., the catalytic subunit of telomerase, e.g., TERT, e.g., hTERT. The cell may be contacted with the nucleic acid before, simultaneous with, or after being contacted with a construct encoding a CAR.

Telomerase expression may be stable (e.g., the nucleic acid may integrate into the cell's genome) or transient (e.g., the nucleic acid does not integrate, and expression declines after a period of time, e.g., several days). Stable expression may be accomplished by transfecting or transducing the cell with DNA encoding the telomerase subunit and a selectable marker, and selecting for stable integrants. Alternatively or in combination, stable expression may be accomplished by site-specific recombination, e.g., using the Cre/Lox or FLP/FRT system.

Transient expression may involve transfection or transduction with a nucleic acid, e.g., DNA or RNA such as mRNA. In some embodiments, transient mRNA transfection avoids the genetic instability sometimes associated with stable transfection with TERT. Transient expression of exogenous telomerase activity is described, e.g., in International Application WO2014/130909, which is incorporated by reference herein in its entirety. In embodiments, mRNA-based transfection of a telomerase subunit is performed according to the messenger RNA Therapeutics™ platform commercialized by Moderna Therapeutics. For instance, the method may be a method described in U.S. Pat. No. 8,710,200, 8,822,663, 8,680,069, 8,754,062, 8,664,194, or 8,680,069.

In an embodiment, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795):

(SEQ ID NO: 108)
MPRAPRCRAVRSLLRSHYREVLPLATFVRRLGPQGWRLVQRGDP
AAFRALVAQCLVCVPWDARPPPAAPSFRQVSCLKELVARVLQRL
CERGAKNVLAFGFALLDGARGGPPEAFTTSVRSYIPNTVTDALR
GSGAWGLLLRRVGDDVLVHLLARCALFVLVAPSCAYQVCGPPLY
QLGAATQARPPPHASGPRRRLGCERAWNHSVREAGVPLGLPAPG
ARRRGGSASRSLPLPKRPRRGAAPEPERTPVGQGSWAHPGRTRG
PSDRGFCVVSPARPAEEATSLEGALSGTRHSHPSVGRQHHAGPP
STSRPPRPWDTPCPPVYAETKHFLYSSGDKEQLRPSFLLSSLRP
SLIGARRLVETIFLGSRPWMPGTPRRLPRLPQRYWQMRPLFLEL
LGNHAQCPYGVLLKTHCPLRAAVTPAAGVCAREKPQGSVAAPEE
EDTDPRRLVQLLRQHSSPWQVYGFVRACLKRLVPFGLWGSRHNE
KRFLRNTKKFISLGKHAKLSLQELTWKMSVRGCAWLRRSPGVGC
VPAAEHRLREEILAKFLHWLMSVYVVELLRSFFYVTETTFQKNR
LFFYRKSVWSKLQSIGIRQHLKRVQLRELSEAEVRQHREARPAL
LTSRLRFIPKPDGLRPIVNMDYVVGARTFRREKRAERLTSRVKA
LFSVLNYERARRPGLLGASVLGLDDIHRAWRTFVLRVRAQDPPP
ELYFVKVDVTGAYDTIPQDRLTEVIASIIKPQNTYCVRRYAVVQ
KAAHGHVRKAFKSHVSTLTDLQPYMRQFVAHLQETSPIRDAVVI
EQSSSLNEASSGLFDVFLRFMCHHAVRIRGKSYVQCQGIPQGSI
LSTLLCSLCYGDMENKLFAGIRRDGLLLRLVDDFLLVTPHLTHA
KTFLRTLVRGVPEYGCVVNLRKTVVNFPVEDEALGGTAFVQMPA
HGLFPWCGLLLDTRTLEVQSDYSSYARTSIRASLTFNRGFKAGR
NMRRKLFGVLRLKCHSLFLDLQVNSLQTVCTNIYKILLLQAYRF
HACVLQLPFHQQVWKNPTFFLRVISDTASLCYSILKAKNAGMSL
GAKGAAGPLPSEAVQWLCHQAFLLKLTRHRVTYVPLLGSLRTAQ
TQLSRKLPGTTLTALEAAANPALPSDFKTILD

In an embodiment, the hTERT has a sequence at least 80%, 85%, 90%, 95%, 96%, 97%, 98%, or 99% identical to the sequence of SEQ ID NO: 108. In an embodiment, the hTERT has a sequence of SEQ ID NO: 108. In an embodiment, the hTERT comprises a deletion (e.g., of no more than 5, 10, 15, 20, or 30 amino acids) at the N-terminus, the C-terminus, or both. In an embodiment, the hTERT comprises a transgenic amino acid sequence (e.g., of no more than 5, 10, 15, 20, or 30 amino acids) at the N-terminus, the C-terminus, or both.

In an embodiment, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795):

```
(SEQ ID NO: 23)
      1 caggcagcgt ggtcctgctg cgcacgtggg aagccctggc cccggccacc cccgcgatgc
    61 cgcgcgctcc ccgctgccga gccgtgcgct ccctgctgcg cagccactac cgcgaggtgc
  121 tgccgctggc cacgttcgtg cggcgcctgg ggccccaggg ctggcggctg gtgcagcgcg
  181 gggacccggc ggctttccgc gcgctggtgg cccagtgcct ggtgtgcgtg ccctgggacg
  241 cacggccgcc ccccgccgcc ccctccttcc gccaggtgtc ctgcctgaag gagctggtgg
  301 cccgagtgct gcagaggctg tgcgagcgcg gcgcgaagaa cgtgctggcc ttcggcttcg
  361 cgctgctgga cggggcccgc gggggccccc ccgaggcctt caccaccagc gtgcgcagct
  421 acctgcccaa cacggtgacc gacgcactgc gggggagcgg ggcgtggggg ctgctgttgc
  481 gccgcgtggg cgacgacgtg ctggttcacc tgctggcacg ctgcgcgctc tttgtgctgg
  541 tggctcccag ctgcgcctac caggtgtgcg ggccgccgct gtaccagctc ggcgctgcca
  601 ctcaggcccg gccoccgcca cacgctagtg gaccccgaag gcgtctggga tgcgaacggg
  661 cctggaacca tagcgtcagg gaggccgggg tccccctggg cctgccagcc ccgggtgcga
  721 ggaggcgcgg gggcagtgcc agccgaagtc tgccgttgcc caagaggccc aggcgtggcg
  781 ctgcccctga gccggagcgg acgcccgttg ggcaggggtc ctgggcccac ccgggcagga
  841 cgcgtggacc gagtgaccgt ggtttctgtg tggtgtcacc tgccagaccc gccgaagaag
  901 ccacctcttt ggagggtgcg ctctctggca cgcgccactc ccacccatcc gtgggccgcc
  961 agcaccacgc gggcccccca tccacatcgc ggccaccacg tccctgggac acgccttgtc
1021 ccccggtgta cgccgagacc aagcacttcc tctactcctc aggcgacaag gagcagctgc
1081 ggccctcctt cctactcagc tctctgaggc ccagcctgac tggcgctcgg aggctcgtgg
1141 agaccatctt tctgggttcc aggccctgga tgccagggac tccccgcagg ttgccccgcc
1201 tgccccagcg ctactggcaa atgcggcccc tgtttctgga gctgcttggg aaccacgcgc
1261 agtgccccta cggggtgctc ctcaagacgc actgcccgct gcgagctgcg gtcaccccag
1321 cagccggtgt ctgtgcccgg gagaagcccc agggctctgt ggcggccocc gaggaggagg
1381 acacagaccc ccgtcgcctg gtgcagctgc tccgccagca cagcagcccc tggcaggtgt
1441 acggcttcgt gcgggcctgc ctgcgccggc tggtgccccc aggcctctgg ggctccaggc
1501 acaacgaacg ccgcttcctc aggaacacca agaagttcat ctccctgggg aagcatgcca
1561 agctctcgct gcaggagctg acgtggaaga tgagcgtgcg gggctgcgct tggctgcgca
1621 ggagcccagg ggttggctgt gttccggccg cagagcaccg tctgcgtgag gagatcctgg
1681 ccaagttcct gcactggctg atgagtgtgt acgtcgtcga gctgctcagg tctttctttt
1741 atgtcacgga gaccacgttt caaaagaaca ggctcttttt ctaccggaag agtgtctgga
1801 gcaagttgca aagcattgga atcagacagc acttgaagag ggtgcagctg cgggagctgt
1861 cggaagcaga ggtcaggcag catcgggaag ccaggcccgc cctgctgacg tccagactcc
1921 gcttcatccc caagcctgac gggctgcggc cgattgtgaa catggactac gtcgtgggag
1981 ccagaacgtt ccgcagagaa aagagggccg agcgtctcac ctcgagggtg aaggcactgt
2041 tcagcgtgct caactacgag cgggcgcggc gccccggcct cctgggcgcc tctgtgctgg
2101 gcctggacga tatccacagg gcctggcgca ccttcgtgct gcgtgtgcgg gcccaggacc
2161 cgccgcctga gctgtacttt gtcaaggtgg atgtgacggg cgcgtacgac accatccccc
2221 aggacaggct cacggaggtc atcgccagca tcatcaaacc ccagaacacg tactgcgtgc
2281 gtcggtatgc cgtggtccag aaggccgccc atgggcacgt ccgcaaggcc ttcaagagcc
2341 acgtctctac cttgacagac ctccagccgt acatgcgaca gttcgtggct cacctgcagg
2401 agaccagccc gctgagggat gccgtcgtca tcgagcagag ctoctocctg aatgaggcca
2461 gcagtggcct cttcgacgtc ttcctacgct tcatgtgcca ccacgccgtg cgcatcaggg
2521 gcaagtccta cgtccagtgc caggggatcc cgcagggctc catcctctcc acgctgctct
2581 gcagcctgtg ctacggcgac atggagaaca agctgtttgc ggggattcgg cgggacgggc
2641 tgctcctgcg tttggtggat gatttcttgt tggtgacacc tcacctcacc cacgcgaaaa
2701 ccttcctcag gaccctggtc cgaggtgtcc ctgagtatgg ctgcgtggtg aacttgcgga
2761 agacagtggt gaacttccct gtagaagacg aggccctggg tggcacggct tttgttcaga
2821 tgccggccca cggcctattc ccctggtgcg gcctgctgct ggatacccgg accctggagg
2881 tgcagagcga ctactccagc tatgcccgga cctccatcag agccagtctc accttcaacc
2941 gcggcttcaa ggctgggagg aacatgcgtc gcaaactctt tggggtcttg cggctgaagt
3001 gtcacagcct gtttctggat ttgcaggtga acagcctcca gacggtgtgc accaacatct
3061 acaagatcct cctgctgcag gcgtacaggt ttcacgcatg tgtgctgcag ctcccatttc
3121 atcagcaagt ttggaagaac cccacatttt tcctgcgcgt catctctgac acggcctccc
3181 tctgctactc catcctgaaa gccaagaacg cagggatgtc gctgggggcc aagggcgccg
3241 ccggccctct gccctccgag gccgtgcagt ggctgtgcca ccaagcattc ctgctcaagc
3301 tgactcgaca ccgtgtcacc tacgtgccac tcctggggtc actcaggaca gcccagacgc
3361 agctgagtcg gaagctoccg gggacgacgc tgactgccct ggaggccgca gccaacccgg
3421 cactgccctc agacttcaag accatcctgg actgatggcc acccgcccac agccaggccg
3481 agagcagaca ccagcagccc tgtcacgccg ggctctacgt cccagggagg gaggggcggc
3541 ccacacccag gcccgcaccg ctgggagtct gaggcctgag tgagtgtttg gccgaggcct
3601 gcatgtccgg ctgaaggctg agtgtccggc tgaggcctga gcgagtgtcc agccaagggc
3661 tgagtgtcca gcacacctgc cgtcttcact tccccacagg ctggcgctcg gctccacccc
3721 agggccagct tttcctcacc aggagcccgg cttccactcc ccacatagga atagtccatc
3781 cccagattcg ccattgttca cccctcgccc tgccctcctt tgccttccac ccccaccatc
3841 caggtggaga ccctgagaag gaccctggga gctctgggaa tttggagtga ccaaaggtgt
3901 gccctgtaca caggcgagga ccctgcacct ggatgggggt ccctgtgggt caaattgggg
3961 ggaggtgctg tgggagtaaa atactgaata tatgagtttt tcagttttga aaaaaaaaaa
4021 aaaaaaa
```

In an embodiment, the hTERT is encoded by a nucleic acid having a sequence at least 80%, 85%, 90%, 95%, 96%, 97%, 98%, or 99% identical to the sequence of SEQ ID NO: 23. In an embodiment, the hTERT is encoded by a nucleic acid of SEQ ID NO: 23.

### Chimeric Antigen Receptor (CAR)

The present invention provides immune effector cells (e.g., T cells, NK cells) that are engineered to contain one or more CARs that direct the immune effector cells to cancer. This is achieved through an antigen binding domain on the CAR that is specific for a cancer associated antigen. There are two classes of cancer associated antigens (tumor antigens) that can be targeted by the CARs described herein: (1) cancer associated antigens that are expressed on the surface of cancer cells; and (2) cancer associated antigens that itself is intracellar, however, a fragment of such antigen (peptide) is presented on the surface of the cancer cells by MHC (major histocompatibility complex).

Accordingly, an immune effector cell, e.g., obtained by a method described herein, can be engineered to contain a CAR that target one of the following cancer associated antigens (tumor antigens): CD19, CD123, CD22, CD30, CD171, CS-1, CLL-1, CD33, EGFRvIII , GD2, GD3, BCMA, Tn Ag, PSMA, ROR1, FLT3, FAP, TAG72, CD38, CD44v6, CEA, EPCAM, B7H3, KIT, IL-13Rα 2, Mesothelin, IL-11Rα, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-I receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-1a, legumain, HPV E6,E7, MAGE-A1, MAGE A1, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Rα s mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, and mut hsp70-2.

Bispecific CARs

In an embodiment a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In certain embodiments, the antibody molecule is a multi-specific (e.g., a bispecific or a trispecific) antibody molecule. Protocols for generating bispecific or heterodimeric antibody molecules, and various configurations for bispecific antibody molecules, are described in, e.g., paragraphs 455-458 of WO2015/142675, filed Mar. 13, 2015, which is incorporated by reference in its entirety.

In one aspect, the bispecific antibody molecule is characterized by a first immunoglobulin variable domain sequence, e.g., a scFv, which has binding specificity for CD19, e.g., comprises a scFv as described herein, or comprises the light chain CDRs and/or heavy chain CDRs from a scFv described herein, and a second immunoglobulin variable domain sequence that has binding specificity for a second epitope on a different antigen.

Chimeric TCR

In one aspect, the antibodies and antibody fragments of the present invention (e.g., CD19 antibodies and fragments) can be grafted to one or more constant domain of a T cell receptor (“TCR”) chain, for example, a TCR alpha or TCR beta chain, to create a chimeric TCR. Without being bound by theory, it is believed that chimeric TCRs will signal through the TCR complex upon antigen binding. For example, an scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment may be grafted into a TCR alpha and/or beta chain to create a chimeric TCR. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced, e.g., by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

Non-Antibody Scaffolds

In embodiments, the antigen binding domain comprises a non-antibody scaffold, e.g., a fibronectin, ankyrin, domain antibody, lipocalin, small modular immuno-pharmaceutical, maxybody, Protein A, or affilin. The non-antibody scaffold has the ability to bind to target antigen on a cell. In embodiments, the antigen binding domain is a polypeptide or fragment thereof of a naturally occurring protein expressed on a cell. In some embodiments, the antigen binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, MA), ankyrin (Molecular Partners AG, Zurich, Switzerland), domain antibodies (Domantis, Ltd., Cambridge, Mass., and Ablynx nv, Zwijnaarde, Belgium), lipocalin (Pieris Proteolab AG, Freising, Germany), small modular immuno-pharmaceuticals (Trubion Pharmaceuticals Inc., Seattle, Wash.), maxybodies (Avidia, Inc., Mountain View, Calif.), Protein A (Affibody AG, Sweden), and affilin (gamma-crystallin or ubiquitin) (Scil Proteins GmbH, Halle, Germany).

In an embodiment the antigen binding domain comprises the extracellular domain, or a counter-ligand binding fragment thereof, of molecule that binds a counterligand on the surface of a target cell.

The immune effector cells can comprise a recombinant DNA construct comprising sequences encoding a CAR, wherein the CAR comprises an antigen binding domain (e.g., antibody or antibody fragment, TCR or TCR fragment) that binds specifically to a tumor antigen, e.g., an tumor antigen described herein, and an intracellular signaling domain. The intracellular signaling domain can comprise a costimulatory signaling domain and/or a primary signaling domain, e.g., a zeta chain. As described elsewhere, the methods described herein can include transducing a cell, e.g., from the population of T regulatory-depleted cells, with a nucleic acid encoding a CAR, e.g., a CAR described herein.

In specific aspects, a CAR comprises a scFv domain, wherein the scFv may be preceded by an optional leader sequence such as provided in SEQ ID NO: 1, and followed by an optional hinge sequence such as provided in SEQ ID NO:2 or SEQ ID NO:36 or SEQ ID NO:38, a transmembrane region such as provided in SEQ ID NO:6, an intracellular signaling domain that includes SEQ ID NO:7 or SEQ ID NO:16 and a CD3 zeta sequence that includes SEQ ID NO:9 or SEQ ID NO:10, e.g., wherein the domains are contiguous with and in the same reading frame to form a single fusion protein.

In one aspect, an exemplary CAR constructs comprise an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), and an intracellular stimulatory domain (e.g., an intracellular stimulatory domain described herein). In one aspect, an exemplary CAR construct comprises an optional leader sequence (e.g., a leader sequence described herein), an extracellular antigen binding domain (e.g., an antigen binding domain described herein), a hinge (e.g., a hinge region described herein), a transmembrane domain (e.g., a transmembrane domain described herein), an intracellular costimulatory signaling domain (e.g., a costimulatory signaling domain described herein) and/or an intracellular primary signaling domain (e.g., a primary signaling domain described herein).

An exemplary leader sequence is provided as SEQ ID NO: 1. An exemplary hinge/spacer sequence is provided as SEQ ID NO: 2 or SEQ ID NO:36 or SEQ ID NO:38. An exemplary transmembrane domain sequence is provided as SEQ ID NO:6. An exemplary sequence of the intracellular signaling domain of the 4-1BB protein is provided as SEQ ID NO: 7. An exemplary sequence of the intracellular signaling domain of CD27 is provided as SEQ ID NO:16. An exemplary CD3zeta domain sequence is provided as SEQ ID NO: 9 or SEQ ID NO:10.

In one aspect, the immune effector cell comprises a recombinant nucleic acid construct comprising a nucleic acid molecule encoding a CAR, wherein the nucleic acid molecule comprises a nucleic acid sequence encoding an antigen binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, e.g., CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

Nucleic acids encoding a CAR can be introduced into the immune effector cells using, e.g., a retroviral or lentiviral vector construct.

Nucleic acids encoding a CAR can also be introduced into the immune effector cell using, e.g., an RNA construct that can be directly transfected into a cell. A method for generating mRNA for use in transfection involves in vitro transcription (IVT) of a template with specially designed primers, followed by polyA addition, to produce a construct containing 3′ and 5′ untranslated sequence (“UTR”) (e.g., a 3′ and/or 5′ UTR described herein), a 5′ cap (e.g., a 5′ cap described herein) and/or Internal Ribosome Entry Site (IRES) (e.g., an IRES described herein), the nucleic acid to be expressed, and a polyA tail, typically 50-2000 bases in length (e.g., described in the Examples, e.g., SEQ ID NO:35). RNA so produced can efficiently transfect different kinds of cells. In one embodiment, the template includes sequences for the CAR. In an embodiment, an RNA CAR vector is transduced into a cell, e.g., a T cell by electroporation.

### Antigen Binding Domain

In one aspect, a plurality of the immune effector cells, e.g., the population of T regulatory-depleted cells, include a nucleic acid encoding a CAR that comprises a target-specific binding element otherwise referred to as an antigen binding domain. The choice of binding element depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR described herein include those associated with viral, bacterial and parasitic infections, autoimmune disease and cancer cells.

In one aspect, the portion of the CAR comprising the antigen binding domain comprises an antigen binding domain that targets a tumor antigen, e.g., a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, e.g., single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

In an embodiment, the antigen binding domain comprises an anti-CD19 antibody, or fragment thereof, e.g., an scFv. For example, the antigen binding domain comprises a variable heavy chain and a variable light chain listed in Table 1. The linker sequence joining the variable heavy and variable light chains can be, e.g., any of the linker sequences described herein, or alternatively, can be GSTSGSGKPGSGEGSTKG (SEQ ID NO:104).

TABLE 1
Anti-CD19 antibody binding domains

| | | |
|---|---|---|
| CD19 | huscFv1 | EIVMTQSPATLSLSPGERATLSCRASQDISKYLNWYQQKPGQAPRLLIYHTSRL |
| | (SEQ ID | HSGIPARFSGSGSGTDYTLTISSLQPEDFAVYFCQQGNTLPYTFGQGTKLEIKG |
| | NO: 39) | GGGSGGGGSGGGGSQVQLQESGPGLVKPSETLSLTCTVSGVSLPDYGVSWIRQP |
| | | PGKGLEWIGVIWGSETTYYSSSLKSRVTISKDNSKNQVSLKLSSVTAADTAVYY |
| | | CAKHYYYGGSYAMDYWGQGTLVTVSS |
| CD19 | huscFv2 | Eivmtqspatlslspgeratlscrasqdiskylnwyqqkpgqaprlliyhtsrlhsgip |
| | (SEQ ID | arfsgsgsgtdytltisslqpedfavyfcqqgntlpytfgqgtkleikggggsggggsg |
| | NO: 40) | gggsqvqlqesqpqlvkpsetlsltctvsgvslpdyqvswirqppqkqlewiqviwqse |
| | | ttyyqsslksrvtiskdnsknqvslklssvtaadtavyycakhyyyggsyamdywgqgt |
| | | Ivtvss |
| CD19 | huscFv3 | Qvqlqesgpglvkpsetlsltctvsgvslpdygvswirqppgkglewigviwgsettvy |
| | (SEQ ID | ssslksrvtiskdnsknqvslklssvtaadtavyycakhyyyggsyamdywgqgtlvtv |
| | NO: 41) | ssggggsggggsggggseivmtqspatlslspqeratlscrasqdiskylnwyqqkpqq |
| | | aprlliyhtsrlhsgiparfsgsgsgtdytltisslqpedfavyfcqqgntlpytfgqg |
| | | tkleik |
| CD19 | huscFv4 | Qvqlqesgpglvkpsetlsltctvsgvslpdygvswirqppgkglewigviwgsettyy |
| | (SEQ ID | qsslksrvtiskdnsknqvslklssvtaadtavyycakhyyyggsyamdywgqgtlvtv |
| | NO: 42) | ssggggsggggsggggseivmtqspatlslspgeratlscrasqdiskylnwyqqkpgq |
| | | aprlliyhtsrlhsgiparfsgsgsgtdytltisslqpedfavyfcqqgntlpytfgqg |
| | | tkleik |
| CD19 | huscFv5 | Eivmtqspatlslspgeratlscrasqdiskylnwyqqkpgqaprlliyhtsrlhsgip |
| | (SEQ ID | arfsgsgsgtdytltisslqpedfavyfcqqgntlpytfgqgtkleikgggqsggggsg |
| | NO: 43) | gggsggggsqvqlqesqpqlvkpsetlsltctvsqvslpdyqvswirqppqkqlewiqv |
| | | iwgsettyyssslksrvtiskdnsknqvslklssvtaadtavyycakhyyyggsyamdy |
| | | wgqgtlvtvss |
| CD19 | huscFv6 | Eivmtqspatlsispgeratlscrasqdiskylnwyqqkpgqaprlliyhtsrlhsgip |
| | (SEQ ID | arfsqsqsqtdytltisslqpedfavyfcqqqntlpytfqqqtkleikggggsggggsg |
| | NO: 44) | gggsggggsqvqlqesgpglvkpsetlsltctvsgvslpdygvswirqppgkglewigv |
| | | iwgsettyyqsslksrvtiskdnsknqvslklssvtaadtavyycakhyyyggsyandy |
| | | wgqgtlvtvss |
| CD19 | huscFv7 | Qvqlqesgpglvkpsetlsltctvsgvslpdygvswirqppgkglewigviwgsettyy |
| | (SEQ ID | ssslksrvtiskdnsknqvslklssvtaadtavyycakhyyyggsyamdywgqgtivtv |
| | NO: 45) | ssggggsggggsggggsggggseivmtqspatlslspgeratlscrasqdiskylnwyq |
| | | qkpgqaprlliyhtsrlhsgiparfsgsgsgtdytltisslqpedfavyfcqqgntlpy |
| | | tfgqgtkleik |
| CD19 | huscFv8 | Qvqlqesgpglvkpsetlsltctvsgvslpdygvswirqppgkglewigviwgsettyy |
| | (SEQ ID | qsslksrvtiskdnsknqvslklssvtaadtavyycakhyyyggsyamdywgqgtlvtv |
| | NO: 46) | ssgggqsggggsggggsggggseivmtqspatlslspqeratlscrasqdiskylnwyq |
| | | qkpgqaprlliyhtsrlhsgiparfsgsgsgtdytltisslqpedfavyfcqqgntlpy |
| | | tfgqgtkleik |
| CD19 | huscFv9 | Eivmtqspatlslspgeratlscrasqdiskylnwyqqkpgqaprlliyhtsrlhsgip |
| | (SEQ ID | arfsgsgsgtdytltisslqpedfavyfcqqgntlpytfgqgtkleikggggsggggsg |
| | NO: 47) | gggsggggsqvqlqesqpqlvkpsetlsltctvsqvslpdyqvswirqppqkqlewiqv |
| | | iwgsettyynsslksrvtiskdnsknqvslklssvtaadtavyycakhyyyggsyandy |
| | | wgqgtlvtvss |
| CD19 | HuscFv10 | QvqlqesgpglvkpseLlslLctvsgvslpdygvswiiqppgkglewigviwgseLLyy |
| | (SEQ ID | nsslksrvtiskdnsknqvslklssvtaadtavyycakhyyyggsyamdywgqgtlvtv |
| | NO: 48) | ssggggsggggsggggsggggseivrntqspatlslspgeratlscrasqdiskylnwyq |
| | | qkpgqaprlliyhtsrlhsgiparfsgsgsgtdytltisslqpedfavyfcqqgntlpy |
| | | tfgqgtkleik |
| CD19 | HuscFv11 | Eivmtqspatlslspgeratlscrasqdiskylnwyqqkpgqaprlliyhtsrlhsgip |
| | (SEQ ID | arfsgsgsgtdytitisslqpedfavyfcqqgntlpytfgqgtkleikggggsggggsg |
| | NO: 49) | gggsqvqlqesqpqlvkpsetlsltctvsqvslpdyqvswirqppqkqlewiqviwqse |
| | | ttyynsslksrvtiskdnsknqvslklssvtaadtavyycakhyyyggsyamdywgqgt |
| | | lvtvss |
| CD19 | HuscFv12 | Qvqlqesgpglvkpsetlsltctvsgvslpdygvswirqppgkglewigviwgsettyy |
| | (SEQ ID | nsslksrvtiskdnsknqvslklssvtaadtavyycakhyyyggsyamdywgqgtlvtv |
| | NO: 50) | ssggggsggggsggggseivmtqspatlslspgeratlscrasqdiskylnwyqqkpgq |
| | | aprlliyhtsrlhsgiparfsgsgsgtdytitisslqpedfavyfcqqgntlpytfgqg |
| | | tkleik |
| CD19 | muCTL019 | Diqntqttsslsaslgdrvtiscrasqdiskylnwyqqkpdgtvklliyhtsrlhsgvp |
| | (SEQ ID | srfsgsgsgtdysltisnleqediatyfcqqgntlpytfgggtkleitggggsggggsg |
| | NO: 51) | gggsevklqesqpqlvapsqslsvtctvsqvslpdyqvswirqpprkqlewlqviwqse |
| | | ttyynsalksrltiikdnsksqvflkmnslqtddtaiyycakhyyyggsyamdywgqgt |
| | | svtvss |

TABLE 2
Additional anti-CD19 antibody
binding domains

| Antibody | VH Sequence | VL Sequence |
|---|---|---|
| SSJ25-C1 | QVQLLESGAELVRPGS | ELVLTQSPKFMSTSVGD |
| | SVKISCKASGYAFSSY | RVSVTCKASQNVGTNVA |
| | WMNWVKQRPGQGLEWI | WYQQKPGQSPKPLIYSAT |
| | GQIYPGDGDTNYNGKF | YRNSGVPDRFTGSGSGTD |
| | KGQATLTADKSSSTAY | FTLTITNVQSKDLADYFY |
| | MQLSGLTSEDSAVYSC | FCQYNRYPYTSGGGTKLE |
| | ARKTISSVVDFYFDYW | IKRRS |
| | GQGTTVT | (SEQ ID NO: 4) |
| | (SEQ ID NO: 3) | |

TABLE 2A
Additional murine anti-CD19 antibody binding domains and CARs

| | | |
|---|---|---|
| mCAR1 | SEQ ID | QVQLLESGAELVRPGSSVKISCKASGYAFSSYWMNWVKQRPGQGLEWIGQIYPGDG |
| scFv | NO: 124 | DTNYNGKFKGQATLIADKSSSTAYMQLSGLTSEDSAVYSCARKTISSVVDFYFDYW |
| | | GQGTTVTGGGSGGGSGGGSGGGSELVLIQSPKFMSTSVGDRVSVTCKASQNVGTNV |
| | | AWYQQKPGQSPKPLIYSATYRNSGVPDRFTGSGSGTDFTLTITNVQSKDLADYFCQ |
| | | YNRYPYTSFFFTKLEIKRRS |
| mCAR1 | | QVQLLESGAELVRPGSSVKISCKASGYAFSSYWMNWVKQRPGQGLEWIGQIYPGDG |
| Full-aa | | DTNYNGKFKGQATLTADKSSSTAYMQLSGLTSEDSAVYSCARKTISSVVDFYFDYW |
| | | GQGTTVTGGGSGGGSGGGSGGGSELVLIQSPKFMSTSVGDRVSVTCKASQNVGTNV |
| | | AWYQQKPGQSPKPLIYSATYRNSGVPDRFTGSGSGTDFTLTITNVQSKDLADYFCQ |
| | | YNRYPYTSFFFTKLEIKRRSKIEVMYPPPYLDNEKSNGTIIHVKGKHLCPSPLFPG |
| | | PSKPFWVLVVVGGVLACYSLLVTVAFTIFWVRSKRSRLLHSDYMNMTPRRPGPTRK |
| | | HYQPYAPPRDFAAYRSRVKFSRSADAPAYQQGQNQLYNELNLORREEYDVLDKRRG |
| | | RDPEMGGKPRRKNPQEGLYNELQKDKMAEAYSEIGMKGERRROKGHDGLYQGLSTA |
| | | TKDTYDALHMQALPPR |
| mCAR2 | SEQ ID | DIQMTQTTSSLSASLGDRVTISCRASQDISKYLNWYQQKPDGTVKLLIYHTSRLHS |
| scFv | NO: 125 | GVPSRFSGSGSGTDYSLTLSNLEQEDIATYFCQQGNTLPYTFOGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAKHY |
| | | YYGGSYAMDYWGQGTSVTVSSE |
| mCAR2 | | DIQMTQTTSSLSASLGDRVTISCRASQDISKYLNWYQQKPDGTVKLLIYHTSRLHS |
| CAR-aa | | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIY |
| | | YCAKHYYYGGSYAMDYWGQGTSVTVSSESKYGPPCPPCPMFWVLVVVGGVLACYSL |
| | | LVTVAFTIFWVKRGRKKLLYIFKQPFMRPVQTTQEEDGCSCRFEEEEGGCELRVKF |
| | | SRSADAPAYQQGQNQLYNELNLGRREEYDVLDKRRGRDPEMGGKPRRKNPQEGLYN |
| | | ELQKDKMAEAYSEIGMKGERRRGKGHDGLYQGLSTATKDTYDALHMQALPPRL |
| mCAR2 | | DIQMTQTT SSLSASLGDR VTISCRASQD ISKYLNWYQQ KPDGTVKLLI |
| Full-aa | | YHTSRLHSGV PSRFSGSGSG TDYSLTISNL EQEDIACYFC QQGNTLPYTF |
| | | GGGTKLEITG STSGSGKPGS GEGSTKGEVK LQESGPGLVA PSQSLSVTCT |
| | | VSGVSLPDYG VSWIRQPPRK GLEWLGVIWG SETTYYNSAL KSRLTIIKDN |
| | | SKSQVFLKMN SLQTDDTAIY YCAKHYYYGG SYAMDYWGQG TSVTVSSESK |
| | | YGPPCPPCPM FWVLVVVGGV LACYSLLVTV |
| | | AFIIFWVKRG RKKLLYIFKQ PFMRPVQTTQ EEDGCSCRFE EEEGGCELRV |
| | | KSRSADAPA YQQGQNQLYN ELNLGRREEY DVLDKRRGRD PEMGGKPRRK |
| | | NPQEGLYNEL QKDKMAEAYS EIGMKGERRR GKGHDGLYQG LSTATKDTYD |
| | | ALHMQALPPR LEGGGEGRGS LLTCGDVEEN PGPRMLLLVT SLLLCELFHP |
| | | AFLLIPRKVC NGLGIGEFKD SLSINATNIK HFKNCTSISG DLHILPVAFR |
| | | GDSFTHTPPL DPQELDILKT VKEITGFLLI QAWPENRTDL HAFENLEIIR |
| | | GRTKQHGQFS LAVVSLNITS LGLRSLKEIS DGDVIISGNK NLCYANTINW |
| | | KKLFGTSGQK TKLISNRGEN SCKATGQVCH ALCSPEGCWG PEPRDCVSCR |
| | | NVSRGRECVD KCNLLEGEPR EFVENSECIQ CHPECLPQAM NITCTGRGPD |
| | | NCIQCAHYID GPHCVKTCPA GVMGENNTLV WKYADAGHVC HLCHPNCTYG |
| | | CTGPGLEGCP TNGPKIPSIA TGMVGALLLL LWALGIGLF M |
| mCAR3 | SEQ ID | DIQMTQTTSSLSASLGDRVTISCRASQDISKYINWYQQKPDGTVKLLIYHTSRLHS |
| scFv | NO: 126 | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | CLEWLCVIWCSETTYYNSALKSRLTIIKDNSKSQVFLKMNSLQTDDTAIYYCAKHY |
| | | YYGGSYAMDYWGQGTSVTVSS |
| mCAR3 | | DIQMTQTTSSLSASLCDRVTISCRASQDISKYLNWYQQKPDCTVKLLIYHTSRLHS |
| Full-aa | | GVPSRFSGSGSGTDYSLTISNLEQEDIATYFCQQGNTLPYTFGGGTKLEITGSTSG |
| | | SGKPGSGEGSTKGEVKLQESGPGLVAPSQSLSVTCTVSGVSLPDYGVSWIRQPPRK |
| | | GLEWLGVIWGSETTYYNSALKSRLTIIKDNSKSQVFLKMNSLQTDDTAIYYCAKHY |
| | | YYGGSYAMDYWGQGTSVTVSSAAAIEVMYPPPYLDNEKSNGTIIHVKGKHLCPSPL |
| | | FPGPSKPFWVLVWGGVLACYSLLVTVAFIIFWVRSKRSRLLHSDYMNMTPRRPGP |
| | | TRKHYQPYAPPRDFAAYRSRVKFSRSADAPAYQQGQNQLYNELNLGRREEYDVLDK |
| | | RRGRDPEMGGKPRRKNPQEGLYNELQKDKMAEAYSEIGMKGERRRGKGHDGLYQGL |
| | | STATKDTYDALHMQALPPR |

In some embodiments, the antigen binding domain comprises a HC CDR1, a HC CDR2, and a HC CDR3 of any heavy chain binding domain amino acid sequences listed in Table 1. In embodiments, the antigen binding domain further comprises a LC CDR1, a LC CDR2, and a LC CDR3. In embodiments, the antigen binding domain comprises a LC CDR1, a LC CDR2, and a LC CDR3 of any light chain binding domain amino acid sequences listed in Table 1.

In some embodiments, the antigen binding domain comprises one, two or all of LC CDR1, LC CDR2, and LC CDR3 of any light chain binding domain amino acid sequences listed in Table 1. and one, two or all of HC CDR1, HC CDR2, and HC CDR3 of any heavy chain binding domain amino acid sequences listed in Table 1.

Any CD19 CAR, e.g., the CD19 antigen binding domain of any known CD19 CAR, can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Exemplary target antigens that can be targeted using the CAR-expressing cells, include, but are not limited to, CD19, CD123, EGFRvIII, mesothelin, among others, as described in, for example, WO 2014/130635, WO 2014/130657, and WO 2015/090230, each of which is herein incorporated by reference in its entirety.

In one embodiment, the CAR T cell that specifically binds to CD19 has the USAN designation TISAGENLECLEUCEL-T. CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CAR-expressing cells can specifically bind to human CD19, e.g., can include a CAR molecule, or an antigen binding domain (e.g., a humanized antigen binding domain) according to Table 3 of WO2014/153270, incorporated herein by reference.

In other embodiments, the CAR-expressing cells can specifically bind to CD123, e.g., can include a CAR molecule (e.g., any of the CAR1-CAR8), or an antigen binding domain according to Tables 1-2 of WO 2014/130635, incorporated herein by reference.

In an embodiment, the CAR molecule comprises a CD123 CAR described herein, e.g., a CD123 CAR described in US2014/0322212A1 or US2016/0068601A1, both incorporated herein by reference. In embodiments, the CD123 CAR comprises an amino acid, or has a nucleotide sequence shown in US2014/0322212A1 or US2016/0068601A1, both incorporated herein by reference.

In other embodiments, the CAR-expressing cells can specifically bind to EGFRvIII, e.g., can include a CAR molecule, or an antigen binding domain according to Table 2 or SEQ ID NO:11 of WO 2014/130657, incorporated herein by reference.

In an embodiment, the CAR molecule comprises an EGFRvIII CAR molecule described herein, e.g., an EGFRvIII CAR described US2014/0322275A1, incorporated herein by reference. In embodiments, the EGFRvIII CAR comprises an amino acid, or has a nucleotide sequence shown in US2014/0322275A1, incorporated herein by reference.

In other embodiments, the CAR-expressing cells can specifically bind to mesothelin, e.g., can include a CAR molecule, or an antigen binding domain according to Tables 2-3 of WO 2015/090230, incorporated herein by reference.

In an embodiment, the CAR molecule comprises a mesothelin CAR described herein, e.g., a mesothelin CAR described in WO 2015/090230, incorporated herein by reference. In embodiments, the mesothelin CAR comprises an amino acid, or has a nucleotide sequence shown in WO 2015/090230, incorporated herein by reference.

In one embodiment, CAR molecule comprises a BCMA CAR molecule described herein, e.g., a BCMA CAR described in US-2016-0046724-A1. In embodiments, the BCMA CAR comprises an amino acid, or has a nucleotide sequence shown in US-2016-0046724-A1, incorporated herein by reference.

In an embodiment, the CAR molecule comprises a CLL1 CAR described herein, e.g., a CLL1 CAR described in US2016/0051651A1, incorporated herein by reference. In embodiments, the CLL1 CAR comprises an amino acid, or has a nucleotide sequence shown in US2016/0051651A1, incorporated herein by reference.

In an embodiment, the CAR molecule comprises a CD33 CAR described herein, e.g a CD33 CAR described in US2016/0096892A1, incorporated herein by reference. In embodiments, the CD33 CAR comprises an amino acid, or has a nucleotide sequence shown in US2016/0096892A1, incorporated herein by reference.

In accordance with any method or composition described herein, in embodiments, a CAR molecule comprises a CD123 CAR described herein, e.g., a CD123 CAR described in US2014/0322212A1 or US2016/0068601A1, both incorporated herein by reference. In embodiments, the CD123 CAR comprises an amino acid, or has a nucleotide sequence shown in US2014/0322212A1 or US2016/0068601A1, both incorporated herein by reference. In other embodiments, a CAR molecule comprises a CD19 CAR molecule described herein, e.g., a CD19 CAR molecule described in US-2015-0283178-A1, e.g., CTL019. In embodiments, the CD19 CAR comprises an amino acid, or has a nucleotide sequence shown in US-2015-0283178-A1, incorporated herein by reference. In one embodiment, CAR molecule comprises a BCMA CAR molecule described herein, e.g., a BCMA CAR described in US-2016-0046724-A1. In embodiments, the BCMA CAR comprises an amino acid, or has a nucleotide sequence shown in US-2016-0046724-A1, incorporated herein by reference. In an embodiment, the CAR molecule comprises a CLL1 CAR described herein, e.g., a CLL1 CAR described in US2016/0051651A1, incorporated herein by reference. In embodiments, the CLL1 CAR comprises an amino acid, or has a nucleotide sequence shown in US2016/0051651A1, incorporated herein by reference. In an embodiment, the CAR molecule comprises a CD33 CAR described herein, e.g., a CD33 CAR described in US2016/0096892A1, incorporated herein by reference. In embodiments, the CD33 CAR comprises an amino acid, or has a nucleotide sequence shown in US2016/0096892A1, incorporated herein by reference. In an embodiment, the CAR molecule comprises an EGFRvIII CAR molecule described herein, e.g., an EGFRvIII CAR described US2014/0322275A1, incorporated herein by reference. In embodiments, the EGFRvIII CAR comprises an amino acid, or has a nucleotide sequence shown in US2014/0322275A1, incorporated herein by reference. In an embodiment, the CAR molecule comprises a mesothelin CAR described herein, e.g., a mesothelin CAR described in WO 2015/090230, incorporated herein by reference. In embodiments, the mesothelin CAR comprises an amino acid, or has a nucleotide sequence shown in WO 2015/090230, incorporated herein by reference.

Exemplary CD19 CARs include CD19 CARs described herein, e.g., in one or more tables described herein, or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

In one embodiment, the antigen binding domain comprises one, two three (e.g., all three) heavy chain CDRs, HC CDR1, HC CDR2 and HC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (e.g., all three) light chain CDRs, LC CDR1, LC CDR2 and LC CDR3, from an antibody described herein (e.g., an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In one embodiment, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

In embodiments, the antigen binding domain targets BCMA and is described in US-2016-0046724-A1.

In embodiments, the antigen binding domain targets CD19 and is described in US-2015-0283178-A1.

In embodiments, the antigen binding domain targets CD123 and is described in US2014/0322212A1, US2016/0068601A1.

In embodiments, the antigen binding domain targets CLL1 and is described in US2016/0051651A1.

In embodiments, the antigen binding domain targets CD33 and is described in US2016/0096892A1.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In other embodiments, the CAR-expressing cells can specifically bind to humanized CD19, e.g., can include a CAR molecule, or an antigen binding domain (e.g., a humanized antigen binding domain) according to Table 3 of WO2014/153270, incorporated herein by reference. The amino acid and nucleotide sequences encoding the CD19 CAR molecules and antigen binding domains (e.g., including one, two, three VH CDRs; and one, two, three VL CDRs according to Kabat or Chothia), are specified in WO2014/153270.

In other embodiments, the CAR-expressing cells can specifically bind to CD123, e.g., can include a CAR molecule (e.g., any of the CAR1 to CAR8), or an antigen binding domain according to Tables 1-2 of WO 2014/130635, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO 2014/130635.

In other embodiments, the CAR-expressing cells can specifically bind to CD123, e.g., can include a CAR molecule (e.g., any of the CAR123-1 to CAR123-4 and hzCAR123-1 to hzCAR123-32), or an antigen binding domain according to Tables 2, 6, and 9 of WO2016/028896, incorporated herein by reference. The amino acid and nucleotide sequences encoding the CD123 CAR molecules and antigen binding domains (e.g., including one, two, three VH CDRs; and one, two, three VL CDRs according to Kabat or Chothia), are specified in WO2016/028896.

In other embodiments, the CAR-expressing cells can specifically bind to EGFRvIII, e.g., can include a CAR molecule, or an antigen binding domain according to Table 2 or SEQ ID NO:11 of WO 2014/130657, incorporated herein by reference. The amino acid and nucleotide sequences encoding the EGFRvIII CAR molecules and antigen binding domains (e.g., including one, two, three VH CDRs; and one, two, three VL CDRs according to Kabat or Chothia), are specified in WO 2014/130657.

In other embodiments, the CAR-expressing cells can specifically bind to CD33, e.g., can include a CAR molecule (e.g., any of CAR33-1 to CAR-33-9), or an antigen binding domain according to Table 2 or 9 of WO2016/014576, incorporated herein by reference. The amino acid and nucleotide sequences encoding the CD33 CAR molecules and antigen binding domains (e.g., including one, two, three VH CDRs; and one, two, three VL CDRs according to Kabat or Chothia), are specified in WO2016/014576.

In other embodiments, the CAR-expressing cells can specifically bind to mesothelin, e.g., can include a CAR molecule, or an antigen binding domain according to Tables 2-3 of WO 2015/090230, incorporated herein by reference. The amino acid and nucleotide sequences encoding the mesothelin CAR molecules and antigen binding domains (e.g., including one, two, three VH CDRs; and one, two, three VL CDRs according to Kabat or Chothia), are specified in WO 2015/090230.

In other embodiments, the CAR-expressing cells can specifically bind to BCMA, e.g., can include a CAR molecule, or an antigen binding domain according to Table 1 or 16, SEQ ID NO: 271 or SEQ ID NO: 273 of WO2016/014565, incorporated herein by reference. The amino acid and nucleotide sequences encoding the BCMA CAR molecules and antigen binding domains (e.g., including one, two, three VH CDRs; and one, two, three VL CDRs according to Kabat or Chothia), are specified in WO2016/014565.

In other embodiments, the CAR-expressing cells can specifically bind to CLL-1, e.g., can include a CAR molecule, or an antigen binding domain according to Table 2 of WO2016/014535, incorporated herein by reference. The amino acid and nucleotide sequences encoding the CLL-1 CAR molecules and antigen binding domains (e.g., including one, two, three VH CDRs; and one, two, three VL CDRs according to Kabat or Chothia), are specified in WO2016/014535.

In other embodiments, the CAR-expressing cells can specifically bind to GFR ALPHA-4, e.g., can include a CAR molecule, or an antigen binding domain according to Table 2 of WO2016/025880, incorporated herein by reference. The amino acid and nucleotide sequences encoding the GFR ALPHA-4 CAR molecules and antigen binding domains (e.g., including one, two, three VH CDRs; and one, two, three VL CDRs according to Kabat or Chothia), are specified in WO2016/025880.

In one embodiment, the antigen binding domain of any of the CAR molecules described herein (e.g., any of CD19, CD123, EGFRvIII, CD33, mesothelin, BCMA, and GFR ALPHA-4) comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antigen binding domain listed above. In one embodiment, the antigen binding domain comprises a heavy chain variable region and/or a variable light chain region of an antibody listed or described above.

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed or described above.

In some embodiments, the tumor antigen is a tumor antigen described in International Application WO2015/142675, filed Mar. 13, 2015, which is herein incorporated by reference in its entirety. In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bDGalp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-I receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-1a); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B 1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In one embodiment, the antigen binding domain comprises one, two three (e.g., all three) heavy chain CDRs, HC CDR1, HC CDR2 and HC CDR3, from an antibody listed above, and/or one, two, three (e.g., all three) light chain CDRs, LC CDR1, LC CDR2 and LC CDR3, from an antibody listed above. In one embodiment, the antigen binding domain comprises a heavy chain variable region and/or a variable light chain region of an antibody listed or described above.

In one aspect, the anti-tumor antigen binding domain is a fragment, e.g., a single chain variable fragment (scFv). In one aspect, the anti-a cancer associate antigen as described herein binding domain is a Fv, a Fab, a (Fab′)2, or a bi-functional (e.g. bi-specific) hybrid antibody (e.g., Lanzavecchia et al., Eur. J. Immunol. 17, 105 (1987)). In one aspect, the antibodies and fragments thereof of the invention binds a cancer associate antigen as described herein protein with wild-type or enhanced affinity.

In some instances, scFvs can be prepared according to a method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (e.g., a Ser-Gly linker) with an optimized length and/or amino acid composition. The linker length can greatly affect how the variable regions of a scFv fold and interact. In fact, if a short polypeptide linker is employed (e.g., between 5-10 amino acids) intrachain folding is prevented. Interchain folding is also required to bring the two variable regions together to form a functional epitope binding site. For examples of linker orientation and size see, e.g., Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, which are incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In another embodiment, the linker sequence comprises sets of glycine and serine repeats such as (Gly₄Ser)n, where n is a positive integer equal to or greater than 1 (SEQ ID NO:25). In one embodiment, the linker can be (Gly₄Ser)₄ (SEQ ID NO:27) or (Gly₄Ser)₃(SEQ ID NO:28). Variation in the linker length may retain or enhance activity, giving rise to superior efficacy in activity studies.

In another aspect, the antigen binding domain is a T cell receptor (“TCR”), or a fragment thereof, for example, a single chain TCR (scTCR). Methods to make such TCRs are known in the art. See, e.g., Willemsen RA et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (e.g., a flexible peptide). This approach is very useful to cancer associated target that itself is intracellar, however, a fragment of such antigen (peptide) is presented on the surface of the cancer cells by MHC.

### Additional Exemplary Antigen Binding Domains

In one embodiment, an antigen binding domain against CD22 comprises an antigen binding portion, e.g., CDRs, of an antibody described in, e.g., Haso et al., Blood, 121(7): 1165-1174 (2013); Wayne et al., Clin Cancer Res 16(6): 1894-1903 (2010); Kato et al., Leuk Res 37(1):83-88 (2013); Creative BioMart (creativebiomart.net): MOM-18047-S(P).

In one embodiment, an antigen binding domain against CS-1 comprises an antigen binding portion, e.g., CDRs, of Elotuzumab (BMS), see e.g., Tai et al., 2008, Blood 112(4):1329-37; Tai et al., 2007, Blood. 110(5):1656-63.

In one embodiment, an antigen binding domain against GD2 comprises an antigen binding portion, e.g., CDRs, of an antibody described in, e.g., Mujoo et al., Cancer Res. 47(4):1098-1104 (1987); Cheung et al., Cancer Res 45(6):2642-2649 (1985), Cheung et al., J Clin Oncol 5(9):1430-1440 (1987), Cheung et al., J Clin Oncol 16(9):3053-3060 (1998), Handgretinger et al., Cancer Immunol Immunother 35(3):199-204 (1992). In some embodiments, an antigen binding domain against GD2 is an antigen binding portion of an antibody selected from mAb 14.18, 14G2a, ch14.18, hu14.18, 3F8, hu3F8, 3G6, 8B6, 60C3, 10B8, ME36.1, and 8H9, see e.g., WO2012033885, WO2013040371, WO2013192294, WO2013061273, WO2013123061, WO2013074916, and WO201385552. In some embodiments, an antigen binding domain against GD2 is an antigen binding portion of an antibody described in US Publication No.: 20100150910 or PCT Publication No.: WO 2011160119.

In one embodiment, an antigen binding domain against Tn antigen comprises an antigen binding portion, e.g., CDRs, of an antibody described in, e.g., U.S. Pat. No. 8,440,798, Brooks et al., PNAS 107(22):10056-10061 (2010), and Stone et al., OncoImmunology 1(6):863-873(2012).

In one embodiment, an antigen binding domain against PSMA comprises an antigen binding portion, e.g., CDRs, of an antibody described in, e.g., Parker et al., Protein Expr Purif 89(2):136-145 (2013), US 20110268656 (J591 ScFv); Frigerio et al, European J Cancer 49(9):2223-2232 (2013) (scFvD2B); WO 2006125481 (mAbs 3/A12, 3/E7 and 3/F11) and single chain antibody fragments (scFv A5 and D7).

In one embodiment, an antigen binding domain against ROR1 comprises an antigen binding portion, e.g., CDRs, of an antibody described in, e.g., Hudecek et al., Clin Cancer Res 19(12):3153-3164 (2013); WO 2011159847; and US20130101607.

In one embodiment, an antigen binding domain against FLT3 comprises an antigen binding portion, e.g., CDRs, of an antibody described in, e.g., WO2011076922, U.S. Pat. No. 5,777,084, EP0754230, US20090297529, and several commercial catalog antibodies (R&D, ebiosciences, Abcam).

In one embodiment, an antigen binding domain against TAG72 comprises an antigen binding portion, e.g., CDRs, of an antibody described in, e.g., Hombach et al., Gastroenterology 113(4):1163-1170 (1997); and Abcam ab691.

In one embodiment, an antigen binding domain against FAP comprises an antigen binding portion, e.g., CDRs, of an antibody described in, e.g., Ostermann et al., Clinical Cancer Research 14:4584-4592 (2008) (FAP5), US Pat. Publication No. 2009/0304718; sibrotuzumab (see e.g., Hofheinz et al., Oncology Research and Treatment 26(1), 2003); and Tran et al., J Exp Med 210(6):1125-1135 (2013).

In one embodiment, an antigen binding domain against CD38 comprises an antigen binding portion, e.g., CDRs, of daratumumab (see, e.g., Groen et al., Blood 116(21):1261-1262 (2010); MOR202 (see, e.g., U.S. Pat. No. 8,263,746); or antibodies described in U.S. Pat. No. 8,362,211.

In one embodiment, an antigen binding domain against CD44v6 comprises an antigen binding portion, e.g., CDRs, of an antibody described in, e.g., Casucci et al., Blood 122(20):3461-3472 (2013).

In one embodiment, an antigen binding domain against CEA comprises an antigen binding portion, e.g., CDRs, of an antibody described in, e.g., Chmielewski et al., Gastoenterology 143(4):1095-1107 (2012).

In one embodiment, an antigen binding domain against EPCAM comprises an antigen binding portion, e.g., CDRS, of an antibody selected from MT110, EpCAM-CD3 bispecific Ab (see, e.g., clinicaltrials.gov/ct2/show/NCT00635596); Edrecolomab; 3622W94; ING-1; and adecatumumab (MT201).

In one embodiment, an antigen binding domain against PRSS21 comprises an antigen binding portion, e.g., CDRs, of an antibody described in U.S. Pat. No. 8,080,650.

In one embodiment, an antigen binding domain against B7H3 comprises an antigen binding portion, e.g., CDRs, of an antibody MGA271 (Macrogenics).

In one embodiment, an antigen binding domain against KIT comprises an antigen binding portion, e.g., CDRs, of an antibody described in, e.g., U.S. Pat. No. 7,915,391, US20120288506 , and several commercial catalog antibodies.

In one embodiment, an antigen binding domain against IL-13Rα 2 comprises an antigen binding portion, e.g., CDRs, of an antibody described in, e.g., WO2008/146911, WO2004087758, several commercial catalog antibodies, and WO2004087758.

In one embodiment, an antigen binding domain against CD30 comprises an antigen binding portion, e.g., CDRs, of an antibody described in, e.g., U.S. Pat. No. 7,090,843 B1, and EP0805871.

In one embodiment, an antigen binding domain against GD3 comprises an antigen binding portion, e.g., CDRs, of an antibody described in, e.g., U.S. Pat. Nos. 7,253,263; 8,207,308; US 20120276046; EP1013761; WO2005035577; and U.S. Pat. No. 6,437,098.

In one embodiment, an antigen binding domain against CD171 comprises an antigen binding portion, e.g., CDRs, of an antibody described in, e.g., Hong et al., J Immunother 37(2):93-104 (2014).

In one embodiment, an antigen binding domain against IL-11Rα comprises an antigen binding portion, e.g., CDRs, of an antibody available from Abcam (cat #ab55262) or Novus Biologicals (cat #EPR5446). In another embodiment, an antigen binding domain again IL-11Ra is a peptide, see, e.g., Huang et al., Cancer Res 72(1):271-281 (2012).

In one embodiment, an antigen binding domain against PSCA comprises an antigen binding portion, e.g., CDRs, of an antibody described in, e.g., Morgenroth et al., Prostate 67(10):1121-1131 (2007) (scFv 7F5); Nejatollahi et al., J of Oncology 2013(2013), article ID 839831 (scFv C5-II); and US Pat Publication No. 20090311181.

In one embodiment, an antigen binding domain against VEGFR2 comprises an antigen binding portion, e.g., CDRs, of an antibody described in, e.g., Chinnasamy et al., J Clin Invest 120(11):3953-3968 (2010).

In one embodiment, an antigen binding domain against LewisY comprises an antigen binding portion, e.g., CDRs, of an antibody described in, e.g., Kelly et al., Cancer Biother Radiopharm 23(4):411-423 (2008) (hu3S193 Ab (scFvs)); Dolezal et al., Protein Engineering 16(1):47-56 (2003) (NC10 scFv).

In one embodiment, an antigen binding domain against CD24 comprises an antigen binding portion, e.g., CDRs, of an antibody described in, e.g., Maliar et al., Gastroenterology 143(5):1375-1384 (2012).

In one embodiment, an antigen binding domain against PDGFR-beta comprises an antigen binding portion, e.g., CDRs, of an antibody Abcam ab32570.

In one embodiment, an antigen binding domain against SSEA-4 comprises an antigen binding portion, e.g., CDRs, of antibody MC813 (Cell Signaling), or other commercially available antibodies.

In one embodiment, an antigen binding domain against CD20 comprises an antigen binding portion, e.g., CDRs, of the antibody Rituximab, Ofatumumab, Ocrelizumab, Veltuzumab, or GA101.

In one embodiment, an antigen binding domain against Folate receptor alpha comprises an antigen binding portion, e.g., CDRs, of the antibody IMGN853, or an antibody described in US20120009181; U.S. Pat. No. 4,851,332, LK26: U.S. Pat. No. 5,952,484.

In one embodiment, an antigen binding domain against ERBB2 (Her2/neu) comprises an antigen binding portion, e.g., CDRs, of the antibody trastuzumab, or pertuzumab.

In one embodiment, an antigen binding domain against MUC1 comprises an antigen binding portion, e.g., CDRs, of the antibody SAR566658.

In one embodiment, the antigen binding domain against EGFR comprises antigen binding portion, e.g., CDRs, of the antibody cetuximab, panitumumab, zalutumumab, nimotuzumab, or matuzumab.

In one embodiment, an antigen binding domain against NCAM comprises an antigen binding portion, e.g., CDRs, of the antibody clone 2-2B: MAB5324 (EMD Millipore)

In one embodiment, an antigen binding domain against Ephrin B2 comprises an antigen binding portion, e.g., CDRs, of an antibody described in, e.g., Abengozar et al., Blood 119(19):4565-4576 (2012).

In one embodiment, an antigen binding domain against IGF-I receptor comprises an antigen binding portion, e.g., CDRs, of an antibody described in, e.g., U.S. Pat. No. 8,344,112 B2; EP2322550 A1; WO 2006/138315, or PCT/US2006/022995.

In one embodiment, an antigen binding domain against CAIX comprises an antigen binding portion, e.g., CDRs, of the antibody clone 303123 (R&D Systems).

In one embodiment, an antigen binding domain against LMP2 comprises an antigen binding portion, e.g., CDRs, of an antibody described in, e.g., U.S. Pat. No. 7,410,640, or US20050129701.

In one embodiment, an antigen binding domain against gp100 comprises an antigen binding portion, e.g., CDRs, of the antibody HMB45, NKIbetaB, or an antibody described in WO2013165940. or US20130295007

In one embodiment, an antigen binding domain against tyrosinase comprises an antigen binding portion, e.g., CDRs, of an antibody described in, e.g., U.S. Pat. No. 5,843,674; or US19950504048.

In one embodiment, an antigen binding domain against EphA2 comprises an antigen binding portion, e.g., CDRs, of an antibody described in, e.g., Yu et al., Mol Ther 22(1):102-111 (2014).

In one embodiment, an antigen binding domain against GD3 comprises an antigen binding portion, e.g., CDRs, of an antibody described in, e.g., U.S. Pat. Nos. 7,253,263; 8,207,308; US 20120276046; EP1013761 A3; 20120276046; WO2005035577; or U.S. Pat. No. 6,437,098.

In one embodiment, an antigen binding domain against fucosyl GM1 comprises an antigen binding portion, e.g., CDRs, of an antibody described in, e.g., US20100297138; or WO2007/067992.

In one embodiment, an antigen binding domain against sLe comprises an antigen binding portion, e.g., CDRs, of the antibody G193 (for lewis Y), see Scott AM et al, Cancer Res 60: 3254-61 (2000), also as described in Neeson et al, J Immunol May 2013 190 (Meeting Abstract Supplement) 177.10.

In one embodiment, an antigen binding domain against GM3 comprises an antigen binding portion, e.g., CDRs, of the antibody CA 2523449 (mAb 14F7).

In one embodiment, an antigen binding domain against HMWMAA comprises an antigen binding portion, e.g., CDRs, of an antibody described in, e.g., Kmiecik et al., Oncoimmunology 3(1):e27185 (2014) (PMID: 24575382) (mAb9.2.27); U.S. Pat. No. 6,528,481; WO2010033866; or US 20140004124.

In one embodiment, an antigen binding domain against o-acetyl-GD2 comprises an antigen binding portion, e.g., CDRs, of the antibody 8B6.

In one embodiment, an antigen binding domain against TEM1/CD248 comprises an antigen binding portion, e.g., CDRs, of an antibody described in, e.g., Marty et al., Cancer Lett 235(2):298-308 (2006); Zhao et al., J Immunol Methods 363(2):221-232 (2011).

In one embodiment, an antigen binding domain against CLDN6 comprises an antigen binding portion, e.g., CDRs, of the antibody IMAB027 (Ganymed Pharmaceuticals), see e.g., clinicaltrial.gov/show/NCT02054351.

In one embodiment, an antigen binding domain against TSHR comprises an antigen binding portion, e.g., CDRs, of an antibody described in, e.g., U.S. Pat. No. 8,603,466; U.S. Pat. No. 8,501,415; or U.S. Pat. No. 8,309,693.

In one embodiment, an antigen binding domain against GPRC5D comprises an antigen binding portion, e.g., CDRs, of the antibody FAB6300A (R&D Systems); or LS-A4180 (Lifespan Biosciences).

In one embodiment, an antigen binding domain against CD97 comprises an antigen binding portion, e.g., CDRs, of an antibody described in, e.g., U.S. Pat. No. 6,846,911; de Groot et al., J Immunol 183(6):4127-4134 (2009); or an antibody from R&D:MAB3734.

In one embodiment, an antigen binding domain against ALK comprises an antigen binding portion, e.g., CDRs, of an antibody described in, e.g., Mino-Kenudson et al., Clin Cancer Res 16(5):1561-1571 (2010).

In one embodiment, an antigen binding domain against polysialic acid comprises an antigen binding portion, e.g., CDRs, of an antibody described in, e.g., Nagae et al., J Biol Chem 288(47):33784-33796 (2013).

In one embodiment, an antigen binding domain against PLAC1 comprises an antigen binding portion, e.g., CDRs, of an antibody described in, e.g., Ghods et al., Biotechnol Appl Biochem 2013 doi:10.1002/bab.1177.

In one embodiment, an antigen binding domain against GloboH comprises an antigen binding portion of the antibody VK9; or an antibody described in, e.g., Kudryashov V et al, Glycoconj J.15(3):243-9 (1998), Lou et al., Proc Natl Acad Sci USA 111(7):2482-2487 (2014); MBr1: Bremer E-G et al. J Biol Chem 259:14773-14777 (1984).

In one embodiment, an antigen binding domain against NY-BR-1 comprises an antigen binding portion, e.g., CDRs of an antibody described in, e.g., Jager et al., Appl Immunohistochem Mol Morphol 15(1):77-83 (2007).

In one embodiment, an antigen binding domain against WT-1 comprises an antigen binding portion, e.g., CDRs, of an antibody described in, e.g., Dao et al., Sci Transl Med 5(176):176ra33 (2013); or WO2012/135854.

In one embodiment, an antigen binding domain against MAGE-A1 comprises an antigen binding portion, e.g., CDRs, of an antibody described in, e.g., Willemsen et al., J Immunol 174(12):7853-7858 (2005) (TCR-like scFv).

In one embodiment, an antigen binding domain against sperm protein 17 comprises an antigen binding portion, e.g., CDRs, of an antibody described in, e.g., Song et al., Target Oncol 2013 Aug. 14 (PMID: 23943313); Song et al., Med Oncol 29(4):2923-2931 (2012).

In one embodiment, an antigen binding domain against Tic 2 comprises an antigen binding portion, e.g., CDRs, of the antibody AB33 (Cell Signaling Technology).

In one embodiment, an antigen binding domain against MAD-CT-2 comprises an antigen binding portion, e.g., CDRs, of an antibody described in, e.g., PMID: 2450952; U.S. Pat. No. 7,635,753.

In one embodiment, an antigen binding domain against Fos-related antigen 1 comprises an antigen binding portion, e.g., CDRs, of the antibody 12F9 (Novus Biologicals).

In one embodiment, an antigen binding domain against MelanA/MART1 comprises an antigen binding portion, e.g., CDRs, of an antibody described in, EP2514766 A2; or U.S. Pat. No. 7,749,719.

In one embodiment, an antigen binding domain against sarcoma translocation breakpoints comprises an antigen binding portion, e.g., CDRs, of an antibody described in, e.g., Luo et al, EMBO Mol. Med. 4(6):453-461 (2012).

In one embodiment, an antigen binding domain against TRP-2 comprises an antigen binding portion, e.g., CDRs, of an antibody described in, e.g., Wang et al, J Exp Med. 184(6):2207-16 (1996).

In one embodiment, an antigen binding domain against CYP1B1 comprises an antigen binding portion, e.g., CDRs, of an antibody described in, e.g., Maecker et al, Blood 102 (9): 3287-3294 (2003).

In one embodiment, an antigen binding domain against RAGE-1 comprises an antigen binding portion, e.g., CDRs, of the antibody MAB5328 (EMD Millipore).

In one embodiment, an antigen binding domain against human telomerase reverse transcriptase comprises an antigen binding portion, e.g., CDRs, of the antibody cat no: LS-B95-100 (Lifespan Biosciences)

In one embodiment, an antigen binding domain against intestinal carboxyl esterase comprises an antigen binding portion, e.g., CDRs, of the antibody 4F12: cat no: LS-B6190-50 (Lifespan Biosciences).

In one embodiment, an antigen binding domain against mut hsp70-2 comprises an antigen binding portion, e.g., CDRs, of the antibody Lifespan Biosciences: monoclonal: cat no: LS-C133261-100 (Lifespan Biosciences).

In one embodiment, an antigen binding domain against CD79a comprises an antigen binding portion, e.g., CDRs, of the antibody Anti-CD79a antibody [HM47/A9] (ab3121), available from Abcam; antibody CD79A Antibody #3351 available from Cell Signaling Technology; or antibody HPA017748-Anti-CD79A antibody produced in rabbit, available from Sigma Aldrich.

In one embodiment, an antigen binding domain against CD79b comprises an antigen binding portion, e.g., CDRs, of the antibody polatuzumab vedotin, anti-CD79b described in Dornan et al., “Therapeutic potential of an anti-CD79b antibody-drug conjugate, anti-CD79b-vc-MMAE, for the treatment of non-Hodgkin lymphoma” Blood. 2009 Sep. 24; 114(13):2721-9. doi: 10.1182/blood-2009-02-205500. Epub 2009 Jul. 24, or the bispecific antibody Anti-CD79b/CD3 described in “4507 Pre-Clinical Characterization of T Cell-Dependent Bispecific Antibody Anti-CD79b/CD3 As a Potential Therapy for B Cell Malignancies” Abstracts of 56^{th} ASH Annual Meeting and Exposition, San Francisco, Calif. Dec. 6-9, 2014.

In one embodiment, an antigen binding domain against CD72 comprises an antigen binding portion, e.g., CDRs, of the antibody J3-109 described in Myers, and Uckun, “An anti-CD72 immunotoxin against therapy-refractory B-lineage acute lymphoblastic leukemia.” Leuk Lymphoma. 1995 June; 18(1-2):119-22, or anti-CD72 (10D6.8.1, mIgG1) described in Poison et al., “Antibody-Drug Conjugates for the Treatment of Non-Hodgkin's Lymphoma: Target and Linker-Drug Selection” Cancer Res Mar. 15, 2009 69; 2358.

In one embodiment, an antigen binding domain against LAIR1 comprises an antigen binding portion, e.g., CDRs, of the antibody ANT-301 LAIR1 antibody, available from ProSpec; or anti-human CD305 (LAIR1) Antibody, available from BioLegend.

In one embodiment, an antigen binding domain against FCAR comprises an antigen binding portion, e.g., CDRs, of the antibody CD89/FCARAntibody (Catalog #10414-H08H), available from Sino Biological Inc.

In one embodiment, an antigen binding domain against LILRA2 comprises an antigen binding portion, e.g., CDRs, of the antibody LILRA2 monoclonal antibody (M17), clone 3C7, available from Abnova, or Mouse Anti-LILRA2 antibody, Monoclonal (2D7), available from Lifespan Biosciences.

In one embodiment, an antigen binding domain against CD300LF comprises an antigen binding portion, e.g., CDRs, of the antibody Mouse Anti-CMRF35-like molecule 1 antibody, Monoclonal[UP-D2], available from BioLegend, or Rat Anti-CMRF35-like molecule 1 antibody, Monoclonal[234903], available from R&D Systems.

In one embodiment, an antigen binding domain against CLEC12A comprises an antigen binding portion, e.g., CDRs, of the antibody Bispecific T cell Engager (BiTE) scFv-antibody and ADC described in Noordhuis et al., “Targeting of CLEC12A In Acute Myeloid Leukemia by Antibody-Drug-Conjugates and Bispecific CLL-1xCD3 BiTE Antibody” 53^{rd} ASH Annual Meeting and Exposition, Dec. 10-13, 2011, and MCLA-117 (Merus).

In one embodiment, an antigen binding domain against BST2 (also called CD317) comprises an antigen binding portion, e.g., CDRs, of the antibody Mouse Anti-CD317 antibody, Monoclonal[3H4], available from Antibodies-Online or Mouse Anti-CD317 antibody, Monoclonal[696739], available from R&D Systems.

In one embodiment, an antigen binding domain against EMR2 (also called CD312) comprises an antigen binding portion, e.g., CDRs, of the antibody Mouse Anti-CD312 antibody, Monoclonal[LS-B8033] available from Lifespan Biosciences, or Mouse Anti-CD312 antibody, Monoclonal[494025] available from R&D Systems.

In one embodiment, an antigen binding domain against LY75 comprises an antigen binding portion, e.g., CDRs, of the antibody Mouse Anti-Lymphocyte antigen 75 antibody, Monoclonal[HD30] available from EMD Millipore or Mouse Anti-Lymphocyte antigen 75 antibody, Monoclonal[A15797] available from Life Technologies.

In one embodiment, an antigen binding domain against GPC3 comprises an antigen binding portion, e.g., CDRs, of the antibody hGC33 described in Nakano K, Ishiguro T, Konishi H, et al. Generation of a humanized anti-glypican 3 antibody by CDR grafting and stability optimization. Anticancer Drugs. 2010 November; 21(10):907-916, or MDX-1414, HN3, or YP7, all three of which are described in Feng et al., “Glypican-3 antibodies: a new therapeutic target for liver cancer.” FEBS Lett. 2014 Jan. 21; 588(2):377-82.

In one embodiment, an antigen binding domain against FCRL5 comprises an antigen binding portion, e.g., CDRs, of the anti-FcRL5 antibody described in Elkins et al., “FcRL5 as a target of antibody-drug conjugates for the treatment of multiple myeloma” Mol Cancer Ther. 2012 October; 11(10):2222-32.

In one embodiment, an antigen binding domain against IGLL1 comprises an antigen binding portion, e.g., CDRs, of the antibody Mouse Anti-Immunoglobulin lambda-like polypeptide 1 antibody, Monoclonal[AT1G4] available from Lifespan Biosciences, Mouse Anti-Immunoglobulin lambda-like polypeptide 1 antibody, Monoclonal[HSL11] available from BioLegend.

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect, the amino acid sequence of the transmembrane domain may be modified or substituted so as to minimize interactions with the binding domains of the native binding partner present in the same CART.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this invention may include at least the transmembrane region(s) of e.g., the alpha, beta or zeta chain of the T-cell receptor, CD28, CD27, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIR2DS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R a, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile). CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, or a CD8a hinge. In one embodiment, the hinge or spacer comprises (e.g., consists of) the amino acid sequence of SEQ ID NO:2. In one aspect, the transmembrane domain comprises (e.g., consists of) a transmembrane domain of SEQ ID NO: 6.

In one aspect, the hinge or spacer comprises an IgG4 hinge. For example, in one embodiment, the hinge or spacer comprises a hinge of the amino acid sequence

(SEQ ID NO: 36)
ESKYGPPCPPCPAPEFLGGPSVFLFPPKPKDTLMISRTPEVTCVVVDVSQ
EDPEVQFNWYVDGVEVHNAKTKPREEQFNSTYRVVSVLTVLHQDWLNGKE
YKCKVSNKGLPSSIEKTISKAKGQPREPQVYTLPPSQEEMTKNQVSLTCL
VKGFYPSDIAVEWESNGQPENNYKTTPPVLDSDGSFFLYSRLTVDKSRWQ
EGNVFSCSVMHEALHNHYTQKSLSLSLGKM.

In some embodiments, the hinge or spacer comprises a hinge encoded by a nucleotide sequence of

(SEQ ID NO: 37)
GAGAGCAAGTACGGCCCTCCCTGCCCCCCTTGCCCTGCCCCCGAGTTCCT
GGGCGGACCCAGCGTGTTCCTGTTCCCCCCCAAGCCCAAGGACACCCTGA
TGATCAGCCGGACCCCCGAGGTGACCTGTGTGGTGGTGGACGTGTCCCAG
GAGGACCCCGAGGTCCAGTTCAACTGGTACGTGGACGGCGTGGAGGTGCA
CAACGCCAAGACCAAGCCCCGGGAGGAGCAGTTCAATAGCACCTACCGGG
TGGTGTCCGTGCTGACCGTGCTGCACCAGGACTGGCTGAACGGCAAGGAA
TACAAGTGTAAGGTGTCCAACAAGGGCCTGCCCAGCAGCATCGAGAAAAC
CATCAGCAAGGCCAAGGGCCAGCCTCGGGAGCCCCAGGTGTACACCCTGC
CCCCTAGCCAAGAGGAGATGACCAAGAACCAGGTGTCCCTGACCTGCCTG
GTGAAGGGCTTCTACCCCAGCGACATCGCCGTGGAGTGGGAGAGCAACGG
CCAGCCCGAGAACAACTACAAGACCACCCCCCCTGTGCTGGACAGCGACG
GCAGCTTCTTCCTGTACAGCCGGCTGACCGTGGACAAGAGCCGGTGGCAG
GAGGGCAACGTCTTTAGCTGCTCCGTGATGCACGAGGCCCTGCACAACCA
CTACACCCAGAAGAGCCTGAGCCTGTCCCTGGGCAAGATG.

In one aspect, the hinge or spacer comprises an IgD hinge. For example, in one embodiment, the hinge or spacer comprises a hinge of the amino acid sequence

(SEQ ID NO: 38)
RWPESPKAQASSVPTAQPQAEGSLAKATTAPATTRNTGRGGEEKKKEKEK
EEQEERETKTPECPSHTQPLGVYLLTPAVQDLWLRDKATFTCFVVGSDLK
DAHLTWEVAGKVPTGGVEEGLLERHSNGSQSQHSRLTLPRSLWNAGTSVT
CTLNHPSLPPQRLMALREPAAQAPVKLSLNLLASSDPPEAASWLLCEVSG
FSPPNILLMWLEDQREVNTSGFAPARPPPQPGSTTFWAWSVLRVPAPPSP
QPATYTCVVSHEDSRTLLNASRSLEVSYVTDH.

In some embodiments, the hinge or spacer comprises a hinge encoded by a nucleotide sequence of

(SEQ ID NO: 103)
AGGTGGCCCGAAAGTCCCAAGGCCCAGGCATCTAGTGTTCCTACTGCACA
GCCCCAGGCAGAAGGCAGCCTAGCCAAAGCTACTACTGCACCTGCCACTA
CGCGCAATACTGGCCGTGGCGGGGAGGAGAAGAAAAAGGAGAAAGAGAAA
GAAGAACAGGAAGAGAGGGAGACCAAGACCCCTGAATGTCCATCCCATAC
CCAGCCGCTGGGCGTCTATCTCTTGACTCCCGCAGTACAGGACTTGTGGC
TTAGAGATAAGGCCACCTTTACATGTTTCGTCGTGGGCTCTGACCTGAAG
GATGCCCATTTGACTTGGGAGGTTGCCGGAAAGGTACCCACAGGGGGGGT
TGAGGAAGGGTTGCTGGAGCGCCATTCCAATGGCTCTCAGAGCCAGCACT
CAAGACTCACCCTTCCGAGATCCCTGTGGAACGCCGGGACCTCTGTCACA
TGTACTCTAAATCATCCTAGCCTGCCCCCACAGCGTCTGATGGCCCTTAG
AGAGCCAGCCGCCCAGGCACCAGTTAAGCTTAGCCTGAATCTGCTCGCCA
GTAGTGATCCCCCAGAGGCCGCCAGCTGGCTCTTATGCGAAGTGTCCGGC
TTTAGCCCGCCCAACATCTTGCTCATGTGGCTGGAGGACCAGCGAGAAGT
GAACACCAGCGGCTTCGCTCCAGCCCGGCCCCCACCCCAGCCGGGTTCTA
CCACATTCTGGGCCTGGAGTGTCTTAAGGGTCCCAGCACCACCTAGCCCC
CAGCCAGCCACATACACCTGTGTTGTGTCCCATGAAGATAGCAGGACCCT
GCTAAATGCTTCTAGGAGTCTGGAGGTTTCCTACGTGACTGACCATT.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO: 5). In some embodiments, the linker is encoded by a nucleotide sequence of

(SEQ ID NO: 8)
GGTGGCGGAGGTTCTGGAGGTGGAGGTTCC.

In one aspect, the hinge or spacer comprises a KIR2DS2 hinge.

### Cytoplasmic Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in a CAR described herein include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta , CD3 epsilon, CD5, CD22, CD79a, CD79b, CD278 (also known as “ICOS”), FcεRI, DAP10, DAP12, and CD66d. In one embodiment, a CAR of the invention comprises an intracellular signaling domain, e.g., a primary signaling domain of CD3-zeta, e.g., a CD3-zeta sequence described herein.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

Costimulatory Signaling Domain

The intracellular signaling domain of the CAR can comprise the CD3-zeta signaling domain by itself or it can be combined with any other desired intracellular signaling domain(s) useful in the context of a CAR of the invention. For example, the intracellular signaling domain of the CAR can comprise a CD3 zeta chain portion and a costimulatory signaling domain. The costimulatory signaling domain refers to a portion of the CAR comprising the intracellular domain of a costimulatory molecule. In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

A costimulatory molecule can be a cell surface molecule other than an antigen receptor or its ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include CDS, ICAM-1, GITR, BAFFR, HVEM (LIGHTR), SLAMF7, NKp80 (KLRF1), NKp30, NKp44, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, NKG2D, NKG2C and PAG/Cbp.

The intracellular signaling sequences within the cytoplasmic portion of the CAR may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequences. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO: 7. In one aspect, the signaling domain of CD3-zeta is a signaling domain of SEQ ID NO: 9.

In one aspect, the intracellular signaling domain is designed to comprise the signaling domain of CD3-zeta and the signaling domain of CD27. In one aspect, the signaling domain of CD27 comprises an amino acid sequence of QRRKYRSNKGESPVEPAEPCRYSCPREEEGSTIPIQEDYRKPEPACSP (SEQ ID NO:16). In one aspect, the signaling domain of CD27 is encoded by a nucleic acid sequence of AGGAGTAAGAGGAGCAGGCTCCTGCACAGTGACTACATGAACATGACTCCCCGCC GCCCCGGGCCCACCCGCAAGCATTACCAGCCCTATGCCCCACCACGCGACTTCGCA GCCTATCGCTCC (SEQ ID NO:14).

In one aspect, the CAR-expressing cell described herein can further comprise a second CAR, e.g., a second CAR that includes a different antigen binding domain, e.g., to the same target or a different target (e.g., a target other than a cancer associated antigen described herein or a different cancer associated antigen described herein, e.g., CD19, CD33, CLL-1, CD34, FLT3, or folate receptor beta). In one embodiment, the second CAR includes an antigen binding domain to a target expressed the same cancer cell type as the cancer associated antigen. In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be hound by theory, placement of a costimulatory signaling domain, e.g., 4-1BB, CD28, ICOS, CD27 or OX-40, onto the first CAR, and the primary signaling domain, e.g., CD3 zeta, on the second CAR can limit the CAR activity to cells where both targets are expressed. In one embodiment, the CAR expressing cell comprises a first cancer associated antigen CAR that includes an antigen binding domain that binds a target antigen described herein, a transmembrane domain and a costimulatory domain and a second CAR that targets a different target antigen (e.g., an antigen expressed on that same cancer cell type as the first target antigen) and includes an antigen binding domain, a transmembrane domain and a primary signaling domain. In another embodiment, the CAR expressing cell comprises a first CAR that includes an antigen binding domain that binds a target antigen described herein, a transmembrane domain and a primary signaling domain and a second CAR that targets an antigen other than the first target antigen (e.g., an antigen expressed on the same cancer cell type as the first target antigen) and includes an antigen binding domain to the antigen, a transmembrane domain and a costimulatory signaling domain.

In another aspect, the disclosure features a population of CAR-expressing cells, e.g., CART cells. In some embodiments, the population of CAR-expressing cells comprises a mixture of cells expressing different CARs.

For example, in one embodiment, the population of CART cells can include a first cell expressing a CAR having an antigen binding domain to a cancer associated antigen described herein, and a second cell expressing a CAR having a different antigen binding domain, e.g., an antigen binding domain to a different a cancer associated antigen described herein, e.g., an antigen binding domain to a cancer associated antigen described herein that differs from the cancer associate antigen bound by the antigen binding domain of the CAR expressed by the first cell.

As another example, the population of CAR-expressing cells can include a first cell expressing a CAR that includes an antigen binding domain to a cancer associated antigen described herein, and a second cell expressing a CAR that includes an antigen binding domain to a target other than a cancer associate antigen as described herein. In one embodiment, the population of CAR-expressing cells includes, e.g., a first cell expressing a CAR that includes a primary intracellular signaling domain, and a second cell expressing a CAR that includes a secondary signaling domain.

In another aspect, the disclosure features a population of cells wherein at least one cell in the population expresses a CAR having an antigen binding domain to a cancer associated antigen described herein, and a second cell expressing another agent, e.g., an agent which enhances the activity of a CAR-expressing cell. For example, in one embodiment, the agent can be an agent which inhibits an inhibitory molecule. Inhibitory molecules, e.g., PD-1, can, in some embodiments, decrease the ability of a CAR-expressing cell to mount an immune effector response. Examples of inhibitory molecules include PD-1, PD-L1, PD-L2, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta). In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD-1, PD-L1, CTLA4, TIM3, CEACAM (CEACAM-1, CEACAM-3, and/or CEACAM-5), LAG3, VISTA, BTLA, TIGIT, LAIR1, CD160, 2B4 and TGF beta, or a fragment of any of these, and a second polypeptide which is an intracellular signaling domain described herein (e.g., comprising a costimulatory domain (e.g., 41BB, CD27, OX40 or CD28, e.g., as described herein) and/or a primary signaling domain (e.g., a CD3 zeta signaling domain described herein). In one embodiment, the agent comprises a first polypeptide of PD-1 or a fragment thereof, and a second polypeptide of an intracellular signaling domain described herein (e.g., a CD28 signaling domain described herein and/or a CD3 zeta signaling domain described herein).

The sequences of anti-CD19 binding domains are provided herein in Table 1. Full CAR constructs can be generated using any of the antigen binding domains described in Table 1 with one or more additional CAR component provided below.

leader (amino acid sequence)
(SEQ ID NO: 1)
MALPVTALLLPLALLLHAARP
leader (nucleic acid sequence)
(SEQ ID NO: 12)
ATGGCCCTGCCTGTGACAGCCCTGCTGCTGCC
TCTGGCTCTGCTGCTGCATGCCGCTAGACCC
leader (nucleic acid sequence 2)
(SEQ ID NO: 127)
ATGGCCCTCCCTGTCACCGCCCTGCTGCTTCC
GCTGGCTCTTCTGCTCCACGCCGCTCGGCCC
leader (nucleic acid sequence 3)
(SEQ ID NO: 128)
ATGGCCTTACCAGTGACCGCCTTGCTCCTGCC
GCTGGCCTTGCTGCTCCACGCCGCCAGGCCG
CD8 hinge (amino acid sequence)
(SEQ ID NO: 2)
TTTPAPRPPTPAPTIASQPLSLRPEACRPAAGGAVHTRGLDFACD
CD8 hinge (nucleic acid sequence)
(SEQ ID NO: 13)
ACCACGACGCCAGCGCCGCGACCACCAACACCGGCGCCCACCATCGCGTCG
CAGCCCCTGTCCCTGCGCCCAGAGGCGTGCCGGCCAGCGGCGGGGGGCGC
AGTGCACACGAGGGGGCTGGACTTCGCCTGTGAT
CD8 hinge (nucleic acid sequence 2)
(SEQ ID NO: 129)
ACCACTACCCCAGCACCGAGGCCACCCACCCCGGCTCCTACCATCGCCTCC
CAGCCTCTGTCCCTGCGTCCGGAGGCATGTAGACCCGCAGCTGGTGGGGCC
GTGCATACCCGGGGTCTTGACTTCGCCTGCGAT
CD8 transmembrane (amino acid sequence)
(SEQ ID NO: 6)
IYIWAPLAGTCGVLLLSLVITLYC
transmembrane (nucleic acid sequence)
(SEQ ID NO: 17)
ATCTACATCTGGGCGCCCTTGGCCGGGACTTGTGGG
GTCCTTCTCCTGTCACTGGTTATCACCCTTTACTGC
transmembrane (nucleic acid sequence 2)
(SEQ ID NO: 130)
ATCTACATTTGGGCCCCTCTGGCTGGTACTTGCGGG
GTCCTGCTGCTTTCACTCGTGATCACTCTTTACTGT
4-1BB Intracellular domain (amino acid sequence)
(SEQ ID NO: 7)
KRGRKKLLYIFKQPFMRPVQTTQEEDGCSCRFPEEEEGGCEL
4-1BB Intracellular domain (nucleic acid sequence)
(SEQ ID NO: 18)
AAACGGGGCAGAAAGAAACTCCTGTATATATTCAAACAACCATTTATGAG
ACCAGTACAAACTACTCAAGAGGAAGATGGCTGTAGCTGCCGATTTCCAGA
AGAAGAAGAAGGAGGATGTGAACTG
4-1BB Intracellular domain (nucleic acid
sequence 2)
(SEQ ID NO: 131)
AAGCGCGGTCGGAAGAAGCTGCTGTACATCTTTAAGCAACCCTTCATGAGG
CCTGTGCAGACTACTCAAGAGGAGGACGGCTGTTCATGCCGGTTCCCAGAG
GAGGAGGAAGGCGGCTGCGAACTG
CD3 zeta domain (amino acid sequence)
(SEQ ID NO: 9)
RVKFSRSADAPAYKQGQNQLYNELNLGRREEYDVLDKRRGRDPEMGGKPRR
KNPQEGLYNELQKDKMAEAYSEIGMKGERRRGKGHDGLYQGLSTATKDTYD
ALHMQALPPR
CD3 zeta (nucleic acid sequence)
(SEQ ID NO: 20)
AGAGTGAAGTTCAGCAGGAGCGCAGACGCCCCCGCGTACAAGCAGGGCCA
GAACCAGCTCTATAACGAGCTCAATCTAGGACGAAGAGAGGAGTACGATGT
TTTGGACAAGAGACGTGGCCGGGACCCTGAGATGGGGGGAAAGCCGAGAAG
GAAGAACCCTCAGGAAGGCCTGTACAATGAACTGCAGAAAGATAAGATGGC
GGAGGCCTACAGTGAGATTGGGATGAAAGGCGAGCGCCGGAGGGGCAAGGG
GCACGATGGCCTTTACCAGGGTCTCAGTACAGCCACCAAGGACACCTACGA
CGCCCTTCACATGCAGGCCCTGCCCCCTCGC
CD3 zeta (nucleic acid sequence 2)
(SEQ ID NO: 132)
CGCGTGAAATTCAGCCGCAGCGCAGATGCTCCAGCCTACAAGCAGGGGCA
GAACCAGCTCTACAACGAACTCAATCTTGGTCGGAGAGAGGAGTACGACGT
GCTGGACAAGCGGAGAGGACGGGACCCAGAAATGGGCGGGAAGCCGCGCAG
AAAGAATCCCCAAGAGGGCCTGTACAACGAGCTCCAAAAGGATAAGATGGC
AGAAGCCTATAGCGAGATTGGTATGAAAGGGGAACGCAGAAGAGGCAAAGG
CCACGACGGACTGTACCAGGGACTCAGCACCGCCACCAAGGACACCTATGA
CGCTCTTCACATGCAGGCCCTGCCGCCTCGG
CD3 zeta domain (amino acid sequence;
NCBI Reference Sequence NM_000734.3)
(SEQ ID NO: 10)
RVKFSRSADAPAYQQGQNQLYNELNLGRREEYDVLDKRRGRDPEMGGKPRR
KNPQEGLYNELQKDKMAEAYSEIGMKGERRRGKGHDGLYQGLSTATKDTYD
ALHMQALPPR
CD3 zeta (nucleic acid sequence;
NCBI Reference Sequence NM_000734.3);
(SEQ ID NO: 21)
agagtgaagttcagcaggagcgcagacgcccccgcgtaccagcagggccag
aaccagctctataacgagctcaatctaggacgaagagaggagtacgatgtt
ttggacaagagacgtggccgggaccctgagatggggggaaagccgagaagg
aagaaccctcaggaaggcctgtacaatgaactgcagaaagataagatggcg
gaggcctacagtgagattgggatgaaaggcgagcgccggaggggcaagggg
cacgatggcctttaccagggtctcagtacagccaccaaggacacctacgac
gcccttcacatgcaggccctgccccctcgc
IgG4 Hinge (amino acid sequence)
(SEQ ID NO: 36)
ESKYGPPCPPCPAPEFLGGPSVFLFPPKPKDTLMISRTPEVTCVVVDVSQE
DPEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVKG
FYPSDIAVEWESNGQPENNYKTTPPVLDSDGSFFLYSRLTVDKSRWQEGNV
FSCSVMHEALHNHYTQKSLSLSLGKM
IgG4 Hinge (nucleotide sequence)
(SEQ ID NO: 37)
GAGAGCAAGTACGGCCCTCCCTGCCCCCCTTGCCCTGCCCCCGAGTTCCTG
GGCGGACCCAGCGTGTTCCTGTTCCCCCCCAAGCCCAAGGACACCCTGATG
ATCAGCCGGACCCCCGAGGTGACCTGTGTGGTGGTGGACGTGTCCCAGGAG
GACCCCGAGGTCCAGTTCAACTGGTACGTGGACGGCGTGGAGGTGCACAAC
GCCAAGACCAAGCCCCGGGAGGAGCAGTTCAATAGCACCTACCGGGTGGTG
TCCGTGCTGACCGTGCTGCACCAGGACTGGCTGAACGGCAAGGAATACAAG
TGTAAGGTGTCCAACAAGGGCCTGCCCAGCAGCATCGAGAAAACCATCAGC
AAGGCCAAGGGCCAGCCTCGGGAGCCCCAGGTGTACACCCTGCCCCCTAGC
CAAGAGGAGATGACCAAGAACCAGGTGTCCCTGACCTGCCTGGTGAAGGGC
TTCTACCCCAGCGACATCGCCGTGGAGTGGGAGAGCAACGGCCAGCCCGAG
AACAACTACAAGACCACCCCCCCTGTGCTGGACAGCGACGGCAGCTTCTTC
CTGTACAGCCGGCTGACCGTGGACAAGAGCCGGTGGCAGGAGGGCAACGTC
TTTAGCTGCTCCGTGATGCACGAGGCCCTGCACAACCACTACACCCAGAAG
AGCCTGAGCCTGTCCCTGGGCAAGATG
EF1 alpha promoter
(SEQ ID NO: 11)
CGTGAGGCTCCGGTGCCCGTCAGTGGGCAGAGCGCACATCGCCCACAGTCC
CCGAGAAGTTGGGGGGAGGGGTCGGCAATTGAACCGGTGCCTAGAGAAGGT
GGCGCGGGGTAAACTGGGAAAGTGATGTCGTGTACTGGCTCCGCCTTTTTC
CCGAGGGTGGGGGAGAACCGTATATAAGTGCAGTAGTCGCCGTGAACGTTC
TTTTTCGCAACGGGTTTGCCGCCAGAACACAGGTAAGTGCCGTGTGTGGTT
CCCGCGGGCCTGGCCTCTTTACGGGTTATGGCCCTTGCGTGCCTTGAATTA
CTTCCACCTGGCTGCAGTACGTGATTCTTGATCCCGAGCTTCGGGTTGGAA
GTGGGTGGGAGAGTTCGAGGCCTTGCGCTTAAGGAGCCCCTTCGCCTCGTG
CTTGAGTTGAGGCCTGGCCTGGGCGCTGGGGCCGCCGCGTGCGAATCTGGT
GGCACCTTCGCGCCTGTCTCGCTGCTTTCGATAAGTCTCTAGCCATTTAAA
ATTTTTGATGACCTGCTGCGACGCTTTTTTTCTGGCAAGATAGTCTTGTAA
ATGCGGGCCAAGATCTGCACACTGGTATTTCGGTTTTTGGGGCCGCGGGCG
GCGACGGGGCCCGTGCGTCCCAGCGCACATGTTCGGCGAGGCGGGGCCTGC
GAGCGCGGCCACCGAGAATCGGACGGGGGTAGTCTCAAGCTGGCCGGCCTG
CTCTGGTGCCTGGCCTCGCGCCGCCGTGTATCGCCCCGCCCTGGGCGGCAA
GGCTGGCCCGGTCGGCACCAGTTGCGTGAGCGGAAAGATGGCCGCTTCCCG
GCCCTGCTGCAGGGAGCTCAAAATGGAGGACGCGGCGCTCGGGAGAGCGGG
CGGGTGAGTCACCCACACAAAGGAAAAGGGCCTTTCCGTCCTCAGCCGTCG
CTTCATGTGACTCCACGGAGTACCGGGCGCCGTCCAGGCACCTCGATTAGT
TCTCGAGCTTTTGGAGTACGTCGTCTTTAGGTTGGGGGGAGGGGTTTTATG
CGATGGAGTTTCCCCACACTGAGTGGGTGGAGACTGAAGTTAGGCCAGCTT
GGCACTTGATGTAATTCTCCTTGGAATTTGCCCTTTTTGAGTTTGGATCTT
GGTTCATTCTCAAGCCTCAGACAGTGGTTCAAAGTTTTTTTCTTCCATTTC
AGGTGTCGTGA.
Gly/Ser
(SEQ ID NO: 25)
GGGGS
Gly/Ser (SEQ ID NO: 26):
This sequence may encompass 1-6
“Gly Gly Gly Gly Ser” repeating units
GGGGSGGGGS GGGGSGGGGS GGGGSGGGGS
Gly/Ser
(SEQ ID NO: 27)
GGGGSGGGGS GGGGSGGGGS
Gly/Ser
(SEQ ID NO: 28)
GGGGSGGGGS GGGGS
Gly/Ser
(SEQ ID NO: 29)
GGGS
PolyA (SEQ ID NO: 30): A5000
PolyA (SEQ ID NO: 31): A100
PolyT (SEQ ID NO: 32): T5000
PolyA (SEQ ID NO: 33): A5000
PolyA (SEQ ID NO: 34): A400
PolyA (SEQ ID NO: 35)″ A2000
Gly/Ser (SEQ ID NO: 15):
This sequence may encompass
1-10 “Gly Gly Gly Ser” repeating units
GGGSGGGSGG GSGGGSGGGS GGGSGGGSGG GSGGGSGGGS

Exemplary CD19 CAR constructs that can be used in the methods described herein are shown in Table 3:

TABLE 3
CD19 CAR Constructs

| | | |
|---|---|---|
| Name | SEQ ID | Sequence |

CAR 1

| | | |
|---|---|---|
| CAR1 scFv | 39 | EIVMTQSPATLSLSPGERATLSGRASQDISKYLNWYQQKPGQAPRLLIYHT |
| domain | | SRLHSGIPARFSGSGSGTDYTLTISSLQPEDFAVYFCQQGNTLPYTFGQGT |
| | | KLEIKGGGGSGGGGSGGGGSQVQLQESGPGLVKPSETLSLTCTVSGVSLPD |
| | | YGVSWIRQPPGKGLEWIGVIWGSETTYYSSSLKSRVTISKDNSKNQVSLKL |
| | | SSVTAADTAVYYCAKHYYYGGSYAMDYWGQGTLVTVSS |
| 103101 | 52 | atggccctccctgtcaccgccctgctgcttccgctggctcttctgctccacgccgc |
| CAR1 | | tcggcccgaaattgtgatgacccagtcacccgccactcttagcctttcacccggtg |
| Soluble | | agcgcgcaaccctgtcttgcagagcctcccaagacatctcaaaataccttaattgg |
| scFV - nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactactcttcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagccaccaccatcatcaccatcaccat |
| 103101 | 64 | MALPVTALLLPLALLLHAARPeivmtqspatlslspgeratlscrasqdiskylnw |
| CAR1 | | yqqkpgqaprlliyhtsrlhsgiparfsgsgsgtdytltisslqpedfavyfcqqg |
| Soluble | | ntlpytfgqgtkleikggggsggggsggggsqvqlqesgpglvkpsetlsltctvs |
| scF17 - aa | | gvslpdygvswirqppgkglewigviwgsettyyssslksrvtiskdnsknqvslk |
| | | lssvtaadtavyycakhyyyggsyamdywgqgtlvtvsshhhhhhhh |
| 104875 | 90 | atggccctccctgtcaccgccctgctgcttccgctggctcttctgctccacgccgc |
| CAR 1 - | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactactcttcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggagggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104875 | 77 | MALPVTALLLPLALLLHAARPeivmtqspatlslspgeratlscrasqdiskylnw |
| CAR 1 - | | yqqkpgqaprlliyhtsrlhsgiparfsgsgsgtdytltisslqpedfavyfcqqg |
| Full - aa | | ntlpytfgqgtkleikggggsggggsggggsqvqlqesgpglvkpsetlsltctvs |
| | | gvslpdygvswirqppgkglewigviwgsettyyssslksrvtiskdnsknqvslk |
| | | lssvtaadtavyycakhyyyggsyamdywgqgtlvtvsstttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR 2

| | | |
|---|---|---|
| CAR2 scFv | 40 | eivmtqspatlslspgeratlscrasqdiskylnwyqqkpgqaprlliyhtsrlhs |
| domain | | giparfsgsgsgtdytltisslqpedfavyfcqqgntlpytfgqgtk1eikggggs |
| | | ggggsggggsqvqlqesgpglvkpsetlsltctvsgvslpdygvswirqppgkgle |
| | | wigviwgsettyyqsslksrvtiskdnsknqvslklssvtaadtavyycakhyyyg |
| | | gsyamdywgqgtlvtvss |
| 103102 | 53 | atggccctccctgtcaccgccctgctgcttccgctggctcttctgctccacgccgc |
| CAR2 - | | tcggcccgaaattgtgatgacccagtcacccgccactcttagcctttcacccggtg |
| Soluble | | agcgcgcaaccctgtcttgcagagcctcccaagacatctcaaaataccttaattgg |
| scFv - nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactaccaatcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagccaccaccatcatcaccatcaccat |
| 10312 | 65 | MALPVTALLLPLALLLHAARPeivmtqspatlslspgeratlscrasqdiskylnw |
| CAR2 - | | yqqkpgqaprlliyhtsrlhsgiparfsgsgsgtdytltisslqpedfavyfcqqg |
| Soluble | | ntlpytfgqgtkleikggggsggggsggggsqvqlqesgpglvkpsetlsltctvs |
| scFv - aa | | gvslpdygvswirqppgkglewigviwgsettyyqsslksrvtiskdnsknqvslk |
| | | lssvtaadtavyycakhyyyggsyamdywgqgtlvtvsshhhhhhhh |
| 104876 | 91 | atggccctccctgtcaccgccctgctgcttccgctggctcttctgctccacgccgc |
| CAR 2 - | | tcggcccgaaattgtgatgacccagtcacccgccactcttagcctttcacccggtg |
| Full -nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactaccaatcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104876 | 78 | MALPVTALLLPLALLLHAARPeivmtqspatlslspgeratlscrasqdiskylnw |
| CAR 2 - | | yqqkpgqaprlliyhtsrlhsgiparfsgsgsgtdytltisslqpedfavyfcqqg |
| Full - aa | | ntlpytfgqgtkleikggggsggggsggggsqvqlqesgpglvkpsetlsltctvs |
| | | gvslpdygvswirqppgkglewigviwgsettyyqsslksrvtiskdnsknqvslk |
| | | lssvtaadtavyycakhyyyggsyamdywgqgtlvtvsstttpaprpptpaptias |
| | | qplslrpeacrpaaggavntrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR 3

| | | |
|---|---|---|
| CAR3 scFv | 41 | qvqlqesgpglvkpsetlsltctvsgvslpdygvswirqppgkglewigviwgset |
| domain | | tyyssslksrvtiskdnsknqvslklssvtaadtavyycakhyyyggsyamdywgq |
| | | gtlvtvssggggsggggsggggseivmtqspatlslspgeratlscrasqdiskyl |
| | | nwyqqkpgqaprlliyhtsrlhsgiparfsgsgsgtdytltisslqpedfavyfcq |
| | | qgntlpytfgqgtkleik |
| 103104 | 54 | atggctctgcccgtgaccgcactcctcctgccactggctctgctgcttcacgccgc |
| CAR 3 - | | tcgcccacaagtccagcttcaagaatcagggcctggtctggtgaagccatctgaga |
| Soluble | | ctctgtccctcacttgcaccgtgagcggagtgtccctcccagactacggagtgagc |
| scFv - nt | | tggattagacagcctcccggaaagggactggagtggatcggagtgatttggggtag |
| | | cgaaaccacttactattcatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaacatcaccaccatcatcaccatcac |
| 103104 | 66 | MALPVTALLLPLALLLHAARPqvqlqesgpglvkpsetlsltctvsgvslpdygvs |
| CAR 3 - | | wirqppgkglewigviwgsettyyssslksrvtiskdnsknqvslklssvtaadta |
| Soluble | | vyycakhyyyggsyamdywgqgtlvtvssggggsggggsggggseivmtqspatls |
| scFv - aa | | lspgeratlscrasqdiskylnwyqqkpgqaprlliyhtsrlhsgiparfsgsgsg |
| | | tdytltisslqpedfavyfcqqgntlpytfgqgtkleikhhhhhhhh |
| 104877 | 92 | atggctctgcccgtgaccgcactcctcctgccactggctctgctgcttcacgccgc |
| CAR 3 - | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactattcatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaaaccactactcccgctccaaggccacccacccctgccccgaccatcgcctct |
| | | cagccgctttccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104877 | 79 | MALPVTALLLPLALLLHAARPqvqlqesgpglvkpsetlsltctvsgvslpdygvs |
| CAR 3 - | | wirqppgkglewigviwgsettyyssslksrvtiskdnsknqvslklssvtaadta |
| Full - aa | | vyycakhyyyggsyamdywgqgtlvtvssggggsggggsggggseivmtqspatls |
| | | lspgeratlscrasqdiskylnwyqqkpgqaprlliyhtsrlhsgiparfsgsgsg |
| | | tdytltisslqpedfavyfcqqgntlpytfgqgtkleiktttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR 4

| | | |
|---|---|---|
| CAR4 scFv | 42 | qvqlqesgpglvkpsetlsltctvsgvslpdygyswirqppgkglewigviwgset |
| domain | | tyyqsslksrvtiskdnsknqvslklssvtaadtavyycakhyyyggsyamdywgq |
| | | gtlvtvssggggsggggsggggseivmtqspatlslspgeratlscrasqdiskyl |
| | | nwyqqkpgqaprlliyhtsrlhsgiparfsgsgsgtdytltisslqpedfavyfcq |
| | | qgntlpytfgqgtkleik |
| 103106 | 55 | atggctctgcccgtgaccgcactcctcctgccactggctctgctgcttcacgccgc |
| CAR4 - | | tcgcccacaagtccagcttcaagaatcagggcctggtctggtgaagccatctgaga |
| Soluble | | ctctgtccctcacttgcaccgtgagcggagtgtccctcccagactacggagtgagc |
| scFv - nt | | tggattagacagcctcccggaaagggactggagtggatcggagtgatttggggtag |
| | | cgaaaccacttactatcaatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaacatcaccaccatcatcaccatcac |
| 103106 | 67 | MALPVTALLLPLALLLHAARPqvqlqesgpglvkpsetlsltctvsgvslpdygvs |
| CAR4- | | wirqppgkglewigviwgsettyyqsslksrvtiskdnsknqvslklssvtaadta |
| Soluble | | vyycakhyyyggsyamdywgqgtlvtvssggggsggggsggggseivmtqspatls |
| scF17 - aa | | lspgeratlscrasqdiskylnwyqqkpgqaprlliyhtsrlhsgiparfsgsgsg |
| | | tdytltisslqpedfavyfcqqgntlpytfgqgtkleikhhhhhhhh |
| 104878 | 93 | atggctctgcccgtgaccgcactcctcctgccactggctctgctgcttcacgccgc |
| CAR 4 - | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactatcaatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaaaccactactcccgctccaaggccacccacccctgccccgaccatcgcctct |
| | | cagccgctttccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 104878 | 80 | MALPVTALLLPLALLLHAARPqvqlqesgpglvkpsetlsltctvsgvslpdygvs |
| CAR 4 - | | wirqppgkglewigviwgsettyyqsslksrvtiskdnsknqvslklssvtaadta |
| Full - aa | | vyycakhyyyggsyamdywgqgtlvtvssggggsggggsggggseivmtqspatls |
| | | lspgeratlscrasqdiskylnwyqqkpgqaprlliyhtsrlhsgiparfsgsgsg |
| | | tdytltisslqpedfavyfcqqgntlpytfgqgtkleiktttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |

CAR 5

| | | |
|---|---|---|
| CAR5 scFv | 43 | eivmtqspatlslspgeratlscrasqdiskylnwyqqkpgqaprlliyhtsrlhs |
| domain | | giparfsgsgsgtdytltisslqpedfavyfcqqgntlpytfgqgtkleikggggs |
| | | ggggsggggsggggsqvqlqesgpglvkpsetlsltctvsgvslpdygvswirqpp |
| | | gkglewigviwgsettyyssslksrvtiskdnsknqvslklssvtaadtavyycak |
| | | hyyyggsyamdywgqgtlvtvss |
| 99789 | 56 | atggccctcccagtgaccgctctgctgctgcctctcgcacttcttctccatgccgc |
| CAR5 - | | tcggcctgagatcgtcatgacccaaagccccgctaccctgtccctgtcacccggcg |
| Soluble | | agagggcaaccctttcatgcagggccagccaggacatttctaagtacctcaactgg |
| scFv - nt | | tatcagcagaagccagggcaggctcctcgcctgctgatctaccacaccagccgcct |
| | | ccacagcggtatccccgccagattttccgggagcgggtctggaaccgactacaccc |
| | | tcaccatctcttctctgcagcccgaggatttcgccgtctatttctgccagcagggg |
| | | aatactatgccgtacaccttcggtcaaggtaccaagctggaaatcaagggaggcgg |
| | | aggatcaggcggtggcggaagcggaggaggtggctccggaggaggaggttcccaag |
| | | tgcagcttcaagaatcaggacccggacttgtgaagccatcagaaaccctctccctg |
| | | acttgtaccgtgtccggtgtgagcctccccgactacggagtctcttggattcgcca |
| | | gcctccggggaagggtcttgaatggattggggtgatttggggatcagagactactt |
| | | actactcttcatcacttaagtcacgggtcaccatcagcaaagataatagcaagaac |
| | | caagtgtcacttaagctgtcatctgtgaccgccgctgacaccgccgtgtactattg |
| | | tgccaaacattactattacggagggtcttatgctatggactactggggacagggga |
| | | ccctggtgactgtctctagccatcaccatcaccaccatcatcac |
| 99789 | 68 | MALPVTALLLPLALLLHAARPeivmtqspatlslspgeratlscrasqdiskylnw |
| CAR5 - | | yqqkpgqaprlliyhtsrlhsgiparfsgsgsgtdytltisslqpedfavyfcqqg |
| Soluble | | ntlpytfgqgtkleikggggsggggsggggsggggsqvqlqesgpglvkpsetlsl |
| scFv - aa | | tctvsgvslpdygvswirqppgkglewigviwgsettyyssslksrvtiskdnskn |
| | | qvslklssvtaadtavyycakhyyyggsyamdywgqgtlvtvsshhhhhhhh |
| 104879 | 94 | atggccctccctgtcaccgccctgctgcttccgctggctcttctgctccacgccgc |
| CAR 5 | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcatgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggcggaggcgggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actactcttcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104879 | 81 | MALPVTALLLPLALLLHAARPeivmtqspatlslspgeratlscrasqdiskylnw |
| CAR 5 - | | yqqkpgqaprlliyhtsrlhsgiparfsgsgsgtdytltisslqpedfavyfcqqg |
| Full - aa | | ntlpytfgqgtkleikggggsggggsggggsggggsqvqlqesgpglvkpsetlsl |
| | | tctvsgvslpdygvswirqppgkglewigviwgsettyyssslksrvtiskdnskn |
| | | qvslklssvtaadtavyycakhyyyggsyamdywgqgtlvtvsstttpaprpptpa |
| | | ptiasqplslrpeacrpaaggavhtrgldfacdiyiwaplagtcgvlllslvitly |
| | | ckrgrkkllyifkqpfmrpvqttqeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 6

| | | |
|---|---|---|
| CAR6 | 44 | eivmtqspatlslspgeratlscrasqdiskylnwyqqkpgqaprlliyhtsrlhs |
| scFv | | giparfsgsgsgtdytltisslqpedfavyfcqqgntlpytfgqgtkleikggggs |
| domain | | ggggsggggsggggsqvqlqesgpglvkpsetlsltctvsgvslpdygvswirqpp |
| | | gkglewigviwgsettyyqsslksrvtiskdnsknqvslklssvtaadtavyycak |
| | | hyyyggsyamdywgqgtlvtvss |
| 99790 | 57 | atggccctcccagtgaccgctctgctgctgcctctcgcacttcttctccatgccgc |
| CAR6 - | | tcggcctgagatcgtcatgacccaaagccccgctaccctgtccctgtcacccggcg |
| Soluble | | agagggcaaccctttcatgcagggccagccaggacatttctaagtacctcaactgg |
| SCFv - nt | | tatcagcagaagccagggcaggctcctcgcctgctgatctaccacaccagccgcct |
| | | ccacagcggtatccccgccagattttccgggagcgggtctggaaccgactacaccc |
| | | tcaccatctcttctctgcagcccgaggatttcgccgtctatttctgccagcagggg |
| | | aatactctgccgtacaccttcggtcaaggtaccaagctggaaatcaagggaggcgg |
| | | aggatcaggcggtggcggaagcggaggaggtggctccggaggaggaggttcccaag |
| | | tgcagcttcaagaatcaggacccggacttgtgaagccatcagaaaccctctccctg |
| | | acttgtaccgtgtccggtgtgagcctcaccgactacggagtctcttggattcgcca |
| | | gcctccggggaagggtcttgaatggattggggtgatttggggatcagagactactt |
| | | actaccagtcatcacttaagtcacgggtcaccatcagcaaagataatagcaagaac |
| | | caagtgtcacttaagctgtcatctgtgaccgccgctgacaccgccgtgtactattg |
| | | tgccaaacattactattacggagggtcttatgctatggactactggggacagggga |
| | | ccctggtgactgtctctagccatcaccatcaccaccatcatcac |
| 99790 | 69 | MALPVTALLLPLALLLHAARPeivmtqspatlslspgeratlscrasqdiskylnw |
| CAR6 - | | yqqkpgqaprlliyhtsrlhsgiparfsgsgsgtdytltisslqpedfavyfcqqg |
| Soluble | | ntlpytfgqgtkleikggggsggggsggggsggggsqvqlqesgpglvkpsetlsl |
| scFv - aa | | tctvsgvslpdygvswirqppgkglewigviwgsettyyqsslksrvtiskdnskn |
| | | qvslklssvtaadtavyycakhyyyggsyamdywgqgtlvtvsshhhhhhhh |
| 104880 | 95 | atggccctccctgtcaccgccctgctgcttccgctggctcttctgctccacgccgc |
| CAR6 - | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggaggcggagggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actaccaatcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104880 | 82 | MALPVTALLLPLALLLHAARPeivmtqspatlslspgeratlscrasqdiskylnw |
| CAR6 - | | yqqkpgqaprlliyhtsrlhsgiparfsgsgsgtdytltisslqpedfavyfcqqg |
| Full - aa | | ntlpytfgqgtkleikggggsggggsggggsggggsqvqlqesgpglvkpsetlsl |
| | | tctvsgvslpdygvswirqppgkglewigviwgsettyyqsslksrvtiskdnskn |
| | | qvslklssvtaadtavyycakhyyyggsyamdywgqgtlvtvsstttpaprpptpa |
| | | ptiasqplslrpeacrpaaggavntrgldfacdiyiwaplagtcgvlllslvitly |
| | | ckrgrkkllyifkqpfmrpvqttqeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 7

| | | |
|---|---|---|
| CAR7 scFv | 45 | qvqlqesgpglvkpsetlsltctvsgvslpdygvswirqppgkglewigviwgset |
| domain | | tyyssslksrvtiskdnsknqvslklssvtaadtavyycaknyyyggsyamdywgq |
| | | gtlvtvssggggsggggsggggsggggseivmtqspatlslspgeratlscrasqd |
| | | iskylnwyqqkpgqaprlliyhtsrlhsgiparfsgsgsgtdytltisslqpedfa |
| | | vyfcqqgntlpytfgqgtkleik |
| 100796 | 58 | atggcactgcctgtcactgccctcctgctgcctctggccctccttctgcatgccgc |
| CAR7 - | | caggccccaagtccagctgcaagagtcaggacccggactggtgaagccgtctgaga |
| Soluble | | ctctctcactgacttgtaccgtcagcggcgtgtccctccccgactacggagtgtca |
| scFv - nt | | tggatccgccaacctcccgggaaagggcttgaatggattggtgtcatctggggttc |
| | | tgaaaccacctactactcatcttccctgaagtccagggtgaccatcagcaaggata |
| | | attccaagaaccaggtcagccttaagctgtcatctgtgaccgctgctgacaccgcc |
| | | gtgtattactgcgccaagcactactattacggaggaagctacgctatggactattg |
| | | gggacagggcactctcgtgactgtgagcagcggcggtggagggtctggaggtggag |
| | | gatccggtggtggtgggtcaggcggaggagggagcgagattgtgatgactcagtca |
| | | ccagccaccctttctctttcacccggcgagagagcaaccctgagctgtagagccag |
| | | ccaggacatttctaagtacctcaactggtatcagcaaaaaccggggcaggcccctc |
| | | gcctcctgatctaccatacctcacgccttcactctggtatccccgctcggtttagc |
| | | ggatcaggatctggtaccgactacactctgaccatttccagcctgcagccagaaga |
| | | tttcgcagtgtatttctgccagcagggcaatacccttccttacaccttcggtcagg |
| | | gaaccaagctcgaaatcaagcaccatcaccatcatcaccaccat |
| 100796 | 70 | MALPVTALLLPLALLLHAARPqvqlqesgpglvkpsetlsltctvsgvslpdygvs |
| CAR7 - | | wirqppgkglewigviwgsettyyssslksrvtiskdnsknqvslklssvtaadta |
| Soluble | | vyycakhyyyggsyamdywgqgtlvtvssggggsggggsggggsggggseivmtqs |
| scFv - aa | | patlslspgeratlscrasqdiskylnwyqqkpgqaprlliyhtsrlhsgiparfs |
| | | gsgsgtdytltisslqpedfavyfcqqgntlpytfgqgtkleikhhhhhhhh |
| 104881 | 96 | atggctatgcccgtgaccgcactcctcctgccactggctctgctgcttcacgccgc |
| CAR 7 | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactattcatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccggaggtggcggaagcgaaatcgtgatgacccagagc |
| | | cctgcaaccctgtccctttctcccggggaacgggctaccctttcttgtcgggcatc |
| | | acaagatatctcaaaatacctcaattggtatcaacagaagccgggacaggccccta |
| | | ggcttcttatctaccacacctctcgcctgcatagcgggattcccgcacgctttagc |
| | | gggtctggaagcgggaccgactacactctgaccatctcatctctccagcccgagga |
| | | cttcgccgtctacttctgccagcagggtaacaccctgccgtacaccttcggccagg |
| | | gcaccaagcttgagatcaaaaccactactcccgctccaaggccacccacccctgcc |
| | | ccgaccatcgcctctcagccgctttccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104881 | 83 | MALPVTALLLPLALLLHAARPgvqlgesgpglvkpsetlsltctvsgvslpdygvs |
| CAR 7 | | wirqppgkglewigviwgsettyyssslksrvtiskdnsknqvslklssvtaadta |
| Full - aa | | vyycakhyyyggsyamdywgqgtlvtvssggggsggggsggggsggggseivmtqs |
| | | patlslspgeratlscrasqdiskylnwyqqkpgqaprlliyhtsrlhsgiparfs |
| | | gsgsgtdytltisslqpedfavyfcqqgntlpytfgqgtkleiktttpaprpptpa |
| | | ptiasqplslrpeacrpaaggavhtrgldfacdiyiwaplagtcgvlllslvitly |
| | | ckrgrkkllyifkqpfmrpvqttgeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 8

| | | |
|---|---|---|
| CAR8 scFv | 46 | qvqlqesgpglvkpsetlsltctvsgvslpdygvswirgppgkglewigviwgset |
| domain | | tyyqsslksrvtiskdnsknqvslklssvtaadtavyycakhyyyggsyamdywgq |
| | | gtlvtvssggggsggggsggggsggggseivmtqspatlslspgeratlscrasqd |
| | | iskylnwyqqkpgqaprlliyhtsrlhsgiparfsgsgsgtdytltisslqpedfa |
| | | vyfcqqgntlpytfgqgtkleik |
| 100798 | 59 | atggcactgcctgtcactgccctcctgctgcctctggccctccttctgcatgccgc |
| CAR8 - | | caggccccaagtccagctgcaagagtcaggacccggactggtgaagccgtctgaga |
| Soluble | | ctctctcactgacttgtaccgtcagcggcgtgtccctccccgactacggagtgtca |
| scFv - nt | | tggatccgccaacctcccgggaaagggcttgaatggattggtgtcatctggggttc |
| | | tgaaaccacctactaccagtcttccctgaagtccagggtgaccatcagcaaggata |
| | | attccaagaaccaggtcagccttaagctgtcatctgtgaccgctgctgacaccgcc |
| | | gtgtattactgcgccaagcactactattacggaggaagctacgctatggactattg |
| | | gggacagggcactctcgtgactgtgagcagcggcggtggagggtctggaggtggag |
| | | gatccggtggtggtgggtcaggcggaggagggagcgagattgtgatgactcagtca |
| | | ccagccaccctttctctttcacccggcgagagagcaaccctgagctgtagagccag |
| | | ccaggacatttctaagtacctcaactggtatcagcaaaaaccggggcaggcccctc |
| | | gcctcctgatctaccatacctcacgccttcactctggtatccccgctcggtttagc |
| | | ggatcaggatctggtaccgactacactctgaccatttccagcctgcagccagaaga |
| | | tttcgcagtgtatttctgccagcagggcaatacccttccttacaccttcggtcagg |
| | | gaaccaagctcgaaatcaagcaccatcaccatcatcatcaccac |
| 100798 | 71 | MALPVTALLLPLALLLHAARPqvqlqesgpglvkpsetlsltctvsgvslpdygvs |
| CAR8 - | | wirqppgkglewigviwgsettyyqsslksrvtiskdnsknqvslklssvtaadta |
| Soluble | | vyycakhyyyggsyamdywgqgtlvtvssggggsggggsggggsggggseivmtqs |
| scFv - aa | | patlslspgeratlscrasqdiskylnwyqqkpgqaprlliyhtsrlhsgiparfs |
| | | gsgsgtdytltisslqpedfavyfcqqgntlpytfgqgtkleikhhhhhhhh |
| 104882 | 97 | atggctctgcccgtgaccgcactcctcctgccactggctctgctgcttcacgccgc |
| CAR 8 - | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactatcaatcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccggaggcggtgggtcagaaatcgtgatgacccagagc |
| | | cctgcaaccctgtccctttctcccggggaacgggctaccctttcttgtcgggcatc |
| | | acaagatatctcaaaatacctcaattggtatcaacagaagccgggacaggccccta |
| | | ggcttcttatctaccacacctctcgcctgcatagcgggattcccgcacgctttagc |
| | | gggtctggaagcgggaccgactacactctgaccatctcatctctccagcccgagga |
| | | cttcgccgtctacttctgccagcagggtaacaccctgccgtacaccttcggccagg |
| | | gcaccaagcttgagatcaaaaccactactcccgctccaaggccacccacccctgcc |
| | | ccgaccatcgcctctcagccgctttccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 104882 | 84 | MALPVTALLLPLALLLHAARPqvqlqesgpglvkpsetlsltctvsgvslpdygvs |
| CAR 8 - | | wirqppgkglewigviwgsettyyqsslksrvtiskdnsknqvslklssvtaadta |
| Full - aa | | vyycakhyyyggsyamdywgqgtlvtvssggggsggggsggggsggggseivmtqs |
| | | patlslspgeratlscrasqdiskylnwyqqkpgqaprlliyhtsrlhsgiparfs |
| | | gsgsgtdytltisslqpedfavyfcqqgntlpytfgqgtkleiktttpaprpptpa |
| | | ptiasqplslrpeacrpaaggavhtrgldfacdiyiwaplagtcgvlllslvitly |
| | | ckrgrkkllyifkqpfmrpvqttqeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR 9

| | | |
|---|---|---|
| CAR9 scFv | 47 | eivmtqspatlslspgeratlscrasqdiskylnwyqqkpgqaprlliyhtsrlhs |
| domain | | giparfsgsgsgtdytltisslqpedfavyfcqqgntlpytfgqgtkleikggggs |
| | | ggggsggggsggggsqvqlqesgpglvkpsetlsltctvsgvslpdygvswirqpp |
| | | gkglewigviwgsettyynsslksrvtiskdnsknqvslklssvtaadtavyycak |
| | | hyyyggsyamdywgqtlvtvss |
| 99789 | 60 | atggccctcccagtgaccgctctgctgctgcctctcgcacttcttctccatgccgc |
| CAR9 - | | tcggcctgagatcgtcatgacccaaagccccgctaccctgtccctgtcacccggcg |
| Soluble | | agagggcaaccctttcatgcagggccagccaggacatttctaagtacctcaactgg |
| scFv - nt | | tatcagcagaagccagggcaggctcctcgcctgctgatctaccacaccagccgcct |
| | | ccacagcggtatccccgccagattttccgggagcgggtctggaaccgactacaccc |
| | | tcaccatctcttctctgcagcccgaggatttcgccgtctatttctgccagcagggg |
| | | aatactctgccgtacaccttcggtcaaggtaccaagctggaaatcaagggaggcgg |
| | | aggatcaggcggtggcggaagcggaggaggtggctccggaggaggaggttcccaag |
| | | tgcagcttcaagaatcaggacccggacttgtgaagccatcagaaaccctctccctg |
| | | acttgtaccgtgtccggtgtgagcctccccgactacggagtctcttggattcgcca |
| | | gcctccggggaagggtcttgaatggattggggtgatttggggatcagagactactt |
| | | actacaattcatcacttaagtcacgggtcaccatcagcaaagataatagcaagaac |
| | | caagtgtcacttaagctqtcatctgtgaccgccgctgacaccgccgtqtactattg |
| | | tgccaaacattactattacggagggtcttatgctatggactactggggacagggga |
| | | ccctggtgactgtctctagccatcaccatcaccaccatcatcac |
| 99789 | 72 | MALPVTALLLPLALLLHAARPeivmtqspatlslspgeratlscrasqdiskylnw |
| CAR9 - | | yqqkpgqaprlliyhtsrlhsgiparfsgsgsgtdytltisslqpedfavyfcqqg |
| Soluble | | ntlpytfgqgtkleikggggsggggsggggsggggsqvqlqesgpglvkpsetlsl |
| scFv - aa | | tctvsgvslpdygvswirqppgkglewigviwgsettyynsslksrvtiskdnskn |
| | | qvslklssvtaadtavyycakhyyyggsyamdywgqgtlvtvsshhhhhhhh |
| 105974 | 98 | atggccctccctgtcaccgccctgctgcttccgctggctcttctgctccacgccgc |
| CAR 9 - | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggaggcggtgggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actacaactcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 105974 | 85 | MALPVTALLLPLALLLHAARPeivmtqspatlslspgeratlscrasqdiskylnw |
| CAR 9 - | | yqqkpgqaprlliyhtsrlhsgiparfsgsgsgtdytltisslqpedfavyfcqqg |
| Full - aa | | ntlpytfgqgtkleikggggsggggsggggsggggsqvqlqesgpglvkpsetlsl |
| | | tctvsgvslpdygvswirqppgkglewigviwgsettyynsslksrvtiskdnskn |
| | | qvslklssvtaadtavyycakhyyyggsyamdywgqgtlvtvsstttpaprpptpa |
| | | ptiasqplslrpeacrpaaggavhtrgldfacdiyiwaplagtcgvlllslvitly |
| | | ckrgrkkllyifkqpfmrpvqttgeedgcscrfpeeeeggcelrvkfsrsadapay |
| | | kqgqnqlynelnlgrreeydvldkrrgrdpemggkprrknpqeglynelqkdkmae |
| | | ayseigmkgerrrgkghdglyqglstatkdtydalhmqalppr |

CAR10

| | | |
|---|---|---|
| CAR10 | 48 | qvqlqesgpglvkpsetlsltctvsgvslpdygvswirqppgkglewigviwgset |
| scFv | | tyynsslksrvtiskdnsknqvslklssvtaadtavyycakhyyyggsyamdywgq |
| domain | | gtlvtvssggggsggggsggggsggggseivmtqspatlslspgeratlscrasqd |
| | | iskylnwyqqkpgqaprlliyhtsrlhsgiparfsgsgsgtdytltisslqpedfa |
| | | vyfcqqgntlpytfgqgtkleik |
| 100796 | 61 | atggcactgcctgtcactgccctcctgctgcctctggccctccttctgcatgccgc |
| CAR10 - | | caggccccaagtccagctgcaagagtcaggacccggactggtgaagccgtctgaga |
| Soluble | | ctctctcactgacttgtaccgtcagcggcgtgtccctccccgactacggagtgtca |
| scFv - nt | | tggatccgccaacctcccgggaaagggcttgaatggattggtgtcatctggggttc |
| | | tgaaaccacctactacaactcttccctgaagtccagggtgaccatcagcaaggata |
| | | attccaagaaccaggtcagccttaagctgtcatctgtgaccgctgctgacaccgcc |
| | | gtgtattactgcgccaagcactactattacggaggaagctacgctatggactattg |
| | | gggacagggcactctcgtgactgtgagcagcggcggtggagggtctggaggtggag |
| | | gatccggtggtggtgggtcaggcggaggagggagcgagattgtgatgactcagtca |
| | | ccagccaccctttctctttcacccggcgagagagcaaccctgagctgtagagccag |
| | | ccaggacatttctaagtacctcaactggtatcagcaaaaaccggggcaggcccctc |
| | | gcctcctgatctaccatacctcacgccttcactctggtatccccgctcggtttagc |
| | | ggatcaggatctggtaccgactacactctgaccatttccagcctgcagccagaaga |
| | | tttcgcagtgtatttctgccagcagggcaatacccttccttacaccttcggtcagg |
| | | gaaccaagctcgaaatcaagcaccatcaccatcatcaccaccat |
| 100796 | 73 | MALPVTALLLPLALLLHAARPqvqlqesgpglvkpsetlsltctvsgvslpdygvs |
| CAR10 - | | wirqppgkglewigviwgsettyynsslksrvtiskdnsknqvslklssvtaadta |
| Soluble | | vyycakhyyyggsyamdywgqgtlvtvssggggsggggsggggsggggseivmtqs |
| scFv - aa | | patlslspgeratlscrasqdiskylnwyqqkpgqaprlliyhtsrlhsgiparfs |
| | | gsgsgtdytltisslqpedfavyfcqqgntlpytfgqgtkleikhhhhhhhh |
| 105975 | 99 | atggccctccctgtcaccgccctgctgcttccgctggctcttctgctccacgccgc |
| CAR 10 | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagcggaggcggtgggagccagg |
| | | tccaactccaagaaagcggaccgggtcttgtgaagccatcagaaactctttcactg |
| | | acttgtactgtgagcggagtgtctctccccgattacggggtgtcttggatcagaca |
| | | gccaccggggaagggtctggaatggattggagtgatttggggctctgagactactt |
| | | actacaactcatccctcaagtcacgcgtcaccatctcaaaggacaactctaagaat |
| | | caggtgtcactgaaactgtcatctgtgaccgcagccgacaccgccgtgtactattg |
| | | cgctaagcattactattatggcgggagctacgcaatggattactggggacagggta |
| | | ctctggtcaccgtgtccagcaccactaccccagcaccgaggccacccaccccggct |
| | | cctaccatcgcctcccagcctctgtccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 105975 | 86 | MALPVTALLLPLALLLHAARPEIVMTQSPATLSLSPGERATLSCRASQDISKYLNW |
| CAR 10 | | YQQKPGQAPRLLIYHTSRLHSGIPARFSGSGSGTDYTLTISSLQPEDFAVYFCQQG |
| Full - aa | | NTLPYTFGQGTKLEIKGGGGSGGGGSGSGGSGGGGSQVQLQESQPQLVKPSETLSL |
| | | TCTVSGVSLPDYGVSWIRQPPGKGLEWIGVIWGSETTYYNSSLKSRVTISKDNSKN |
| | | QVSLKLSSVTAADTAVYYCAKHYYYGGSYAMDYWGQGTLVTVSSTTTPAPRPPTPA |
| | | PTIASQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSADAPAY |
| | | KQGQNQLYNELNLGRREEYDVLDKRRGRDPEMGGKPRRKNPQEGLYNELQKDKMAE |
| | | AYSEIGMKGERRRGKGHDGLYQSLSTATKDTYDALHMQALPPR |

CAR11

| | | |
|---|---|---|
| CAR11 | 49 | eivmtqspatlslspgeratlscrasqdiskylnwyqqkpgqaprlliyhtsrlhs |
| scFv | | giparfsgsgsgtdytltisslqpedfavyfcqqgntlpytfgqgtkleikggggs |
| domain | | ggggsggggsqvqlqesgpglvkpsetlsltctvsgvslpdygvswirqppgkgle |
| | | wigviwgsettyynsslksrvtiskdnsknqvslklssvtaadtavyycakhyyyg |
| | | gsyamdywgqgtlvtvss |
| 103101 | 62 | Atggccctccctgtcaccgccctgctgcttccgctggctcttctgctccacgccgc |
| CAR11 - | | tcggcccgaaattgtgatgacccagtcacccgccactcttagcctttcacccggtg |
| Soluble | | agcgcgcaaccctgtcttgcagagcctcccaagacatctcaaaataccttaattgg |
| scFv - nt | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactacaattcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagccaccaccatcatcaccatcaccat |
| 103101 | 74 | MALPVTALLLPLALLLHAARPeivmtqspatlslspgeratlscrasqdiskylnw |
| CAR11 - | | yqqkpgqaprlliyhtsrlhsgiparfsgsgsgtdytltisslqpedfavyfcqqg |
| Soluble | | ntlpytfgqgtkleikggggsggggsggggsqvqlqesgpglvkpsetlsltctvs |
| scFv - aa | | gvslpdygvswirqppgkglewigviwgsettyynsslksrvtiskdnsknqvslk |
| | | lssvtaadtavyycakhyyyggsyamdywgqgtlvtvsshhhhhhhh |
| 105976 | 100 | atggctctgcccgtgaccgcactcctcctgccactggctctgctgcttcacgccgc |
| CAR 11 | | tcgcccacaagtccagcttcaagaatcagggcctggtctggtgaagccatctgaga |
| Full - nt | | ctctgtccctcacttgcaccgtgagcggagtgtccctcccagactacggagtgagc |
| | | tggattagacagcctcccggaaagggactggagtggatcggagtgatttggggtag |
| | | cgaaaccacttactataactcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccggaggtggcggaagcgaaatcgtgatgacccagagc |
| | | cctgcaaccctgtccctttctcccggggaacgggctaccctttcttgtcgggcatc |
| | | acaagatatctcaaaatacctcaattggtatcaacagaagccgggacaggccccta |
| | | ggcttcttatctaccacacctctcgcctgcatagcgggattcccgcacgctttagc |
| | | gggtctggaagcgggaccgactacactctgaccatctcatctctccagcccgagga |
| | | cttcgccgtctacttctgccagcagggtaacaccctgccgtacaccttcggccagg |
| | | gcaccaagcttgagatcaaaaccactactcccgctccaaggccacccacccctgcc |
| | | ccgaccatcgcctctcagccgctttccctgcgtccggaggcatgtagacccgcagc |
| | | tggtggggccgtgcatacccggggtcttgacttcgcctgcgatatctacatttggg |
| | | cccctctggctggtacttgcggggtcctgctgctttcactcgtgatcactctttac |
| | | tgtaagcgcggtcggaagaagctgctgtacatctttaagcaacccttcatgaggcc |
| | | tgtgcagactactcaagaggaggacggctgttcatgccggttcccagaggaggagg |
| | | aaggcggctgcgaactgcgcgtgaaattcagccgcagcgcagatgctccagcctac |
| | | aagcaggggcagaaccagctctacaacgaactcaatcttggtcggagagaggagta |
| | | cgacgtgctggacaagcggagaggacgggacccagaaatgggcgggaagccgcgca |
| | | gaaagaatccccaagagggcctgtacaacgagctccaaaaggataagatggcagaa |
| | | gcctatagcgagattggtatgaaaggggaacgcagaagaggcaaaggccacgacgg |
| | | actgtaccagggactcagcaccgccaccaaggacacctatgacgctcttcacatgc |
| | | aggccctgccgcctcgg |
| 105976 | 87 | MALPVTALLLPLALLLHAARPQVQLQESGPGLVKPSETLSLTCTVSGVSLPDYGVS |
| CAR 11 | | WIRQPPGKGLEWIGVIWGSETTYYNSSLKSRVTISKENSKNQVSLKLSSVTAADTA |
| Full - aa | | VYYCAKHYYYGGSYAMDYWGQGTLVTVSSGGGGSGGGGSGGGGSGGGGSEIVMTQS |
| | | PATLSLSPGERATLSCRASQDISKYLNWYQQKPGQAPRLLIYHTSRLHSGIPARFS |
| | | GSGSGTDYTLTISSLQPEDFAVYFCQQGNTLPYTFGQGTKLEIKTTTPAPRPPTPA |
| | | PTIASQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSADAPAY |
| | | KQGQNQLYNELNLGRREEYDVLDKRRGRDPEMGGKERRKNPQEGLYNELQKDKMAE |
| | | AYSEIGMKGERRRGKGHDGLYQGLSTATKDTYDALHMQALPPR |

CAR12

| | | |
|---|---|---|
| CAR12 | 50 | qvqlqesgpglvkpsetlsltctvsgvslpdygvswirqppgkglewigviwgset |
| scFv | | tyynsslksrvtiskdnsknqvslklssvtaadtavyycakhyyyggsyamdywgq |
| domain | | gtlvtvssggggsggggsggggseivmtqspatlslspgeratlscrasqdiskyl |
| | | nwyqqkpgqaprlliyhtsrlhsgiparfsgsgsgtdytltisslqpedfavyfcq |
| | | qgntlpytfgqgtkleik |
| 103104 | 63 | atggctctgcccgtgaccgcactcctcctgccactggctctgctgcttcacgccgc |
| CAR12 - | | tcgcccacaagtccagcttcaagaatcagggcctggtctggtgaagccatctgaga |
| Soluble | | ctctgtccctcacttgcaccgtgagcggagtgtccctcccagactacggagtgagc |
| scFv - nt | | tggattagacagcctcccggaaagggactggagtggatcggagtgatttggggtag |
| | | cgaaaccacttactataactcttccctgaagtcacgggtcaccatttcaaaggata |
| | | actcaaagaatcaagtgagcctcaagctctcatcagtcaccgccgctgacaccgcc |
| | | gtgtattactgtgccaagcattactactatggagggtcctacgccatggactactg |
| | | gggccagggaactctggtcactgtgtcatctggtggaggaggtagcggaggaggcg |
| | | ggagcggtggaggtggctccgaaatcgtgatgacccagagccctgcaaccctgtcc |
| | | ctttctcccggggaacgggctaccctttcttgtcgggcatcacaagatatctcaaa |
| | | atacctcaattggtatcaacagaagccgggacaggcccctaggcttcttatctacc |
| | | acacctctcgcctgcatagcgggattcccgcacgctttagcgggtctggaagcggg |
| | | accgactacactctgaccatctcatctctccagcccgaggacttcgccgtctactt |
| | | ctgccagcagggtaacaccctgccgtacaccttcggccagggcaccaagcttgaga |
| | | tcaaacatcaccaccatcatcaccatcac |
| 103104 | 75 | MALPVTALLLPLALLLHAARPqvqlqesgpglvkpsetlsltctvsgvslpdygys |
| CAR12 - | | wirqppgkglewigviwgsettyynsslksrvtiskdnsknqvslklssvtaadta |
| Soluble | | vyycakhyyyggsyamdywgqgtlvtvssggggsggggsggggseivmtgspatls |
| scFv -aa | | lspgeratlscrasqdiskylnwyqqkpgqaprlliyhtsrlhsgiparfsgsgsg |
| | | tdytltisslqpedfavyfcqqgntlpytfgqgtkleikhhhhhhhh |
| 105977 | 101 | atggccctccctgtcaccgccctgctgcttccgctggctcttctgctccacgccgc |
| CAR 12 - | | tcggcccgaaattgtgatgacccagtcacccgccactcttagcctttcacccggtg |
| Full - nt | | agcgcgcaaccctgtcttgcagagcctcccaagacatctcaaaataccttaattgg |
| | | tatcaacagaagcccggacaggctcctcgccttctgatctaccacaccagccggct |
| | | ccattctggaatccctgccaggttcagcggtagcggatctgggaccgactacaccc |
| | | tcactatcagctcactgcagccagaggacttcgctgtctatttctgtcagcaaggg |
| | | aacaccctgccctacacctttggacagggcaccaagctcgagattaaaggtggagg |
| | | tggcagcggaggaggtgggtccggcggtggaggaagccaggtccaactccaagaaa |
| | | gcggaccgggtcttgtgaagccatcagaaactctttcactgacttgtactgtgagc |
| | | ggagtgtctctccccgattacggggtgtcttggatcagacagccaccggggaaggg |
| | | tctggaatggattggagtgatttggggctctgagactacttactacaactcatccc |
| | | tcaagtcacgcgtcaccatctcaaaggacaactctaagaatcaggtgtcactgaaa |
| | | ctgtcatctgtgaccgcagccgacaccgccgtgtactattgcgctaagcattacta |
| | | ttatggcgggagctacgcaatggattactggggacagggtactctggtcaccgtgt |
| | | ccagcaccactaccccagcaccgaggccacccaccccggctcctaccatcgcctcc |
| | | cagcctctgtccctgcgtccggaggcatgtagacccgcagctggtggggccgtgca |
| | | tacccggggtcttgacttcgcctgcgatatctacatttgggcccctctggctggta |
| | | cttgcggggtcctgctgctttcactcgtgatcactctttactgtaagcgcggtcgg |
| | | aagaagctgctgtacatctttaagcaacccttcatgaggcctgtgcagactactca |
| | | agaggaggacggctgttcatgccggttcccagaggaggaggaaggcggctgcgaac |
| | | tgcgcgtgaaattcagccgcagcgcagatgctccagcctacaagcaggggcagaac |
| | | cagctctacaacgaactcaatcttggtcggagagaggagtacgacgtgctggacaa |
| | | gcggagaggacgggacccagaaatgggcgggaagccgcgcagaaagaatccccaag |
| | | agggcctgtacaacgagctccaaaaggataagatggcagaagcctatagcgagatt |
| | | ggtatgaaaggggaacgcagaagaggcaaaggccacgacggactgtaccagggact |
| | | cagcaccgccaccaaggacacctatgacgctcttcacatgcaggccctgccgcctc |
| | | gg |
| 105977 | 88 | MALPVTALLLPLALLLHAARPEIVMTQSPATLSLSPGERATLSCRASQDISKYLNW |
| CAR 12 - | | YQQKPGQAPRLLIYHTSRLHSGIPARFSGSGSGTDYTLTISSLQPEDFAVYFCQQG |
| Full - aa | | NTLPYTFGQGTKLEIKGGGGSGGGGSGGGGSQVQLQESGPGLVKPSETLSLTCTVS |
| | | GVSLPDYGVSWIRQPPGKGLEWIGVIWGSETTYYNSSLKSRVTISKDNSKNQVSLK |
| | | LSSVTAADTAVYYCAKHYYYGGSYAMDYWGQGTLVTVSSTTTPAPRPPTPAPTIAS |
| | | QPLSLRPEACRPAAGGAVHTRGLDFACDIYIWAPLAGTCGVLLLSLVITLYCKRGR |
| | | KKLLYIFKQPFMRPVQTTQEEDGCSCRFPEEEEGGCELRVKFSRSADAPAYKQGQN |
| | | QLYNELNLGRREEYDVLDKRRGRDPEMGGKPRRKNPQEGLYNELQKDKMAEAYSEI |
| | | GMKGERRRGKGHDGLYQGLSTATKDTYDALHMQALPPR |

CTL019

| | | |
|---|---|---|
| CTL019 - | 22 | atggccctgcccgtcaccgctctgctgctgccccttgctctgcttcttcatgcagc |
| Soluble | | aaggccggacatccagatgacccaaaccacctcatccctctctgcctctcttggag |
| scFv-Histag- | | acagggtgaccatttcttgtcgcgccagccaggacatcagcaagtatctgaactgg |
| nt | | tatcagcagaagccggacggaaccgtgaagctcctgatctaccatacctctcgcct |
| | | gcatagcggcgtgccctcacgcttctctggaagcggatcaggaaccgattattctc |
| | | tcactatttcaaatcttgagcaggaagatattgccacctatttctgccagcagggt |
| | | aataccctgccctacaccttcggaggagggaccaagctcgaaatcaccggtggagg |
| | | aggcagcggcggtggagggtctggtggaggtggttctgaggtgaagctgcaagaat |
| | | caggccctggacttgtggccccttcacagtccctgagcgtgacttgcaccgtgtcc |
| | | ggagtctccctgcccgactacggagtgtcatggatcagacaacctccacggaaagg |
| | | actggaatggctcggtgtcatctggggtagcgaaactacttactacaattcagccc |
| | | tcaaaagcaggctgactattatcaaggacaacagcaagtcccaagtctttcttaag |
| | | atgaactcactccagactgacgacaccgcaatctactattgtgctaagcactacta |
| | | ctacggaggatcctacgctatggattactggggacaaggtacttccgtcactgtct |
| | | cttcacaccatcatcaccatcaccatcac |
| CTL019 - | 76 | MALPVTALLLPLALLLHAARPdiqmtqttsslsaslgdrvtiscrasqdiskylnw |
| Soluble | | yqqkpdgtvklliyhtsrlhsgvpsrfsgsgsgtdysltisnleqediatyfcqqg |
| scFv-Histag- | | ntlpytfgggtkleitggggsggggsggggsevklqesgpglvapsqslsvtctvs |
| aa | | gvslpdygvswirqpprkglewlgviwgsettyynsalksrltiikdnsksqvflk |
| | | mnslqtddtaiyycakhyyyggsyamdywgqgtsvtvsshhhhhhhh |
| CTL019 | 102 | atggccttaccagtgaccgccttgctcctgccgctggccttgctgctccacgccgc |
| Full - nt | | caggccggacatccagatgacacagactacatcctccctgtctgcctctctgggag |
| | | acagagtcaccatcagttgcagggcaagtcaggacattagtaaatatttaaattgg |
| | | tatcagcagaaaccagatggaactgttaaactcctgatctaccatacatcaagatt |
| | | acactcaggagtcccatcaaggttcagtggcagtgggtctggaacagattattctc |
| | | tcaccattagcaacctggagcaagaagatattgccacttacttttgccaacagggt |
| | | aatacgcttccgtacacgttcggaggggggaccadgctggagatcacaggtggcgg |
| | | tggctcgggcggtggtgggtcgggtggcggcggatctgaggtgaaactgcaggagt |
| | | caggacctggcctggtggcgccctcacagagcctgtccgtcacatgcactgtctca |
| | | ggggtctcattacccgactatggtgtaagctggattcgccagcctccacgaaaggg |
| | | tctggagtggctgggagtaatatggggtagtgaaaccacatactataattcagctc |
| | | tcaaatccagactgaccatcatcaaggacaactccaagagccaagttttcttaaaa |
| | | atgaacagtctgcaaactgatgacacagccatttactactgtgccaaacattatta |
| | | ctacggtggtagctatgctatggactactggggccaaggaacctcagtcaccgtct |
| | | cctcaaccacgacgccagcgccgcgaccaccaacaccggcgcccaccatcgcgtcg |
| | | cagcccctgtccctgcgcccagaggcgtgccggccagcggcggggggcgcagtgca |
| | | cacgagggggctggacttcgcctgtgatatctacatctgggcgcccttggccggga |
| | | cttgtggggtccttctcctgtcactggttatcaccctttactgcaaacggggcaga |
| | | aagaaactcctgtatatattcaaacaaccatttatgagaccagtacaaactactca |
| | | agaggaagatggctgtagctgccgatttccagaagaagaagaaggaggatgtgaac |
| | | tgagagtgaagttcagcaggagcgcagacgcccccgcgtacaagcagggccagaac |
| | | cagctctataacgagctcaatctaggacgaagagaggagtacgatgttttggacaa |
| | | gagacgtggccgggaccctgagatggggggaaagccgagaaggaagaaccctcagg |
| | | aaggcctgtacaatgaactgcagaaagataagatggcggaggcctacagtgagatt |
| | | gggatgaaaggcgagcgccggaggggcaaggggcacgatggcctttaccagggtct |
| | | cagtacagccaccaaggacacctacgacgcccttcacatgcaggccctgccccctc |
| | | gc |
| CTL019 | 89 | MALPVTALLLPLALLLHAARPdiqmtqttsslsaslgdrvtiscrasqdiskylnw |
| Full - aa | | yqqkpdgtvklliyhtsrlhsgvpsrfsgsgsgtdysltisnleqediatyfcqqg |
| | | ntlpytfgggtkleitggggsggggsggggsevklqesgpglvapsqslsvtctvs |
| | | gvslpdygvswirqpprkglewlgviwgsettyynsalksrltiikdnsksqvflk |
| | | mnslqtddtaiyycakhyyyggsyamdywgqgtsvtvsstttpaprpptpaptias |
| | | qplslrpeacrpaaggavhtrgldfacdiyiwaplagtcgvlllslvitlyckrgr |
| | | kkllyifkqpfmrpvqttqeedgcscrfpeeeeggcelrvkfsrsadapaykqgqn |
| | | qlynelnlgrreeydvldkrrgrdpemggkprrknpqeglynelqkdkmaeaysei |
| | | gmkgerrrgkghdglyqglstatkdtydalhmqalppr |
| CTL019 | 51 | diqmtqttsslsaslgdrvtiscrasqdiskylnwyqqkpdgtvklliyhtsrlhs |
| scFv | | gvpsrfsgsgsgtdysltisnleqediatyfcqqgntlpytfgggtkleitggggs |
| domain | | ggggsggggsevklqesgpglvapsqslsvtctvsgvslpdygvswirqpprkgle |
| | | wlgviwgsettyynsalksrltiikdnsksqvflkmnslqtddtaiyycakhyyyg |
| | | gsyamdywgqgtsvtvss |

In some embodiments, the antigen binding domain comprises a HC CDR1, a HC CDR2, and a HC CDR3 of any heavy chain binding domain amino acid sequences listed in Table 3. In embodiments, the antigen binding domain further comprises a LC CDR1, a LC CDR2, and a LC CDR3. In embodiments, the antigen binding domain comprises a LC CDR1, a LC CDR2, and a LC CDR3 of any light chain binding domain amino acid sequences listed in Table 3.

In some embodiments, the antigen binding domain comprises one, two or all of LC CDR1, LC CDR2, and LC CDR3 of any light chain binding domain amino acid sequences listed in Table 3, and one, two or all of HC CDR1, HC CDR2, and HC CDR3 of any heavy chain binding domain amino acid sequences listed in Table 3.

In some embodiments, the CDRs are defined according to the Kabat numbering scheme, the Chothia numbering scheme, or a combination thereof.

The sequences of humanized CDR sequences of the scFv domains are shown in Table 3A for the heavy chain variable domains and in Table 3B for the light chain variable domains. “ID” stands for the respective SEQ ID NO for each CDR.

TABLE 3A
Heavy Chain Variable Domain CDRs (Kabat)

| Candidate | FW | HCDR1 | ID | HCDR2 | ID | HCDR3 | ID |
|---|---|---|---|---|---|---|---|
| murine_CART19 | | DYGVS | 133 | VIWGSETT | 134 | HYYYGG | 138 |
| | | | | YYNSALKS | | SYAMDY | |
| humanized_CART19 a | VH4 | DYGVS | 133 | VIWGSETT | 135 | HYYYGG | 138 |
| | | | | YYSSSLKS | | SYAMDY | |
| humanized_CART19 b | VH4 | DYCVS | 133 | VIWCSETT | 136 | HYYYCC | 138 |
| | | | | YYQSSLKS | | SYAMDY | |
| humanized_CART19 c | VH4 | DYGVS | 133 | VIWGSETT | 137 | HYYYGG | 138 |
| | | | | YYNSSLKS | | SYAMDY | |

TABLE 3B
Light Chain Variable Domain CDRs

| Candidate | FW | LCDR1 | ID | LCDR2 | ID | LCDR3 | ID |
|---|---|---|---|---|---|---|---|
| murine_CART19 | | RASQDISKYLN | 139 | HTSRLHS | 140 | QQGNTLPYT | 141 |
| humanized_CART19 a | VK3 | RASQDISKYLN | 139 | HTSRLHS | 140 | QQGNTLPYT | 141 |
| humanized_CART19 b | VK3 | RASQDISKYLN | 139 | HTSRLHS | 140 | QQGNTLPYT | 141 |
| humanized_CART19 c | VK3 | RASQDISKYLN | 139 | HTSRLHS | 140 | QQGNTLPYT | 141 |

Co-Expression of CAR with Other Molecules or Agents

Co-Expression of a Second CAR

In one aspect, the CAR-expressing cell described herein can further comprise a second CAR, e.g., a second CAR that includes a different antigen binding domain, e.g., to the same target (e.g., CD19) or a different target (e.g., a target other than CD19, e.g., a target described herein). In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, e.g., 4-1BB, CD28, CD27, OX-40 or ICOS, onto the first CAR, and the primary signaling domain, e.g., CD3 zeta, on the second CAR can limit the CAR activity to cells where both targets are expressed. In one embodiment, the CAR expressing cell comprises a first CAR that includes an antigen binding domain, a transmembrane domain and a costimulatory domain and a second CAR that targets another antigen and includes an antigen binding domain, a transmembrane domain and a primary signaling domain. In another embodiment, the CAR expressing cell comprises a first CAR that includes an antigen binding domain, a transmembrane domain and a primary signaling domain and a second CAR that targets another antigen and includes an antigen binding domain to the antigen, a transmembrane domain and a costimulatory signaling domain.

In one embodiment, the CAR-expressing cell comprises an XCAR described herein and an inhibitory CAR. In one embodiment, the inhibitory CAR comprises an antigen binding domain that binds an antigen found on normal cells but not cancer cells, e.g., normal cells that also express X. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GALS, adenosine, and TGF (e.g., TGF beta).

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidac and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (e.g., selected by phage display).

It has also been discovered, that cells having a plurality of chimeric membrane embedded receptors comprising an antigen binding domain that interactions between the antigen binding domain of the receptors can be undesirable, e.g., because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising an antigen binding domains that minimize such interactions, as well as methods of making and using such cells and nucleic acids. In an embodiment the antigen binding domain of one of the first and the second non-naturally occurring chimeric membrane embedded receptor, comprises an scFv, and the other comprises a single VH domain, e.g., a camelid, shark, or lamprey single VH domain, or a single VH domain derived from a human or mouse sequence.

In some embodiments, a composition herein comprises a first and second CAR, wherein the antigen binding domain of one of the first and the second CAR does not comprise a variable light domain and a variable heavy domain. In some embodiments, the antigen binding domain of one of the first and the second CAR is an scFv, and the other is not an scFv. In some embodiments, the antigen binding domain of one of the first and the second CAR comprises a single VH domain, e.g., a camelid, shark, or lamprey single VH domain, or a single VH domain derived from a human or mouse sequence. In some embodiments, the antigen binding domain of one of the first and the second CAR comprises a nanobody. In some embodiments, the antigen binding domain of one of the first and the second CAR comprises a camelid VHH domain.

In some embodiments, the antigen binding domain of one of the first and the second CAR comprises an scFv, and the other comprises a single VH domain, e.g., a camelid, shark, or lamprey single VH domain, or a single VH domain derived from a human or mouse sequence. In some embodiments, the antigen binding domain of one of the first and the second CAR comprises an scFv, and the other comprises a nanobody. In some embodiments, the antigen binding domain of one of the first and the second CAR comprises an scFv, and the other comprises a camelid VHH domain.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is at least 85%, 90%, 95%, 96%, 97%, 98% or 99%, e.g., 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first and the second CAR, associate with one another less than if both were scFv antigen binding domains. In some embodiments, the antigen binding domains of the first and the second CAR, associate with one another at least 85%, 90%, 95%, 96%, 97%, 98% or 99% less than, e.g., 85%, 90%, 95%, 96%, 97%, 98% or 99% less than if both were scFv antigen binding domains.

Co-Expression of an Agent that Enhances CAR Activity

In another aspect, the CAR-expressing cell described herein can further express another agent, e.g., an agent that enhances the activity or fitness of a CAR-expressing cell.

For example, in one embodiment, the agent can be an agent which inhibits a molecule that modulates or regulates, e.g., inhibits, T cell function. In some embodiments, the molecule that modulates or regulates T cell function is an inhibitory molecule. Inhibitory molecules, e.g., PD1, can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KTR, A2aR, MHC class T, MHC class IT, GAL9, adenosine, or TGF beta.

In embodiments, an agent, e.g., an inhibitory nucleic acid, e.g., a dsRNA, e.g., an siRNA or shRNA; or e.g., an inhibitory protein or system, e.g., a clustered regularly interspaced short palindromic repeats (CRISPR), a transcription-activator like effector nuclease (TALEN), or a zinc finger endonuclease (ZFN), e.g., as described herein, can be used to inhibit expression of a molecule that modulates or regulates, e.g., inhibits, T-cell function in the CAR-expressing cell. In an embodiment the agent is an shRNA, e.g., an shRNA described herein. In an embodiment, the agent that modulates or regulates, e.g., inhibits. T-cell function is inhibited within a CAR-expressing cell. For example, a dsRNA molecule that inhibits expression of a molecule that modulates or regulates, e.g., inhibits, T-cell function is linked to the nucleic acid that encodes a component, e.g., all of the components, of the CAR.

In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (e.g., at least a portion of an extracellular domain of any of these), and a second polypeptide which is an intracellular signaling domain described herein (e.g., comprising a costimulatory domain (e.g., 41BB, CD27 or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et al. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-Ll.

In one embodiment, the agent comprises the extracellular domain (ECD) of an inhibitory molecule, e.g., Programmed Death 1 (PD1), can be fused to a transmembrane domain and intracellular signaling domains such as 41BB and CD3 zeta (also referred to herein as a PD1 CAR). In one embodiment, the PD1 CAR, when used in combinations with an XCAR described herein, improves the persistence of the T cell. In one embodiment, the CAR is a PD1 CAR comprising the extracellular domain of PD1 indicated as underlined in SEQ ID NO: 105. In one embodiment, the PD1 CAR comprises the amino acid sequence of SEQ ID NO:105.

(SEQ ID NO: 105)
Malpvtalllplalllhaarppgwfldspdrpwnpptfspallvvtegdn
atftcsfsntsesfvlnwyrmspsnqtdklaafpedrsqpgqdcrfrvtq
lpngrdfhmsvvrarrndsgtylcgalslapkaqikeslraelryterra
evptahpspsprpagqfqtlvtttpaprpptpaptiasqplslrpeacrp
aaggavhtrgldfacdiyiwaplagtcgvlllslvitlyckrgrkkllyi
fkqpfmrpvqttqeedgcscrfpeeeeggcelrvkfsrsadapaykqgqn
qlynelnlgrreeydvldkrrgrdpemggkprrknpqeglynelqkdkma
eayseigmkgerrrgkghdglyqglstatkdtydalhmqalppr.

In one embodiment, the PD1 CAR comprises the amino acid sequence provided below (SEQ ID NO:106).

(SEQ ID NO: 106)
pgwfldspdrpwnpptfspallvvtegdnatftcsfsntsesfvlnwyrm
spsnqtdklaafpedrsqpgqdcrfrytqlpngrdfhmsyyrarrndsgt
ylcgaislapkaqikeslraelryterraeyptahpspsprpagqfqtlv
tttpaprpptpaptiasqplslrpeacrpaaggavhtrgldfacdiyiwa
plagtcgvillslvitlycicrgrkkllyifkqpfmrpvqttqeedgcsc
rfpeeeeggcelrvkfsrsadapaykqgqnqlynelnlgrreeydvldla
rgrdpemggkprrknpqeglynelqkdkmaeayseigmkgerrrgkghdg
lyqglstatkdtydalhmqalppr.

In one embodiment, the agent comprises a nucleic acid sequence encoding the PD1 CAR, e.g., the PD1 CAR described herein. In one embodiment, the nucleic acid sequence for the PD1 CAR is shown below, with the PD1 ECD underlined below in SEQ ID NO: 107

(SEQ ID NO: 107)
atggccctccctgtcactgccctgcttctccccctcgcactcctgctcca
cgccgctagaccacccggatggtttctggactctccggatcgcccgtgga
atcccccaaccttctcaccggcactcttggttgtgactgagggcgataat
gcgaccttcacgtgctcgttctccaacacctccgaatcattcgtgctgaa
ctggtaccgcatgagcccgtcaaaccagaccgacaagctcgccgcgtttc
cggaagatcggtcgcaaccgggacaggattgteggaccgcgtgactcaac
tgccgaatggcagagacttccacatgagcgtggtccgcgctaggcgaaac
gactccgggacctacctgtgcggagccatctcgctggcgcctaaggccca
aatcaaagagagcttgagggccgaactgagagtgaccgagcgcagagctg
aggtgccaactgcacatccatccccatcgcctcggcctgcggggcagttt
cagaccctggtcacgaccactccggcgccgcgcccaccgactccggcccc
aactatcgcgagccagcccctgtcgctgaggccggaagcatgccgccctg
ccgccggaggtgctgtgcatacccggggattggacttcgcatgcgacatc
tacatttgggctcctctcgccggaacttgtggcgtgctccttctgtccct
ggtcatcaccctgtactgcaagcggggtcggaaaaagcttctgtacattt
tcaagcagcccttcatgaggcccgtgcaaaccacccaggaggaggacggt
tgctectgccggttccccgaagaggaagaaggaggttgcgagctgcgcgt
gaagttctcccggagcgccgacgcccccgcctataagcagggccagaacc
agctgtacaacgaactgaacctgggacggcgggaagagtacgatgtgctg
gacaagcggcgcggccgggaccccgaaatgggcgggaagcctagaagaaa
gaaccctcaggaaggcctgtataacgagctgcagaaggacaagatggccg
aggcctactccgaaattgggatgaagggagagcggcggaggggaaagggg
cacgacggcctgtaccaaggactgtccaccgccaccaaggacacatacga
tgccctgcacatgcaggcccttccccctcgc.

In another example, in one embodiment, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, e.g., as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In an embodiment, the costimulatory molecule ligand is 4-1BBL. In an embodiment, the costimulatory ligand is CD80 or CD86. In an embodiment, the costimulatory molecule ligand is CD70. In embodiments, a CAR-expressing immune effector cell described herein can be further engineered to express one or more additional costimulatory molecules or costimulatory molecule ligands.

Co-Expression of CAR with a Chemokine Receptor

In embodiments, the CAR-expressing cell described herein, e.g., CD19 CAR-expressing cell, further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, e.g., solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (e.g., CAR-Tx) described herein include a CXC chemokine receptor (e.g., CXCR1, CXCR2, CXCR3, CXCR4, CXCR5, CXCR6, or CXCR7), a CC chemokine receptor (e.g., CCR1, CCR2, CCR3, CCR4, CCR5, CCR6, CCR7, CCR8, CCR9, CCR10, or CCR11), a CX3C chemokine receptor (e.g., CX3CR1), a XC chemokine receptor (e.g., XCR1), or a chemokine-binding fragment thereof. In one embodiment, the chemokine receptor molecule to be expressed with a CAR described herein is selected based on the chemokine(s) secreted by the tumor. In one embodiment, the CAR-expressing cell described herein further comprises, e.g., expresses, a CCR2b receptor or a CXCR2 receptor. In an embodiment, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Nucleic Acid Constructs Encoding a CAR

The present invention also provides an immune effector cell, e.g., made by a method described herein, that includes a nucleic acid molecules encoding one or more CAR constructs described herein. In one aspect, the nucleic acid molecule is provided as a messenger RNA transcript. In one aspect, the nucleic acid molecule is provided as a DNA construct.

The nucleic acid molecules described herein can be a DNA molecule, an RNA molecule, or a combination thereof. In one embodiment, the nucleic acid molecule is an mRNA encoding a CAR polypeptide as described herein. In other embodiments, the nucleic acid molecule is a vector that includes any of the aforesaid nucleic acid molecules.

In one aspect, the antigen binding domain of a CAR of the invention (e.g., a scFv) is encoded by a nucleic acid molecule whose sequence has been codon optimized for expression in a mammalian cell. In one aspect, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, e.g., methods disclosed in at least U.S. Pat. Nos. 5,786,464 and 6,114,148.

Accordingly, in one aspect, an immune effector cell, e.g., made by a method described herein, includes a nucleic acid molecule encoding a chimeric antigen receptor (CAR), wherein the CAR comprises an antigen binding domain that binds to a tumor antigen described herein, a transmembrane domain (e.g., a transmembrane domain described herein), and an intracellular signaling domain (e.g., an intracellular signaling domain described herein) comprising a stimulatory domain, e.g., a costimulatory signaling domain (e.g., a costimulatory signaling domain described herein) and/or a primary signaling domain (e.g., a primary signaling domain described herein, e.g., a zeta chain described herein).

The present invention also provides vectors in which a nucleic acid molecule encoding a CAR, e.g., a nucleic acid molecule described herein, is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, e.g., a gammaretroviral vector. A gammaretroviral vector may include, e.g., a promoter, a packaging signal (ψ), a primer binding site (PBS), one or more (e.g., two) long terminal repeats (LTR), and a transgene of interest, e.g., a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, e.g., in Tobias Maetzig et al., “Gammaretroviral Vectors: Biology, Technology and Application” Viruses. 2011 June; 3(6): 677-713.

In another embodiment, the vector comprising the nucleic acid encoding the desired CAR is an adenoviral vector (A5/35). In another embodiment, the expression of nucleic acids encoding CARs can be accomplished using of transposons such as sleeping beauty, crisper, CAS9, and zinc finger nucleases. See below June et al. 2009 Nature Reviews Immunology 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, N.Y.), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

Additional promoter elements, e.g., enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

An example of a promoter that is capable of expressing a CAR encoding nucleic acid molecule in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from nucleic acid molecules cloned into a lentiviral vector. See, e.g., Milone et al., Mol. Ther. 17(8): 1453-1464 (2009). In one aspect, the EF1a promoter comprises the sequence provided in the Examples.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but arc not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (e.g., a PGK promoter with one or more, e.g., 1, 2, 5, 10, 100, 200, 300, or 400, nucleotide deletions when compared to the wild-type PGK promoter sequence) may be desired.

The nucleotide sequences of exemplary PGK promoters are provided below.

WT PGK Promoter:
(SEQ ID NO: 109)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCGCGGCGACGCAAAGGGCCTTGGTGCGGGT
CTCGTCGGCGCAGGGACGCGTTTGGGTCCCGACGGAACCTTTTCCGCGTT
GGGGTTGGGGCACCATAAGCT
Exemplary truncated PGKP romoters:
PGK100:
(SEQ ID NO: 110)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:
(SEQ ID NO: 111)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:
(SEQ ID NO: 112)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:
(SEQ ID NO: 113)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, e.g., a signal sequence to facilitate secretion, a polyadenylation signal and transcription terminator (e.g., from Bovine Growth Hormone (BGH) gene), an element allowing episomal replication and replication in prokaryotes (e.g. SV40 origin and ColE1 or others known in the art) and/or elements to allow selection (e.g., ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In other aspects, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, e.g., enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (e.g., Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, e.g., a CAR described herein, e.g., a CD19 CAR, and a second CAR, e.g., an inhibitory CAR or a CAR that specifically binds to an antigen other than CD19. In such embodiments, the two or more nucleic acid sequences encoding the CAR are encoded by a single nucleic molecule in the same frame and as a single polypeptide chain. In this aspect, the two or more CARs, can, e.g., be separated by one or more peptide cleavage sites. (e.g., an auto-cleavage site or a substrate for an intracellular protease). Examples of peptide cleavage sites include T2A, P2A, E2A, or F2A sites.

Methods of introducing and expressing genes into a cell are known in the art. In the context of an expression vector, the vector can be readily introduced into a host cell, e.g., mammalian, bacterial, yeast, or insect cell by any method, e.g., one known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, N.Y.). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, e.g., human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In another aspect, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL.). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, e.g., by immunological means (ELISAs and Western blots) or by assays described herein to identify agents falling within the scope of the invention.

Natural Killer Cell Receptor (NKR) CARs

In an embodiment, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12. Exemplary configurations and sequences of CAR molecules comprising NKR components are described in International Publication No. WO2014/145252, the contents of which are hereby incorporated by reference.

### Split CAR

In some embodiments, the CAR-expressing cell uses a split CAR. The split CAR approach is described in more detail in publications WO2014/055442 and WO2014/055657. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens.

### Strategies for Regulating Chimeric Antigen Receptors

In some embodiments, a regulatable CAR (RCAR) where the CAR activity can be controlled is desirable to optimize the safety and efficacy of a CAR therapy. There are many ways CAR activities can be regulated. For example, inducible apoptosis using, e.g., a caspase fused to a dimerization domain (see, e.g., Di Stasa et al., N Engl. J. Med. 2011 Nov. 3; 365(18):1673-1683), can be used as a safety switch in the CAR therapy of the instant invention. In one embodiment, the cells (e.g., T cells or NK cells) expressing a CAR of the present invention further comprise an inducible apoptosis switch, wherein a human caspase (e.g., caspase 9) or a modified version is fused to a modification of the human FKB protein that allows conditional dimerization. In the presence of a small molecule, such as a rapalog (e.g., AP 1903, AP20187), the inducible caspase (e.g., caspase 9) is activated and leads to the rapid apoptosis and death of the cells (e.g., T cells or NK cells) expressing a CAR of the present invention. Examples of a caspase-based inducible apoptosis switch (or one or more aspects of such a switch) have been described in, e.g., US2004040047; US20110286980; US20140255360; WO1997031899; WO2014151960; WO2014164348; WO2014197638; WO2014197638: all of which are incorporated by reference herein.

In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (OD) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or complement-induced cell death. For example, CAR expressing cells described herein may also express a receptor capable of being targeted by an antibody or antibody fragment. Examples of such receptors include EpCAM, VEGFR, integrins (e.g., integrins αvβ3, α4, αI¾β3, α4β7, α5β1, αvβ3, αv), members of the TNF receptor superfamily (e.g., TRAIL-R1 , TRAIL-R2), PDGF Receptor, interferon receptor, folate receptor, GPNMB, ICAM-1 , HLA-DR, CEA, CA-125, MUC1 , TAG-72, IL-6 receptor, 5T4, GD2, GD3, CD2, CD3, CD4, CD5, CD11, CD11a/LFA-1 , CD15, CD18/ITGB2, CD19, CD20, CD22, CD23/1gE Receptor, CD25, CD28, CD30, CD33, CD38, CD40, CD41 , CD44, CD51 , CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

For example, a CAR-expressing cell described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH, a monoclonal anti-CD52 antibody that selectively binds and targets mature lymphocytes, e.g., CAR-expressing cells, for destruction, e.g., by inducing ADCC. In other embodiments, the CAR-expressing cell can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In other embodiments, a CAR-expressing cell described herein may also express a target protein recognized by the T cell depleting agent. In one embodiment, the target protein is CD20 and the T cell depleting agent is an anti-CD20 antibody, e.g., rituximab. In such embodiment, the T cell depleting agent is administered once it is desirable to reduce or eliminate the CAR-expressing cell, e.g., to mitigate the CAR induced toxicity. In other embodiments, the T cell depleting agent is an anti-CD52 antibody, e.g., alemtuzumab, as described in the Examples herein.

In other embodiments, an RCAR comprises a set of polypeptides, typically two in the simplest embodiments, in which the components of a standard CAR described herein, e.g., an antigen binding domain and an intracellular signaling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signaling domain. In one embodiment, a CAR of the present invention utilizes a dimerization switch as those described in, e.g., WO2014127261, which is incorporated by reference herein. Additional description and exemplary configurations of such regulatable CARs are provided herein and in, e.g., paragraphs 527-551 of International Publication No. WO 2015/090229 filed Mar. 13, 2015, which is incorporated by reference in its entirety. In some embodiments, an RCAR involves a switch domain, e.g., a FKBP switch domain, as set out SEQ ID NO: 114, or comprise a fragment of FKBP having the ability to bind with FRB, e.g., as set out in SEQ ID NO: 115. In some embodiments, the RCAR involves a switch domain comprising a FRB sequence, e.g., as set out in SEQ ID NO: 116, or a mutant FRB sequence, e.g., as set out in any of SEQ ID Nos. 117-122.

(SEQ ID NO: 114)
DVPDYASLGGPSSPKKKRKVSRGVQVETISPGDGRTFPKRGQTCVVHYTG
MLEDGKKFDSSRDRNKPFKFMLGKQEVIRGWEEGVAQMSVGQRAKLT1SP
DYAYGATGHPGIIPPHATLVFDVELLKLETSY
(SEQ ID NO: 115)
VQVETISPGDGRTFPKRGQTCVVHYTGMLEDGKKFDSSRDRNKPFKFMLG
KQEVIRGWEEGVAQMSVGQRAKLTISPDYAYGATGHPGIIPPHATLVFDV
ELLKLETS
(SEQ ID NO: 116)
ILWHEMWHEGLEEASRLYFGERNVKGMFEVLEPLHAMMERGPQTLKETSF
NQAYGRDLMEAQEWCRKYMKSGNVKDLTQAWDLYYHVFRRISK

TABLE 13
Exemplary mutant FRB having increased
affinity for a dimerization molecule.

| | | SEQ |
|---|---|---|
| FRB | | ID |
| mutant | Amino Acid Sequence | NO: |
| E2032I | ILWHEMWHEGLIEASRLYFCERNVKGMFEVLEPLHAMMER | 117 |
| mutant | CPQTLKETSFNQAYGRDLMEAQEWCRKYMKSGNVKDLTQA | |
| | WDLYYHVFRRISKTS | |
| E2032L | ILWHEMWHEGLLEASRIYFGERNVKGMFEVLEPIHAMMER | 118 |
| mutant | GPQILKETSFNQAYGRDLMEAQEWCRKYMKSGNVKDLTQA | |
| | WDLYYHVFRRISKTS | |
| T2098L | ILWHEMWHEGLEEASRLYFGERNVKGMFEVLEPLHAMMER | 119 |
| mutant | GPQTLKETSFNQAYGRDLMEAQEWCRKYMKSGNVKDLLQA | |
| | WDLYYHVFRRISKTS | |
| E2032, | ILWHEMWHEGLXEASRIYFGERNVKGMFEVLEPLHAMMER | 120 |
| T2098 | GPQTLKETSFNQAYGRDIMEAQEWCRKYMKSGNVKDLXQA | |
| mutant | WDLYYHVFRRISKTS | |
| E2032I, | ILWHEMWHEGLIEASRLYFGERNVKGMFEVLEPLHAMMER | 121 |
| T2098L | GPQILKETSFNQAYGRDLMEAQEWCRKYMKSGNVKDLLQA | |
| mutant | WDLYYHVFRRISKTS | |
| E2032L, | ILWHEMWHEGLLEASRLYFGERNVKGMFEVLEPLHAMMER | 122 |
| T2098L | GPQTLKEGSFNQAYGRDLMEAQEWCRKYMKSGNVKDLLQA | |
| mutant | WDLYYHVFRRISKTS | |

### RNA Transfection

Disclosed herein are methods for producing an in vitro transcribed RNA CAR. RNA CAR and methods of using the same are described, e.g., in paragraphs 553-570 of in International Application WO2015/142675, filed Mar. 13, 2015, which is herein incorporated by reference in its entirety.

An immune effector cell can include a CAR encoded by a messenger RNA (mRNA). In one aspect, the mRNA encoding a CAR described herein is introduced into an immune effector cell, e.g., made by a method described herein, for production of a CAR-expressing cell.

In one embodiment, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR described herein. For example, the template for the RNA CAR comprises an extracellular region comprising a single chain variable domain of an antibody to a tumor associated antigen described herein; a hinge region (e.g., a hinge region described herein), a transmembrane domain (e.g., a transmembrane domain described herein such as a transmembrane domain of CD8a); and a cytoplasmic region that includes an intracellular signaling domain, e.g., an intracellular signaling domain described herein, e.g., comprising the signaling domain of CD3-zeta and the signaling domain of 4-1BB.

In one embodiment, the DNA to be used for PCR contains an open reading frame. The DNA can be from a naturally occurring DNA sequence from the genome of an organism. In one embodiment, the nucleic acid can include some or all of the 5′ and/or 3′ untranslated regions (UTRs). The nucleic acid can include exons and introns. In one embodiment, the DNA to be used for PCR is a human nucleic acid sequence. In another embodiment, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In one embodiment, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA in embodiments has 5′ and 3′ UTRs. In one embodiment, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In one embodiment, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In one embodiment, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In an embodiment, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem. 270:1485-65 (2003).

The conventional method of integration of polyA/T stretches into a DNA template is molecular cloning. However polyA/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with polyA/T 3′ stretch without cloning highly desirable.

The polyA/T segment of the transcriptional DNA template can be produced during PCR by using a reverse primer containing a polyT tail, such as 100T tail (SEQ ID NO: 123) (size can be 50-5000 T (SEQ ID NO: 32)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In one embodiment, the poly(A) tail is between 100 and 5000 adenosines (e.g., SEQ ID NO: 33).

Poly(A) tails of RNAs can be further extended following in vitro transcription with the use of a poly(A) polymerase, such as E. coli polyA polymerase (E-PAP). In one embodiment, increasing the length of a poly(A) tail from 100 nucleotides to between 300 and 400 nucleotides (SEQ ID NO: 34) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In an embodiment, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Non-Viral Delivery Methods

In some aspects, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac (PB) transposon system. See, e.g., Aronovich et al. Hum. Mol. Genet. 20.R1(2011):R14-20; Singh et al. Cancer Res. 15(2008):2961-2971; Huang et al. Mol. Ther. 16(2008):580-589; Grabundzija et al. Mol. Ther. 18(2010):1200-1209; Kebriaei et al. Blood. 122.21(2013):166; Williams. Molecular Therapy 16.9(2008):1515-16; Bell et al. Nat. Protoc. 2.12(2007):3153-65; and Ding et al. Cell. 122.3(2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, e.g., Aronovich et al. supra.

Exemplary transposons include a pT2-based transposon. See, e.g., Grabundzija et al. Nucleic Acids Res. 41.3(2013):1829-47; and Singh et al. Cancer Res. 68.8(2008): 2961-2971, all of which are incorporated herein by reference. Exemplary transposases include a Tcl/mariner-type transposase, e.g., the SB10 transposase or the SB11 transposase (a hyperactive transposase which can be expressed, e.g., from a cytomegalovirus promoter). See, e.g., Aronovich et al.; Kebriaei et al.; and Grabundzija et al., all of which are incorporated herein by reference.

Use of the SBTS permits efficient integration and expression of a transgene, e.g., a nucleic acid encoding a CAR described herein. Provided herein are methods of generating a cell, e.g., T cell or NK cell, that stably expresses a CAR described herein, e.g., using a transposon system such as SBTS.

In accordance with methods described herein, in some embodiments, one or more nucleic acids, e.g., plasmids, containing the SBTS components are delivered to a cell (e.g., T or NK cell). For example, the nucleic acid(s) are delivered by standard methods of nucleic acid (e.g., plasmid DNA) delivery, e.g., methods described herein, e.g., electroporation, transfection, or lipofection. In some embodiments, the nucleic acid contains a transposon comprising a transgene, e.g., a nucleic acid encoding a CAR described herein. In some embodiments, the nucleic acid contains a transposon comprising a transgene (e.g., a nucleic acid encoding a CAR described herein) as well as a nucleic acid sequence encoding a transposase enzyme. In other embodiments, a system with two nucleic acids is provided, e.g., a dual-plasmid system, e.g., where a first plasmid contains a transposon comprising a transgene, and a second plasmid contains a nucleic acid sequence encoding a transposase enzyme. For example, the first and the second nucleic acids are co-delivered into a host cell.

In some embodiments, cells, e.g., T or NK cells, are generated that express a CAR described herein by using a combination of gene insertion using the SBTS and genetic editing using a nuclease (e.g., Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, e.g., T or NK cells, and direct infusion of the cells into a subject. Advantages of non-viral vectors include but are not limited to the ease and relatively low cost of producing sufficient amounts required to meet a patient population, stability during storage, and lack of immunogenicity.

Methods of Manufacture/Production

In some embodiments, the methods disclosed herein further include administering a T cell depleting agent after treatment with the cell (e.g., an immune effector cell as described herein), thereby reducing (e.g., depleting) the CAR-expressing cells (e.g., the CD19CAR-expressing cells). Such T cell depleting agents can be used to effectively deplete CAR-expressing cells (e.g., CD19CAR-expressing cells) to mitigate toxicity. In some embodiments, the CAR-expressing cells were manufactured according to a method herein, e.g., assayed (e.g., before or after transfection or transduction) according to a method herein.

In some embodiments, the T cell depleting agent is administered one, two, three, four, or five weeks after administration of the cell, e.g., the population of immune effector cells, described herein.

In one embodiment, the T cell depleting agent is an agent that depletes CAR-expressing cells, e.g., by inducing antibody dependent cell-mediated cytotoxicity (ADCC) and/or complement-induced cell death. For example, CAR-expressing cells described herein may also express an antigen (e.g., a target antigen) that is recognized by molecules capable of inducing cell death, e.g., ADCC or complement-induced cell death. For example, CAR expressing cells described herein may also express a target protein (e.g., a receptor) capable of being targeted by an antibody or antibody fragment. Examples of such target proteins include, but are not limited to, EpCAM, VEGFR, integrins (e.g., integrins αvβ3, α4, αI¾β3, α4β7, α5β1, αvβ3, αv), members of the TNF receptor superfamily (e.g., TRAIL-R1 , TRAIL-R2), PDGF Receptor, interferon receptor, folate receptor, GPNMB, ICAM-1, HLA-DR, CEA, CA-125, MUC1, TAG-72, IL-6 receptor, 5T4, GD2, GD3, CD2, CD3, CD4, CD5, CD11 , CD11a/LFA-1, CD15, CD18/ITGB2, CD19, CD20, CD22, CD23/1gE Receptor, CD25, CD28, CD30, CD33, CD38, CD40, CD41 , CD44, CD51 , CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain).

In some embodiments, the CAR expressing cell co-expresses the CAR and the target protein, e.g., naturally expresses the target protein or is engineered to express the target protein. For example, the cell, e.g., the population of immune effector cells, can include a nucleic acid (e.g., vector) comprising the CAR nucleic acid (e.g., a CAR nucleic acid as described herein) and a nucleic acid encoding the target protein.

In one embodiment, the T cell depleting agent is a CD52 inhibitor, e.g., an anti-CD52 antibody molecule, e.g., alemtuzumab.

In other embodiments, the cell, e.g., the population of immune effector cells, expresses a CAR molecule as described herein (e.g., CD19CAR) and the target protein recognized by the T cell depleting agent. In one embodiment, the target protein is CD20. In embodiments where the target protein is CD20, the T cell depleting agent is an anti-CD20 antibody, e.g., rituximab.

In further embodiments of any of the aforesaid methods, the methods further include transplanting a cell, e.g., a hematopoietic stem cell, or a bone marrow, into the mammal.

In another aspect, the invention features a method of conditioning a mammal prior to cell transplantation. The method includes administering to the mammal an effective amount of the cell comprising a CAR nucleic acid or polypeptide, e.g., a CD19 CAR nucleic acid or polypeptide. In some embodiments, the cell transplantation is a stem cell transplantation, e.g., a hematopoietic stem cell transplantation, or a bone marrow transplantation. In other embodiments, conditioning a subject prior to cell transplantation includes reducing the number of target-expressing cells in a subject, e.g., CD19− expressing normal cells or CD19− expressing cancer cells.

### Activation and Expansion of Immune Effector Cells (e.g., T Cells)

Immune effector cells such as T cells generated or enriched by the methods described herein may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, a population of immune effector cells, e.g., T regulatory cell depleted cells, may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is hound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect, more anti-CD28 antibody is bound to the particles than anti-CD3 antibody, i.e., the ratio of CD3:CD28 is less than one. In certain aspects, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain suitable values include 1:100, 1:50, 1:40, 1:30, 1:20, 1:10, 1:9, 1:8, 1:7, 1:6, 1:5, 1:4, 1:3, 1:2. 1:1, 2:1, 3:1, 4:1, 5:1, 6:1, 7:1, 8:1, 9:1, 10:1, and 15:1 with one suitable ratio being at least 1:1 particles per T cell. In one aspect, a ratio of particles to cells of 1:1 or less is used. In one particular aspect, a suitable particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In one aspect the cells (for example, 10⁴ to 10⁹ T cells) and beads (for example, DYNABEADS® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In certain aspects, it may be desirable to significantly decrease the volume in which particles and cells are mixed together (i.e., increase the concentration of cells), to ensure maximum contact of cells and particles. For example, in one aspect, a concentration of about 10 billion cells/ml, 9 billion/ml, 8 billion/ml, 7 billion/ml, 6 billion/ml, 5 billion/ml, or 2 billion cells/ml is used. In one aspect, greater than 100 million cells/ml is used. In a further aspect, a concentration of cells of 10, 15, 20, 25, 30, 35, 40, 45, or 50 million cells/ml is used. In yet one aspect, a concentration of cells from 75, 80, 85, 90, 95, or 100 million cells/ml is used. In further aspects, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in certain aspects. For example, using high concentration of cells allows more efficient selection of CD8+ T cells that normally have weaker CD28 expression.

In one embodiment, cells transduced with a nucleic acid encoding a CAR, e.g., a CAR described herein, e.g., a CD19 CAR described herein, are expanded, e.g., by a method described herein. In one embodiment, the cells are expanded in culture for a period of several hours (e.g., about 2, 3, 4, 5, 6, 7, 8, 9, 10, 15, 18, 21 hours) to about 14 days (e.g., 1, 2, 3, 4, 5, 6, 7, 8, 9, 10, 11, 12, 13 or 14 days). In one embodiment, the cells are expanded for a period of 4 to 9 days. In one embodiment, the cells are expanded for a period of 8 days or less, e.g., 7, 6 or 5 days. In one embodiment, the cells are expanded in culture for 5 days, and the resulting cells are more potent than the same cells expanded in culture for 9 days under the same culture conditions. Potency can be defined, e.g., by various T cell functions, e.g. proliferation, target cell killing, cytokine production, activation, migration, or combinations thereof. In one embodiment, the cells, e.g., a CD19 CAR cell described herein, expanded for 5 days show at least a one, two, three or four fold increase in cells doublings upon antigen stimulation as compared to the same cells expanded in culture for 9 days under the same culture conditions. In one embodiment, the cells, e.g., the cells expressing a CD19 CAR described herein, are expanded in culture for 5 days, and the resulting cells exhibit higher proinflammatory cytokine production, e.g., IFN-γ and/or GM-CSF levels, as compared to the same cells expanded in culture for 9 days under the same culture conditions. In one embodiment, the cells, e.g., a CD19 CAR cell described herein, expanded for 5 days show at least a one, two, three, four, five, ten fold or more increase in pg/ml of proinflammatory cytokine production, e.g., IFN-γ and/or GM-CSF levels, as compared to the same cells expanded in culture for 9 days under the same culture conditions.

Several cycles of stimulation may also be desired such that culture time of T cells can be 60 days or more. Conditions appropriate for T cell culture include an appropriate media (e.g., Minimal Essential Media or RPMI Media 1640 or, X-vivo 15, (Lonza)) that may contain factors necessary for proliferation and viability, including serum (e.g., fetal bovine or human serum), interleukin-2 (IL-2), insulin, IFN-γ, IL-4, IL-7. GM-CSF, IL-10, IL-12, IL-15, TGFβ, and TNF-α or any other additives for the growth of cells known to the skilled artisan. Other additives for the growth of cells include, but are not limited to, surfactant, plasmanate, and reducing agents such as N-acetyl-cysteine and 2-mercaptoethanol. Media can include RPMI 1640, AIM-V, DMEM, MEM, α-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, e.g., penicillin and streptomycin, are included only in experimental cultures, not in cultures of cells that are to be infused into a subject. The target cells are maintained under conditions necessary to support growth, for example, an appropriate temperature (e.g., 37° C.) and atmosphere (e.g., air plus 5% CO₂).

In one embodiment, the cells are expanded in an appropriate media (e.g., media described herein) that includes one or more interleukin that result in at least a 200-fold (e.g., 200-fold, 250-fold, 300-fold, 350-fold) increase in cells over a 14 day expansion period, e.g., as measured by a method described herein such as flow cytometry. In one embodiment, the cells are expanded in the presence IL-15 and/or IL-7 (e.g., IL-15 and IL-7).

In embodiments, methods described herein, e.g., CAR-expressing cell manufacturing methods, comprise removing T regulatory cells, e.g., CD25+ T cells, from a cell population, e.g., using an anti-CD25 antibody, or fragment thereof, or a CD25-binding ligand. IL-2. Methods of removing T regulatory cells, e.g., CD25+ T cells, from a cell population are described herein. In embodiments, the methods, e.g., manufacturing methods, further comprise contacting a cell population (e.g., a cell population in which T regulatory cells, such as CD25+ T cells, have been depleted; or a cell population that has previously contacted an anti-CD25 antibody, fragment thereof, or CD25-binding ligand) with IL-15 and/or IL-7. For example, the cell population (e.g., that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide e.g., hetIL-15, during the manufacturing of the CAR-expressing cell, e.g., ex vivo. In embodiments, a CAR-expressing cell described herein is contacted with a composition comprising a IL-15 polypeptide during the manufacturing of the CAR-expressing cell, e.g., ex vivo. In embodiments, a CAR-expressing cell described herein is contacted with a composition comprising a combination of both a IL-15 polypeptide and a IL-15 Ra polypeptide during the manufacturing of the CAR-expressing cell, e.g., ex vivo. In embodiments, a CAR-expressing cell described herein is contacted with a composition comprising hetIL-15 during the manufacturing of the CAR-expressing cell, e.g., ex vivo.

In one embodiment the CAR-expressing cell described herein is contacted with a composition comprising hetlL-15 during ex vivo expansion. In an embodiment, the CAR-expressing cell described herein is contacted with a composition comprising an IL-15 polypeptide during ex vivo expansion. In an embodiment, the CAR-expressing cell described herein is contacted with a composition comprising both an IL-15 polypeptide and an IL-15Rα polypeptide during ex vivo expansion. In one embodiment the contacting results in the survival and proliferation of a lymphocyte subpopulation, e.g., CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR of the present invention are described in further detail below

Western blot analysis of CAR expression in primary T cells can be used to detect the presence of monomers and dimers, e.g., as described in paragraph 695 of International Application WO2015/142675, filed Mar. 13, 2015, which is herein incorporated by reference in its entirety.

In vitro expansion of CAR⁺ T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-la, ubiquitin C, or phosphoglycerokinase (PGK) promoters. GFP fluorescence is evaluated on day 6 of culture in the CD4⁺ and/or CD8⁺ T cell subsets by flow cytometry. See, e.g., Milone et al., Molecular Therapy 17(8): 1453-1464 (2009). Alternatively, a mixture of CD4⁺ and CD8⁺ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either a cancer associated antigen as described herein⁺ K562 cells (K562-expressing a cancer associated antigen as described herein), wild-type K562 cells (K562 wild type) or K562 cells expressing hCD32 and 4-1BBL in the presence of antiCD3 and anti-CD28 antibody (K562-BBL-3/28) following washing. Exogenous IL-2 is added to the cultures every other day at 100 IU/ml. GFP⁺ T cells are enumerated by flow cytometry using bead-based counting. See, e.g., Milone et al., Molecular Therapy 17(8): 1453-1464 (2009).

Sustained CAR⁺ T cell expansion in the absence of re-stimulation can also be measured. See, e.g., Milone et al., Molecular Therapy 17(8): 1453-1464 (2009). Briefly, mean T cell volume (fl) is measured on day 8 of culture using a Coulter Multisizer III particle counter, a Nexcelom Cellometer Vision or Millipore Scepter, following stimulation with αCD3/αCD28 coated magnetic beads on day 0, and transduction with the indicated CAR on day 1.

Animal models can also be used to measure a CAR-expressing cell activity, e.g., as described in paragraph 698 of International Application WO2015/142675, filed Mar. 13, 2015, which is herein incorporated by reference in its entirety.

Dose dependent CAR treatment response can be evaluated, e.g., as described in paragraph 699 of International Application WO2015/142675, filed Mar. 13, 2015, which is herein incorporated by reference in its entirety.

Assessment of cell proliferation and cytokine production has been previously described, as described in paragraph 700 of International Application WO2015/142675, filed Mar. 13, 2015, which is herein incorporated by reference in its entirety.

Cytotoxicity can be assessed by a standard 51Cr-release assay, e.g., as described in paragraph 701 of International Application WO2015/142675, filed Mar. 13, 2015. which is herein incorporated by reference in its entirety.

Cytotoxicity can also be assessed by measuring changes in adherent cell's electrical impedance, e.g., using an xCELLigence real time cell analyzer (RTCA). In some embodiments, cytotoxicity is measured at multiple time points.

Imaging technologies can be used to evaluate specific trafficking and proliferation of CARs in tumor-bearing animal models, e.g., as described in paragraph 702 of International Application WO2015/142675, filed Mar. 13, 2015, which is herein incorporated by reference in its entirety.

Other assays, including those described in the Example section herein as well as those that are known in the art can also be used to evaluate the CARs described herein.

Alternatively, or in combination to the methods disclosed herein, methods and compositions for one or more of: detection and/or quantification of CAR-expressing cells (e.g., in vitro or in vivo (e.g., clinical monitoring)); immune cell expansion and/or activation; and/or CAR-specific selection, that involve the use of a CAR ligand, are disclosed. In one exemplary embodiment, the CAR ligand is an antibody that binds to the CAR molecule, e.g., binds to the extracellular antigen binding domain of CAR (e.g., an antibody that binds to the antigen binding domain, e.g., an anti-idiotypic antibody; or an antibody that hinds to a constant region of the extracellular binding domain). In other embodiments, the CAR ligand is a CAR antigen molecule (e.g., a CAR antigen molecule as described herein).

In one aspect, a method for detecting and/or quantifying CAR-expressing cells is disclosed. For example, the CAR ligand can be used to detect and/or quantify CAR-expressing cells in vitro or in vivo (e.g., clinical monitoring of CAR-expressing cells in a patient, or dosing a patient). The method includes:

providing the CAR ligand (optionally, a labeled CAR ligand, e.g., a CAR ligand that includes a tag, a bead, a radioactive or fluorescent label);

acquiring the CAR-expressing cell (e.g., acquiring a sample containing CAR-expressing cells, such as a manufacturing sample or a clinical sample);

contacting the CAR-expressing cell with the CAR ligand under conditions where binding occurs, thereby detecting the level (e.g., amount) of the CAR-expressing cells present. Binding of the CAR-expressing cell with the CAR ligand can be detected using standard techniques such as FACS, ELISA and the like.

In another aspect, a method of expanding and/or activating cells (e.g., immune effector cells) is disclosed. The method includes:

providing a CAR-expressing cell (e.g., a first CAR-expressing cell or a transiently expressing CAR cell);

contacting said CAR-expressing cell with a CAR ligand, e.g., a CAR ligand as described herein), under conditions where immune cell expansion and/or proliferation occurs, thereby producing the activated and/or expanded cell population.

In certain embodiments, the CAR ligand is present on a substrate (e.g., is immobilized or attached to a substrate, e.g., a non-naturally occurring substrate). In some embodiments, the substrate is a non-cellular substrate. The non-cellular substrate can be a solid support chosen from, e.g., a plate (e.g., a microtiter plate), a membrane (e.g., a nitrocellulose membrane), a matrix, a chip or a bead. In embodiments, the CAR ligand is present in the substrate (e.g., on the substrate surface). The CAR ligand can be immobilized, attached, or associated covalently or non-covalently (e.g., cross-linked) to the substrate. In one embodiment, the CAR ligand is attached (e.g., covalently attached) to a bead. In the aforesaid embodiments, the immune cell population can be expanded in vitro or ex vivo. The method can further include culturing the population of immune cells in the presence of the ligand of the CAR molecule, e.g., using any of the methods described herein.

In other embodiments, the method of expanding and/or activating the cells further comprises addition of a second stimulatory molecule, e.g., CD28. For example, the CAR ligand and the second stimulatory molecule can be immobilized to a substrate, e.g., one or more beads, thereby providing increased cell expansion and/or activation.

In yet another aspect, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In one embodiment, the CAR ligand is coupled to a toxic agent (e.g., a toxin or a cell ablative drug). In another embodiment, the anti-idiotypic antibody can cause effector cell activity, e.g., ADCC or ADC activities.

Exemplary anti-CAR antibodies that can be used in the methods disclosed herein are described, e.g., in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference.

In some aspects and embodiments, the compositions and methods herein are optimized for a specific subset of T cells, e.g., as described in US Serial No. PCT/US2015/043219 filed Jul. 31, 2015, the contents of which are incorporated herein by reference in their entirety. In some embodiments, the optimized subsets of T cells display an enhanced persistence compared to a control T cell, e.g., a T cell of a different type (e.g., CD8+ or CD4+) expressing the same construct.

In some embodiments, a CD4+ T cell comprises a CAR described herein, which CAR comprises an intracellular signaling domain suitable for (e.g., optimized for, e.g., leading to enhanced persistence in) a CD4+ T cell, e.g., an ICOS domain. In some embodiments, a CD8+ T cell comprises a CAR described herein, which CAR comprises an intracellular signaling domain suitable for (e.g., optimized for, e.g., leading to enhanced persistence of) a CD8+ T cell, e.g., a 4-1BB domain, a CD28 domain, or another costimulatory domain other than an ICOS domain. In some embodiments, the CAR described herein comprises an antigen binding domain described herein, e.g., a CAR comprising an antigen binding domain.

In an aspect, described herein is a method of treating a subject, e.g., a subject having cancer. The method includes administering to said subject, an effective amount of:

1) a CD4+ T cell comprising a CAR (the CARCD4+) comprising:

an antigen binding domain, e.g., an antigen binding domain described herein;

a transmembrane domain; and

an intracellular signaling domain, e.g., a first costimulatory domain, e.g., an ICOS domain; and

2) a CD8+ T cell comprising a CAR (the CARCD8+) comprising:

an antigen binding domain, e.g., an antigen binding domain described herein;

a transmembrane domain; and

an intracellular signaling domain, e.g., a second costimulatory domain, e.g., a 4-1BB domain, a CD28 domain, or another costimulatory domain other than an ICOS domain;

wherein the CARCD4+ and the CARCD8+ differ from one another.

Optionally, the method further includes administering:

3) a second CD8+ T cell comprising a CAR (the second CARCD8+) comprising:

an antigen binding domain, e.g., an antigen binding domain described herein;

a transmembrane domain; and

an intracellular signaling domain, wherein the second CARCD8+ comprises an intracellular signaling domain, e.g., a costimulatory signaling domain, not present on the CARCD8+, and, optionally, does not comprise an ICOS signaling domain.

### Biopolymer Delivery Methods

In some embodiments, one or more CAR-expressing cells as disclosed herein can be administered or delivered to the subject via a biopolymer scaffold, e.g., a biopolymer implant. Biopolymer scaffolds can support or enhance the delivery, expansion, and/or dispersion of the CAR-expressing cells described herein. A biopolymer scaffold comprises a biocompatible (e.g., does not substantially induce an inflammatory or immune response) and/or a biodegradable polymer that can be naturally occurring or synthetic. Exemplary biopolymers are described, e.g., in paragraphs 1004-1006 of International Application WO2015/142675, filed Mar. 13, 2015, which is herein incorporated by reference in its entirety.

### Pharmaceutical Compositions and Treatments

In some aspects, the disclosure provides a method of treating a patient, comprising administering CAR-expressing cells produced as described herein, optionally in combination with one or more other therapies. In some aspects, the disclosure provides a method of treating a patient, comprising administering a reaction mixture comprising CAR-expressing cells as described herein, optionally in combination with one or more other therapies. In some aspects, the disclosure provides a method of shipping or receiving a reaction mixture comprising CAR-expressing cells as described herein. In some aspects, the disclosure provides a method of treating a patient, comprising receiving a CAR-expressing cell that was produced as described herein, and further comprising administering the CAR-expressing cell to the patient, optionally in combination with one or more other therapies. In some aspects, the disclosure provides a method of treating a patient, comprising producing a CAR-expressing cell as described herein, and further comprising administering the CAR-expressing cell to the patient, optionally in combination with one or more other therapies. The other therapy may be, e.g., a cancer therapy such as chemotherapy.

In an embodiment, cells expressing a CAR described herein are administered to a subject in combination with a molecule that decreases the Treg cell population. Methods that decrease the number of (e.g., deplete) Treg cells are known in the art and include, e.g., CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (e.g., Tregs) in the tumor microenvironment and reduces the subject's risk of relapse.

In one embodiment, a therapy described herein, e.g., a CAR-expressing cell, is administered to a subject in combination with a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In one embodiment, the GITR binding molecules and/or molecules modulating GITR functions (e.g., GITR agonist and/or Treg depleting GITR antibodies) are administered prior to the CAR-expressing cell. For example, in one embodiment, a GITR agonist can be administered prior to apheresis of the cells. In embodiments, cyclophosphamide is administered to the subject prior to administration (e.g., infusion or re-infusion) of the CAR-expressing cell or prior to apheresis of the cells. In embodiments, cyclophosphamide and an anti-GITR antibody are administered to the subject prior to administration (e.g., infusion or re-infusion) of the CAR-expressing cell or prior to apheresis of the cells. In one embodiment, the subject has cancer (e.g., a solid cancer or a hematological cancer such as ALL or CLL). In one embodiment, the subject has CLL. In embodiments, the subject has ALL. In embodiments, the subject has a solid cancer, e.g., a solid cancer described herein. Exemplary GITR agonists include, e.g., GITR fusion proteins and anti-GITR antibodies (e.g., bivalent anti-GITR antibodies) such as, e.g., a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, e.g., in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In one embodiment, a CAR expressing cell described herein is administered to a subject in combination with a GITR agonist, e.g., a GITR agonist described herein. In one embodiment, the GITR agonist is administered prior to the CAR-expressing cell. For example, in one embodiment, the GITR agonist can be administered prior to apheresis of the cells. In one embodiment, the subject has CLL.

The methods described herein can further include formulating a CAR-expressing cell in a pharmaceutical composition. Pharmaceutical compositions may comprise a CAR-expressing cell, e.g., a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives. Compositions can be formulated, e.g., for intravenous administration.

In one embodiment, the pharmaceutical composition is substantially free of, e.g., there are no detectable levels of a contaminant, e.g., selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In one embodiment, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-cancer effective amount,” “a cancer-inhibiting effective amount,” or “therapeutic amount” is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the immune effector cells (e.g., T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

In some embodiments, a dose of CAR cells (e.g., CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (e.g., CD19 CAR cells) comprises about 1.1×10⁶-1.8×10⁷ cells/kg. In some embodiments, a dose of CAR cells (e.g., CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (e.g., CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

In certain aspects, it may be desired to administer activated immune effector cells (e.g., T cells, NK cells) to a subject and then subsequently redraw blood (or have an apheresis performed), activate immune effector cells (e.g., T cells, NK cells) therefrom, and reinfuse the patient with these activated and expanded immune effector cells (e.g., T cells, NK cells). This process can be carried out multiple times every few weeks. In certain aspects, immune effector cells (e.g., T cells, NK cells) can be activated from blood draws of from 10 cc to 400 cc. In certain aspects, immune effector cells (e.g., T cells, NK cells) are activated from blood draws of 20 cc, 30 cc, 40 cc, 50 cc, 60 cc, 70 cc, 80 cc, 90 cc, or 100 cc.

The administration of the subject compositions may be carried out in any convenient manner. The compositions described herein may be administered to a patient trans arterially, subcutaneously, intradermally, intratumorally, intranodally, intramedullary, intramuscularly, by intravenous (i.v.) injection, or intraperitoneally, e.g., by intradermal or subcutaneous injection. The compositions of immune effector cells (e.g., T cells, NK cells) may be injected directly into a tumor, lymph node, or site of infection.

Exemplification of embodiments disclosed herein, e.g., are described in Examples 1-10, on pages 204-219 of International Application WO 2007/117112 filed on Dec. 27, 2016, which is hereby expressly incorporated by reference, including the figures and figure legends associated with said Examples.

### Exemplary Glycolysis Inhibitors

In some embodiments, glycolysis can be inhibited, e.g., suppressed, with an inhibitor of glycolysis, e.g., a small molecule inhibitor of glycolysis described herein. In some embodiments, an inhibitor of glycolysis is chosen from: an inhibitor of hexokinase (HK), an inhibitor of mitochondrial hexokinase 2 (HK2), an inhibitor of phosphofructokinase 2 (PFK2), an inhibitor of phosphoglycerate mutase (PGAM1), and inhibitor of pyruvate kinase 2 (PKM2), an inhibitor of pyruvate dehydrogenase kinase (PDK), an inhibitor of lactate dehydrogenase A (LDHA), an inhibitor of glucose-6-phosphate dehydrogenase (G6PD), an inhibitor of transketolase like 1 (TKTL1), or an inhibitor disclosed in Table 5.

In some embodiments, an inhibitor of glycolysis, e.g., a small molecule inhibitor of glycolysis, is chosen from: 2-deoxy-D-glucose (2-DG), 3-bromopyruvate (3-BP), Lonidamine. (2E)-3-(3-Pyridinyl)-1-(4-pyridinyl)-2-propen-1-one (3P0), N4A, YZ9, PGMI-004A, MJE3, TT-23, Shikonin/alkannin, FX11, Quinoline 3-sulfonamide, Dichloroacetate (DCA), 6-aminonicotinamide (6-AN), or Oxythiamine

In some embodiments, the inhibitor of glycolysis is 2-DG. 2-DG, which is also known as 2-Deoxy-D-mannose, or 2-Deoxy-D-arabino-hexose, is a glucose analog and acts, e.g., as a competitive inhibitor of glucose metabolism. 2-DG has the chemical name: (4R,5S,6R)-6-(hydroxymethyl)oxane-2,4,5-triol, and the following chemical structure:

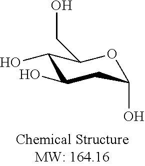

TABLE 5
Glycolysis inhibitors

| Compound name | Target protein |
|---|---|
| 2-deoxy-D-glucose(2-DG) | Inhibits HK |
| 3-bromopyruvate (3-BP) | Inhibits HK |
| Lonidamine | Inhibits mitochondrial HK2 |
| (2E)-3-(3-Pyridinyl)-1-(4-pyridinyl)-2- | Inhibits PFK2 |
| propen-1-one (3PO) | |
| N4A, YZ9 | Inhibits PFK2 |
| PGMI-004A | Inhibits PGAM1 |
| MJE3 | Inhibits PGAM1 |
| TT-232 | Inhibits PKM2 |
| Shikonin/alkannin | Inhibits PKM2 |
| FX11 | Inhibits LDHA |
| Quinoline 3-sulfonamides | Inhibit LDHA |
| Dichloroacetate (DCA) | Inhibits PDK |
| 6-aminonicotinamide (6-AN) | Inhibits G6PD |
| Oxythiamine | Inhibits TKTL1 |

Exemplary glycolysis inhibitors are disclosed in Qian Y. et al., (2014)World J Transl Med; 3(2): 37-57, the entire contents of which are herein incorporated by reference.

In embodiments, a method, reaction mixture or composition disclosed herein comprises 2-DG at a concentration in the range of 0.5-20 mM, 1-10 mM, or 1.5-2.5 mM. In some embodiments, the concentration of 2-DG is at least 0.5, 1.0, 1.5, 2.0 or 2.5 mM. In other embodiments the concentration of 2-DG is 2 mM.

In embodiments of any of the methods, reaction mixtures, or compositions disclosed herein, a CAR expressing immune effector cell population is contacted with 2-DG, e.g., 1 mM of 2-DG, for at least 1. 2, 3, 4, 5, 6, 7, 8, 9, 10, 12, 14, 16, 18, 20, 22, 24, 26, 28, 30, 32, 34, 36, 38, 40, 44, 48, 52, 60, 70, 80, 90, 100 hours or longer (e.g., up to about 2 or 3 weeks). In some embodiments, the CAR expressing immune effector cell population is contacted with 2-DG, e.g., 2 mM of 2-DG, for at least 48 hours.

Inhibition of glycolysis with 2-DG is disclosed, e.g., in Huang C C et al., (2015) Disease Models & Mechanisms 8: 1247-1254, the entire contents of which arc hereby incorporated by reference.

### Stat3 Activators and Stat3 Molecules

In embodiments of any of the methods, uses, or reaction mixtures disclosed herein a Stat3 activator comprises:

i) a gp130 activator, e.g., an antibody molecule that binds to gp130, e.g., an anti-gp130 antibody as described herein, or an IL-6 molecule, an IL-11 molecule, an IL-27 molecule, a CNTF molecule, a CT-1 molecule, a CLC molecule, a LIF molecule, a NP molecule, an OSM molecule;

ii) a soluble IL-6 receptor (sIL-6R), e.g., as described herein;

iii) an IL-6/IL-6R complex, e.g., a dimer, e.g., as described herein;

iv) an IL-6 family cytokine (e.g., an IL-6 molecule, an IL-11 molecule, an IL-27 molecule, an IL-31 molecule, a CNTF molecule, a CT-1 molecule, a CLC molecule, a LW molecule, a NP molecule or an OSM molecule);

v) a CCL20 molecule;

vi) an IL-10R2 receptor (IL-10R2) activator, e.g., an IL-10 molecule, an IL-22 molecule, an IL-26 molecule, an IL-28A molecule, an IL-28B molecule, an IL-29 molecule, or an antibody molecule that binds to IL-10R2, e.g., as described herein;

vii) an IL-10 family cytokine (e.g., an IL-10 molecule, an IL-19 molecule, an IL-20 molecule, an IL-22 molecule, an IL-24 molecule, an IL-26 molecule, an IL-28A molecule, an IL-28B molecule or an IL-29 molecule);

viii) an IL-17 family cytokine (e.g., an IL17A molecule, an IL17B molecule, an IL17C molecule, an IL17D molecule, an IL17E molecule or an IL17F molecule); or

ix) an IL-23 molecule.

In some embodiments, the Stat3 activator is a gp130 activator, e.g., an antibody molecule that binds to gp130, e.g., an anti-gp130 antibody as described herein.

In some embodiments, the Stat3 activator is a soluble IL-6 receptor (sIL-6R), e.g., as described herein.

In some embodiments, the Stat3 activator is an IL-6/IL-6R complex, e.g., a dimer, e.g., as described herein.

In some embodiments, the Stat3 activator is an IL-6 family cytokine (e.g., an IL-6 molecule, an IL-11 molecule, an IL-27 molecule, an IL-31 molecule, a CNTF molecule, a CT-1 molecule, a CLC molecule, a LIF molecule, a NP molecule or an OSM molecule).

In some embodiments, the Stat3 activator is a CCL20 molecule.

In some embodiments, the Stat3 activator is an IL-10R2 receptor (IL-10R2) activator, e.g., an IL-10 molecule, an IL-22 molecule, an IL-26 molecule, an IL-28A molecule, an IL-28B molecule, an IL-29 molecule, or an antibody molecule that binds to IL-10R2, e.g., as described herein.

In some embodiments, the Stat3 activator is an IL-17 family cytokine (e.g., an IL17A molecule, an IL17B molecule, an IL17C molecule, an IL17D molecule, an IL17E molecule or an IL17F molecule).

In some embodiments, the Stat3 activator is an IL-23 molecule.

In embodiments of any of the methods, uses, or reaction mixtures disclosed herein, a Stat3 molecule comprises an amino acid sequence having at least 80%, 85%, 90%, 95%, 96%, 97%, 98%, or 99% sequence identity to the amino acid sequence of SEQ ID NO: 1000. In some embodiments, the Stat3 molecule comprises the amino acid sequence of SEQ ID NO: 1000.

In some embodiments, a Stat3 molecule disclosed herein is encoded by a nucleotide sequence having at least 80%, 85%, 90%, 95%, 96%, 97%, 98%, or 99% sequence identity to the nucleotide sequence of SEQ ID NO: 1001. In some embodiments, the Stat3 molecule is encoded by the nucleotide sequence of SEQ ID NO: 1001, or nucleotides 241 to 2553 of SEQ ID NO: 1001.

In some embodiments, a cell described herein, e.g., a CAR-expressing cell, comprises a nucleic acid sequence, e.g., a transgene, comprising the sequence of SEQ ID NO: 1001, or nucleotides 241 to 2553 of SEQ ID NO: 1001.

Stat3 amino acid sequence; NCBI Ref Seq.
NP_644805.1
(SEQ ID NO: 1000)

| | |
|---|---|
| 1 | MAQWNQLQQL DTRYLEQLHQ LYSDSFPMEL RQFLAPWIES |
| | QDWAYAASKE SHATLVFHNL |
| 61 | LGEIDQQYSR FLQESNVLYQ HNLRRIKQFL QSRYLEKPME |
| | IARIVARCLW EESRLLQTAA |
| 121 | TAAQQGGQAN HPTAAVVTEK QQMLEQHLQD VRKRVQDLEQ |
| | KMKVVENLQD DFDFNYKTLK |
| 181 | SQGDMQDLNG NNQSVTRQKM QQLEQMLTAL DQMRRSIVSE |
| | LAGLLSAMEY VQKTLTDEEL |
| 241 | ADWKRRQQIA CIGGPPNICL DRLENWITSL AESQLQTRQQ |
| | IKKLEELQQK VSYKGDPIVQ |
| 301 | HRPMLEERIV ELFRNLMKSA FVVERQPCMP MHPURYLVIK |
| | IGVQFTIKVR LLVKFPELNY |
| 361 | QLKIKVCIDK DSGDVAALRG SRKFNILGTN TKVMNMEESN |
| | NGSLSAEFKH LTLREQRCGN |
| 421 | GGRANCDASL IVTEELHL1T FETEVYHQGL KIDLETHSLP |
| | VVVISNICQM PNAWASILWY |
| 481 | NMLTNNPKNV NFFTKPPIGT WDQVAEVLSW QFSSTTKRGL |
| | SIEQLTTLAE KLLGPGVNYS |
| 541 | GCQITWAKFC KENMAGKGFS FWVWLDNIID LVKKYILALW |
| | NEGYIMGFIS KERERAILST |
| 601 | KPPGTFLLRF SESSKEGGVT FTWVEKDISG KTQIQSVEPY |
| | TKQQLNNMSF AEIIMGYKIM |
| 661 | DATNILVSPL VYLYPDIPKE EAFGKYCRPE SQEHPEADPG |
| | SAAPYLKTKF 1CVIPTICSN |
| 721 | TIDLPMSPRT LDSLMQFGNN GEGAEPSAGG QFESLTFDME |
| | LTSECATSPM |

Stat3 nucleotide sequence; NCBI Ref Seq:
NM_1392762
(SEQ ID NO: 1001)

| | |
|---|---|
| 1 | ggtttccgga gctgcggcgg cgcagactgg gagggggagc |
| | cgggggttcc gacgtcgcag |
| 61 | ccgagggaac aagocccaac cggatcctgg acaggcaccc |
| | cggcttggcg ctgtctctcc |
| 121 | ccctcggctc ggagaggccc ttcggcctga gggagcctcg |
| | ccgcccgtcc ccggcacacg |
| 181 | cgcagccccg gcctctoggc ctctgccgga gaaacagttg |
| | ggacccctga ttttagcagg |
| 241 | atggcccaat ggaatcagct acagcagctt gacacacggt |
| | acctggagca gctccatcag |
| 301 | ctctacagtg acagcttccc aatggagctg cggcagtttc |
| | tggccccttg gattgagagt |
| 361 | caagattggg catatgcggc cagcaaagaa tcacatgcca |
| | ctttggtgtt tcataatctc |
| 421 | ctgggagaga ttgaccagca gtatagccgc ttcctgcaag |
| | agtcgaatgt tctctatcag |
| 481 | cacaatctac gaagaatcaa gcagtttctt cagagcaggt |
| | atcttgagaa gccaatggag |
| 541 | attgcccgga ttgtggcccg gtgcctgtgg gaagaatcac |
| | gccttctaca gactgcagcc |
| 601 | actgcggccc agcaaggggg ccaggccaac caccccacag |
| | cagccgtggt gacggagaag |
| 661 | cagcagatgc tggagcagca ccttcaggat gtccggaaga |
| | gagtgcagga tctagaacag |
| 721 | aaaatgaaag tggtagagaa tctccaggat gactttgatt |
| | tcaactataa aaccctcaag |
| 781 | agtcaaggag acatgcaaga tctgaatgga aacaaccagt |
| | cagtgaccag gcagaagatg |
| 841 | cagcagctgg aacagatgct cactgcgctg gaccagatgc |
| | ggagaagcat cgtgagtgag |
| 901 | ctggcggggc ttttgtcagc gatggagtac gtgcagaaaa |
| | ctctcacgga cgaggagctg |
| 961 | gctgactgga agaggcggca acagattgcc tgcattggag |
| | goccgcccaa catctgccta |
| 1021 | gatcggctag aaaactggat aacgtcatta gcagaatctc |
| | aacttcagac ccgtcaacaa |
| 1081 | attaagaaac tggaggagtt gcagcaaaaa gtttcctaca |
| | aaggggaccc cattgtacag |
| 1141 | caccggccga tgctggagga gagaatcgtg gagctgttta |
| | gaaacttaat gaaaagtgcc |
| 1201 | tttgtggtgg agcggcagcc ctgcatgccc atgcatcctg |
| | accggcccct cgtcatcaag |
| 1261 | accggcgtcc agttcactac tadaytcagg ttgctggtca |
| | aattccctga gttgaattat |
| 1321 | cagcttaaaa ttaaagtgtg cattgacaaa gactctgggg |
| | acgttgcagc tctcagagga |
| 1381 | tcccggaaat ttaacattct gggcacaaac acaaaagtga |
| | tgaacatgga agaatccaac |
| 1441 | aacggcagcc tctctgcaga attcaaacac ttgaccctga |
| | gggagcagag atgtgggaat |
| 1501 | gggggccgag ccaattgtga tgcttccctg attgtgactg |
| | aggagctgca cctgatcacc |
| 1561 | tttgagaccg aggtgtatca ccaaggcctc aagattgacc |
| | tagagacoca ctccttgcca |
| 1621 | gttgtggtga tctccaacat ctgtcagatg ccaaatgcct |
| | gggcgtccat cctgtggtac |
| 1681 | aacatgctga ccaacaatcc caagaatgta aactttttta |
| | ccaagccocc aattggaacc |
| 1741 | tgggatcaag tggccgaggt cctgagctgg cagttctcct |
| | ccaccaccaa gcgaggactg |
| 1801 | agcatcgagc agctgactac actggcagag aaactcttgg |
| | gacctggtgt gaattattca |
| 1861 | gggtgtcaga tcacatgggc taaattttgc aaagaaaaca |
| | tggctggcaa gggcttctcc |
| 1921 | ttctgggtct ggctggacaa tatcattgac cttgtgaaaa |
| | agtacatcct ggccctttgg |
| 1981 | aacgaagggt acatcatggg ctttatcagt aaggagcggg |
| | agcgggccat cttgagcact |
| 2041 | aagcctccag gcaccttcct gctaagattc agtgaaagca |
| | gcaaagaagg aggcgtcact |
| 2101 | ttcacttggg tggagaagga catcagcggt aagacccaga |
| | tccagtccgt ggaaccatac |
| 2161 | acaaagcagc agctgaacaa catgtcattt gctgaaatca |
| | tcatgggcta taagatcatg |
| 2221 | gatgctacca atatcctggt gtctccactg gtctatctct |
| | atcctgacat tcccaaggag |
| 2281 | gaggcattcg gaaagtattg tcggccagag agccaggagc |
| | atcctgaagc tgacccaggt |
| 2341 | agcgctgccc catacctgaa gaccaagttt atctgtgtga |
| | caccaacgac ctgcagcaat |
| 2401 | accattgacc tgccgatgtc cocccgcact ttagattcat |
| | tgatgcagtt tggaaataat |
| 2461 | ggtgaaggtg ctgaaccctc agcaggaggg cagtttgagt |
| | coctcacctt tgacatggag |
| 2521 | ttgacctcgg agtgcgctac ctcccccatg tgaggagctg |
| | agaacggaag ctgcagaaag |
| 2581 | atacgactga ggcgcctacc tgcattctgc cacccctcac |
| | acagccaaac cccagatcat |
| 2641 | ctgaaactac taactttgtg gttccagatt ttttttaatc |
| | tcctacttct gctatctttg |
| 2701 | agcaatctgg gcacttttaa aaatagagaa atgagtgaat |
| | gtgggtgatc tgcttttatc |
| 2761 | taaatgcaaa taaggatgtg ttctctgaga cccatgatca |
| | ggggatgtgg cggggggtgg |
| 2821 | ctagagggag aaaaaggaaa tgtcttgtgt tgttttgttc |
| | coctgccotc ctttctcagc |
| 2881 | agctttttgt tattgttgtt gttgttctta gacaagtgcc |
| | tcctggtgcc tgcggcatcc |
| 2941 | ttctgcctgt ttctgtaagc aaatgccaca ggccacctat |
| | agctacatac tcctggcatt |
| 3001 | gcacttttta accttgctga catccaaata gaagatagga |
| | ctatctaagc cctaggtttc |
| 3061 | tttttaaatt aayaaataat aacaattaaa gggcaaaaaa |
| | cactgtatca gcatagcctt |
| 3121 | tctgtattta agaaacttaa gcagccgggc atggtggctc |
| | acgcctgtaa tcccagcact |
| 3181 | ttgggaggcc gaggcggatc ataaggtcag gagatcaaga |
| | ccatcctggc taacacggtg |
| 3241 | aaaccccgtc tctactaaaa gtacaaaaaa ttagctgggt |
| | gtggtggtgg gcgcctgtag |
| 3301 | tcccagctac tcgggaggct gaggcaggag aatcgcttga |
| | acctgagagg cggaggttgc |
| 3361 | agtgagccaa aattgcacca ctgcacactg cactccatcc |
| | tgggcgacag tctgagactc |
| 3421 | tgtctcaaaa aaaaaaaaaa aaaaaagaaa cttcagttaa |
| | cagcctoctt ggtgctttaa |
| 3481 | gcattcagct tccttcaggc tggtaattta tataatccct |
| | gaaacgggct tcaggtcaaa |
| 3541 | cccttaagac atctgaagct gcaacctggc ctttggtgtt |
| | gaaataggaa ggtttaagga |
| 3601 | gaatctaagc attttagact tttttttata aatagactta |
| | ttttcctttg taatgtattg |
| 3661 | gccttttagt gagtaaggct gggcagaggg tgcttacaac |
| | cttgactocc tttctccctg |
| 3721 | gacttgatct gctgtttcag aggctaggtt gtttctgtgg |
| | gtgccttatc agggctggga |
| 3781 | tacttctgat tctggcttcc ttcctgcccc accctcccga |
| | ccocagtocc cctgatcctg |
| 3841 | ctagaggcat gtotccttgc gtgtctaaag gtccctcatc |
| | ctgtttgttt taggaatcct |
| 3901 | ggtctcagga cctcatggaa gaagaggggg agagagttac |
| | aggttggaca tgatgcacac |
| 3961 | tatggggccc cagcgacgtg tctggttgag ctcagggaat |
| | atggttctta gccagtttct |
| 4021 | tggtgatatc caytggcact tgtaatggcg tcttcattca |
| | gttcatgcag ggcaaaggct |
| 4081 | tactgataaa cttgagtctg ccctcgtatg agggtgtata |
| | cctggcctcc ctctgaggct |
| 4141 | ggtgactcct ccctgctggg gccccacagg tgaggcagaa |
| | cagctagagg gcctccccgc |
| 4201 | ctgcccgcct tggctggcta gctcgcctct cctgtgcgta |
| | tgggaacacc tagcacgtgc |
| 4261 | tggatgggct gcctctgact cagaggcatg gccggatttg |
| | gcaactcaaa accaccttgc |
| 4321 | ctcagctgat cagagtttct gtggaattct gtttgttaaa |
| | tcaaattagc tggtctctga |
| 4381 | attaaggggg agacgacctt ctctaagatg aacagggttc |
| | gccccagtcc tcctgcctgg |
| 4441 | agacagttga tgtgtcatgc agagctctta cttctccagc |
| | aacactcttc agtacataat |
| 4501 | aagcttaact gataaacaga atatttagaa aggtgagact |
| | tgggcttacc attgggttta |
| 4561 | aatcataggg acctagggcg agggttcagg gcttctctgg |
| | agcagatatt gtcaagttca |
| 4621 | tggccttagg tagcatgtat ctggtcttaa ctctgattgt |
| | agcaaaagtt ctgagaggag |
| 4681 | ctgagccctg ttgtggccca ttaaagaaca gggtcctcag |
| | gocctgccog cttcctgtcc |
| 4741 | actgccccct ccccatcccc agcccagccg agggaatccc |
| | gtgggttgct tacctaccta |
| 4801 | taaggtggtt tataagctgc tgtcctggcc actgcattca |
| | aattccaatg tgtacttcat |
| 4861 | agtgtaaaaa tttatattat tgtgaggttt tttgtctttt |
| | tttttttttt ttttttttgg |
| 4921 | tatattgctg tatctacttt aacttccaga aataaacgtt |
| | atataggaac cgtaaaaa |

In some embodiments, a Stat3 molecule disclosed herein is a Stat3 polypeptide, e.g., a wild-type Stat3 or a constitutively active Stat3 polypeptide, e.g., STAT3C. A constitutive form of Stat3 is encoded by the Stat3-C mutant form of the Stat3 gene. In Stat3-C the substitution of two cysteine residues within the C-terminal loop of the SH2 domain of Stat3 produces a molecule that dimerizes spontaneously, binds to DNA, and activates transcription, thus giving rise to a constitutively active molecule (Bromberg et al., (1999) Cell 98:295-303).

In embodiments of any of the methods, uses, or reaction mixtures disclosed herein, an IL-6 molecule comprises an amino acid sequence having at least 80%, 85%, 90%, 95%, 96%, 97%, 98%, or 99% sequence identity to the amino acid sequence of SEQ ID NO: 1002. In some embodiments, the IL-6 molecule comprises the amino acid sequence of SEQ ID NO: 1002.

In some embodiments, an IL-6 molecule disclosed herein is encoded by a nucleotide sequence having at least 80%, 85%, 90%, 95%, 96%, 97%, 98%, or 99% sequence identity to the nucleotide sequence of SEQ ID NO: 1003. In some embodiments, the IL-6 molecule is encoded by the nucleotide sequence of SEQ ID NO: 1003, or nucleotides 122 to 760 of SEQ ID NO: 1003.

In some embodiments, a cell described herein, e.g., a CAR-expressing cell, comprises a nucleic acid sequence, e.g., a transgene, comprising the sequence of SEQ ID NO: 1003, or nucleotides 122 to 760 of SEQ ID NO: 1003.

IL-6 amino acid sequence; GenBank Acc.
No. CAA68278.1
(SEQ ID NO: 1002)

| | |
|---|---|
| 1 | MNSFSISAFG PVAFSLGLLL VLPAAFPAPV PPGEDSKDVA |
| | APHRQPLZSS ERIDKQIRYI |
| 61 | LDGISALRKE TCNKSNMCES SKEALAENNL NLPKMAEKDG |
| | CFQSGFNEET CLVKIITGLL |
| 121 | EFEVYLEYLQ NRFESSEEQA RAVQMSTKVL IQFLQKKAKN |
| | LDAITTPDPT TNASLLTKLQ |
| 181 | AQNQWLQDMT THLILRSFKE FLQSSLRALR QM |

IL-6 nucleotide sequence; NCBI Ref Seq
NM_000600.4
(SEQ ID NO: 1003)

| | |
|---|---|
| 1 | gtctcaatat tagagtctca accoccaata aatataggac |
| | tggagatgtc tgaggctcat |
| 61 | tctgccctcg agcccaccgg gaacgaaaga gaagctctat |
| | ctoccotcca ggagcccagc |
| 121 | tatgaactcc ttctccacaa gcgccttcgg tccagttgcc |
| | ttctccctgg ggctgctcct |
| 181 | ggtgttgcct gctgccttcc ctgccccagt acccccagga |
| | gaagattcca aagatgtagc |
| 241 | cgccccacac agacagccac tcacctcttc agaacgaatt |
| | gacaaacaaa ttcggtacat |
| 301 | cctcgacggc atctcagccc tgagaaagga gacatgtaac |
| | aagagtaaca tgtgtgaaag |
| 361 | cagcaaagag gcactggcag aaaacaacct gaaccttcca |
| | aagatggctg aaaaagatgg |
| 421 | atgcttccaa tctggattca atgaggagac ttgcctggtg |
| | aaaatcatca ctggtctttt |
| 481 | ggagtttgag gtatacctag agtacctcca gaacagattt |
| | gagagtagtg aggaacaagc |
| 541 | cagagctgtg cagatgagta caaaagtcct gatccagttc |
| | ctgcagaaaa aggcaaagaa |
| 601 | tctagatgca ataaccaccc ctgacccaac cacaaatgcc |
| | agcctgctga cgaagctgca |
| 661 | ggcacagaac cagtggctgc aggacatgac aactcatctc |
| | attctgcgca gctttaagga |
| 721 | gttcctgcag tccagcctga gggctcttcg gcaaatgtag |
| | catgggcacc tcagattgtt |
| 781 | gttgttaatg ggcattcctt cttctggtca gaaacctgtc |
| | cactgggoac agaacttatg |
| 841 | ttgttctcta tggagaacta aaagtatgag cgttaggaca |
| | ctattttaat tatttttaat |
| 901 | ttattaatat ttaaatatgt gaagctgagt taatttatgt |
| | aagtcatatt tatattttta |
| 961 | agaagtacca cttgaaacat tttatgtatt agttttgaaa |
| | taataatgga aagtggctat |
| 1021 | gcagtttgaa tatcotttgt ttcagagcca gatcatttct |
| | tggaaagtgt aggcttacct |
| 1081 | caaataaatg gctaacttat acatattttt aaagaaatat |
| | ttatattgta tttatataat |
| 1141 | gtataaatgg tttttatacc aataaatggc attttaaaaa |
| | attcagcaaa aaaaaaa |

In embodiments of any of the methods, uses or reaction mixtures disclosed herein, a gp130 molecule comprises an amino acid sequence having at least 80%, 85%, 90%, 95%, 96%, 97%, 98%, or 99% sequence identity to the amino acid sequence of SEQ ID NO: 1004. In some embodiments, the gp130 molecule comprises the amino acid sequence of SEQ ID NO: 1004.

In some embodiments, a gp130 molecule disclosed herein is encoded by a nucleotide sequence having at least 80%, 85%, 90%, 95%, 96%, 97%, 98%, or 99% sequence identity to the nucleotide sequence of SEQ ID NO: 1005. In some embodiments, the gp130 molecule is encoded by the nucleotide sequence of SEQ ID NO: 1005, or nucleotides 113-2869 of SEQ ID NO: 1005.

In some embodiments, a cell described herein, e.g., a CAR-expressing cell, comprises a nucleic acid sequence, e.g., a transgene, comprising the sequence of SEQ ID NO: 1005, or nucleotides 113-2869 of SEQ ID NO: 1005.

gp130 amino acid sequence; GenBank Acc. No.
AAI17403.1
(SEQ ID NO: 1004)

| | |
|---|---|
| 1 | MLTLQTWLVQ ALFIFLTTES TGELLDPCGY ISPESPVVQL |
| | HSNFTAVCVL KEKCMDYFHV |
| 61 | NANYIVWKTN HFTIPKEQYT IINRTASSVT FTDIASLNIQ |
| | LTCNILTFGQ LEQNVYGITI |
| 121 | ISGLPPEKPK NLSCIVNEGK KMRCEWDRGR ETHLETNFTL |
| | KSEWATHKFA DCKAKRDTPT |
| 181 | SCTVDYSTVY FVNIEVWVEA ENALGKVTSD HINFDPVYKV |
| | KPNPPHNLSV INSEELSSIL |
| 241 | KLTWTNPSIK SVIILKYNIQ YRTKDASTWS QIPPEDTAST |
| | RSSFTVQDLK PFTEYVFRIR |
| 301 | CMKEDGKGYW SDWSEEASGT TYEDRPSKAP SFWYKIDPSH |
| | TQGYRTVQLV WKTLPPFEAN |
| 361 | GKILDYEVTL TRWKSHLQNY TVNATKLTVN LINDRYVAIL |
| | TVRNLVGKSD AAVLITPACD |
| 421 | FQATHPVMDL KAFPKDNMLW VEWTTPRESV KKYILEWCVL |
| | SDKAPCIIDW QQEDGTVHRT |
| 481 | YLRGNLAESK CYLTIVTPVY ADGPGSPEST KAYLKQAPPS |
| | KGPTVRTKKV GKNEAVLEWD |
| 541 | QLPVDVQNGF TRNYTIFYRT TIGNETAVNV DSSHTEYTLS |
| | SLTSDTLYMV RMAAYTDEGG |
| 601 | KDGPEFTFTT PKFAQGEIEA IVVPVCLAFL LTTLLGVLFC |
| | FNKRDLIKKH IWPNVPDPSK |
| 661 | SHIAQWSPHT PPRHNFNSKD QMYSDGNFTD VSVVETEAND |
| | KKPFPEDLKS LDLFKKEKIN |
| 721 | TEGHSSGIGG SSCMSSSRPS ISSSDENESS QNTSSTVQYS |
| | TVVHSGYRHQ VPSVQVFSRS |
| 781 | ESTQPLLDSE ERPEDLQLVD HVDGGDGILP RQQYFKQNCS |
| | QHESSPDISH FERSKQVSSV |
| 841 | NEEDFVRLKQ QISDHISQSC GSGQMKMFQE VSAADAFGPG |
| | TEGQVERFET VGMEAATDES |
| 901 | MPKSYLPQTV RQGGYMPQ |

gp130 nucleotide sequence; GenBank Acc. No.
BC117402.1
(SEQ ID NO: 1005)

| | |
|---|---|
| 1 | cggcctgagt gaaacccaat ggaaaaagca tgacatttag |
| | aagtagaaga cttagcttca |
| 61 | aatccctact cottoactta ctaattttgt gatttggaaa |
| | tatccgcgca agatgttgac |
| 121 | gttgcagact tggctagtgc aagccttgtt tattttcctc |
| | accactgaat ctacaggtga |
| 181 | acttctagat ccatgtggtt atatcagtcc tgaatctcca |
| | gttgtacaac ttcattctaa |
| 241 | tttcactgca gtttgtgtgc taaaggaaaa atgtatggat |
| | tattttcatg taaatgctaa |
| 301 | ttacattgtc tggaaaacaa accattttac tattcctaag |
| | gagcaatata ctatcataaa |
| 361 | cagaacagca tccagtgtca cctttacaga tatagcttca |
| | ttaaatattc agctcacttg |
| 421 | caacattctt acattcggac agcttgaaca gaatgtttat |
| | ggaatcacaa taatttcagg |
| 481 | cttgcctcca gaaaaaccta aaaatttgag ttgcattgtg |
| | aacgagggga agaaaatgag |
| 541 | gtgtgagtgg gatcgtggaa gggaaacaca cttggagaca |
| | aacttcactt taaaatctga |
| 601 | atgggcaaca cacaagtttg ctgattgcaa agcaaaacgt |
| | gacaccccca cctcatgcac |
| 661 | tgttgattat tctactgtgt attttgtcaa cattgaagtc |
| | tgggtagaag cagagaatgc |
| 721 | ccttgggaag gttacatcag atcatatcaa ttttgatcct |
| | gtatataaag tgaagcccaa |
| 781 | tccgccacat aatttatcag tgatcaactc agaggaactg |
| | tctagtatct taaaattgac |
| 841 | atggaccaac ccaagtatta agagtgttat aatactaaaa |
| | tataacattc aatataggac |
| 901 | caaagatgcc tcaacttgga gccagattcc tcctgaagac |
| | acagcatcca cccgatcttc |
| 961 | attcactgtc caagacctta aaccttttac agaatatgtg |
| | tttaggattc gctgtatgaa |
| 1021 | ggaagatggt aagggatact ggagtgactg gagtgaagaa |
| | gcaagtggga tcacctatga |
| 1081 | agatagacca tctaaagcac caagtttctg gtataaaata |
| | gatccatccc atactcaagg |
| 1141 | ctacagaact gtacaactcg tgtggaagac attgcctcct |
| | tttgaagcca atggaaaaat |
| 1201 | cttggattat gaagtgactc tcacaagatg gaaatcacat |
| | ttacaaaatt acacagttaa |
| 1261 | tgccacaaaa ctgacagtaa atctcacaaa tgatcgctat |
| | gtagcaaccc taacagtaag |
| 1321 | aaatcttgtt ggcaaatcag atgcagctgt tttaactatc |
| | cctgcctgtg actttcaagc |
| 1381 | tactcaccct gtaatggatc ttaaagcatt ccccaaagat |
| | aacatgcttt gggtggaatg |
| 1441 | gactactcca agggaatctg taaagaaata tatacttgag |
| | tggtgtgtgt tatcagataa |
| 1501 | agcaccctgt atcacagact ggcaacaaga agatggtacc |
| | gtgcatcgca cctatttaag |
| 1561 | agggaactta gcagagagca aatgctattt gataacagtt |
| | actccagtat atgctgatgg |
| 1621 | accaggaagc cctgaatcca taaaggcata ccttaaacaa |
| | gctccacctt ccaaaggacc |
| 1681 | tactgttcgg acaaaaaaag tagggaaaaa cgaagctgtc |
| | ttagagtggg accaacttcc |
| 1741 | tgttgatgtt cagaatggat ttatcagaaa ttatactata |
| | ttttatagaa ccatcattgg |
| 1801 | aaatgaaact gctgtgaatg tggattcttc ccacacagaa |
| | tatacattgt cctctttgac |
| 1861 | tagtgacaca ttgtacatgg tacgaatggc agcatacaca |
| | gatgaaggtg ggaaggatgg |
| 1921 | tccagaattc acttttacta ccccaaagtt tgctcaagga |
| | gaaattgaag ccatagtcgt |
| 1981 | gcctgtttgc ttagcattcc tattgacaac tcttctggga |
| | gtgctgttct gctttaataa |
| 2041 | gcgagaccta attaaaaaac acatctggcc taatgttcca |
| | gatccttcaa agagtcatat |
| 2101 | tgcccagtgg tcacctcaca ctcctccaag gcacaatttt |
| | aattcaaaag atcaaatgta |
| 2161 | ttcagatggc aatttcactg atgtaagtgt tgtggaaata |
| | gaagcaaatg acaaaaagcc |
| 2221 | ttttccagaa gatctgaaat cattggacct gttcaaaaag |
| | gaaaaaatta atactgaagg |
| 2281 | acacagcagt ggtattgggg ggtcttcatg catgtcatct |
| | tctaggccaa gcatttctag |
| 2341 | cagtgatgaa aatgaatctt cacaaaacac ttcgagcact |
| | gtccagtatt ctaccgtggt |
| 2401 | acacagtggc tacagacacc aagttccgtc agtccaagtc |
| | ttctcaagat ccgagtctac |
| 2461 | ccagcccttg ttagattcag aggagcggcc agaagatcta |
| | caattagtag atcatgtaga |
| 2521 | tggcggtgat ggtattttgc ccaggcaaca gtacttcaaa |
| | cagaactgca gtcagcatga |
| 2581 | atccagtcca gatatttcac attttgaaag gtcaaagcaa |
| | gtttcatcag tcaatgagga |
| 2641 | agattttgtt agacttaaac agcagatttc agatcatatt |
| | tcacaatcct gtggatctgg |
| 2701 | gcaaatgaaa atgtttcagg aagtttctgc agcagatgct |
| | tttggtccag gtactgaggg |
| 2761 | acaagtagaa agatttgaaa cagttggcat ggaggctgcg |
| | actgatgaag gcatgcctaa |
| 2821 | aagttactta ccacagactg tacggcaagg cggctacatg |
| | cctcagtgaa ggactagtag |
| 2881 | ttcctgctac aacttcagca gtacctataa agtaaagcta |
| | aaatgatttt atctgtgaat |
| 2941 | tcagatttta aaaagtcttc actctctgaa gatgatcatt |
| | tgccc |

### Anti-gp130 Antibodies

In some embodiments, the Stat3 activator is a gp130 activator, e.g., an antibody molecule that binds to gp130, e.g., an anti-gp130 antibody as described herein.

Also disclosed herein, are anti-gp130 antibody molecules. Anti-gp130 antibody molecules are disclosed, e.g., in Gu Z. J. et al., Leukemia (2000) 14:188-197, the entire contents of which are hereby incorporated by reference.

In embodiments of any of the methods, uses, or reaction mixtures described herein, a anti-gp130 antibody molecule comprises a B-S12 anti-gp130 antibody or a B-P8 anti-gp130 antibody, or both a B-S12 and B-P8 antibody. Without wishing to be bound by theory, it is believed that in some embodiments, a B-S12 and/or B-P8 antibody molecule results in dimerization of gp130.

In some embodiments, the anti-gp130 antibody has one, two, three, four or more (e.g., all) of the following characteristics:

i) induces gp130 mediated signaling, as measured by phosphorylation of STAT1, STAT3, ERK1 or ERK2;

ii) is not inhibited by an IL-6 inhibitor, e.g., Sttatic, or an inhibitor described herein;

iii) promotes growth of myeloma cell lines;

iv) supports survival of primary myeloma cells; or

v) induces dimerization, e.g., homodimerization of gp130, or heterodimerization of gp130, e.g., with LIF, OSM or CNTF.

In some embodiments, a gp130 antibody molecule disclosed herein comprises an antibody molecule having at least 1, 2, 3, 4, 5, or 6 CDRs from B-S12 or B-P8. In some embodiments, the CDRs are defined according to Chothia, Kabat or a combination thereof.

In some embodiments, a gp130 antibody molecule comprises 1, 2, 3, 4, 5, or 6 CDRs from B-S12. In some embodiments, the gp130 antibody molecule comprises one or more of a light chain complementarity determining region 1 (LC CDR1), a light chain complementarity determining region 2 (LC CDR2), and a light chain complementarity determining region 3 (LC CDR3) of B-S12. In some embodiments, the gp130 antibody molecule comprises one or more of a heavy chain complementarity determining region 1 (HC CDR1), a heavy chain complementarity determining region 2 (HC CDR2), and a heavy chain complementarity determining region 3 (HC CDR3) of B-S12. In some embodiments, the gp130 antibody molecule comprises one or more of a light chain complementarity determining region 1 (LC CDR1), a light chain complementarity determining region 2 (LC CDR2), and a light chain complementarity determining region 3 (LC CDR3) of B-S12 and one or more of a heavy chain complementarity determining region 1 (HC CDR1), a heavy chain complementarity determining region 2 (HC CDR2), and a heavy chain complementarity determining region 3 (HC CDR3) of B-S12.

In some embodiments, a gp130 antibody molecule comprises 1, 2, 3, 4, 5, or 6 CDRs from B-P8. In some embodiments, the gp130 antibody molecule comprises one or more of a light chain complementarity determining region 1 (LC CDR1), a light chain complementarity determining region 2 (LC CDR2), and a light chain complementarity determining region 3 (LC CDR3) of B-P8. In some embodiments, the gp130 antibody molecule comprises one or more of a heavy chain complementarity determining region 1 (HC CDR1), a heavy chain complementarity determining region 2 (HC CDR2), and a heavy chain complementarity determining region 3 (HC CDR3) of B-P8. In some embodiments, the gp130 antibody molecule comprises one or more of a light chain complementarity determining region 1 (LC CDR1), a light chain complementarity determining region 2 (LC CDR2), and a light chain complementarity determining region 3 (LC CDR3) of B-P8 and one or more of a heavy chain complementarity determining region 1 (HC CDR1), a heavy chain complementarity determining region 2 (HC CDR2), and a heavy chain complementarity determining region 3 (HC CDR3) of B-P8.

In some embodiments, the gp130 antibody, e.g., a B-S12 or B-P8 antibody molecule, is provided at an amount of at least 0.5, 0.6, 0.7, 0.8, 0.9, 1.0, 1.1, 1.2, 1.3, 1.4, 1.5, 1.6, 1.7, 1.8, 1.9, or 2 ug/ml, and optionally up to about 2 ug/ml, e.g., about 1 ug/ml. In some embodiments, the gp130 antibody, e.g., a B-S12 or B-P8 antibody molecule is provided at an amount of 1 ug/ml.

In some embodiments, the anti-gp130 antibody molecule comprises a first and a second anti-gp130 antibody molecule, e.g., B-S12 and B-P8 antibody molecules. In some embodiments, the B-S12 and B-P8 antibody molecules are provided at a ratio of about 1:1. In some embodiments, B-S12 and B-P8 antibodies are provided at a combined amount of 0.1-1000, 0.5-500, or 1-100 ug/ml. In some embodiments, the B-S12 and B-P8 antibody molecules are each provided at an amount of at least 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2 ug/ml, and optionally up to about 2 ug/ml, e.g., about 0.5 ug/ml each. In some embodiments, the B-S12 and B-P8 antibody molecules are each provided at an amount of 0.5 ug/ml.

In some embodiments, the Stat3 activator is an antibody molecule that binds to gp130, e.g., an anti-gp130 antibody as described herein.

In some embodiments, a method of making, e.g., a method of manufacturing, e.g., as described herein, results in a population of T cells, e.g., CD4+ or CD8+ T cells, that is enriched for (e.g., has increased levels of), e.g., early memory T cells or non-exhausted early memory T cells.

In some embodiments, the method results in enrichment of CD4+ or CD8+ early memory T cells, e.g., as described herein. In some embodiments, early memory T cells have one or both of the following characteristics: CD27+ and/or CD45RO^{dim/neg}, e.g., CD27+ CD45RO^{dim/neg}. In some embodiments, the method results in enrichment of CD4+ or CD8+ non-exhausted early memory T cells, e.g., as described herein.

In some embodiments, the method results in enrichment of CD4+ or CD8+ non-exhausted early memory T cells, e.g., as described herein. In some embodiments, non-exhausted early memory T cells have one or more, e.g., all, of the following characteristics: (i) PD-1 negative; (ii) CD27^{hi}; (iii) CCR7^{hi}; or (iv) CD45RO^{dim/neg}. In some embodiments, non-exhausted early memory T cells are PD-1 negative CD27^{hi} CCR7^{hi} CD45RO^{dim/neg}.

In some embodiments, the enriched population of T cells, e.g., early memory T cells or non-exhausted early memory T cells, has an increased level of, e.g., at least 5-90% more (e.g., at least 5-10, 10-20, 20-30, 30-50, 50-70, or 70-90% more, or at least 5-90, 10-85, 15-80, 20-75, 25-70, 30-70. 35-65, 40-60, or 45-55% more) early memory T cells or non-exhausted early memory T cells. In some embodiments, the enriched population of T cells, e.g., early memory T cells or non-exhausted early memory T cells, has an increased level of, e.g., at least 5, 6, 7, 8, 9, 10, 11, 12, 13, 14, 15, 16, 17, 18, 19, 20, 21, 22, 23, 24, 25, 26, 27, 28, 29 or 30% more), early memory T cells or non-exhausted early memory T cells.

In some embodiments, the increased level of early memory T cells or non-exhausted early memory T cells is compared to an otherwise similar population of T cells that was not contacted with the Stat3 activator, e.g., as described in Example 2. In some embodiments, the otherwise similar population of T cells that was not contacted with the Stat3 activator is the same population of T cells, e.g., on which the enrichment was performed, e.g., a pre-enrichment population, e.g., a starting population, e.g., as described in Example 2. In some embodiments, the otherwise similar population of T cells that was not contacted with the Stat3 activator is a different population of T cells, e.g., a population on which the enrichment was not performed.

In some embodiments, the enriched population of CD4+ T cells, e.g., early memory T cells or non-exhausted early memory T cells, has an increased level of, e.g., at least 5, 6, 7, 8, 9, 10, 11, 12, 13, 14, 15, 16, 17, 18, 19, or 20% more (e.g., at least 8% more), early memory T cells or non-exhausted early memory T cells compared to an otherwise similar population of T cells that was not contacted with the Stat3 activator.

In some embodiments, the enriched population of CD8+ T cells, e.g., early memory T cells or non-exhausted early memory T cells, has an increased level of, e.g., at least 15, 16, 17, 18, 19, 20, 21, 22, 23, 24, 25, 26, 27, 28, 29 or 30% more (e.g., at least 20% more), early memory T cells or non-exhausted early memory T cells compared to an otherwise similar population of T cells that was not contacted with the Stat3 activator.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: Glycolytic Metabolism of CTL019 Cells

CAR T cell samples from 41 patients with advanced, heavily pre-treated and high-risk CLL who received at least one dose of CD19-directed CAR T cells were evaluated for metabolic changes.

### Results

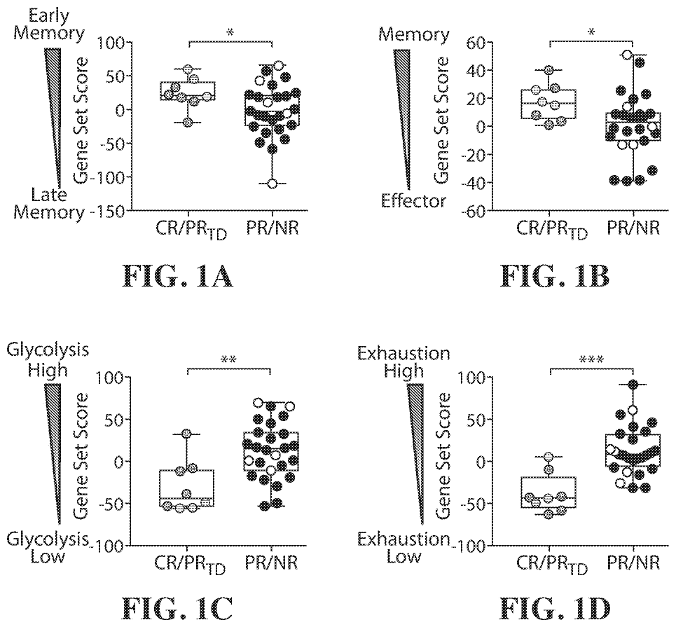

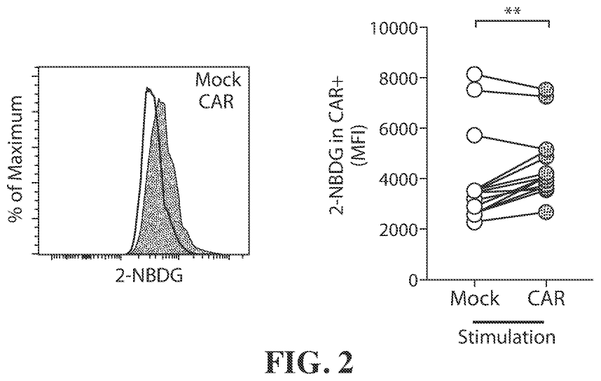

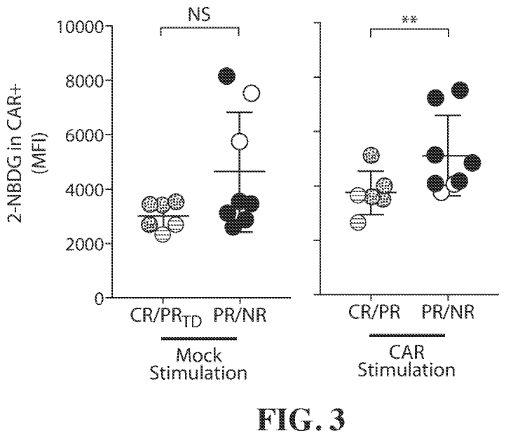

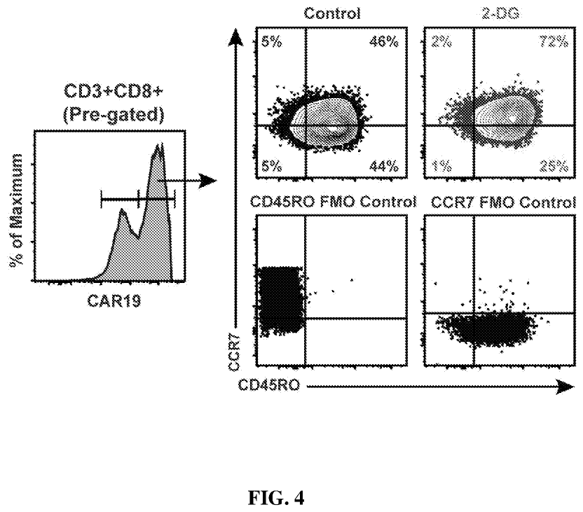

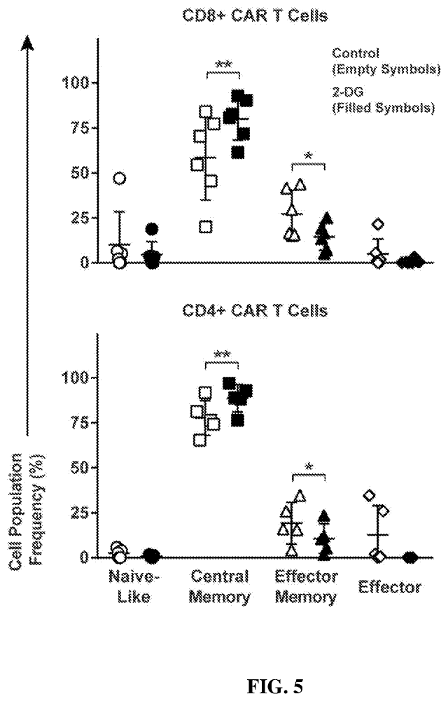

CAR T cells from patients who had a Partial Response (PR) or No Response (NR) demonstrated a glycolytic gene signature. CAR-specific stimulation of retrospective cellular infusion product samples increased glycolysis and the uptake of a glucose analog (FIG. 2), which provides evidence that CLL patient T cells can undergo metabolic modulation by ligation of a synthetic CAR. CAR stimulated T cells from PR/NR relative to CR/PRTD (Partial Response, Transfusion-Dependent) patients demonstrated a higher uptake of a glucose analog (FIG. 3), which is consistent with the transcriptional profiles of these cells (FIGS. 1A-1D). Furthermore, blockade of glycolysis using 2-deoxy-D-glucose (2-DG) decreased effector differentiation and resulted in increased frequencies of CAR T cells with a central memory phenotype (FIG. 4 and FIG. 5).

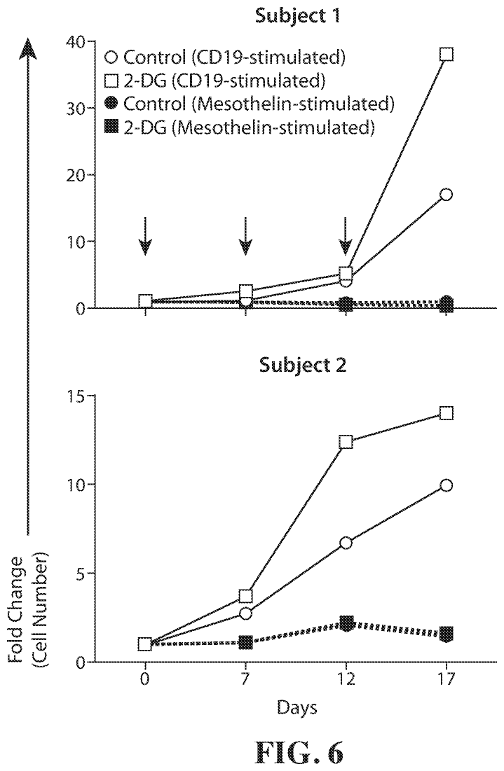

Pharmacologic inhibition of glycolysis concomitantly promoted the formation of memory CTL019 lymphocytes with enhanced proliferative capacity following re-stimulation with CD19− expressing tumor cells (FIG. 6). These results indicate that T cells generated from CR and PRTD patients show a gene expression profile that could confer properties of persistence as well as robust anti-tumor potency, and that decreasing glycolytic metabolism may represent an actionable cellular manufacturing improvement for enhancing CAR T cell potency.

The findings described in this Example underscore the potential utility of increasing the therapeutic efficacy of adoptively transferred T lymphocytes by selecting cultures with high absolute numbers or higher relative abundance of a mechanistically relevant subpopulation responsible for mediating tumor control, and/or providing manufacturing conditions which bias the lymphocyte population (including CAR+ T lymphocyte population) towards higher absolute numbers or relative abundance of these subpopulations. Generation of CAR T cell infusion products with optimal differentiation potential as well as effector activity might also be achieved by minimizing time in culture, the use of alternative cytokines, and metabolic engineering.

### Materials and Methods

### Vector Production, T Cell Isolation and Generation of CTL019 Cells

The lentiviral vector that contains a transgene encoding the CD19-specific CAR with 4-1BB/CD3ζ domains (GeMCRIS0607-793) was constructed and produced. Autologous T cells were collected by leukapheresis and activated with clinical-grade paramagnetic polystyrene beads coated with anti-CD3 and anti-CD28 monoclonal antibodies followed by transduction with the above lentiviral vector. CAR-transduced T cells were expanded ex vivo for 9-11 days. Absolute cell counts during large-scale CTL019 cell culture were obtained using a Coulter Counter (Beckman Coulter). Population doublings were calculated using the equation At=A02n, where n is the number of population doublings, AO is the input number of cells, and At is the total number of cells.

### Flow Cytometry

Routine longitudinal measurements of the expansion and persistence of CTL019 cells, as well as peripheral B-CLL burden were conducted as previously described with a six parameter Accuri C6 flow cytometer (BD Biosciences). For T cell deep immunophenotyping, PBMC or CTL019 infusion products (hulk T cells or specific subsets purified by fluorescence activated cell sorting) were pre-incubated with Aqua Blue dead cell exclusion dye (Invitrogen), followed by surface staining with commercially available flow cytometry antibodies. CAR19 protein expression was detected using an Alexa Fluor 647-conjugated anti-idiotype monoclonal antibody. All antibodies used in this study were titrated prior to use, and fluorescence minus one (FMO) controls were created for each antibody panel to set gates for positive events. Cells were acquired on a custom 17-color, 19-parameter special order LSRFortessa (BD Biosciences). Data were analyzed using FlowJo software (TreeStar).

### Measurement of Glucose Uptake by CLL Patient CAR T Cells

Retrospective CLL patient CTL019 cells were thawed and rested in 24-well tissue culture plates (BD Biosciences) at 1×106 cells/ml, followed by overnight stimulation with anti-idiotypic antibody-coated beads to recapitulate triggering of the anti-CD19BBζ CAR by cognate CD19 antigen. Similarly-conjugated isotype antibody-coated beads were used as the mock control. Beads were added in a ratio of 3 beads:1 cell according to the transduction efficiency of each patient infusion product. Enzymatic diagnostic kits were used in accordance with the manufacturer's (Sigma) instructions. The signals generated from a range of concentrations of this metabolite were used to construct a standard curve. Cellular supernatants from mock- and CAR-stimulated T cells were collected and applied to the instrument. Following overnight stimulation, cells were washed and re-458 fed with fresh RPMI and 2-[N-(7-nitrobenz-2-oxa-1,3-diazol-4-yl) amino]-2-deoxy-D-glucose (2-NBDG; Sigma Aldrich) was added at a final concentration of 500 μM. After a 30-minute incubation with 2-NBDG, cells were placed on ice and stained for flow cytometry to detect CAR+ T cells that had taken up the fluorescently-labeled glucose analog.

### Metabolic Modulation of CAR T Cells

Anti-CD19BBζ CAR T cells were generated as in the presence or absence of the glycolysis inhibitor 2-deoxyglucose (2-DG; Sigma-Aldrich). Following 9 days of culture, the differentiation phenotype of these cells was determined by flow cytometry. CAR+T-cells were sorted on the FACSAria II cell sorter (BD Biosciences) and combined 1:1 with irradiated K562 cells engineered to express CD19 (K562-CD19). CTL019cells were re-stimulated ×3 with K562-CD19 targets. During the proliferation assay, absolute cell counts were obtained using the Luna automated cell counter (Logos Biosystems).

### Example 2: STAT3 Pathway Activation in CD19-Expressing Chimeric Antigen Receptor (CAR) T Cells

CAR T cell samples from patients with advanced, heavily pre-treated and high-risk

CLL who received at least one dose of CD19-directed CART cells were evaluated for STAT3 pathway activation.

### Materials and Methods

### Patient Samples

Samples were obtained from CLL patients enrolled in Institutional Review Board (IRB) of the University of Pennsylvania-approved clinical trials of single-agent CTL019 therapy. All participants provided written informed consent in accordance with the Declaration of Helsinki and the International Conference on Harmonization Guidelines for Good Clinical Practice. These studies are registered at ClinicalTrials.gov (identifiers: NCT01029366, NCT01747486 and NCT02640209).

### Vector Production, T Cell Isolation and Generation of CTL019 Cells

The lentiviral vector that contains a transgene encoding the CD19-specific CAR with 4-1BB/CD3ζ domains (GeMCRIS 377 0607-793) was constructed and produced. Autologous T cells were collected by leukapheresis and activated with clinical-grade paramagnetic polystyrene beads coated with anti-CD3 and anti-CD28 monoclonal antibodies followed by transduction with the above lentiviral vector. CAR-transduced T cells were expanded ex vivo for 9-11 days. Absolute cell counts during large-scale CTL019 cell culture were obtained using a Coulter Counter (Beckman Coulter). Population doublings were calculated using the equation Aₜ=A₀₂n, where n is the number of population doublings, A₀ is the input number of cells, and Aₜ is the total number of cells.

Measurement of pSTAT3 and STAT3 Blockade in CAR T Cells.

CTL019 cells from CLL patient infusion products were thawed and stimulated with anti-idiotypic antibody-coated beads or mock beads. For some experiments, cells were stimulated for 10 minutes with recombinant human IL-6 (Miltenyi Biotec.) at a final concentration of 10 ng/mL. In cell subset evaluation experiments, populations of interest were stained for surface markers and sorted on a FACSAria II cell sorter (BD Biosciences) prior to acute stimulation with IL-6. Following stimulation, cells were fixed with Phosflow Lyse/Fix Buffer (BD Biosciences) for 12 minutes at 37° C., and permeabilized on ice for 30 minutes using Phosflow Perm Buffer III (BD Biosciences). Intracellular staining was performed for 60 minutes at room temperature using an anti-STAT3 (pY705) antibody (BD Biosciences) at the manufacturer's recommended concentration. Samples were immediately acquired on the LSRFortessa (BD Biosciences). Blockade of the STAT3 pathway was carried out by generating anti-CD19BBζ c CAR T cells in the presence or absence of the STAT3-specific inhibitor, Stattic (Selleckchem) at a final concentration of 5 μM. An equivalent amount of dimethyl sulfoxide (DMSO) served as a negative vehicle alone control. The ability of Stattic to inhibit STAT3 signaling was assessed by Phosflow staining as described above. Serial re-stimulation assays using sorted CAR T cells and irradiated K562-CD19 cells were also carried as described above. Absolute cell numbers and viability were simultaneously measured during the course of CAR T cell expansion using the Luna automated cell counter (Logos Biosystems).

### Cytokine Analyses

CTL019 products were cultured overnight with anti-idiotypic antibody-coated beads or isotype control antibody-coated beads as described above and supernatants were collected. Serum was isolated from the whole blood of patients at baseline and at defined post-CTL019 treatment time points by centrifugation. These samples were distributed in single-use aliquots and stored at −80° C. Measurement of cytokines in the above culture supernatants and serum samples was performed using a Luminex bead array platform according to the manufacturer's (Life Technologies) instructions. All samples were analyzed in triplicate and compared to multiple internal standards using a 9-point standard curve. Acquisition of data took place on a FlexMAP-3D system (Luminex Corp.) and analysis was performed using XPonent 4.0 software as well as 5-parameter logistic regression analysis7.

### Flow Cytometry

Routine longitudinal measurements of the expansion 435 and persistence of CTL019 cells, as well as peripheral B-CLL burden were conducted as previously described with a six parameter Accuri C6 flow cytometer (BD Biosciences). For T cell deep immunophenotyping, PBMC or CTL019 infusion products (hulk T cells or specific subsets purified by fluorescence activated cell sorting) were pre-incubated with Aqua Blue dead cell exclusion dye (Invitrogen), followed by surface staining with commercially available flow cytometry antibodies. CAR19 protein expression was detected using an Alexa Fluor 647-conjugated anti-idiotype monoclonal antibody. All antibodies used in this study were titrated prior to use, and fluorescence minus one (FMO) controls were created for each antibody panel to set gates for positive events. Cells were acquired on a custom 17-color, 19-parameter special order LSRFortessa (BD Biosciences). Data were analyzed using FlowJo software (TreeStar).

### Results

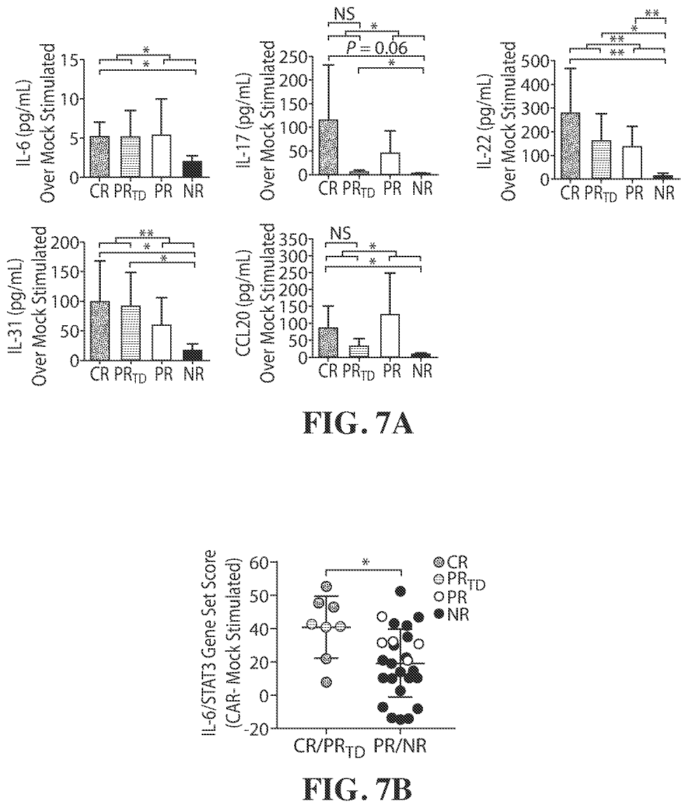

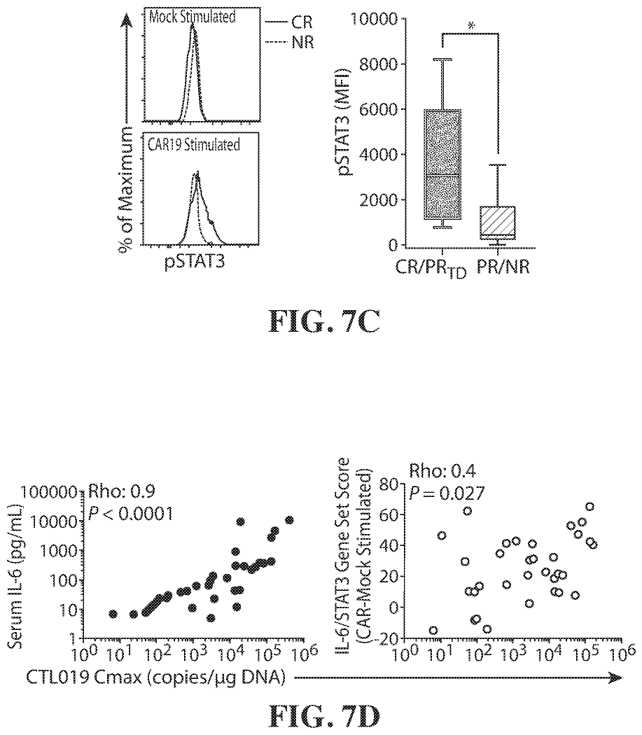

The profile of cytokines and chemokines produced from CTL019 cells derived from CR, PR_{TD}, PR and NR patient infusion products after CAR stimulation were evaluated. As shown in FIG. 7A, CD19-directed T cells manufactured from CR and PR_{TD} subjects showed higher levels of STAT3 signaling mediators and targets, including IL-6, IL-17, IL-22, IL-31 and CCL20 compared to PRs and NRs. This finding was consistent with IL-6/STAT3 pathway upregulation in evaluable CR and PRTD patient CTL019 cells that were CAR-stimulated (FIG. 7B). These findings suggest that activation of STAT3 in CAR T cells might be involved in the generation of less differentiated, multipotent T lymphocytes.

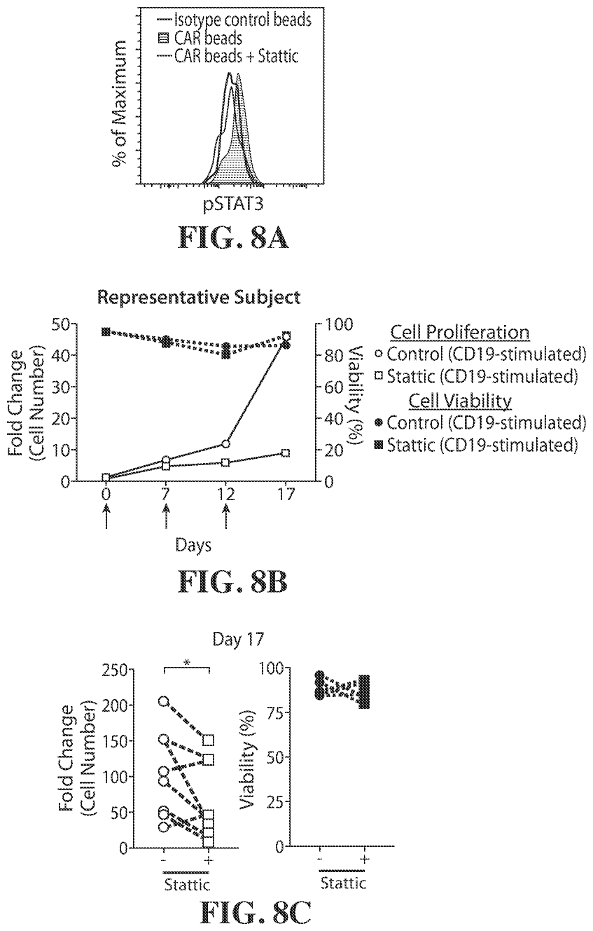

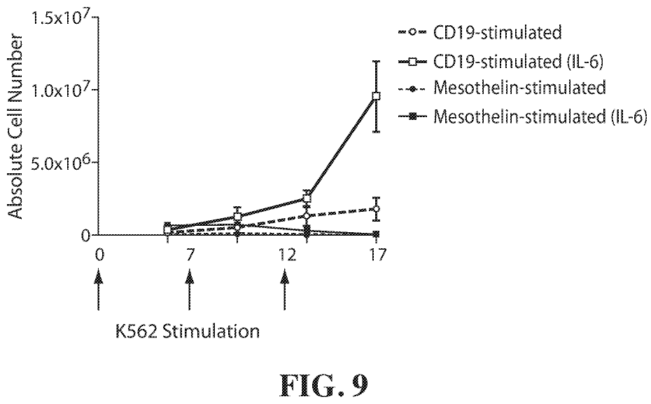

It was then determined whether levels of phosphorylated STAT3 (pSTAT3) could segregate highly potent CR/PRTD patient CAR T cell infusion products from those of PR/NR subjects with poor functionality. CAR-specific pSTAT3 activity was significantly elevated in CTL019 cells from CR/PR_{TD} patients compared to CAR T cells expanded from PR/NR patients (FIG. 7C). In line with these findings, a strong, direct correlation was observed between the maximum degree of in vivo CAR T cell expansion and serum IL-6 as well as IL-6/STAT3 pathway gene enrichment in pre-infusion CTL019 cells following stimulation (FIG. 7D). Accordingly, pharmacologic blockade of STAT3 signaling in CTL019 cells diminished their proliferative capacity upon serial re-stimulation with CD19− expressing leukemia cells (FIG. 8A-8B) without affecting viability (FIG. 8C). Increasing pSTAT3 activity by addition of exogenous IL-6 to culture media during the manufacturing of CTL019 cells elevated the expansion capacity and the absolute numbers of CAR T cells following repeated stimulation with CD19− expressing tumor targets (FIG. 9). Considering the emerging role of STATS signaling in the formation and maintenance of memory T cells, induction of this pathway might not only be a demarcation of less differentiated cells in the CAR T cell product, but also functionally important for their expansion and long-term survival after infusion.

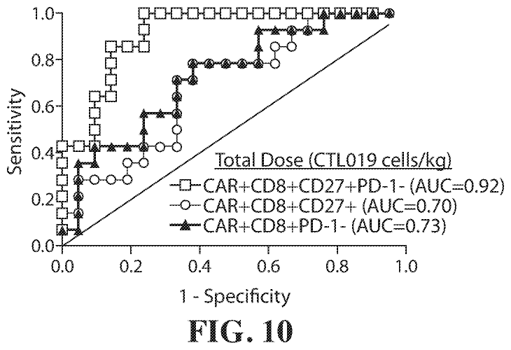

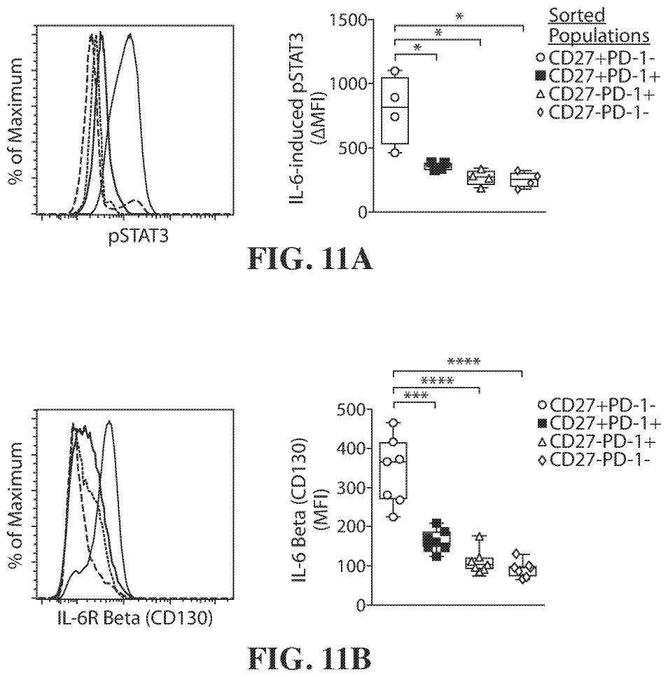

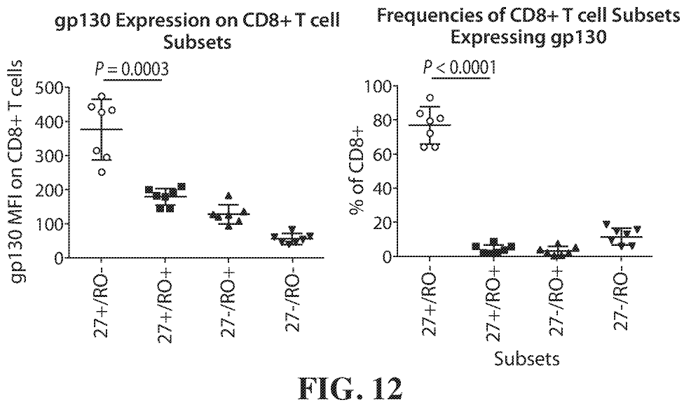

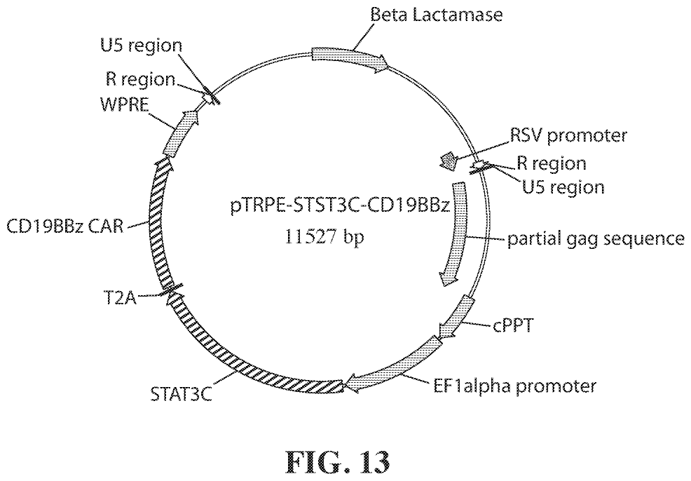

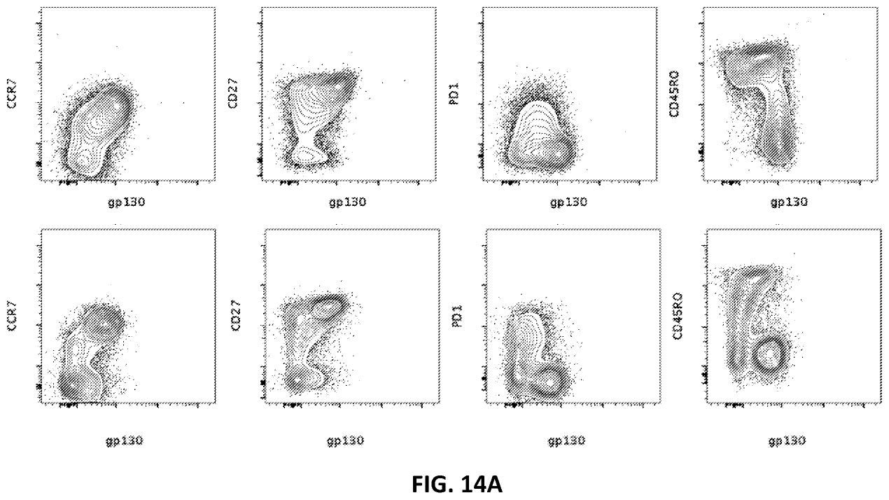

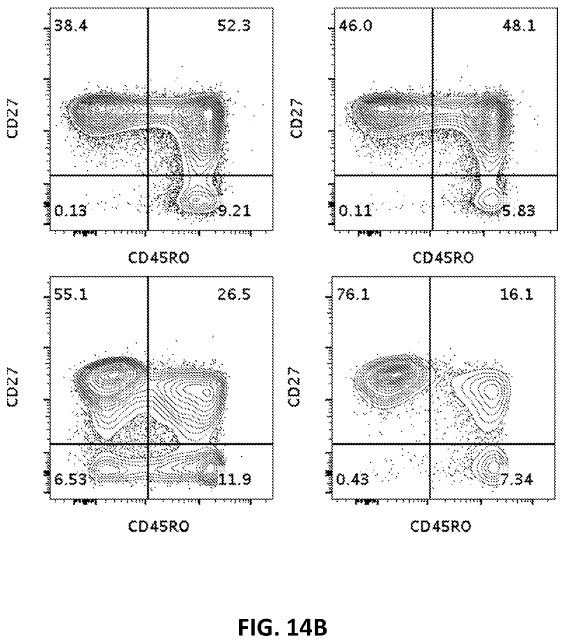

A highly significant association between the likelihood of having a response to CTL019 therapy and the infusion of CTL019 products containing a high dose of CD27+PD-1− CD8+ CAR T cells was observed, as shown in FIG. 10. The CD27+PD-1− CD8+ subset comprises the population that upregulates pSTAT3 in response to IL-6 stimulation (FIG. 11A), resulting from their markedly high levels of the IL-6 receptor-b chain (also known as CD130 or gp130) (FIG. 11B). FIG. 12 (left panel) shows that CD45RO−CD27+ CD8+ T cells express higher levels of gp130 compared to CD45RO+ CD27+ CD8+ T cells, CD45RO+ CD27− CD8+ T cells, or CD45RO− CD27− CD8+ T cells. FIG. 12 (right panel) demonstrates that a higher frequency of gp130 expressing cells is observed in the CD45RO−CD27+ CD8+ T cell population relative to other subsets. Furthermore, as shown in FIG. 14A, CD4+ T cells or CD8+ T cells with a PD-1 negative CD27^{hi} CCR7^{hi} CD45RO^{dim/neg} non-exhausted early memory T cell phenotype express, e.g., selectively express, gp130. Accordingly, a gp130 based positive selection of CD4+ T cells or CD8+ T cells, enriched for, e.g., selectively enriched for, early memory-phenotype T cells (FIG. 14B). The enriched T cells, e.g., early memory-phenotype T cells, can have the phenotype of CD27+ CD45RO^{dim/neg}. As shown in FIG. 14B, gp130 based enrichment in CD4+ T cells and CD8+ T cells resulted in an increase in the population of T cells with the phenotype of CD27+ and CD45RO^{dim/neg} compared to before enrichment. After enrichment with gp130, 46% of the CD4+ T cells were CD27+ CD45RO^{dim/neg}, compared to 38.4% before enrichment. After enrichment with gp130, 76.1% of the CD8+ T cells were CD27+ CD45RO^{dim/neg}, compared to 55.1% before enrichment.

Therefore, these findings suggest that the effectiveness of CAR T cell therapy for CLL may be increased, e.g., by adoptively transferring cultures containing lymphocytes with a higher absolute number of CD27+PD-1− CD8+ cells.

### EQUIVALENTS

The disclosures of each and every patent, patent application, and publication cited herein are hereby incorporated herein by reference in their entirety. While this invention has been disclosed with reference to specific aspects, it is apparent that other aspects and variations of this invention may be devised by others skilled in the art without departing from the true spirit and scope of the invention. The appended claims are intended to be construed to include all such aspects and equivalent variations.

What is claimed is:

1. A method of making a population of Chimeric Antigen Receptor (CAR)-expressing immune effector cells, comprising:
a) providing a population of immune effector cells, e.g., T cells;
b) contacting the population of immune effector cells with a nucleic acid encoding a CAR polypeptide;
c) contacting the population of immune effector cells with a Stat3 activator; and
d) maintaining the cells under conditions that allow expression of the CAR polypeptide,
thereby making a population of CAR-expressing immune effector cells.

2. The method of claim 1, wherein the Stat3 activator is chosen from, one, two, three, four, five, six, seven, eight, or all of, or any combination of:
i) a gp130 activator, e.g., an antibody molecule that binds to gp130, e.g., an anti-gp130 antibody as described herein, or an IL-6 molecule, an IL-11 molecule, an IL-27 molecule, a CNTF molecule, a CT-1 molecule, a CLC molecule, a LIF molecule, a NP molecule, an OSM molecule;
ii) a soluble IL-6 receptor (sIL-6R), e.g., as described herein;
iii) an IL-6/IL-6R complex, e.g., a dimer, e.g., as described herein;
iv) an IL-6 family cytokine (e.g., an IL-6 molecule, an IL-11 molecule, an IL-27 molecule, an IL-31 molecule, a CNTF molecule, a CT-1 molecule, a CLC molecule, a LIF molecule, a NP molecule or an OSM molecule);
v) a CCL20 molecule;
vi) an IL-10R2 receptor (IL-10R2) activator, e.g., an IL-10 molecule, an IL-22 molecule, an IL-26 molecule, an IL-28A molecule, an IL-28B molecule, an IL-29 molecule, or an antibody molecule that binds to IL-10R2, e.g., as described herein;
vii) an IL-10 family cytokine (e.g., an IL-10 molecule, an IL-19 molecule, an IL-20 molecule, an IL-22 molecule, an IL-24 molecule, an IL-26 molecule, an IL-28A molecule, an IL-28B molecule or an IL-29 molecule);
viii) an IL-17 family cytokine (e.g., an IL17A molecule, an IL17B molecule, an IL17C molecule, an IL17D molecule, an IL17E molecule or an IL17F molecule); or
ix) an IL-23 molecule.

3. The method of claim 1 or 2, wherein the method further comprises introducing into at least one cell of the population of immune effector cells:
a gp130 molecule, e.g., by introducing into the at least one cell of the population of immune effector cells a nucleic acid encoding the gp130 molecule under conditions that allow for translation of the gp130 molecule; or
a Stat3 molecule (e.g., a constitutively active Stat3 molecule (STAT3C)), e.g., by introducing into the at least one cell of the population of immune effector cells a nucleic acid encoding the Stat3 molecule under conditions that allow for translation of the Stat3 molecule.

4. A method of making a population of Chimeric Antigen Receptor (CAR)-expressing immune effector cells, comprising:
a) providing a population of immune effector cells, e.g., T cells;
b) contacting the population of immune effector cells with a nucleic acid encoding a CAR polypeptide;
c) introducing into at least one cell of the population of immune effector cells:
a gp130 molecule, e.g., by introducing into the at least one cell of the population of immune effector cells a nucleic acid encoding the gp130 molecule under conditions that allow for translation of the gp130 molecule; or
a Stat3 molecule (e.g., a constitutively active Stat3 molecule (STAT3C)), e.g., by introducing into the at least one cell of the population of immune effector cells a nucleic acid encoding the Stat3 molecule under conditions that allow for translation of the Stat3 molecule; and
d) maintaining the cells under conditions that allow expression of the CAR polypeptide, gp130 molecule or Stat3 molecule,
thereby making a population of CAR-expressing immune effector cells.

5. The method of claim 4, wherein the method further comprises contacting the population of immune effector cells with a Stat3 activator chosen from, one, two, three, four, five, six, seven, eight, or all of, or any combination of:
i) a gp130 activator, e.g., an antibody molecule that binds to gp130, e.g., an anti-gp130 antibody as described herein, or an IL-6 molecule, an IL-11 molecule, an IL-27 molecule, a CNTF molecule, a CT-1 molecule, a CLC molecule, a LIF molecule, a NP molecule, an OSM molecule;
ii) a soluble IL-6 receptor (sIL-6R), e.g., as described herein;
iii) an IL-6/IL-6R complex, e.g., a dimer, e.g., as described herein;
iv) an IL-6 family cytokine (e.g., an IL-6 molecule, an IL-11 molecule, an IL-27 molecule, an IL-31 molecule, a CNTF molecule, a CT-1 molecule, a CLC molecule, a LIF molecule, a NP molecule or an OSM molecule);
v) a CCL20 molecule;
vi) an IL-10R2 receptor (IL-10R2) activator, e.g., an IL-10 molecule, an IL-22 molecule, an IL-26 molecule, an IL-28A molecule, an IL-28B molecule, an IL-29 molecule, or an antibody molecule that binds to IL-10R2, e.g., as described herein;
vii) an IL-10 family cytokine (e.g., an IL-10 molecule, an IL-19 molecule, an IL-20 molecule, an IL-22 molecule, an IL-24 molecule, an IL-26 molecule, an IL-28A molecule, an IL-28B molecule or an IL-29 molecule);
viii) an IL-17 family cytokine (e.g., an IL17A molecule, an IL17B molecule, an IL17C molecule, an IL17D molecule, an IL17E molecule or an IL17F molecule); or
ix) an IL-23 molecule.

6. The method of any of claims 3-5, wherein expression of the gp130 molecule or the Stat3 molecule is transient (e.g., inducible or non-inducible) or constitutive.

7. The method of any of claims, 3-6, wherein the gp130 molecule or the Stat3 molecule is introduced into the population of immune effector cells, prior to, concurrently, or after contacting the population of immune effector cells with:
a nucleic acid encoding a CAR polypeptide; or
a Stat3 activator, e.g., as described herein.

8. The method of claim 3 or 4, wherein the nucleic acid comprising a nucleotide encoding a Stat3 molecule (e.g., a constitutively active Stat3 (STAT3C)), further comprises a nucleotide sequence encoding a CAR, e.g., a CD19 CAR.

9. The method of any of claim 1-3, or 5-8, wherein the Stat3 activator is an antibody molecule that binds to gp130, e.g., an anti-gp130 antibody as described herein.

10. The method of claim 9, which results in a population of T cells, e.g., CD4+ or CD8+ T cells, that is enriched for, e.g., early memory T cells or non-exhausted early memory T cells.

11. The method of claim 10, wherein early memory T cells have one or both of the following characteristics: CD27+ and/or CD45RO^{dim/neg}.

12. The method of claim 10, wherein non-exhausted early memory T cells have one or more, e.g., all, of the following characteristics: (i) PD-1 negative; (ii) CD27^{hi}; (iii) CCR7^{hi}; or (iv) CD45RO^{dim/neg}.

13. The method of any of claims 10-12, wherein the enriched population of T cells, e.g., early memory T cells or non-exhausted early memory T cells, e.g., has an increased level or amount of, e.g., at least 5%, e.g., 5-90% more (e.g., at least 5-10, 10-20, 20-30, 30-50, 50-70, or 70-90% more, e.g., at least 5, 6, 7, 8, 9, 10, 11, 12, 13, 14, 15, 16, 17, 18, 19, 20, 21, 22, 23, 24, 25, 26, 27, 28, 29 or 30% more), early memory T cells or non-exhausted early memory T cells compared to an otherwise similar population of T cells that was not contacted with the Stat3 activator.

14. The method of any of claim 1-3, or 5-8, wherein the Stat3 activator comprises one, two, three, or all of: an IL-6 molecule, an IL-17 molecule, an IL-22 molecule or a CCL20 molecule.

15. The method of any of claim 1-3, or 5-9, wherein the Stat3 activator is a naturally occurring molecule, a recombinant molecule, or a purified molecule.

16. The method of any of claim 1-3, or 5-10, wherein the Stat3 activator is not present in serum, e.g., not present in an amount sufficient to activate Stat3, e.g., phosphorylate Stat3, e.g., on tyrosine 705 (Y705), e.g., as measured by an assay of Example 2.

17. The method of any of claims 1-16, wherein the Stat3 activator is situated, e.g., immobilized, on a substrate, e.g., bead or cell.

18. The method of claim 17, wherein the Stat3 activator is situated on a Stat3 activator cell.

19. The method of claim 18, wherein the Stat3 activator cell is an artificial antigen-presenting cell.

20. The method of any of claims 17-19, wherein the Stat3 activator is expressed by the Stat3 activator cell or is conjugated to the surface of the Stat3 activator cell.

21. The method of any of claim 1-3, 5, or 9-20 wherein the Stat3 activator, e.g., as described herein, is provided in an amount sufficient to activate Stat3, e.g., phosphorylate Stat3, e.g., on tyrosine 705 (Y705), e.g., as measured by an assay of Example 2.

22. The method of any of claim 1-3, 5, or 9-21, wherein the Stat3 activator, e.g., as described herein, is provided in an amount sufficient to expand the population of immune effector cells, by at least 1.1, 1.2, 1.3, 1.4, 1.5, 2, 3, 4, 5, 6, 7, 8, 9 fold or more after a 12 day culture period, e.g., as measured by an assay of Example 2, compared to an otherwise similar population of cells cultured under similar conditions but not contacted with the Stat3 activator.

23. The method of any of claim 1-3, 5, or 9-22, wherein the Stat3 activator, e.g., as described herein, is provided in an amount sufficient to increase the percentage of cells in the immune effector cell population that are CD27+ PD-1−, e.g., by at least 1.1, 1.2, 1.3, 1.4, 1.5, 2, 3, 4, 5, fold or greater, compared to an otherwise similar population of cells cultured under similar conditions but not contacted with the Stat3 activator.

24. The method of any of claim 1-3, 5, or 9-23, wherein the Stat3 activator, e.g., as described herein, is provided in an amount sufficient to increase the expression level of gp130 by at least 1.5, 2, 3, 4, 5, 10 fold or more, in the immune effector cell population, e.g., as measured by an assay of Example 2, compared to an otherwise similar population of cells cultured under similar conditions but not contacted with the Stat3 activator.

25. The method of any of claim 1-3, 5 or 9-24, wherein the Stat3 activator, e.g., as described herein, is chosen from one, two, three, four, or all (e.g., five) of: an IL-6 molecule, an IL-17 molecule, an IL-22 molecule, an IL31 molecule, and a CCL20 molecule.

26. The method of any of claim 1-3, 5, or 9-25 wherein the Stat3 activator, e.g., as described herein, comprises an IL-6 molecule, e.g., recombinant IL-6.

27. The method of claim 21 wherein the IL-6 molecule, e.g., recombinant IL-6 is provided at an amount of at least 1, 5, 10, 15, 20, or 30 ng/ml, or in a range of 1-20, 1-15, or 5-15 ng/ml, e.g., at least 10 ng/ml.

28. The method of claim 2, 5, or 9-13, wherein the anti-gp130 antibody molecule is chosen from B-S12 or B-P8 or an antibody molecule having 1, 2, 3, 4, 5, or 6 CDRs from B-S12 or B-P8.

29. The method of claim 28 which comprises contacting the population of immune effector cells with both of B-S12 and B-P8.

30. The method of any of claim 28 or 29, wherein the total amount of anti-gp130 antibody molecule is about 0.1-1000, 0.5-500, or 1-100 ug/ml.

31. The method of any of claims 28-29, wherein the anti-gp130 antibody molecule is provided at an amount of at least 0.5, 0.6, 0.7, 0.8, 0.9, 1.0, 1.1, 1.2, 1.3, 1.4, 1.5, 1.6, 1.7, 1.8, 1.9, or 2 ug/ml, e.g., about 1 ug/ml.

32. The method of any of claims 28-31, wherein the anti-gp130 antibody:
induces gp130 mediated signaling, as measured by phosphorylation of STATS; or
induces dimerization, e.g., homodimerization of gp130, or heterodimerization of gp130, e.g., with LIF, OSM or CNTF.

33. The method of any of the preceding claims, wherein the population of cells cultured in the presence of the Stat3 activator, e.g., as described herein, exhibits:
activation of Stat3, e.g., phosphorylation of Stat3, e.g., on tyrosine 705 (Y705), e.g., as measured by an assay of Example 2;
expansion of the population of immune effector cells, by at least 1.1, 1.2, 1.3, 1.4, 1.5, 2, 3, 4, 5, 6, 7, 8, 9 fold or more after a 12 day culture period, e.g., as measured by an assay of Example 2;
increase in the percentage of cells in the immune effector cell population that are CD27+ PD-1−, e.g., by at least 1.1, 1.2, 1.3, 1.4, 1.5, 2, 3, 4, 5, fold or greater; and/or
increase in the expression level of gp130 by at least 1.5, 2, 3, 4, 5, or 10 fold or more, in the immune effector cell population, e.g., as measured by an assay of Example 2,
compared to an otherwise similar population of cells cultured under similar conditions but not contacted with the Stat3 activator.

34. The method of any of the preceding claims, comprising expanding the population, e.g., for at least 1, 2, 3, 4, 5, 6, 7, 8, 9, 10, 11, 12, 13, 14, 15, 16, 17, 18, 19, 20, or 21 days or for 1-7, 7-14, or 14-21 days.

35. The method of any of the preceding claims, further comprising assaying Stat3 pathway activation in the population of immune effector cells by measuring the level or activity of Stat3 transcriptional targets, e.g., c-Myc, c-Fos, Sox2, Bcl-2, or RORC to determine a value for Stat3 pathway activation.

36. The method of claim 35, further comprising comparing the Stat3 pathway activation value with a reference value, wherein the reference value is obtained from an otherwise similar population of immune effector cells cultured under similar conditions but not contacted with the Stat3 activator, e.g., as described herein.

37. The method of any of the preceding claims, further comprising, responsive to the comparison of the Stat3 pathway activation value with reference value, performing one or more of:
classifying the population as suitable or not suitable for use as a therapeutic;
formulating or packaging the population, or an aliquot thereof, for therapeutic use; or
altering a culture parameter, e.g., i) altering the length of time in culture or ii) increasing or decreasing the concentration of the Stat3 activator, e.g., as described herein.

38. A method of making a population of Chimeric Antigen Receptor (CAR)-expressing immune effector cells, comprising:
a) providing a population of immune effector cells, e.g., T cells;
b) contacting the population of immune effector cells with a nucleic acid encoding a CAR polypeptide;
c) contacting the population of immune effector cells with an inhibitor of glycolysis, e.g., a small molecule inhibitor of glycolysis, e.g., a small molecule hexokinase inhibitor, e.g., a glucose analog, e.g., 2-deoxy-D-glucose (2-DG), and
d) maintaining the cells under conditions that allow expression of the CAR polypeptide, thereby making a population of CAR-expressing immune effector cells.

39. The method of claim 38, wherein the inhibitor of glycolysis, e.g., a small molecule inhibitor of glycolysis, e.g., a small molecule hexokinase inhibitor, e.g., a glucose analog, e.g., 2-DG, is added in an amount sufficient to:
increase the population of immune effector cells at least 10%, 20%, 30%, 40%, 50%, 60%, 70%, 80%, 90%, 95%, 100% or greater; or
increase the percentage of cells in the immune effector cell population that have a central memory phenotype, e.g., are CD45RO+CCR7+, e.g., by about at least 10%, 20%, 30%, 40%, 50%, 60%, 70%, 80%, 90%, 95%, 100% or greater;
compared to an otherwise similar population of cells cultured under similar conditions but not treated with the inhibitor of glycolysis.

40. The method of claim 38 or 39, wherein the inhibitor of glycolysis, e.g., 2-DG, is added at a concentration of at least 0.5, 1, 1.5, 2, or 2.5 mM, 0.5-2.5 mM, or 1-2 mM.

41. The method of any of claims 38-40, wherein the population of cells cultured in the presence of the glycolysis inhibitor exhibits:
an increase the population of immune effector cells at least 10%, 20%, 30%, 40%, 50%, 60%, 70%, 80%, 90%, 95%, 100% or greater; or
an increase the percentage of cells in the immune effector cell population that have a central memory phenotype, e.g., are CD45RO+CCR7+, e.g., by about at least 10%, 20%, 30%, 40%, 50%, 60%, 70%, 80%, 90%, 95%, 100% or greater;
compared to an otherwise similar population of cells cultured under similar conditions but not treated with the inhibitor of glycolysis.

42. The method of any of claims 38-41, comprising:
expanding the population, e.g., for at least 1, 2, 3, 4, 5, 6, 7, 8, 9, 10, 11, 12, 13, 14, 15, 16, 17, 18, 19, 20, or 21 days or for 1-7, 7-14, or 14-21 days; or
expanding the population, e.g., by at least a 1.5, 2, 2.5, 3, 4, 5, 5, 7, 8, 9, 10, 20, 30, 40, 50-fold change in cell number or more, e.g., up to about 40 or 50-fold, e.g., under growth conditions of Example 1.

43. The method of any of claims 38-42, further comprising assaying glucose metabolism in the population of immune effector cells to determine a glucose metabolism value, e.g., using 2-NBDG uptake assay, e.g., an assay of Example 1.

44. The method of any of claims 38-43, further comprising comparing the glucose metabolism value with a reference value.

45. The method of any of claims 38-44, further comprising, responsive to the comparison of the glucose metabolism value with reference value, performing one or more of:
classifying the population as suitable or not suitable for use as a therapeutic;
formulating or packaging the population, or an aliquot thereof, for therapeutic use; or
altering a culture parameter, e.g., i) altering the length of time in culture or ii) increasing or decreasing the concentration of the inhibitor of glycolysis, e.g., the small molecule inhibitor of glycolysis, e.g., the small molecule hexokinase inhibitor, e.g., the glucose analog, e.g., 2-deoxy-D-glucose (2-DG).

46. The method of any of claims 38-45, further comprising contacting the population of immune effector cells with a Stat3 activator listed in claim 1, or a population of cells listed in claim 3 or 4.

47. The method of any of claims 1-37, further comprising contacting the population of immune effector cells with an inhibitor of glycolysis, e.g., the small molecule inhibitor of glycolysis, e.g., the small molecule hexokinase inhibitor, e.g., a glucose analog, e.g., 2-deoxy-D-glucose (2-DG).

48. The method of any of the preceding claims, wherein (b) is performed before (c), (c) is performed before (b), or (b) and (c) are performed simultaneously.

49. The method of any of the preceding claims, wherein the nucleic acid is DNA or RNA.

50. The method of any of the preceding claims, wherein (b) comprises performing lentiviral transduction to deliver the nucleic acid to the immune effector cells.

51. The method of any of the preceding claims, further comprising contacting the population of immune effector cells with a population of cells that expresses an antigen (e.g., CD19) that binds the CAR.

52. The method of any of the preceding claims, further comprising contacting the population of immune effector cells with an agent that stimulates a CD3/TCR complex associated signal and a ligand that stimulates a costimulatory molecule on the surface of the cells, e.g., wherein the agent is a bead conjugated with an anti-CD3 antibody, or a fragment thereof, and/or an anti-CD28 antibody, or a fragment thereof.

53. The method of any of the preceding claims, wherein the CAR polypeptide is a CD19 CAR, a CD22 CAR, a CD123 CAR, a BCMA CAR, an EGFRvIII CAR, a CLL-1 CAR, a CD20 CAR, or a CD33 CAR.

54. The method of any of the preceding claims, wherein the CAR is a CD19 CAR, e.g., a CAR comprising an scFv amino acid sequence of SEQ ID NO: 39-51 or a CAR comprising the amino acid sequence of SEQ ID NO: 77-89.

55. The method of any of the preceding claims, wherein the CAR comprises an antibody molecule which includes an anti-CD19 binding domain, a transmembrane domain, and an intracellular signaling domain comprising a stimulatory domain, and wherein said anti-CD19 binding domain comprises one or more of light chain complementary determining region 1 (LC CDR1), light chain complementary determining region 2 (LC CDR2), and light chain complementary determining region 3 (LC CDR3) of any anti-CD19 light chain binding domain amino acid sequence listed in Table 3B, and one or more of heavy chain complementary determining region 1 (HC CDR1), heavy chain complementary determining region 2 (HC CDR2), and heavy chain complementary determining region 3 (HC CDR3) of any anti-CD19 heavy chain binding domain amino acid sequence listed in Table 3A.

56. The method of claim 50, wherein the anti-CD19 binding domain comprises a sequence of SEQ ID NO: 40, or SEQ ID NO:51.

57. The method of any of claims 54-56, wherein the CAR comprises a polypeptide having a sequence of SEQ ID NO:78, or SEQ ID NO: 89.

58. A reaction mixture comprising:
a) (i) a population of CAR-expressing immune effector cells (e.g., a CAR-expressing cell described herein, e.g., a CD19 CAR-expressing cell) or (ii) an immune effector cell and a nucleic acid encoding a CAR (e.g., a CAR described herein, e.g., a CD19 CAR); and
b) an agent selected from:
(i) a Stat3 activator;
(ii) a cell or population of cells listed in claim 3 or 4; or
(iii) a gp130 molecule or a Stat3 molecule, or nucleic acid encoding a gp130 molecule or a Stat3 molecule.

59. The reaction mixture of claim 58, wherein the Stat3 activator is chosen from:
b-i-i) a gp130 activator, e.g., an antibody molecule that binds to gp130, e.g., an anti-gp130 antibody as described herein, or an IL-6 molecule, an IL-11 molecule, an IL-27 molecule, a CNTF molecule, a CT-1 molecule, a CLC molecule, a LIF molecule, a NP molecule, an OSM molecule;
b-i-ii) a soluble IL-6 receptor (sIL-6R), e.g., as described herein;
b-i-iii) an IL-6/IL-6R complex, e.g., a dimer, e.g., as described herein;
b-i-iv) an IL-6 family cytokine (e.g., an IL-6 molecule, an IL-11 molecule, an IL-27 molecule, an IL-31 molecule, a CNTF molecule, a CT-1 molecule, a CLC molecule, a LIF molecule, a NP molecule or an OSM molecule);
b-i-v) a CCL20 molecule;
b-i-vi) an IL-10R2 receptor (IL-10R2) activator, e.g., an IL-10 molecule, an IL-22 molecule, an IL-26 molecule, an IL-28A molecule, an IL-28B molecule, an IL-29 molecule, or an antibody molecule that binds to IL-10R2, e.g., as described herein;
b-i-vii) an IL-10 family cytokine (e.g., an IL-10 molecule, an IL-19 molecule, an IL-20 molecule, an IL-22 molecule, an IL-24 molecule, an IL-26 molecule, an IL-28A molecule, an IL-28B molecule or an IL-29 molecule);
b-i-viii) an IL-17 family cytokine (e.g., an IL17A molecule, an IL17B molecule, an IL17C molecule, an IL17D molecule, an IL17E molecule or an IL17F molecule); or
b-i-ix) an IL-23 molecule.

60. The reaction mixture of claim 58 or 59, which comprises (a)(i) a population of CAR-expressing immune effector cells.

61. The reaction mixture of claim 58 or 59, which comprises (a)(ii) a nucleic acid encoding a CAR (e.g., a CAR described herein, e.g., a CD19 CAR).

62. The reaction mixture of any of claims 58-61, which comprises (b)(i) a Stat3 activator listed in claim 59.

63. The reaction mixture of any of claims 58-61, which comprises (b)(ii) the cell or population of cells listed in claim 3 or 4.

64. The reaction mixture of any of claims 58-61, which comprises (b)(iii) a gp130 molecule, or a Stat3 molecule, or nucleic acid encoding same.

65. The reaction mixture of claim 58, which comprises: (a)(i) a population of CAR-expressing immune effector cells; and (b)(i) a Stat3 activator listed in claim 59.

66. The reaction mixture of claim 58, which comprises: (a)(i) a population of CAR-expressing immune effector cells; and (b)(ii) the cell or population of cells listed in claim 3 or 4.

67. The reaction mixture of claim 58, which comprises: (a)(i) a population of CAR-expressing immune effector cells; and(b)(iii) a gp130 molecule, or a Stat3 molecule, or nucleic acid encoding same.

68. The reaction mixture of claim 58, which comprises: (a)(ii) a nucleic acid encoding a CAR;
and (b)(i) a Stat3 activator listed in claim 59.

69. The reaction mixture of claim 58, which comprises: (a)(ii) a nucleic acid encoding a CAR;
and (b)(ii) the cell or population of cells of claim 3 or 4.

70. The reaction mixture of claim 53, which comprises: (a)(ii) a nucleic acid encoding a CAR;
and (b)(iii) a gp130 molecule, or a Stat3 molecule, or nucleic acid encoding a gp130 molecule or a Stat3 molecule.

71. The reaction mixture of any of claim 58-62, 65 or 68, which comprises one or more of: (a)(i) and b-i-i); (a)(i) and b-i-ii); (a)(i) and b-i-iii); (a)(i) and b-i-iv); (a)(i) and b-i-v); (a)(i) and b-i-vi); (a)(i) and b-i-vii); (a)(i) and b-i-viii); (a)(ii) and b-i-i); (a)(ii) and b-i-ii); (a)(ii) and b-i-iii); (a)(ii) and b-i-iv); (a)(ii) and b-i-v); (a)(ii) and b-i-vi); (a)(ii) and b-i-vii); and (a)(ii) and b-i-viii).

72. A reaction mixture comprising:
a) a population of CAR-expressing immune effector cells, e.g., a CAR-expressing cell described herein, e.g., a CD19 CAR-expressing cell, and
b) an inhibitor of glycolysis, e.g., a small molecule inhibitor of glycolysis, e.g., a small molecule hexokinase inhibitor, e.g., a glucose analog, e.g., 2-deoxy-D-glucose (2-DG).

73. A reaction mixture comprising:
a) a population of immune effector cells,
b) a nucleic acid encoding a CAR, e.g., a CAR described herein, e.g., a CD19 CAR, and
c) an inhibitor of glycolysis, e.g., a small molecule inhibitor of glycolysis, e.g., a small molecule hexokinase inhibitor, e.g., a glucose analog, e.g., 2-deoxy-D-glucose (2-DG).

74. The reaction mixture of claim 72 or 73, wherein the inhibitor of glycolysis, e.g., 2-DG, is present at a concentration of at least 0.5, 1, 1.5, 2, 2.5 mM, 0.5-2.5 mM, or 1-2 mM.

75. The reaction mixture of any of claims 72-74, further comprising a Stat3 activator;
a cell or population of cells; or a gp130 molecule, or a Stat3 molecule, or nucleic acid encoding a gp130 molecule or a Stat3 molecule, listed in claim 58 or 59.

76. The reaction mixture of any of claims 59-71, further comprising an inhibitor of glycolysis, e.g., a small molecule inhibitor of glycolysis, e.g., a small molecule hexokinase inhibitor, e.g., a glucose analog, e.g., 2-deoxy-D-glucose (2-DG).

77. The reaction mixture of any of claims 59-76, further comprising a lentivirus, e.g., wherein the nucleic acid encoding a CAR is packaged in a lentivirus.

78. The reaction mixture of any of claims 58-77, wherein the nucleic acid is DNA or RNA.

79. The reaction mixture of any of claims 58-78, further comprising a population of cells that expresses an antigen (e.g., CD19) that binds the CAR.

80. A method of evaluating or predicting the responsiveness of a subject having a cancer (e.g., a cancer described herein), to a therapeutic treatment with a CAR-expressing cell, e.g., prior to administration of the CAR-expressing cell, comprising evaluating in an immune effector cell from the subject:
i) a level of glucose metabolism, wherein:
a level of glucose metabolism that is lower than a glucose metabolism reference value is indicative that the subject is likely to respond to treatment with the CAR-expressing cell, e.g., to exhibit a complete response or a partial response, and
a level of glucose metabolism that is higher than a glucose metabolism reference value is indicative that the subject is less likely to respond to treatment with the CAR-expressing cell, e.g., does not exhibit a complete response or partial response; or
ii) a level of Stat3 activation as measured by, e.g., phosphorylation of Stat3 (e.g., on tyrosine 705 (Y705)) or level or activity of Stat3 transcriptional targets (e.g., c-Myc, c-Fos, Sox2 or Bcl-2), wherein:
a level of Stat3 activation that is higher than a Stat3 activation reference value is indicative that the subject is likely to respond to treatment with the CAR-expressing cell, e.g., to exhibit a complete response or a partial response, and
a level of Stat3 activation that is lower than a Stat3 activation reference value is indicative that the subject is less likely to respond to treatment with the CAR-expressing cell, e.g., does not exhibit a complete response or partial response,
thereby evaluating the subject, or predicting the responsiveness of the subject to the CAR-expressing cell.

81. The method of claim 80, wherein the immune effector cell has not been contacted with a nucleic acid encoding a CAR.

82. The method of claim 80, wherein the immune effector cell has been contacted with a nucleic acid encoding a CAR, e.g., expresses a CAR polypeptide.

83. The method of any of claims 80-82, wherein the immune effector cell has been contacted with:
i) a Stat3 activator listed in claim 1;
ii) a cell or population of cells listed in claim 3 or 4;
iii) a gp130 molecule, or a Stat3 molecule, or nucleic acid encoding a gp130 molecule or a Stat3 molecule; or
iv) an inhibitor of glycolysis, e.g., a small molecule inhibitor of glycolysis, e.g., a small molecule hexokinase inhibitor, e.g., a glucose analog, e.g., 2-deoxy-D-glucose (2-DG) at a concentration of at least 0.5, 1, 1.5, 2, or 2.5 mM.

84. The method of any of claims 80-83, wherein the method further comprises determining a fold change in cell number, e.g., number of CAR-expressing cells.

85. The method of any of claims 80-84, wherein the subject who is less likely to respond to treatment with the CAR-expressing cell is predicted, e.g., to not have a complete response (CR) or a partial response (PR), e.g., to be a non responder (NR).

86. The method of any of claims 80-85, wherein, responsive to determination that:
i) the level of glucose metabolism is lower than the glucose metabolism reference value; or
ii) the level of Stat3 activation is higher than the Stat3 activation reference value,
the subject is selected for administration of, or is administered, a CAR-expressing therapy.

87. The method of any of claims 80-85, wherein, responsive to determination that:
i) the level of glucose metabolism is higher than the glucose metabolism reference value; or
ii) the level of Stat3 activation is lower than the Stat3 activation reference value,
the subject is selected for administration of, or is administered, a therapy other than a CAR-expressing therapy.

88. The method of any of claims 80-87, wherein the glucose metabolism reference value is the glucose metabolism value of a cell of a complete responder subject as described in Example 1, e.g., wherein the cell (e.g., a sample containing the cell) is contacted with mock stimulation, e.g., stimulation with an antigen other than the CAR antigen, e.g., as described in Example 1.

89. The method of any of claims 80-87, wherein the Stat3 activation reference value is the Stat3 activation value of a cell of a non-responder subject, e.g., as described in Example 2.

90. A method of evaluating or predicting the responsiveness of a subject having a cancer (e.g., a cancer described herein), wherein the subject has been treated with a CAR-expressing cell, comprising evaluating in a CAR-expressing cell from the subject:
i) a level of glucose metabolism, wherein:
a level of glucose metabolism that is lower than a glucose metabolism reference value is indicative that the subject is likely to respond to treatment with the CAR-expressing cell, e.g., to exhibit a complete response or a partial response, and
a level of glucose metabolism that is higher than a glucose metabolism reference value is indicative that the subject is less likely to respond to treatment with the CAR-expressing cell, e.g., does not exhibit a complete response or partial response; or
ii) a level of Stat3 activation as measured by, e.g., phosphorylation of Stat3 (e.g., on tyrosine 705 (Y705)) or level or activity of Stat3 transcriptional targets (e.g., c-Myc, c-Fos, Sox2 or Bcl-2), wherein:
a level of Stat3 activation that is higher than a Stat3 activation reference value is indicative that the subject is likely to respond to treatment with the CAR-expressing cell, e.g., to exhibit a complete response or a partial response, and
a level of Stat3 activation that is lower than a Stat3 activation reference value is indicative that the subject is less likely to respond to treatment with the CAR-expressing cell, e.g., does not exhibit a complete response or partial response,
thereby evaluating the subject, or predicting the responsiveness of the subject to the CAR-expressing cell.

91. The method of claim 90, further comprising obtaining the CAR-expressing cell from the subject prior to evaluating the level of glucose metabolism, or the level of Stat3 activation in the CAR-expressing cell.

92. The method of claim 90 or 91, wherein the subject who is less likely to respond to treatment with the CAR-expressing cell is predicted e.g., to not have a complete response (CR) or a partial response (PR), e.g., to be a non-responder (NR).

93. The method of any of claims 90-92, wherein, responsive to determination that:
i) the level of glucose metabolism is lower than the glucose metabolism reference value; or
ii) the level of Stat3 activation is higher than the Stat3 activation reference value,
the subject is selected for administration of, or is administered, one or more additional doses of the CAR-expressing therapy.

94. The method of any of claims 90-93, wherein, responsive to determination that:
i) the level of glucose metabolism is higher than the glucose metabolism reference value; or
ii) the level of Stat3 activation is lower than the Stat3 activation reference value,
the subject is selected for administration of, or is administered, a therapy other than a CAR-expressing therapy.

95. The method of any of claims 90-94, wherein the glucose metabolism reference value is the glucose metabolism value of a cell of a complete responder subject as described in Example 1, e.g., wherein the cell (e.g., a sample containing the cell) is contacted with mock stimulation, e.g., stimulation with an antigen other than the CAR antigen, e.g., as described in Example 1.

96. The method of any of claims 90-95, wherein the Stat3 activation reference value is the Stat3 activation value of a cell of a non-responder subject, e.g., as described in Example 2.

97. A method of evaluating a CAR-expressing cell, e.g., CAR19− expressing cell, (e.g., CTL019), said method comprising evaluating in the CAR-expressing cell in a sample from a subject:
i) a level of glucose metabolism, wherein:
a level of glucose metabolism that is lower than a glucose metabolism reference value is indicative that the sample is suitable for treatment, and
a level of glucose metabolism that is higher than a glucose metabolism reference value is indicative that the sample is less suitable for treatment; or
ii) a level of Stat3 activation, wherein:
a level of Stat3 activation that is higher than a Stat3 activation reference value is indicative that the sample is suitable for treatment, and
a level of Stat3 activation that is lower than a Stat3 activation reference value is indicative that the sample is less suitable for treatment,
thereby evaluating the CAR-expressing cell.

98. The method of claim 97, further comprising obtaining the CAR-expressing cell from the subject prior to evaluating the level of glucose metabolism or Stat3 activation in the CAR-expressing cell.

99. The method of claim 97 or 98, wherein, responsive to determination that:
i) the level of glucose metabolism is lower than the glucose metabolism reference value; or
ii) the level of Stat3 activation is higher than the Stat3 activation reference value,
the sample is selected for administration, or is administered, to the subject.

100. The method of claim 97 or 98, wherein, responsive to determination that:
i) the level of glucose metabolism is higher than the glucose metabolism reference value,
ii) the level of Stat3 activation is lower than the Stat3 activation reference value,
the sample is not selected for administration, or is not administered, to the subject.

101. The method of any of claims 97-100, wherein the glucose metabolism reference value is the glucose metabolism value of a cell of a complete responder subject as described in Example 1, e.g., wherein the cell (e.g., a sample containing the cell) is contacted with mock stimulation, e.g., stimulation with an antigen other than the CAR antigen, e.g., as described in Example 1.

102. The method of any of claims 97-100, wherein the Stat3 activation reference value is the Stat3 activation value of a cell of a non-responder subject, e.g., as described in Example 2.
